(19) United States
(12) Patent Application Publication
(10) Pub. No.: US 2011/0230477 A1
(43) Pub. Date: Sep. 22, 2011

(54) COMPOUNDS, PHARMACEUTICAL COMPOSITION AND METHODS FOR USE IN TREATING METABOLIC DISORDERS

(75) Inventors: Hamid Hoveyda, Bruxelles (BE); Cyrille Evangelos Brantis, Mons (BE); Guillaume Dutheuil, Flenu (BE); Ludivine Zoute, Flenu (BE); Didier Schils, Loupoigne (BE); Jerome Bernard, Valenciennes (FR)

(73) Assignee: Euroscreen S.A., Brussels (BE)

(21) Appl. No.: 13/130,567

(22) PCT Filed: Dec. 7, 2009
(86) PCT No.: PCT/EP2009/066536

§ 371 Date: May 20, 2011

Related U.S. Application Data
(60) Provisional application No. 61/205,735, filed on Jan. 23, 2009.
(60) Provisional application No. 61/152,294, filed on Feb. 13, 2009.
(60) Provisional application No. 61/168,159, filed on Apr. 9, 2009.

(30) Foreign Application Priority Data
Dec. 8, 2008 (EP) 08305896.6

(51) Int. Cl. A61K 31/5383; C07D 277/46; A61K 31/426; C07D 417/12; A61K 31/5377; C07D 285/08; C07D 271/07; A61K 31/4245; A61K 31/433; A61K 31/427; A61K 31/4439; C07D 417/14; C07D 498/04; C07D 471/04; A61K 31/4375; A61P 3/10; A61P 3/06; A61P 3/04; A61P 3/00; A61P 9/12; A61P 7/00; A61P 9/00; A61P 9/10; A61P 13/12; A61P 1/16

(52) U.S. Cl. 514/230.5; 548/195; 514/371; 544/133; 514/236.8; 548/128; 548/133; 514/364; 514/361; 548/132; 546/270.7; 514/342; 544/131; 544/105; 546/122; 514/300

(57) ABSTRACT

The present invention is directed to novel compounds of formula (I) and their use in treating metabolic diseases.

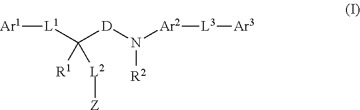

The present invention relates to novel compounds including their pharmaceutically acceptable salts and solvates, which are agonists or partial agonists of G-protein coupled receptor 43 (GPR43) and are useful as therapeutic compounds, particularly in the treatment and/or prevention of Type 2 diabetes mellitus and conditions that are often associated with this disease including, lipid disorders such as dyslipidemia, hypertension, obesity, atherosclerosis and its sequelae.

### BACKGROUND OF THE INVENTION

Under normal conditions, Free Fatty Acids (FFAs) are implicated in numerous physiological processes by serving as fuel in various metabolic pathways and/or acting as signaling molecules in different tissues such as the heart, liver, skeletal muscle, adipocytes and the pancreas (Newsholme et al., Biochem. J., 80 pp 655-662, 1961; Prentki et al., Endocrine Reviews, PubMed print ahead, 2008). Among FFAs, the short-chain fatty acids (SCFAs, carbon length C2-C6) are generated during anaerobic bacterial fermentation of fiber in the gut (Sellin et al., News. Physiol. Sci., 14, pp 58-64, 1999). Long-chain fatty acids (LCFAs, carbon length C₁₄-C₂₄) are products of dietary intake from adipose tissues and liver (McArthur et al., J. Lipid. Res., 40, pp 1371-1383, 1999).

Obesity is an increasing, worldwide public health problem associated with devastating pathologies such as type 2 diabetes (T2D) and dyslipidemia (Wild et al., Diabetes Care 27, pp 1047-1053, 2004). Dyslipidemia is characterized by high levels of triglycerides and/or LDL (bad cholesterol) or low levels of HDL (good cholesterol). Dyslipidemia is a key independent risk factor for cardiovascular diseases. It has long been suggested that FFAs are implicated in the regulation and/or genesis of these diseases (Fraze et al., J. Clin. Endocrinol. Metab., 61, pp 807-811, 1985). It is well established that regular intake of dietary fiber has several beneficial metabolic effects such as lowering of plasma cholesterol and triglyceride levels (Anderson et al., J. Am. Coll. Nutr., 23, pp 5-17, 2004). Specifically, dietary fiber has been shown to increase endogenous levels of SCFAs, leading to the suppression of cholesterol synthesis and improvement in glucose tolerance in rat (Berggren et al., Br. J. Nutr., 76, pp 287-294, 1996), as well as the reduction of hyperglycemia in a diabetic mice model (Sakakibara et al., Biochem. Biophys. Res. Com., 344, pp 597-604, 2006).

Drug therapies are available to address both T2D and dyslipidemia. Specifically, statins, fibrates and nicotinic acid or combinations thereof are often considered as a first line therapy in dyslipidemia whereas metformin, sulphonylureas and thiazolidinediones are three, widely-used classes of oral anti-diabetic drugs (Tenenbaum et al., Cardiovascular Diabetology, 5, pp 20-23, 2006). Although theses therapies are widespread in their use, the common appearance of adverse effects or lack of efficacy after long-term use causes concern. Moreover, the growing patient population suffering from T2D, dyslipidemia and associated metabolic diseases creates a demand for new entrants into this therapeutic market.

GPR43 (also named FFA2R) belongs to a subfamily of G-Protein-Coupled Receptors (GPCRs), including GPR40 and GPR41 that have been identified as receptor for FFAs (Le Poul et al., J. Biol Chem. 278, 25481-489, 2003; Covington et al., Biochemical Society transaction 34, 770-773, 2006). The 3 family members share 30 to 40% sequences identity with specificity toward different fatty acids carbon chain lengths, with SCFAs (short chain fatty acids: six carbons molecules or shorter) activating GPR41 and GPR43 and medium and long chain fatty acids (MCFA, LCFA) activating GPR40 (Rayasam et al., Expert Opinion on therapeutic targets, 11 661-671, 2007). C2 acetate and C3 propionate are the most potent activators of GPR43. GPR43 is mainly coupled with Gq-proteins, with some evidence for its possible coupling with Gi/o pathways as well.

GPR43 is strongly expressed in adipocytes. Also there is evidence suggesting that GPR43 is overexpressed in pancreatic β-cells in prediabetic states as shown in WO2006/036688A2. Recent papers confirmed the GPR43 expression in pancreatic islets (Ahrén, Nature Reviews, 8 pp 396-385; 2009; Regard et al., J; Clin. Invst., 117 pp 4034-4043, 2007). In adipocyte cells, GPR43 is induced during the differentiation process and increased during the high fat feeding in rodents, suggesting that GPR43 may affect adipocyte functions (Hong et al., Endrocrinology, 146 pp 5092-5099, 2005). Indeed, it has been reported that acetate and propionate may stimulate adipogenesis via GPR43. In addition siRNA results hinted that acetate and propionate may inhibit lipolysis in adipocytes via GPR43 activation (Hong et al., Endocrinology, 146 pp 5092-5099, 2005). It is interesting to note that the effect of acetate on reducing plasma free fatty acids level has been documented in humans (Suokas et al., Alcoholism, clinical and experimental research, 12 pp 52-58, 1988; Laurent et al., European journal of clinical nutrition, 49 pp 484-491, 1995). In addition, it has been shown that (i) adipocytes treated with GPR43 endogenous SCFA ligands exhibit a reduction in lipolytic activity and such inhibition of lypolysis is the result of GPR43 activation and (ii) GPR43 activation by acetate results in the reduction of plasma free fatty acids level in vivo (Ge et al., Endocrinology, 149 pp 4519-26, 2008). Recently two GPR43 positive allosteric modulator molecules have been shown able to inhibit the lipolysis in adipocytes similarly to that of GPR43 endogenous SCFA ligands (Lee et al., Mol Pharmacol, 74(6) pp 1599-1609, 2008). Such results suggest a potential role of GPR43 in regulating plasma lipid profiles and aspects of metabolic syndrome.

On this basis, new agonists or partial agonists of GPR43 may be of therapeutic value for T2D mellitus and conditions that are associated with this disease including, lipid disorders such as dyslipidemia, hypertension, obesity, atherosclerosis and its sequelae.

### SUMMARY OF THE INVENTION

The invention encompasses compounds of general Formula I, their pharmaceutically acceptable salts and solvates as well as methods of use of such compounds or compositions comprising such compounds as modulators of GPR43 activity.

In a general aspect, the invention provides compounds of general formula I:

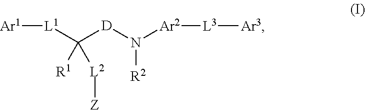

wherein
Ar¹ is a 5- to 6-membered aryl or heteroaryl group, 3- to 8-membered cycloalkyl group, a 3- to 8-membered heterocycloalkyl group, or a linear or branched C₃-C₆ alkyl group, each of the aryl, heteroaryl, cycloalkyl, heterocycloalkyl, or alkyl groups being optionally substituted by one or more groups selected from halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, amino, alkoxyalkoxy, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoyl alkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or two substituents form an alkylenedioxy group or a haloalkylenedioxy group, or two substituents form a cycloalkyl or heterocycloalkyl moiety together with the cycloalkyl or heterocycloalkyl group they are attached to, or fused to the aryl, heteroaryl, cycloalkyl or heterocycloalkyl group may be one or more cycloalkyl, aryl, heterocyclyl or heteroaryl moiety, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl;
L¹ is a single bond, C₁-C₂ alkylene, C₁-C₂ alkenylene, each optionally being substituted by one or more substituents selected from halo, C₁-C₂ alkyl, C₁-C₂ haloalkyl; or L¹ is —N(R^{N})—, wherein R^{N} is H or C₁-C₂ alkyl; or L¹ and R¹ together are ═CH—;
R¹ is H, halo, allyl, or a C₁-C₄ alkyl group, which may optionally be substituted by one or more groups selected from halo or C₁-C₄ alkyl;
L² is a C₁-C₃ alkylene, C₂-C₄ alkenylene, C₃-C₆ cylcloalkylene, each of which being optionally substituted by one or more groups selected from halo, alkyl, alkoxy, or haloalkyl; or L² is —O—CH₂—; or
R¹ and L² together are ═CH—, under the condition that -L¹-Ar¹ is H; or
R¹ and L² together are a 5- to 6-membered saturated or unsaturated carbocyclic or heterocyclic group, preferably a cyclohexenyl group, under the condition that -L¹-Ar¹ is H;
Z is selected from the group consisting of —COOR,

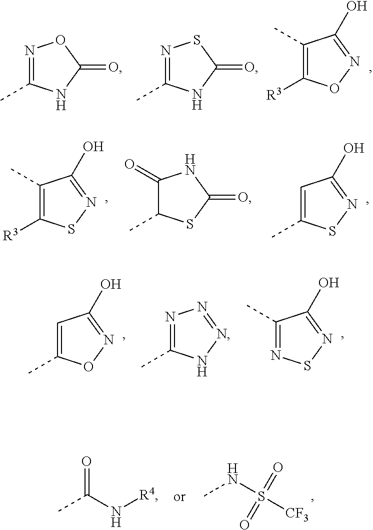

wherein R is H or linear or branched alkyl, aryl, acyloxyalkyl, dioxolene, R³ is H, methyl or ethyl, and R⁴ is hydroxyl —SO₂CH₃, —SO₂cyclopropyl or —SO₂CF₃;
D is CO or SO₂;
R² is H, linear or branched C₁-C₄ alkyl, C₁-C₄ hydroxyalkyl, C₁-C₄ haloalkyl, C₂-C₄ alkenyl, C₂-C₄ alkynyl, C₃-C₆ cycloalkyl, C₃-C₆ cycloalkylalkyl, aryl, arylalkyl, heteroarylalkyl, alkoxycarbonylalkyl, aminocarbonylalkyl, or aralkyloxyalkyl; each of the alkyl, hydroxyalkyl, alkenyl, alkynyl, cycloalkyl, cycloalkylalkyl, aryl, arylalkyl, heteroarylalkyl, alkoxycarbonylalkyl, aminocarbonylalkyl, and aralkyloxyalkyl groups being optionally substituted by one or more substituents selected from halo, cyano, alkyl, hydroxyalkyl, haloalkyl, alkenyl, alkynyl, heteroalkyl, hydroxyl, alkoxy, haloalkoxy, cycloalkyloxy, amino, alkylamino, aminoalkyl, carboxy, alkoxycarbonyl, alkylcarbonyloxy, alkylcarbonylamino, haloalkylcarbonylamino, alkylcarbonylaminoalkyl, acylamino, carbamoyl, hydroxycarbamoyl, alkylcarbamoyl, carbamoylalkyl, carbamoyl amino, alkylcarbamoylamino, alkylsulfonyl, haloalkylsulfonyl, sulfamoyl, alkylsulfamoyl, alkylsulfonylamino, cycloalkylsulfonylamino, haloalkylsulfonylamino, or two substituents form an alkylenedioxy group or a haloalkylenedioxy group;
Ar² is a 5- or 6-membered heterocyclic group or a 5- or 6-membered heteroaryl group, optionally substituted by one or more substituents selected from halo, cyano, alkyl, hydroxyalkyl, haloalkyl, alkenyl, alkynyl, heteroalkyl, hydroxyl, alkoxy, haloalkoxy, cycloalkyloxy, heterocyclyloxy, aryloxy, amino, alkylamino, aminoalkyl, carboxy, alkoxycarbonyl, alkylcarbonyloxy, alkylcarbonylamino, haloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, alkylsulfonyl, haloalkylsulfonyl, sulfamoyl, alkylsulfamoyl, alkylsulfonylamino, cycloalkylsulfonylamino, haloalkylsulfonylamino, or two substituents form an alkylenedioxy group or a haloalkylenedioxy group;
L³ is a single bond, C₁-C₃ alkylene, C₁-C₃ cycloalkylene C₁-C₃ alkenylene or carbonylamino;
Ar³ is an aryl, heteroaryl, or C₁-C₄ alkyl group, each of which being optionally substituted by one or more groups selected from halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, cycloalkylaminocarbamoyl, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or two substituents form an alkylenedioxy group or a haloalkylenedioxy group, or two substituents form a cycloalkyl or heterocycloalkyl moiety together with the cycloalkyl or heterocycloalkyl group they are attached to, or fused to the aryl, heteroaryl, cycloalkyl or heterocycloalkyl group may be one or more cycloalkyl, aryl, heterocyclyl or heteroaryl moiety, each of said substituents being optionally substituted by one or more further substituents selected from halo, alkoxy, alkyl, alkoxyalkyl, alkoxyalkoxy, cycloalkylalkyloxy, amino, alkylamino, alkylaminoalkoxy, cycloalkylamino, aralkylamino, alkylaminoalkyl, alkylaminocarbonyl, alkylcarbonyl, cycloalkylcarbonylamino, alkylheterocyclyl, alkylheteroaryl, alkylsulfonyl, alkylsulfonylamino, aralkyl, aralkyloxy, aryl, arylamino, aryloxy, cyano, haloalkoxy, haloalkyl, heteroaryl, heteroarylalkyl, heteroarylcarbonyl, heterocyclyl, heterocyclyloxy, hydroxyl, oxo, or sulfonyl, or L³-Ar³ form an aryl, preferably phenyl, or heteroaryl group fused to Ar², wherein each of said aryl or heteroaryl groups fused to Ar² are optionally substituted by one or more halo, preferably chloro and fluoro;
with the following provisos:
Ar²-L³-Ar³ is not 4-(4-butylphenyl)thiazol-2-yl, 4-(4-ethylphenyl)thiazol-2-yl, 4-(para-tolyl)thiazol-2-yl, 4-phenylthiazol-2-yl, 4-(4-propylphenyl)thiazol-2-yl, 4-(4-(sec-butyl)phenyl)thiazol-2-yl, 4-(4-isopropylphenyl)thiazol-2-yl, 4-(4-isobutylphenyl)thiazol-2-yl, 4-(4-(tert-butyl)phenyl)thiazol-2-yl, 4-(4-butylphenyl)-5-methylthiazol-2-yl, 4-(4-ethylphenyl)-5-methylthiazol-2-yl, 5-methyl-4-(para-tolyl)thiazol-2-yl, 5-methyl-4-phenylthiazol-2-yl, 5-methyl-4-(4-propylphenyl)thiazol-2-yl, 4-(4-(sec-butyl)phenyl)-5-methylthiazol-2-yl, 4-(4-isopropylphenyl)-5-methylthiazol-2-yl, 4-(4-isobutylphenyl)-5-methylthiazol-2-yl, 4-(4-(tert-butyl)phenyl)-5-methylthiazol-2-yl, 4-(4-butyl-3-methylphenyl)thiazol-2-yl, 4-(4-ethyl-3-methylphenyl)thiazol-2-yl, 4-(3,4-dimethylphenyl)thiazol-2-yl, 4-(meta-tolyl)thiazol-2-yl, 4-(3-methyl-4-propylphenyl)thiazol-2-yl, 4-(4-(sec-butyl)-3-methylphenyl)thiazol-2-yl, 4-(4-isopropyl-3-methylphenyl)thiazol-2-yl, 4-(4-isobutyl-3-methylphenyl)thiazol-2-yl, 4-(4-(tert-butyl)-3-methylphenyl)thiazol-2-yl, 4-(4-butyl-3-methylphenyl)-5-methylthiazol-2-yl, 4-(4-ethyl-3-methylphenyl)-5-methylthiazol-2-yl, 4-(3,4-dimethylphenyl)-5-methylthiazol-2-yl, 5-methyl-4-(meta-tolyl)thiazol-2-yl, 5-methyl-4-(3-methyl-4-propylphenyl)thiazol-2-yl, 4-(4-(sec-butyl)-3-methylphenyl)-5-methylthiazol-2-yl, 4-(4-isopropyl-3-methylphenyl)-5-methylthiazol-2-yl, 4-(4-isobutyl-3-methylphenyl)-5-methylthiazol-2-yl, 4-(4-(tert-butyl)-3-methylphenyl)-5-methylthiazol-2-yl;
Ar³ is not (7H-pyrrolo[2,3-d]pyrimidin)-4yl;
Ar² is not 5-cyano-thiazolyl;
the compound of formula I is none of.
2-[[[4-(4-butylphenyl)-5-methyl-2-thiazolyl]amino]carbonyl]-cyclohexane carboxylic acid, 6-[[(4,5-dimethyl-2-thiazolyl)amino]carbonyl]-3-cyclohexene-1-carboxylic acid, 6-[[[5-(cyclopentylmethyl)-1,3,4-thiadiazol-2-yl]amino]carbonyl]-3-cyclohexene-1-carboxylic acid, 3-cyclohexene-1-carboxylic acid, 6-[[(5-acetyl-4-methyl-2-thiazolyl)amino]carbonyl]-2-[[[4-(4-methoxyphenyl)-5-methyl-2-thiazolyl]amino]carbonyl]-cyclohexanecarboxylic acid, 6-[[[4-(3,4-dimethylphenyl)-5-methyl-2-thiazolyl]amino]carbonyl]-3-cyclohexene-1-carboxylic acid, 6-[[[5-methyl-4-(4-propylphenyl)-2thiazolyl]amino]carbonyl]-3-cyclohexene-1-carboxylic acid, 2-[[[4-(2,4-dichlorophenyl)-5-methyl-2-thiazolyl]amino]carbonyl]-cyclohexanecarboxylic acid, 2-[[[4-(2,5-dimethylphenyl)-5-methyl-2-thiazolyl]amino]carbonyl]-cyclohexanecarboxylic acid, 6-[[[5-(2-chlorophenyl)-1,3,4-thiadiazol-2-yl]amino]carbonyl]-3-cyclohexene-1-carboxylic acid 2-[[[5-[(4-chlorophenoxy)methyl]-1,3,4-thiadiazol-2-yl]amino]carbonyl]-cyclohexanecarboxylic acid, 2-[[[5-methyl-4-(4-propylphenyl)-2-thiazolyl]amino]carbonyl]-cyclohexanecarboxybic acid, 2-[[(5-methyl-1,3,4-thiadiazol-2-yl)amino]carbonyl]-cyclohexanecarboxylic acid, 6-[[[4-[4-(1,1-dimethylethyl)phenyl]-5-methyl-2-thiazolyl]amino]carbonyl]-3-cyclohexene-1-carboxylic acid, 6-[[(5-ethyl-1,3,4-thiadiazol-2-yl)amino]carbonyl]-3-cyclohexene-1-carboxylic acid-1-methylethyl ester 2-[[(5-methyl-4-phenyl-2-thiazolyl)amino]carbonyl]-cyclohexanecarboxylic acid, 2-[[[5-methyl-4-[4-(2-methylpropyl)phenyl]-2-thiazolyl)amino]carbonyl]-cyclohexanecarboxylic acid, 6-[[(5-cyclopropyl-1,3,4-thiadiazol-2-yl)amino]carbonyl]-3-cyclohexene-1-carboxylic acid, 2-[[[5-(cyclopentylmethyl)-1,3,4-thiadiazol-2-yl]amino]carbonyl]-cyclohexanecarboxylic acid, 2-[[[4-(4-chlorophenyl)5-ethyl-2-thiazolyl)amino]carbonyl]-cyclohexanecarboxylic acid, 2-[[[4-(3-methoxyphenyl)-5-methyl-2-thiazolyl]amino]carbonyl]-cyclohexanecarboxylic acid, 6-[[[5-methyl-4-(4-methylphenyl)-2-thiazolyl]amino]carbonyl]-3-cyclohexene-1-carboxylic acid, 2-[[(5-cyclopropyl-1,3,4-thiadiazol-2-yl)amino]carbonyl]-cyclohexanecarboxylic acid, 6-[[[4-(4-chlorophenyl)-5-ethyl-2-thiazolyl]amino]carbonyl]-3-cyclohexene-1-carboxybic acid, 6-[[[4-(2,5-dimethylphenyl)-5-methyl-2-thiazolyl]amino]carbonyl]-3-cyclohexene-1-carboxylic acid, 6-[[(5-phenyl-1,3,4-thiadiazol-2-yl)amino]carbonyl]-3-cyclohexene-1-carboxylic acid, 2-[[[5-(4-methoxyphenyl)-1,3,4-thiadiazol-2yl]amino]carbonyl]-cyclohexanecarboxylic acid, 2-[[(6-carboxy-3-cyclohexen-1-yl)carbonyl]amino]-4-phenyl-5-thiazolecarboxylic acid-5-ethyl ester, 2-[[(4,5-dimethyl-2-thiazolyl)amino]carbonyl]-cyclohexanecarboxylic acid, 6-[[(5-cyclopropyl-1,3,4-oxadiazol-2-yl)amino]carbonyl]-3-cyclohexene-1-carboxylic acid, 6-[[[5-methyl-4-[4-(2-methylpropyl)phenyl]-2-thiazolyl)amino]carbonyl]-3-cyclohexene-1-carboxylic acid, 6-[[(5-ethyl-4-phenyl-2-thiazolyl)amino]carbonyl]-3-cyclohexene-1-carboxylic acid, 6-[[[4-(2,4-dimethylphenyl)-5-methyl-2-thiazolyl)amino]carbonyl]-3-cyclohexene-1-carboxylic acid, 2-[[[4-(3-chlorophenyl)-5-methyl-2-thiazolyl]amino]carbonyl]-cyclohexanecarboxylic acid, 6-[[[5-(1-ethylphenyl)-1,3,4-thiadiazol-2-yl]amino]carbonyl]-3-cyclohexene-1-carboxylic acid, 2-[[[4-(3,4-dimethylpentyl)-5-methyl-2-thiazolyl]amino]carbonyl]-cyclohexanecarboxylic acid, 2-[[[5-(2-thienyl)-1,3,4-thiadiazol-2-yl]amino]carbonyl]-cyclohexanecarboxylic acid, 2-[[(4,5-diphenyl-2-thiazolyl)amino]carbonyl]-cyclohexanecarboxybic acid, 6-[[[4-(4-ethylphenyl)-5-methyl-2-thiazolyl]amino]carbonyl]-3-cyclohexene-1-carboxylic acid, 2-[[(2-carboxycyclohexyl)carbonyl]amino]-4-methyl-5-thiazolecarboxylic acid-5-methyl ester, 2-[[(2-carboxycyclohexyl)carbonyl]amino]-4-methyl-5-thiazolecarboxylic acid-5-ethyl ester, 2-[[(5-ethyl-4-phenyl-2-thiazolyl)amino]carbonyl]-cyclohexanecarboxylic acid, 6-[[(5-methyl-1,3,4-thiadiazol-2-yl)amino]carbonyl]-3-cyclohexene-1-carboxylic acid, 2-[[(5-cyclopropyl-1,3,4-oxadiazol-2-yl)amino]carbonyl]-cyclohexanecarboxybic acid, 2-[[[4-(4-fluorophenyl)-5-methyl-2-thiazolyl]amino]carbonyl]-cyclohexanecarboxylic acid, 2-[[(2-carboxycyclohexyl)carbonyl]amino]-4-methyl-5-thiazoleacetic acid-5-ethyl ester, 2-[[[4-(2,4-dimethylphenyl)-5-methyl-2-thiazolyl]amino]carbonyl]-cyclohexanecarboxylic acid, 6-[[[4-(3-chlorophenyl)-5-methyl-2-thiazolyl]amino]carbonyl]-3-cyclohexene-1-carboxylic acid, 2-((5-cyclohexyl-1,3,4-thiadiazol-2-yl)carbamoyl)cyclohexanecarboxylic acid 2-[[[5-methyl-4-(4-methylphenyl)-2-thiazolyl]amino]carbonyl]-cyclohexanecarboxylic acid, 6-[[[5-(2-thienyl)-1,3,4-thiadiazol-2-yl]amino]carbonyl]-3-cyclohexene-1-carboxylic acid, 6-[[(4,5-diphenyl-2-thiazolyl)amino]carbonyl]-3-cyclohexene-1-carboxylic acid, 2-[[[4-(4-ethylphenyl)-5-methyl-2-thiazolyl]amino]carbonyl]-cyclohexanecarboxylic acid, 6-[[[5-dimethylamino)carbonyl]-4-methyl-2-thiazolyl]amino]carbonyl]-3-cyclohexene-1-carboxylic acid, 2-[[(5-ethyl-1,3,4-thiadiazol-2-yl)amino]carbonyl]-cyclohexanecarboxylic acid, 2-[[(2-carboxycyclohexyl)carbonyl]amino]-4-phenyl-5-thiazolecarboxylic acid-5-ethyl ester, 6-[[(5-ethyl-1,3,4-thiadiazol-2-yl)amino]carbonyl]-3-cyclohexene-1-carboxylic acid, 2-[[(4-ethyl-5-methyl-2-thiazolyl]amino]carbonyl]-cyclohexanecarboxylic acid, 2-[[[5-methyl-4-[4-(1-methylethyl)phenyl]-2-thiazolyl]amino]carbonyl]-cyclohexanecarboxylic acid, 2-[[(5-acetyl-4-methyl-2-thiazolyl)amino]carbonyl]-cyclohexanecarboxylic acid, 6-[[[4-(2,4-dichlorophenyl)-5-methyl-2-thiazolyl]amino]carbonyl]-3-cyclohexene-1-carboxylic acid, 6-[[[4-(4-chlorophenyl)-5-methyl-2-thiazolyl]amino]carbonyl]-3-cyclohexene-1-carboxylic acid, 6-[[(5-cyclohexyl-1,3,4-thiadiazol-2-yl}amino]carbonyl]-3-cyclohexene-1-carboxylic acid, 2-[[[4-(4-chlorophenyl)-5-methyl-2-thiazolyl]amino]carbonyl-cyclohexanecarboxylic acid, 6-[[[4-(4-fluorophenyl)-5-methyl-2-thiazolyl]amino]carbonyl]-3-cyclohexene-1-carboxylic acid, 2-[[(6-carboxy-3-cyclohexen-1-yl)carbonyl-4-methyl-5-thiazolecarboxylic acid-5-methyl ester, 2-[[[4-[4-(1,1-dimethylethyl)phenyl]-5-methyl-2-thiazolyl]amino]carbonyl-cyclohexanecarboxylic acid, 2-[[[5[(dimethylethylamino)carbonyl]-4-methyl-2-thiazolyl]amino]carbonyl-cyclohexanecarboxylic acid, 6-[[(5-methyl-4-phenyl-2-thiazolyl)amino]carbonyl]-3-cyclohexene-1-carboxylic acid, 2-[[(5-methyl-1,3,4-thiadiazol-2-yl]amino]carbonyl]-cyclohexanecarboxylic acid, and 6-[[(5-(2-thienyl)-1,3,4-thiadiazol-2-yl]amino]carbonyl]-3-cyclohexene-1-carboxylic acid.

In another aspect, the present invention provides a pharmaceutical composition comprising at least one compound according to the invention or a pharmaceutically acceptable salt or solvate thereof.

The invention also relates to the use of the above compounds or their pharmaceutically acceptable salts and solvates as modulators of GPR43, preferably as agonists or partial agonists of GPR43.

The invention further provides methods of treatment and/or prevention of type II diabetes, obesity, dyslipidemia such as mixed or diabetic dyslipidemia, hypercholesterolemia, low HDL cholesterol, high LDL cholesterol, hyperlipidemia, hypertriglyceridemia, hypoglycemia, hyperglycemia, glucose intolerance, insulin resistance, hyperinsulinemia hypertension, hyperlipoproteinemia, metabolic syndrome, syndrome X, thrombotic disorders, cardiovascular disease, atherosclerosis and its sequelae including angina, claudication, heart attack, stroke and others, kidney diseases, ketoacidosis, nephropathy, diabetic neuropathy, diabetic retinopathy, nonalcoholic fatty liver diseases such as steatosis or nonalcoholic steatohepatitis (NASH) comprising the administration of a therapeutically effective amount of a compound or pharmaceutically acceptable salt or solvate of formula (I), to a patient in need thereof. Preferably the patient is a warm-blooded animal, more preferably a human.

The invention also provides the use of a compound of formula (I) or a pharmaceutically acceptable salt or solvate thereof as a medicament. Preferably, the medicament is used for the treatment and/or prevention of type II diabetes, obesity, dyslipidemia such as mixed or diabetic dyslipidemia, hypercholesterolemia, low HDL cholesterol, high LDL cholesterol, hyperlipidemia, hypertriglyceridemia, hypoglycemia, hyperglycemia, glucose intolerance, insulin resistance, hyperinsulinemia hypertension, hyperlipoproteinemia, metabolic syndrome, syndrome X, thrombotic disorders, cardiovascular disease, atherosclerosis and its sequelae including angina, claudication, heart attack, stroke and others, kidney diseases, ketoacidosis, nephropathy, diabetic neuropathy, diabetic retinopathy, nonalcoholic fatty liver diseases such as steatosis or nonalcoholic steatohepatitis (NASH).

In a preferred embodiment the disease is type II diabetes, a lipid disorder such as dyslipidemia, hypertension, obesity, or atherosclerosis and its sequelae.

### DETAILED DESCRIPTION OF THE INVENTION

As noted above, the invention relates to compounds of formula I, as well as their pharmaceutically acceptable salts and solvates.

Preferred compounds of formula I and pharmaceutically acceptable salts and solvates thereof are those wherein
D is CO; and/or
Z is —COOR, wherein R is defined as above in respect to formula I, preferably Z is COOH; and/or
R¹ is hydrogen, halogen, or a group selected from C₁₋₄ alkyl optionally substituted by one or more substituents selected from halogen, allyl or alkyl; preferably R¹ is selected from hydrogen, fluoro, methyl, or ethyl, the methyl or ethyl group being optionally substituted with one or more substituents selected from fluoro or alkyl, more preferably R¹ is hydrogen, fluoro or methyl, and most preferably R¹ is hydrogen, and L² is as defined above in respect to formula I, preferably L² is cyclopropylene, ethenylene, n-propylene, —CH₂C(R′R″)—, or —C(R′R″)—, wherein R′ and R″ are independently selected from H, halogen, methyl, and ethyl, more preferably L² is cyclopropylene, ethenylene, methylene, —CHMe-, —CHF—; even more preferably L² is methylene, or R¹ and L² together are ═CH—; and/or
R² is H, linear or branched C₁-C₄ alkyl, C₁-C₄ hydroxyalkyl, allyl, propargyl, cyclopropyl, cyclopentyl, cyclopentylmethyl, cyclopropylmethyl, 1,1,1-trifluoroethyl, —C₂H₄CO₂CH₃, —CH₂CO₂CH₃, or —CH₂CONH₂, benzyl, benzyloxyethyl, methoxyethyl, preferably R² is H, methyl, ethyl, allyl, cyclopropyl, hydroxyethyl, —C₂H₄CO₂CH₃, —CH₂CO₂CH₃, —CH₂CONH₂, more preferably R² is methyl or cyclopropyl; and/or
Ar¹ is a 5- to 6-membered aryl or heteroaryl group, or a 5- to 6-membered cycloalkyl or heterocycloalkyl group, each of which may optionally be substituted by one or more groups selected from halogen, trifluoromethyl, cyano, methoxy, trifluoromethoxy, and methoxyethoxy, and L¹ is a single bond, C₁-C₂ alkylene, or C₂ alkenylene, each optionally being substituted by one or more substituents selected from halo, C₁-C₂ alkyl, C₁-C₂ haloalkyl, preferably L¹ is a single bond, C₁-C₂ alkylene, optionally substituted by C₁-C₂ alkyl, preferably Ar¹ is phenyl or cyclohexyl and L¹ is methylene, optionally substituted by methyl; or Ar¹ is a linear or branched C₃-C₆ alkyl group, optionally substituted by one or more groups selected from halogen, trifluoromethyl, cyano, and methoxy, and L¹ is a single bond, C₁-C₂ alkylene, or C₂ alkenylene, preferably C₁-C₂ alkylene or C₂ alkenylene, and even more preferably C₁-C₂ alkylene, (Z)-ethenylene, or (E)-ethenylene, each optionally being substituted by one or more substituents selected from halo, C₁-C₂ alkyl, C₁-C₂ haloalkyl, preferably L¹ is a single bond or C₁-C₂ alkylene, optionally substituted by C₁-C₂ alkyl or one or more fluoro, more preferably L¹ is CH₂; preferably Ar¹ is isopropyl, butyl, isobutyl, cyclopentyl, cyclohexyl, tetrahydrofuranyl, tetrahydropyranyl, phenyl, furanyl, thiophenyl, thiazolyl or pyridyl, and L¹ is CH₂, more preferably Ar¹ is cyclopentyl, tetrahydrofuranyl, tetrahydropyranyl, phenyl or furanyl and L¹ is CH₂; and/or
Ar² is selected from the group consisting of thiazolylene, 1,2,4-thiadiazolylene, pyridinylene, pyrimidinylene, pyrazinylene, pyridazinylene, triazinylene, oxazolylene, 1,2,4-oxadiazolylene, pyrazolylene, each of which being optionally substituted by one or more substituents selected from halo, cyano, hydroxyl, linear or branched C₁-C₃ alkyl, C₁-C₃ hydroxyalkyl, C₁-C₃ haloalkyl, preferably F, Cl, CH₃, or CF₃, preferably Ar² is thiazolylene, 1,2,4-thiadiazolylene, pyridinylene, more preferably Ar² is thiazolylene linked to the nitrogen of N—R² at position 2 and to L³ of L³-Ar³ at position 4, 1,2,4-thiadiazolylene linked to the nitrogen of N—R² at position 5 and to L³ of L³-Ar³ at position 3, pyridinylene linked to the nitrogen of N—R² at position 2 and to L³ of L³-Ar³ at position 5; and/or
Ar³ is an aryl or heteroaryl group, optionally substituted by one or more substituents selected from halogen, C₁-C₄ alkyl, C₁-C₄ haloalkyl, C₁-C₄ alkoxy, cyano, 5 or 6 membered heteroaryl such as pyridinyl, pyrazinyl, and pyridazinyl, phenyl, methylcarbonylamino, —NH—SO₂CF₃, methylenedioxy and L³ is a single bond or C₁-C₂ alkylene; Ar³ is a C₁-C₄ alkyl group and L³ is a single bond; or -L³-Ar³ is a phenyl group fused to Ar²; preferably Ar³ is an aryl, preferably phenyl, or heteroaryl group, preferably thiophenyl, more preferably thiophen-2-yl, furanyl, more preferably furan-2-yl, each of said aryl or heteroaryl being optionally substituted by one or more substituents selected from halo, C₁-C₄ alkyl, cyclopropyl, C₁-C₄ haloalkyl, C₁-C₄ alkoxy, C₁-C₄ haloalkoxy, cyano, ethoxycarbamoyl, methylenedioxy, 5 or 6 membered aryl, preferably phenyl, 5 or 6 membered heteroaryl, preferably furanyl, thiophenyl, pyridinyl, pyrimidinyl, pyrazinyl, and pyridazinyl, more preferably furan-3-yl, thiophen-3-yl, pyridinyl, still more preferably pyridin-3-yl, each of said 5 or 6 membered aryl or 5 or 6 membered heteroaryl being optionally fused to one or more 5 or 6 membered cycloalkyl, aryl, heterocyclyl or heteroaryl moiety thus forming a fused ring system, and the latter fused ring system being optionally substituted by one or more further substituents selected from halo, hydroxyl, oxo, alkyl, and/or each of said 5 or 6 membered aryl or 5 or 6 membered heteroaryl groups being optionally substituted by one or more substituents selected from cyano, halo, hydroxyl, alkyl, cycloalkyl, heterocyclyl, aryl, heteroaryl, aralkyl, heteroarylalkyl, haloalkyl, alkoxy, haloalkoxy, alkoxyalkyl, alkoxyalkoxy, alkylaminoalkoxy, cycloalkyloxy, cycloalkylalkyloxy, heterocyclyloxy, aryloxy, aralkyloxy, alkylamino, alkylaminoalkyl, cycloalkylamino, arylamino, aralkylamino, alkylaminocarbonyl, heteroarylcarbonyl, alkylcarbonylamino, cycloalkylcarbonylamino, alkylsulfonyl, haloalkylsulfonyl, alkylsulfonylamino, each of said cycloalkyl, heterocyclyl, aryl, heteroaryl, aralkyl, heteroarylalkyl, cycloalkyloxy, cycloalkylalkyloxy, heterocyclyloxy, aryloxy, aralkyloxy, heteroarylcarbonyl, cycloalkylamino, arylamino, aralkylamino, cycloalkylcarbonylamino being optionally substituted by one or more further substituents selected from halo, preferably chloro or fluoro, oxo or alkyl, preferably methyl; more preferably Ar³ is phenyl, thiophenyl, preferably thiophen-2-yl, furanyl, preferably furan-2-yl, each of said phenyl, thiophenyl, furanyl, being optionally substituted by one or more substituents selected from halo, C₁-C₄ alkyl, cyclopropyl, C₁-C₄ haloalkyl, C₁-C₄ alkoxy, C₁-C₄ haloalkoxy, cyano, ethoxycarbamoyl, methylenedioxy, phenyl, pyridin-3-yl, each of said phenyl or pyridin-3-yl being optionally fused to one or more 5 or 6 membered heterocyclyl, phenyl, or heteroaryl moiety, preferably oxopyrrolidinyl, imidazolinyl, piperidinyl, morpholinyl, pyrrolyl, imidazolyl, or pyridinyl, more preferably 2-oxopyrrolidinyl 2-oxoimidazolinyl, 2-oxopiperidinyl or pyrrolyl, thus forming a fused ring system, and the latter fused ring system being optionally substituted by one or more further substituents selected from halo, preferably chloro or fluoro, oxo, alkyl, preferably methyl, and/or each of said phenyl or pyridin-3-yl groups being optionally substituted by one or more substituents selected from halo, alkyl, heterocyclyl, heteroaryl, haloalkyl, alkoxy, haloalkoxy, alkoxyalkyl, alkoxyalkoxy, cycloalkyloxy, cycloalkylalkyloxy, heterocyclyloxy, aralkyloxy, alkylamino, alkylaminoalkyl, cycloalkylamino, aralkylamino, alkylaminocarbonyl, alkylcarbonylamino, cycloalkylcarbonylamino, each of said heterocyclyl, heteroaryl, cycloalkyloxy, cycloalkylalkyloxy, heterocyclyloxy, aralkyloxy, cycloalkylamino, aralkylamino, cycloalkylcarbonylamino being optionally substituted by one or more further substituents selected from fluoro, chloro, oxo or methyl.
Other preferred compounds of formula I are those wherein R¹ and L² together are a 5- to 6-membered saturated or unsaturated carbocyclic or heterocyclic group, preferably a cyclohexenyl group, under the condition that -L¹-Ar¹ is H; and Ar², Ar³, R², and L³ are as defined above.

Still other preferred compounds of formula I are those wherein D is SO₂ and Ar¹, Ar², Ar³, R¹, R², L¹, L², L³, and Z are as defined above in respect to formula I.

In one embodiment, preferred compounds of Formula I are those of formula Ia:

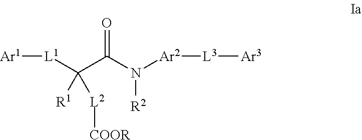

and pharmaceutically acceptable salts, and solvates thereof, wherein
R is H or linear or branched C₁-C₄ alkyl; and
Ar¹, Ar², Ar³, R¹, R², L¹, L² and L³ are as defined above in respect to formula I.

Preferred compounds of formula Ia are those wherein
R¹ is hydrogen and L² is ethenylene, ethylene, n-propylene, —CH(Me)-, —CH₂—, —CHF—, —CF₂—, or cyclopropylene; or R¹ and L² together are ═CH—; and
Ar¹, Ar², Ar³, R², L¹ and L³ are as defined above in respect to formula I.

In another embodiment, preferred compounds of Formula I are those of formula Ib:

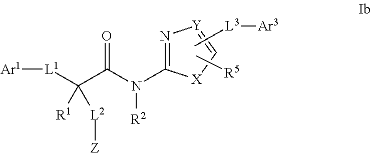

and pharmaceutically acceptable salts, and solvates thereof, wherein
X is S or O, preferably X is S;
Y is CH or N, preferably Y is CH;
L³ is attached to the heterocyclic group

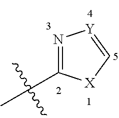

either in position 4 or 5, preferably in position 4; and
if Y is CH, R⁵ is H, halo, cyano, hydroxyl, linear or branched C₁-C₃ alkyl, C₁-C₃ hydroxyalkyl, C₁-C₃ haloalkyl, preferably H, methyl, F, Cl, or CF₃, more preferably H or F and R⁵ is attached to the heterocyclic group either in position 4, if L³ is attached in position 5, or in position 5, if L³ is attached in position 4; preferably R⁵ is attached in position 5;
if Y is N, R⁵ is absent and L³ is attached in position 5; and
Ar¹ and L¹ are as defined above in respect to formula I, preferably Ar¹ is a 5- to 6-membered aryl, preferably phenyl, or heteroaryl group, preferably furanyl, thiophenyl, oxazolyl, isoxazolyl, or thiazolyl optionally substituted by one or more groups selected from halogen, trifluoromethyl, cyano, methoxy trifluoromethoxy, and methoxyethoxy, and L¹ is a single bond, C₁-C₂ alkylene, or C₂ alkenylene, each optionally being substituted by one or more substituents selected from halo, C₁-C₂ alkyl, C₁-C₂ haloalkyl, preferably L¹ is a single bond, or C₁-C₂ alkylene, optionally substituted by C₁-C₂ alkyl, more preferably L¹ is —CH₂; or Ar¹ is a linear or branched C₃-C₆ alkyl group, preferably isopropyl, butyl, isobutyl, optionally substituted by one or more groups selected from halogen, trifluoromethyl, cyano, and methoxy, and L¹ is a single bond; or Ar¹ is cycloalkyl, preferably cyclopropyl, cyclopentyl, cyclohexyl, bicyclo[2.2.1]heptan-2-yl, more preferably cyclopentyl, or heterocycloalkyl, preferably tetrahydrofuranyl or tetrahydropyranyl and L¹ is C₁-C₂ alkylene or C₂ alkenylene, preferably C₁-C₂ alkylene or C₂ alkenylene, and even more preferably —CH₂—, (Z)-ethenylene, or (E)-ethenylene, each optionally being substituted by one or more substituents selected from halo, C₁-C₂ alkyl, C₁-C₂ haloalkyl, preferably L¹ is a single bond or C₁-C₂ alkylene, optionally substituted by C₁-C₂ alkyl, even more preferably L¹ is methylene;
Ar³ is as defined above in respect to formula I, preferably Ar³ is an aryl or heteroaryl group, optionally substituted by one or more substituents selected from halogen, C₁-C₄ alkyl, C₁-C₄ haloalkyl, C₁-C₄ alkoxy, cyano, 5 or 6 membered heteroaryl such as pyridinyl, phenyl, methylcarbonylamino, —NH—SO₂CF₃, and L³ is a single bond or C₁-C₂ alkylene; or Ar³ is a C₁-C₄ alkyl group and L³ is a single bond, more preferably Ar³ is an aryl, preferably phenyl, or heteroaryl group, preferably thiophenyl, more preferably thiophen-2-yl, furanyl, more preferably furan-2-yl, each of said aryl or heteroaryl being optionally substituted by one or more substituents selected from halo, C₁-C₄ alkyl, cyclopropyl, C₁-C₄ haloalkyl, C₁-C₄ alkoxy, C₁-C₄ haloalkoxy, cyano, ethoxycarbamoyl, methylenedioxy, 5 or 6 membered aryl, preferably phenyl, 5 or 6 membered heteroaryl, preferably furanyl, thiophenyl, pyridinyl, pyrimidinyl, pyrazinyl, and pyridazinyl, more preferably furan-3-yl, thiophen-3-yl, pyridinyl, still more preferably pyridinyl, each of said 5 or 6 membered aryl or 5 or 6 membered heteroaryl being optionally fused to one or more 5 or 6 membered cycloalkyl, aryl, heterocyclyl or heteroaryl moiety thus forming a fused ring system, and the latter fused ring system being optionally substituted by one or more further substituents selected from halo, hydroxyl, oxo, alkyl, and/or each of said 5 or 6 membered aryl or 5 or 6 membered heteroaryl groups being optionally substituted by one or more substituents selected from cyano, halo, hydroxyl, alkyl, cycloalkyl, heterocyclyl, aryl, heteroaryl, aralkyl, heteroarylalkyl, haloalkyl, alkoxy, haloalkoxy, alkoxyalkyl, alkoxyalkoxy, alkylaminoalkoxy, cycloalkyloxy, cycloalkylalkyloxy, heterocyclyloxy, aryloxy, aralkyloxy, alkylamino, alkylaminoalkyl, cycloalkylamino, arylamino, aralkylamino, alkylaminocarbonyl, heteroarylcarbonyl, alkylcarbonylamino, cycloalkylcarbonylamino, alkylsulfonyl, haloalkylsulfonyl, alkylsulfonylamino, each of said cycloalkyl, heterocyclyl, aryl, heteroaryl, aralkyl, heteroarylalkyl, cycloalkyloxy, cycloalkylalkyloxy, heterocyclyloxy, aryloxy, aralkyloxy, heteroarylcarbonyl, cycloalkylamino, arylamino, aralkylamino, cycloalkylcarbonylamino being optionally substituted by one or more further substituents selected from halo, preferably chloro or fluoro, oxo or alkyl, preferably methyl; still more preferably Ar³ is phenyl, thiophenyl, preferably thiophen-2-yl, furanyl, preferably furan-2-yl, each of said phenyl, thiophenyl, furanyl, being optionally substituted by one or more substituents selected from halo, C₁-C₄ alkyl, cyclopropyl, C₁-C₄ haloalkyl, C₁-C₄ alkoxy, C₁-C₄ haloalkoxy, cyano, ethoxycarbamoyl, methylenedioxy, phenyl, pyridin-3-yl, each of said phenyl or pyridin-3-yl being optionally fused to one or more 5 or 6 membered heterocyclyl, phenyl, or heteroaryl moiety, preferably oxopyrrolidinyl, imidazolinyl, piperidinyl, morpholinyl, pyrrolyl, imidazolyl, or pyridyl, more preferably 2-oxopyrrolidinyl 2-oxoimidazolinyl, 2-oxopiperidinyl or pyrrolyl, thus forming a fused ring system, and the latter fused ring system being optionally substituted by one or more further substituents selected from halo, preferably chloro or fluoro, oxo, alkyl, preferably methyl, and/or each of said phenyl or pyridin-3-yl groups being optionally substituted by one or more substituents selected from halo, alkyl, heterocyclyl, heteroaryl, haloalkyl, alkoxy, haloalkoxy, alkoxyalkyl, alkoxyalkoxy, cycloalkyloxy, cycloalkylalkyloxy, heterocyclyloxy, aralkyloxy, alkylamino, alkylaminoalkyl, cycloalkylamino, aralkylamino, alkylaminocarbonyl, alkylcarbonylamino, cycloalkylcarbonylamino, each of said heterocyclyl, heteroaryl, cycloalkyloxy, cycloalkylalkyloxy, heterocyclyloxy, aralkyloxy, cycloalkylamino, aralkylamino, cycloalkylcarbonylamino being optionally substituted by one or more further substituents selected from fluoro, chloro, oxo or methyl;
R¹ is as defined above in respect to formula I, preferably R¹ is hydrogen, halogen, allyl, or a group selected from C₁₋₄ alkyl optionally substituted by one or more substituents selected from halogen or alkyl; more preferably R¹ is selected from hydrogen, fluoro, or methyl or ethyl, the methyl or ethyl group being optionally substituted with one or more substituents selected from fluoro or alkyl, even more preferably R¹ is hydrogen, fluoro or methyl, and most preferably R¹ is hydrogen, and L² is as defined above in respect to formula I, preferably L² is cyclopropylene, ethenylene, n-propylene, —C(R′R″)—, wherein R′ and R″ are independently selected from H, halogen, methyl, and ethyl, more preferably L² is cyclopropylene, ethenylene, methylene, —CHMe-, —CHF—, even more preferably L² is methylene; or R¹ and L² together are ═CH—;
Z is as defined above in respect to formula I, preferably Z is —COOR, wherein R is defined as above in respect to formula I, more preferably Z is COOH; and
R² is as defined above in respect to formula I, preferably R² is H, linear or branched C₁-C₄ alkyl, C₁-C₂ hydroxyalkyl, allyl, propargyl, cyclopropyl, cyclopentyl, cyclopentylmethyl, cyclopropylmethyl, benzyl, benzyloxyethyl, methoxyethyl, 1,1,1-trifluoroethyl, —C₂H₄CO₂CH₃, —CH₂CO₂CH₃, or —CH₂CONH₂, more preferably R² is H, methyl, ethyl, allyl, cyclopropyl, hydroxyethyl, —C₂H₄CO₂CH₃, —CH₂CO₂CH₃, or —CH₂CONH₂, more preferably R² is methyl or cyclopropyl.

Preferred compounds of formula Ib are those wherein Z is —COOR, preferably COOH, and R, Ar¹, Ar², Ar³, R¹, R², L¹, L² and L³ are as defined above in respect to formula I.

Particularly preferred compounds of formula Ib are those of formula Ib-1

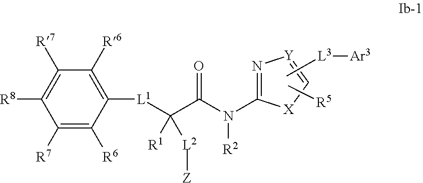

wherein L¹, L², L³, Ar³, X, Y, Z, R¹, R², and R⁵ are as defined above in respect to formula Ib, preferably L¹ is methylene, optionally substituted by C₁-C₂ alkyl or halo, preferably by methyl or fluoro, even more preferably L¹ is methylene; and
R⁶, R⁷, R′⁶, R′⁷ and R⁸ are independently selected from H, halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or R⁶ and R⁷ or R⁷ and R⁸ or R′⁶ and R′⁷ or R′⁷ and R⁸ together form an alkylenedioxy group or a haloalkylenedioxy group, or R⁶ and R⁷ or R⁷ and R⁸ or R′⁶ and R′⁷ or R′⁷ and R⁸ together form a cycloalkyl, aryl, heterocyclyl or heteroaryl moiety fused to the phenyl group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, preferably R⁶, R⁷, R′⁶, R′⁷ and R⁸ are independently selected from H, halo, cyano, alkyl, hydroxyalkyl, haloalkyl, cycloalkyl, cycloalkylalkyl, heteroalkyl, heterocyclyl, heterocyclylalkyl, aryl, heteroaryl, heteroarylalkyl, hydroxyl, alkoxy, alkoxyalkyl, haloalkoxy, cycloalkyloxy, heterocyclyloxy, aryloxy, carboxy, alkylcarbonylamino, haloalkylcarbonylamino, cycloalkylcarbonylamino, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl, alkylsulfonylamino, cycloalkylsulfonylamino, more preferably R⁶, R⁷, R′⁶, R′⁷ and R⁸ are independently selected from H, hydroxyl, halo, alkyl, haloalkyl, alkoxy, alkoxyalkyl preferably methoxyethyl, haloalkoxy preferably —OCF₃, alkylsulfonyl, haloalkylsulfonyl and cyano, even more preferably from H, halo, CF₃, C1-C2 alkyl, C1-C2 alkoxy, and cyano, still more preferably from H, F, Cl, CF₃, methyl, methoxy, and cyano, still more preferably R⁶, R⁷, R′⁶, R′⁷ are H and R⁸ is selected from H, Cl, methyl, hydroxyl and methoxy, and most preferably R⁶, R⁷, R′⁶, R′⁷ are H and R⁸ is selected from H, Cl, methyl, and methoxy.

Preferred compounds of formula Ib-1 are those of formula Ib-1a

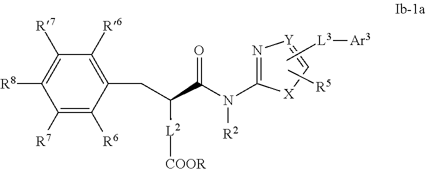

wherein L², L³, Ar³, X, Y, R², R⁵, R⁶, R⁷, R′⁶, R′⁷ and R⁸ are as defined above in respect to formula Ib-1.

Other preferred compounds of formula Ib are selected form the group consisting of formulae Ib-2a, Ib-2b, Ib-2c, Ib-2d, Ib-2e and Ib-2f:

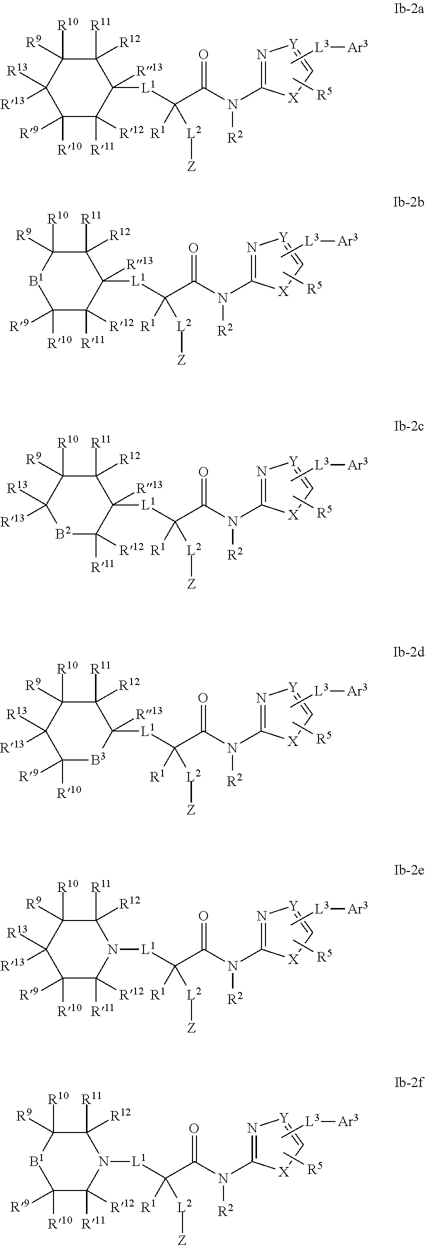

wherein L¹, L², L³, Ar³, X, Y, Z, R¹, R² and R⁵ are as defined above in respect to formula Ib, preferably L¹ is methylene;
B¹, B² and B³ are independently CF₂, O, NR^{a}, CO, or SO₂, wherein R^{a} is H or alkyl, preferably linear or branched C₁-C₄ alkyl; C₁-C₄ alkylcarbonyl, C₁-C₄ alkylsulfonyl, C₁-C₄ alkylaminocarbonyl, C₃-C₆ cycloalkyl; C₃-C₆ cycloalkylcarbonyl, C₃-C₆ cycloalkylsulfonyl, C₃-C₆ cycloalkylaminocarbonyl, aryl, arylcarbonyl, arylsulfonyl or arylaminocarbonyl, heteroaryl, heteroarylcarbonyl, heteroarylsulfonyl or heteroarylaminocarbonyl; preferably B¹, B² and B³ are O and
R⁹, R¹⁰, R¹¹, R¹², R¹³, R′⁹, R′¹⁰, R′¹¹, R′¹², R′¹³ and R″¹³ are independently selected from H, halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or one of R⁹ or R¹⁰ and one of R¹¹, R¹², R¹³, R′⁹, R′¹⁰, R′¹¹, R′¹², R′¹³ or R″¹³, or one of R¹¹ or R¹² and one of R⁹, R¹⁰, R¹³, R′⁹, R′¹⁰, R′¹¹, R′¹², R′¹³ or R″¹³, or one of R¹³ or R′¹³ and one of R⁹, R¹⁰, R¹¹, R¹², R′⁹, R′¹⁰, R′¹¹, R′¹², or R″¹³ together form an alkylenedioxy group or a haloalkylenedioxy group, or one of R⁹ or R¹⁰ and one of R¹¹, R¹², R¹³, R′⁹, R′¹⁰, R′¹¹, R′¹², R′¹³ or R″¹³, or one of R¹¹ or R¹² and one of R⁹, R¹⁰, R¹³, R′⁹, R′¹⁰, R′¹¹, R′¹², R′¹³ or R″¹³, or one of R¹³ or R′¹³ and one of R⁹, R¹⁰, R¹¹, R¹², R′⁹, R′¹⁰, R′¹¹, R′¹², or R″¹³ together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, preferably R⁹, R¹⁰, R¹¹, R¹², R¹³, R′⁹, R′¹⁰, R′¹¹, R′¹², R′¹³ and R″¹³ are independently selected from H, halo, cyano, alkyl, hydroxyalkyl, haloalkyl, cycloalkyl, cycloalkylalkyl, heteroalkyl, heterocyclyl, heterocyclylalkyl, aryl, heteroaryl, heteroarylalkyl, hydroxyl, alkoxy, haloalkoxy, cycloalkyloxy, heterocyclyloxy, aryloxy, carboxy, alkylcarbonylamino, haloalkylcarbonylamino, cycloalkylcarbonylamino, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl, alkylsulfonylamino, cycloalkylsulfonylamino, or one of R⁹ or R¹⁰ and one of R¹¹, R¹², R¹³, R′⁹, R′¹⁰, R′¹¹, R′¹², R′¹³ or R″¹³, or one of R¹¹ or R¹² and one of R⁹, R¹⁰, R¹³, R′⁹, R′¹⁰, R′¹¹, R′¹², R′¹³ or R″¹³, or one of R¹³ or R′¹³ and one of R⁹, R¹⁰, R¹¹, R¹², R′⁹, R′¹⁰, R′¹¹, R′¹², or R″¹³ together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, more preferably R⁹, R¹⁰, R¹¹, R¹², R¹³, R′⁹, R′¹⁰, R′¹¹, R′¹², R′¹³ and R″¹³ are independently selected from H, hydroxyl, C₁-C₃-alkyl, halo, haloalkyl, alkoxy, haloalkoxy, alkylsulfonyl, haloalkylsulfonyl and cyano, even more preferably from H, C₁-C₃-alkyl, halo, CF₃, C1-C2 alkoxy, and cyano, and still more preferably from H, F, Cl, methyl, CF₃, methoxy, and cyano, and most preferably H or methyl.

Particularly preferred compounds of formula Ib-2a are

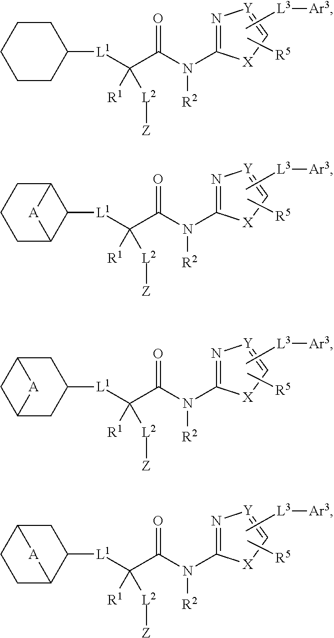

wherein A is —(CH₂)ₙ—O—, —(CH₂)ₙ—NR^{a}—, —(CH₂)ₙ—SO₂—, or —(CH₂)ₘ—, wherein n is equal to 0 or 1, m is equal to 1 or 2, and R^{a} is as defined above in respect to formula Ib-2b, preferably R^{a} is H or alkyl, preferably linear or branched C₁-C₄ alkyl; C₁-C₄ alkylcarbonyl, C₁-C₄ alkylsulfonyl, more preferably linear or branched C₁-C₄ alkyl; and
L¹, L², L³, Ar³, X, Y, Z, R¹, R² and R⁵ are as defined above in respect to formula Ib-2a.

Even more preferred compounds of formula Ib-2a are selected from

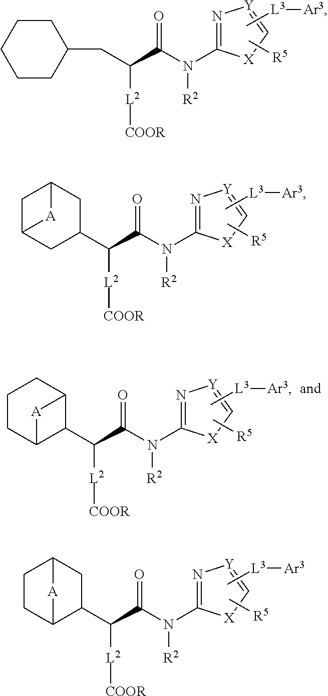

wherein A is —(CH₂)ₙ—O—, —(CH₂)ₙ—NR^{a}—, —(CH₂)ₙ—SO₂—, or —(CH₂)ₘ—, wherein n is equal to 0 or 1, m is equal to 1 or 2, and R^{a} is as defined above in respect to formula Ib-2b, preferably R^{a} is H or alkyl, preferably linear or branched C₁-C₄ alkyl; C₁-C₄ alkylcarbonyl, C₁-C₄ alkylsulfonyl, more preferably linear or branched C₁-C₄ alkyl; and
L², L³, Ar³, X, Y, R, R¹, R² and R⁵ are as defined above in respect to formula Ib-2a.

Further preferred compounds of formula Ib are those of formula Ib-3

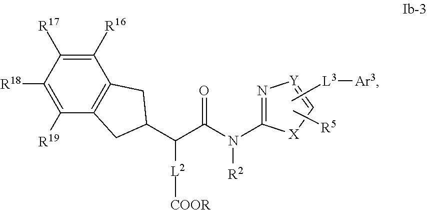

preferably

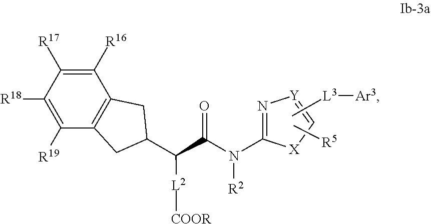

wherein L², L³, Ar³, X, Y, R, R¹, R² and R⁵ are as defined above in respect to formula Ib; and
R¹⁶, R¹⁷, R¹⁸ and R¹⁹ are independently selected from H, halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, alkoxyalkyl, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoyl alkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or R¹⁶ and R¹⁷ or R¹⁷ and R¹⁸ or R¹⁸ and R¹⁹ together form an alkylenedioxy group or a haloalkylenedioxy group, or R¹⁶ and R¹⁷ or R¹⁷ and R¹⁸ or R¹⁸ and R¹⁹ together form a cycloalkyl, aryl, heterocyclyl or heteroaryl moiety fused to the phenyl group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, preferably R¹⁶, R¹⁷, R¹⁸ and R¹⁹ are independently selected from H, halo, cyano, alkyl, hydroxyalkyl, haloalkyl, cycloalkyl, cycloalkylalkyl, heteroalkyl, heterocyclyl, heterocyclylalkyl, aryl, heteroaryl, heteroarylalkyl, hydroxyl, alkoxy, alkoxyalkyl, preferably methoxyethyl, haloalkoxy, preferably trifluoromethoxy, cycloalkyloxy, heterocyclyloxy, aryloxy, carboxy, alkylcarbonylamino, haloalkylcarbonylamino, cycloalkylcarbonylamino, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl, alkylsulfonylamino, cycloalkylsulfonylamino, more preferably R¹⁶, R¹⁷, R¹⁸ and R¹⁹ are independently selected from H, hydroxyl, halo, haloalkyl, alkoxy, haloalkoxy, preferably trifluoromethoxy, alkylsulfonyl, haloalkylsulfonyl and cyano, even more preferably from H, halo, CF₃, methyl, C₁-C₂ alkoxy, and cyano, and most preferably from H, F, Cl, CF₃, methyl, methoxy, and cyano.

Further preferred compounds of formula Ib are those of formula Ib-4

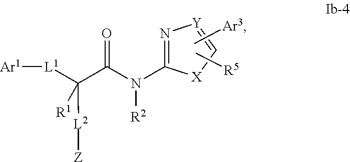

wherein
Ar¹, Ar³, L¹, L², R¹, R², R⁵, X, Y and Z are as defined above in respect to formula Ib.
Preferred compounds of formula Ib-4 are those of formula Ib-4a

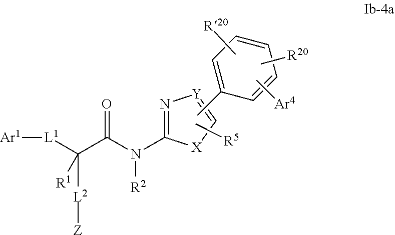

wherein
Ar¹, L¹, L², R¹, R², R⁵, X, Y and Z are as defined above in respect to formula Ib-4,
R²⁰ and R′²⁰ are independently selected from halo (preferably —F and —Cl), cyano, C₁-C₃ alkyl, cyclopropyl, haloalkyl, alkoxy, haloalkoxy, alkoxycarbonylamino, or the two substituents form an alkylenedioxy group or a haloalkylenedioxy group, preferably R²⁰ and R′²⁰ are halo preferably fluoro or chloro, haloalkyl, preferably —CF₃ or —CHF₂, alkoxy preferably methoxy, haloalkoxy preferably —OCF₃ or —OCHF₂;
Ar⁴ is 5 or 6 membered aryl, preferably phenyl, 5 or 6 membered heteroaryl, preferably furanyl, thiophenyl, pyridinyl, pyrimidinyl, pyrazinyl, and pyridazinyl, more preferably furan-3-yl, thiophen-3-yl, pyridinyl, still more preferably pyridin-3-yl, each of said 5 or 6 membered aryl or 5 or 6 membered heteroaryl groups being optionally fused to one or more 5 or 6 membered cycloalkyl, aryl, heterocyclyl or heteroaryl moiety, thus forming a fused ring system, and the latter fused ring system being optionally substituted by one or more further substituents selected from halo, hydroxyl, oxo, alkyl, and/or each of said 5 or 6 membered aryl or 5 or 6 membered heteroaryl groups being optionally substituted by one or more substituents selected from halo, cyano, hydroxyl, alkyl, cycloalkyl, heterocyclyl, aryl, heteroaryl, aralkyl, heteroarylalkyl, haloalkyl, alkoxy, haloalkoxy, alkoxyalkyl, alkoxyalkoxy, alkylaminoalkoxy, cycloalkyloxy, cycloalkylalkyloxy, heterocyclyloxy, aryloxy, aralkyloxy, alkylamino, alkylaminoalkyl, cycloalkylamino, arylamino, aralkylamino, alkylaminocarbonyl, heteroarylcarbonyl, alkylcarbonylamino, cycloalkylcarbonylamino, alkylsulfonyl, haloalkylsulfonyl, alkylsulfonylamino, each of said cycloalkyl, heterocyclyl, aryl, heteroaryl, aralkyl, heteroarylalkyl, cycloalkyloxy, cycloalkylalkyloxy, heterocyclyloxy, aryloxy, aralkyloxy, heteroarylcarbonyl, cycloalkylamino, arylamino, aralkylamino, cycloalkylcarbonylamino being optionally substituted by one or more further substituents selected from halo, preferably chloro or fluoro, oxo or alkyl, preferably methyl; preferably Ar⁴ is phenyl or pyridin-3-yl, each of said phenyl or pyridin-3-yl being optionally fused to one or more 5 or 6 membered heterocyclyl, phenyl, or 5 or 6 membered heteroaryl moiety, preferably oxopyrrolidinyl, imidazolinyl, piperidinyl, morpholinyl, pyrrolyl, imidazolyl, or pyridyl more preferably 2-oxopyrrolidinyl, 2-oxoimidazolinyl, 2-oxopiperidinyl, or pyrrolyl, thus forming a fused ring system, and the latter fused ring system being optionally substituted by one or more further substituents selected from halo, preferably chloro or fluoro, oxo, alkyl, preferably methyl, and/or each of said phenyl or pyridin-3-yl groups being optionally substituted by one or more substituents selected from halo, alkyl, heterocyclyl, heteroaryl, haloalkyl, alkoxy, haloalkoxy, alkoxyalkyl, alkoxyalkoxy, cycloalkyloxy, cycloalkylalkyloxy, heterocyclyloxy, aralkyloxy, alkylamino, alkylaminoalkyl, cycloalkylamino, aralkylamino, alkylaminocarbonyl, alkylcarbonylamino, cycloalkylcarbonylamino, each of said heterocyclyl, heteroaryl, cycloalkyloxy, cycloalkylalkyloxy, heterocyclyloxy, aralkyloxy, cycloalkylamino, aralkylamino, cycloalkylcarbonylamino being optionally substituted by one or more further substituents selected from fluoro, chloro, oxo or methyl.
Preferred compounds of formula Ib-4a are those of formula Ib-4b

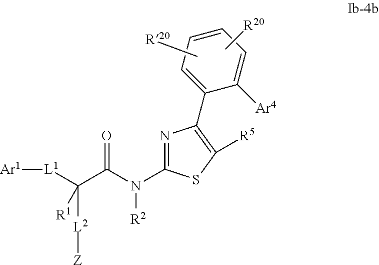

wherein
Ar¹, L¹, L², R¹, R², R⁵, and Z are as defined above in respect to formula Ib, and
Ar⁴, R²⁰ and R′²⁰, are as defined above in respect to formula Ib-4a.
Preferred compounds of formula Ib-4b are those of formula Ib-4c

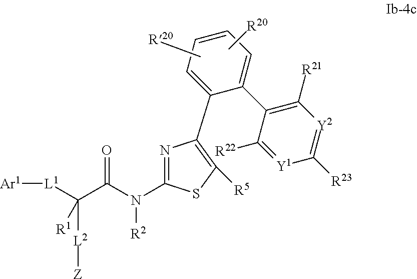

wherein
Ar¹, L¹, L², R¹, R², R⁵, and Z are as defined above in respect to formula Ib;
R²⁰ and R′²⁰, are as defined above in respect to formula Ib-4a;
R²¹ and R²² are independently selected from H, halo, preferably fluoro or chloro, alkoxy, preferably methoxy, preferably R²¹ and R²² are H;
R²³ is selected from halo, cyano, hydroxyl, alkyl, cycloalkyl, heterocyclyl, aryl, heteroaryl, aralkyl, heteroarylalkyl, haloalkyl, alkoxy, haloalkoxy, alkoxyalkyl, alkoxyalkoxy, alkylaminoalkoxy, preferably dimethylaminoethoxy, cycloalkyloxy, cycloalkylalkyloxy, heterocyclyloxy, aryloxy, aralkyloxy, alkylamino, alkylaminoalkyl, cycloalkylamino, arylamino, aralkylamino, alkylaminocarbonyl, heteroarylcarbonyl, alkylcarbonylamino, cycloalkylcarbonylamino, alkylsulfonyl, preferably C₁-C₃ alkylsulfonyl, more preferably methylsulfonyl, haloalkylsulfonyl, alkylsulfonylamino preferably N-methyl(methylsulfonyl)amino, each of said cycloalkyl, heterocyclyl, aryl, heteroaryl, aralkyl, heteroarylalkyl, cycloalkyloxy, cycloalkylalkyloxy, heterocyclyloxy, aryloxy, aralkyloxy, heteroarylcarbonyl, cycloalkylamino, arylamino, aralkylamino, cycloalkylcarbonylamino being optionally substituted by one or more further substituents selected from halo, preferably chloro or fluoro, oxo or alkyl, preferably methyl preferably R²³ is selected from halo, preferably chloro or fluoro, alkyl, preferably linear or branched C₁-C₅ alkyl, more preferably methyl or isopropyl, 5 or 6-membered heterocyclyl, preferably pyrrolidin-1-yl, 2-oxopyrrolidin-1-yl, 1-methyl-2-oxoimidazolin-3-yl, 1-methylpiperazin-4-yl, morpholin-4-yl, heteroaryl, preferably 1,3,4-triazol-1-yl, haloalkyl, C₁-C₃ alkoxy, preferably methoxy, haloalkoxy, alkoxyalkyl, preferably methoxymethyl, alkoxyalkoxy, preferably methoxyethoxy, cycloalkyloxy, cycloalkylalkyloxy, preferably cyclopropylmethyloxy, heterocyclyloxy, preferably (tetrahydropyran-4-yl)oxy, aralkyloxy, preferably benzyloxy, C₁-C₃ alkylamino, preferably dimethylamino, alkylaminoalkyl, cycloalkylamino, preferably N-methylcyclohexylamino, aralkylamino, preferably N-methylbenzylamino, C₁-C₆ alkylaminocarbonyl preferably dimethylaminocarbonyl, C₁-C₆ alkylcarbonylamino, preferably methylcarbonylamino, cycloalkylcarbonylamino, each of said 5 or 6-membered heterocyclyl, heteroaryl, cycloalkyloxy, cycloalkylalkyloxy, heterocyclyloxy, aralkyloxy, cycloalkylamino, aralkylamino, cycloalkylcarbonylamino being optionally substituted by one or more further substituents selected from fluoro, chloro, oxo or methyl, even more preferably R²³ is selected from chloro, fluoro, isopropyl, 5 or 6-membered heterocyclyl preferably pyrrolidin-1-yl, 2-oxopyrrolidin-1-yl, morpholin-4-yl, 1-methyl-2-oxoimidazolin-3-yl, C₁-C₃ alkoxy preferably methoxy, alkyloxyalkoxy, preferably methoxyethoxy, aralkyloxy, preferably benzyloxy, C₁-C₃ alkylamino preferably dimethylamino, each of said 5 or 6-membered heterocyclyl, aralkyloxy being optionally substituted by one or more further substituents selected from fluoro, chloro, oxo, or methyl;
Y¹ is N or C—R²⁴ where R²⁴ is H, halo, alkoxy, alkyl, heterocyclyl, preferably pyrrolidinyl, imidazolinyl, piperidinyl, morpholinyl, more preferably 2-oxopyrrolidin-1-yl, 2-oxoimidazolin-1-yl, 2-oxopiperidin-1-yl, or morpholin-4-yl, each of said substituents being optionally substituted by one or more further substituents selected from halo, preferably chloro or fluoro, oxo, alkyl, preferably methyl, preferably R²⁴ is H, halo, methoxy, more preferably H, chloro or fluoro, or
Y¹ is C—R²⁴ and R²⁴ and R²³ together form a 5 or 6 membered cycloalkyl, aryl, heterocyclyl or heteroaryl moiety, preferably 2-oxopyrrolidinyl, morpholinyl, 2-oxopiperidinyl, furanyl, pyrrolyl, imidazolyl, thus forming a fused ring system, the latter fused ring system being optionally substituted by one or more group selected from oxo, alkyl or halo; and
Y² is N or C—R²⁵ where R²⁵ is H, halo, alkoxy, alkyl, heterocyclyl, preferably pyrrolidinyl, imidazolinyl, piperidinyl or morpholinyl, more preferably 2-oxopyrrolidin-1-yl, 2-oxoimidazolin-1-yl, 2-oxopiperidin-1yl or morpholin-4-yl, each of said substituents being optionally substituted by one or more further substituents selected from halo, preferably chloro or fluoro, oxo, alkyl, preferably methyl, preferably R²⁵ is H, halo, methoxy, more preferably H, chloro or fluoro, or
Y² is C—R²⁵ and R²⁵ and R²³ together form a 5 or 6 membered cycloalkyl, aryl, heterocyclyl or heteroaryl moiety, preferably 2-oxopyrrolidinyl, morpholinyl, 2-oxopiperidinyl, furanyl, pyrrolyl, imidazolyl, furanyl, thus forming a fused ring system, the latter fused ring system being optionally substituted by one or more group selected from oxo, alkyl or halo, under the condition that R²⁴ and R²³ together do not form a 5 or 6 membered cycloalkyl, aryl, heterocyclyl or heteroaryl moiety.
Preferred compounds of formula Ib-4c are those of formula Ib-4d

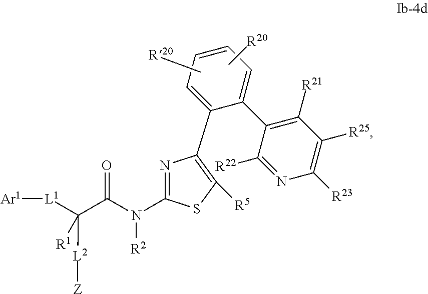

wherein,
Ar¹, L¹, L², R¹, R², R⁵, and Z are as defined above in respect to formula Ib;
R²⁰ and R′²⁰, are as defined above in respect to formula Ib-4a; and
R²¹, R²², R²³ and R²⁵ are as defined above in respect to formula Ib-4c.
Preferred compounds of formula Ib-4d are those of formula Ib-4e

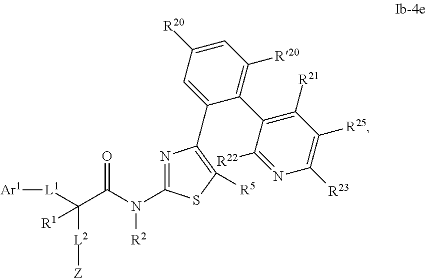

wherein,
Ar¹, L¹, L², R¹, R², R⁵, and Z are as defined above in respect to formula Ib;
R²⁰ and R′²⁰, are as defined above in respect to formula Ib-4a; and
R²¹, R²², R²³ and R²⁵ are as defined above in respect to formula Ib-4c.
Other preferred compounds of formula Ib-4d are those of Ib-4f

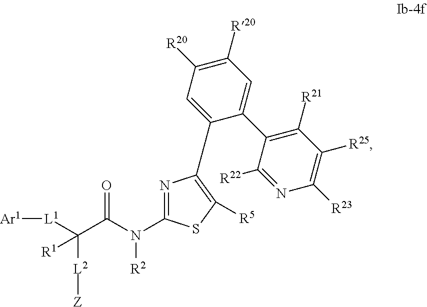

wherein
Ar¹, L¹, L², R¹, R², R⁵, and Z are as defined above in respect to formula Ib;
R²⁰ and R′²⁰, are as defined above in respect to formula Ib-4a; and
R²¹, R²², R²³ and R²⁵ are as defined above in respect to formula Ib-4c.
Still other preferred compounds of formula Ib-4d are those of formula Ib-4g

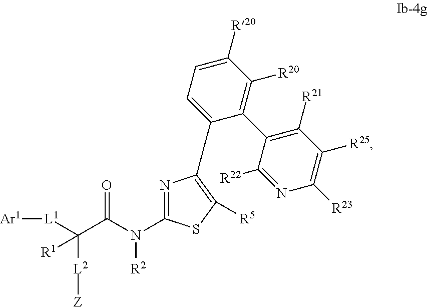

wherein
Ar¹, L¹, L², R¹, R², R⁵, and Z are as defined above in respect to formula Ib;
R²⁰ and R′²⁰, are as defined above in respect to formula Ib-4a; and
R²¹, R²², R²³ and R²⁵ are as defined above in respect to formula Ib-4c.
Other preferred compounds of formula Ib-4c are those of formula Ib-4d′

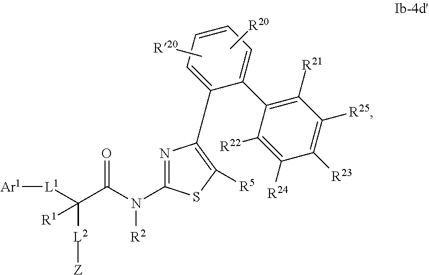

wherein
Ar¹, L¹, L², R¹, R², R⁵, and Z are as defined above in respect to formula Ib;
R²⁰ and R′²⁰, are as defined above in respect to formula Ib-4a; and
R²¹, R²², R²³ and R²⁵ are as defined above in respect to formula Ib-4c.
Preferred compounds of formula Ib-4d′ are those of formula Ib-4e′

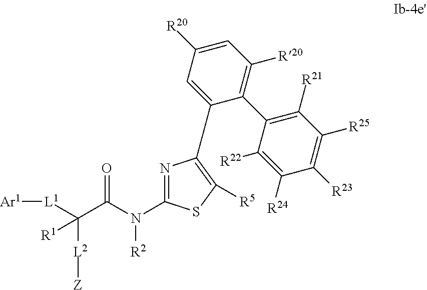

wherein
Ar¹, L¹, L², R¹, R², R⁵, and Z are as defined above in respect to formula Ib;
R²⁰ and R′²⁰, are as defined above in respect to formula Ib-4a; and
R²¹, R²², R²³ and R²⁵ are as defined above in respect to formula Ib-4c.
Other preferred compounds of formula Ib-4d′ are those of formula Ib-4f′

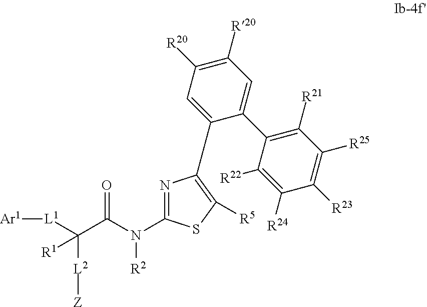

wherein
Ar¹, L¹, L², R¹, R², R⁵, and Z are as defined above in respect to formula Ib;
R²⁰ and R′²⁰, are as defined above in respect to formula Ib-4a; and
R²¹, R²², R²³ and R²⁵ are as defined above in respect to formula Ib-4c.
Still other preferred compounds of formula Ib-4d′ are those of formula Ib-4g′

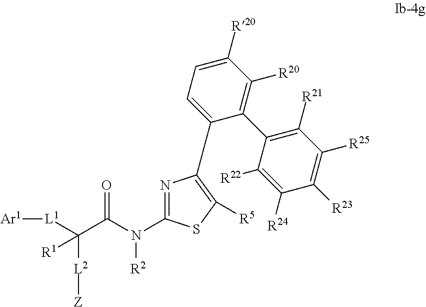

wherein,
Ar¹, L¹, L², R¹, R², R⁵, and Z are as defined above in respect to formula Ib;
R²⁰ and R′²⁰, are as defined above in respect to formula Ib-4a;
R²¹, R²², R²³ and R²⁵ are as defined above in respect to formula Ib-4c.
In another embodiment of the invention, preferred compounds of formula Ib-4a are those of formula Ib-4h,

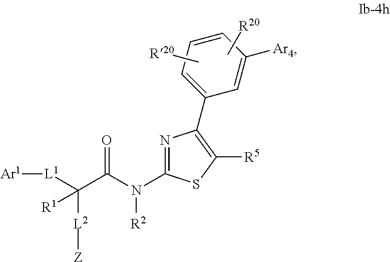

wherein
Ar¹, L¹, L², R¹, R², R⁵, and Z are as defined above in respect to formula Ib; and
Ar⁴, R²⁰ and R′²⁰, are as defined above in respect to formula Ib-4a.
Preferred compounds of formula Ib-4h are those of formula Ib-4i

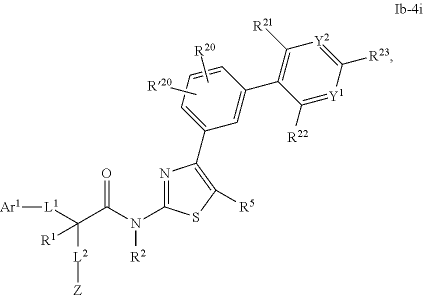

wherein
Ar¹, L¹, L², R¹, R², R⁵, and Z are as defined above in respect to formula Ib;
R²⁰ and R′²⁰, are as defined above in respect to formula Ib-4a; and
R²¹, R²², R²³, Y¹ and Y² are as defined above in respect to formula Ib-4c.
Preferred compounds of formula Ib-4i are those of formula Ib-4j

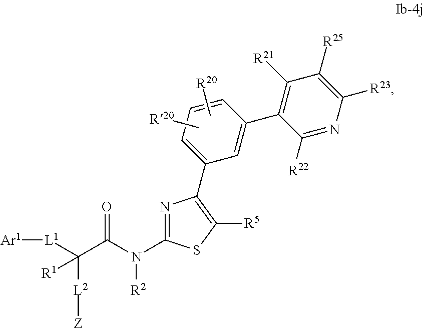

wherein
Ar¹, L¹, L², R¹, R², R⁵, and Z are as defined above in respect to formula Ib;
R²⁰ and R′²⁰, are as defined above in respect to formula Ib-4a; and
R²¹, R²², R²³ and R²⁵ are as defined above in respect to formula Ib-4c.
Other preferred compounds of formula Ib-4 are those of formula Ib-4k,

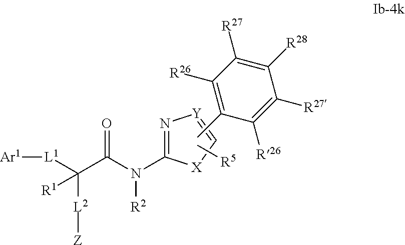

wherein
Ar¹, L¹, L², R¹, R², R⁵, X, Y, and Z are as defined above in respect to formula Ib;
R²⁶, R′²⁶, R²⁷, R′²⁷, R²⁸ are independently selected from H, halo, cyano, alkyl, haloalkyl, cycloalkyl, cycloalkylalkyl, hydroxyl, alkoxy, haloalkoxy, cycloalkyloxy, alkylamino, carboxy, alkoxycarbonyl,=alkylcarbonylamino, haloalkylcarbonylamino, cycloalkylcarbonylamino, acylamino, carbamoyl, alkoxycarbamoyl, cycloalkylcarbamoyl, alkylcarbamoylamino, cycloalkylaminocarbamoyl, alkylsulfonyl, haloalkylsulfonyl, sulfamoyl, alkylsulfamoyl, alkylsulfonylamino, haloalkylsulfonylamino, or two substituents form an alkylenedioxy group or a haloalkylenedioxy group, preferably R²⁶, R′²⁶, R²⁷, R′²⁷, R²⁸ are independently selected from H, halo, preferably chloro or fluoro, more preferably chloro, cyano, alkyl, preferably methyl, haloalkyl, preferably —CF₃ or —CHF₂, cycloalkyl, preferably cyclopropyl, alkoxy, preferably methoxy or isopropyloxy, haloalkoxy, preferably —OCF₃ or —OCHF₂, alkoxycarbamoyl, or two substituents form an methylenedioxy group, more preferably R²⁶, R′²⁶, R²⁷, R′²⁷, R²⁸ are independently selected from H, halo, preferably chloro or fluoro, more preferably chloro, haloalkyl, preferably —CF₃ or —CHF₂, alkoxy, preferably methoxy.
Preferred compounds of formula Ib-4k are those of formula Ib-4l

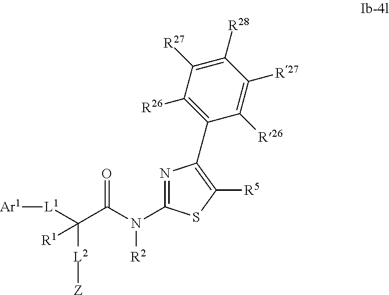

wherein
Ar¹, L¹, L², R¹, R², R⁵ and Z are as defined above in respect to formula Ib; and
R²⁶, R′²⁶, R²⁷, R′²⁷ and R²⁸ are as defined above in respect to formula Ib-4k.
Preferred compounds of formula Ib-4l are those of formula Ib-4m

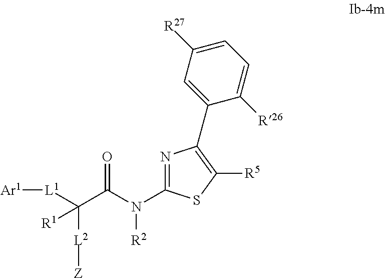

wherein,
Ar¹, L¹, L², R¹, R², R⁵ and Z are as defined above in respect to formula Ib; and
R′²⁶ and R²⁷ are as defined above in respect to formula Ib-4k, preferably R′²⁶ and R²⁷ are independently selected from H, halo, haloalkyl, haloalkoxy, preferably chloro, fluoro CF₃, CHF₂, OCF₃ or OCHF₂, preferably R′²⁶ is chloro and R²⁷ is selected from H, halo, CF₃, CHF₂, OCF₃ or OCHF₂, preferably chloro and fluoro.
Other preferred compounds of formula Ib-4l are those of formula Ib-4n,

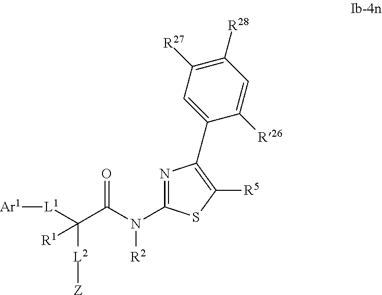

wherein,
Ar¹, L¹, L², R¹, R², R⁵ and Z are as defined above in respect to formula Ib; and
R′²⁶, R²⁷ and R²⁸ are as defined above in respect to formula Ib-4k, preferably R′²⁶, R²⁷ and R²⁸ are independently selected from H, halo, haloalkyl, haloalkoxy, preferably chloro, fluoro, CF₃, or CHF₂, preferably OCF₃ or OCHF₂.
Other preferred compounds of formula Ib-4l are those of formula Ib-4o

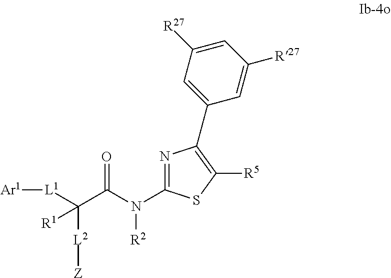

wherein
Ar¹, L¹, L², R¹, R², R⁵ and Z are as defined above in respect to formula Ib; and
R²⁷ and R′²⁷ are as defined above in respect to formula Ib-4k, preferably R²⁷ and R′²⁷ are independently selected from H, halo, haloalkyl, haloalkoxy, preferably chloro, fluoro, CF₃, CHF₂OCF₃ or OCHF₂.
Other preferred compounds of formula Ib-4l are those of formula Ib-4p

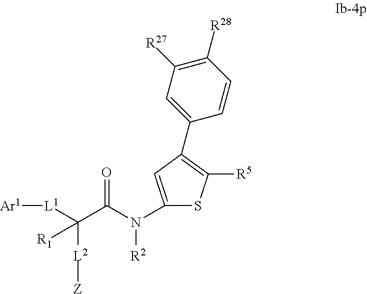

wherein,
Ar¹, L¹, L², R¹, R², R⁵ and Z are as defined above in respect to formula Ib; and
R²⁷ and R²⁸ are as defined above in respect to formula Ib-4k, preferably R²⁷ and R²⁸ are independently selected from H, halo, haloalkyl, alkoxy, haloalkoxy, preferably chloro, fluoro, CF₃, CHF₂, methoxy, OCF₃ or OCHF₂.
Still other preferred compounds of formula Ib-4l are those of formula Ib-4q

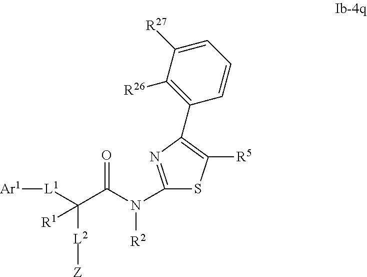

wherein,
Ar¹, L¹, L², R¹, R², R⁵ and Z are as defined above in respect to formula Ib; and
R²⁶ and R²⁷ are as defined above in respect to formula Ib-4k, preferably R²⁶ and R²⁷ are independently selected from H, halo, haloalkyl, alkoxy, haloalkoxy, preferably chloro, fluoro, CF₃, or CHF₂, methoxy, OCF₃ or OCHF₂.

In yet another embodiment, preferred compounds of formula I are those of formula Ic

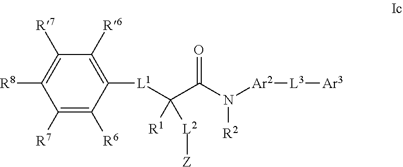

and pharmaceutically acceptable salts, and solvates thereof, wherein
wherein Ar², Ar³, R¹, R², L¹, L², L³ and Z are as defined above in respect to formula I; and
R⁶, R⁷, R′⁶, R′⁷ and R⁸ are independently selected from H, halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, alkoxyalkyl, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoyl alkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or R⁶ and R⁷ or R⁷ and R⁸ or R′⁶ and R′⁷ or R′⁷ and R⁸ together form an alkylenedioxy group or a haloalkylenedioxy group, or R⁶ and R⁷ or R⁷ and R⁸ or R′⁶ and R′⁷ or R′⁷ and R⁸ together form a cycloalkyl, aryl, heterocyclyl or heteroaryl moiety fused to the phenyl group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, preferably R⁶, R⁷, R′⁶, R′⁷ and R⁸ are independently selected from H, halo, cyano, alkyl, hydroxyalkyl, haloalkyl, cycloalkyl, cycloalkylalkyl, heteroalkyl, heterocyclyl, heterocyclylalkyl, aryl, heteroaryl, heteroarylalkyl, hydroxyl, alkoxy, alkoxyalkyl, haloalkoxy, cycloalkyloxy, heterocyclyloxy, aryloxy, carboxy, alkylcarbonylamino, haloalkylcarbonylamino, cycloalkylcarbonylamino, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl, alkylsulfonylamino, cycloalkylsulfonylamino, more preferably R⁶, R⁷, R′⁶, R′⁷ and R⁸ are independently selected from H, hydroxyl, halo, alkyl, haloalkyl, alkoxy, alkoxyalkyl preferably methoxyethyl, haloalkoxy, preferably —OCF₃, alkylsulfonyl, haloalkylsulfonyl and cyano, even more preferably from H, halo, C1-C2 alkyl, CF₃, C1-C2 alkoxy, and cyano, still more preferably from H, F, Cl, CF₃, methyl, methoxy, and cyano, even more preferably R⁶, R⁷, R′⁶, R′⁷ are H and R⁸ is selected from H, Cl, methyl, hydroxyl, and methoxy, and most preferably R⁶, R⁷, R′⁶, R′⁷ are H and R⁸ is selected from H, Cl, methyl, and methoxy.

Preferred compounds of formula Ic are those wherein
Z is —COOH;
R¹ is H;
L² is cyclopropylene, ethenylene, methylene, —CHMe-, —CHF—;
L¹ is as defined above in respect to formula I, preferably methylene, ethylene, or a single bond; and
Ar², Ar³, R², R⁶, R⁷, R′⁶, R′⁷ R⁸ and L³ are as defined above in respect to formula I.

Particularly preferred compounds of formula Ic are those of formula Ic-1

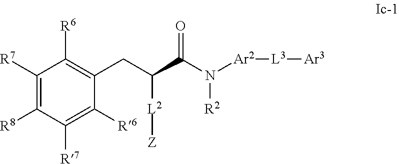

and pharmaceutically acceptable salts, and solvates thereof, wherein
Ar², Ar³, R², R⁶, R⁷, R′⁶, R′⁷ R⁸, L², L³, and Z are as defined above in respect to formula Ic.

Preferred compounds of formula Ic-1 are those wherein
Z is —COOH;
L² is cyclopropylene, ethenylene, methylene, —CHMe-, —CHF—; and
Ar², Ar³, R², R⁶, R⁷, R′⁶, R′⁷ R⁸, and L³ are as defined above in respect to formula Ic.

In yet another embodiment, preferred compounds of formula I are those of formula Id

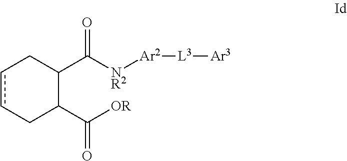

and pharmaceutically acceptable salts, esters, esters, amides, phosphates, and solvates thereof, wherein
the dotted line is present or absent; and
Ar², Ar³, R, R² and L³ are as defined above in respect to formula I.

In one variant of the compounds of formula Id the dotted line is present.

Preferred compounds of formula Id are those of formula Id-1

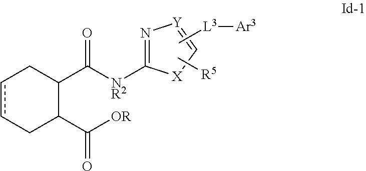

wherein
the dotted line is present or absent, preferably the dotted line is present;
X is S or O;
Y is CH or N;
L³ is attached to the heterocyclic group either in position 4 or 5, preferably in position 4;
If Y is CH, R⁵ is halo, cyano, hydroxyl, linear or branched C₁-C₃ alkyl, C₁-C₃ hydroxyalkyl, C₁-C₃ haloalkyl, preferably F, Cl, or CF₃ and R⁵ is attached to the heterocyclic group either in position 4, if L³ is attached in position 5, or in position 5, if L³ is attached in position 4; preferably R⁵ is attached in position 5;
If Y is N, R⁵ is absent and L³ is attached in position 5; and
Ar³ is as defined above in respect to formula I, preferably Ar³ is an aryl or heteroaryl group, optionally substituted by one or more substituents selected from halogen, C₁-C₄ alkyl, C₁-C₄ haloalkyl, C₁-C₄ alkoxy, cyano, 5 or 6 membered heteroaryl such as pyridinyl, phenyl, methylcarbonylamino, —NH—SO₂CF₃, and L³ is a single bond or C₁-C₂ alkylene; or Ar³ is a C₁-C₄ alkyl group and L³ is a single bond, more preferably Ar³ is an aryl, preferably phenyl, or heteroaryl group, preferably thiophenyl, more preferably thiophen-2-yl, furanyl, more preferably furan-2-yl, each of said aryl or heteroaryl being optionally substituted by one or more substituents selected from halo, C₁-C₄ alkyl, cyclopropyl, C₁-C₄ haloalkyl, C₁-C₄ alkoxy, C₁-C₄ haloalkoxy, cyano, ethoxycarbamoyl, methylenedioxy, 5 or 6 membered aryl, preferably phenyl, 5 or 6 membered heteroaryl, preferably furanyl, thiophenyl, pyridinyl, pyrimidinyl, pyrazinyl, and pyridazinyl, more preferably furan-3-yl, thiophen-3-yl, pyridinyl, still more preferably pyridin-3-yl each of said 5 or 6 membered aryl or 5 or 6 membered heteroaryl being optionally fused to one or more 5 or 6 membered cycloalkyl, aryl, heterocyclyl or heteroaryl moiety, thus forming a fused ring system, and the latter fused ring being optionally substituted by one or more further substituents selected from halo, hydroxyl, oxo, alkyl, and/or each of said 5 or 6 membered aryl or 5 or 6 membered heteroaryl groups being optionally substituted by one or more substituents selected from halo, cyano, hydroxyl, alkyl, cycloalkyl, heterocyclyl, aryl, heteroaryl, aralkyl, heteroarylalkyl, haloalkyl, alkoxy, haloalkoxy, alkoxyalkyl, alkoxyalkoxy, alkylaminoalkoxy, cycloalkyloxy, cycloalkylalkyloxy, heterocyclyloxy, aryloxy, aralkyloxy, alkylamino, alkylaminoalkyl, cycloalkylamino, arylamino, aralkylamino, alkylaminocarbonyl, heteroarylcarbonyl, alkylcarbonylamino, cycloalkylcarbonylamino, alkylsulfonyl, haloalkylsulfonyl, alkylsulfonylamino, each of said cycloalkyl, heterocyclyl, aryl, heteroaryl, aralkyl, heteroarylalkyl, cycloalkyloxy, cycloalkylalkyloxy, heterocyclyloxy, aryloxy, aralkyloxy, heteroarylcarbonyl, cycloalkylamino, arylamino, aralkylamino, cycloalkylcarbonylamino being optionally substituted by one or more further substituents selected from halo, preferably chloro or fluoro, oxo or alkyl, preferably methyl; still more preferably Ar³ is phenyl, thiophenyl, furanyl, preferably phenyl, thiophen-2-yl, furan-2-yl, each of said phenyl, thiophenyl, furanyl, being optionally substituted by one or more substituents selected from halo, C₁-C₄ alkyl, cyclopropyl, C₁-C₄ haloalkyl, C₁-C₄ alkoxy, C₁-C₄ haloalkoxy, cyano, ethoxycarbamoyl, methylenedioxy, phenyl, pyridin-3-yl, each of said phenyl or pyridin-3-yl being optionally fused to one or more 5 or 6 membered heterocyclyl, phenyl, or 5 or 6 membered heteroaryl moiety, preferably oxopyrrolidinyl, imidazolinyl, piperidinyl, morpholinyl, pyrrolyl, imidazolyl, or pyridyl, more preferably 2-oxopyrrolidinyl, 2-oxoimidazolinyl, 2-oxopiperidinyl or pyrrolyl, thus forming a fused ring system, and the latter fused ring being optionally substituted by one or more further substituents selected from halo, preferably chloro or fluoro, oxo, alkyl, preferably methyl, and/or each of said phenyl or pyridin-3-yl groups being optionally substituted by one or more substituents selected from halo, alkyl, heterocyclyl, heteroaryl, haloalkyl, alkoxy, haloalkoxy, alkoxyalkyl, alkoxyalkoxy, cycloalkyloxy, cycloalkylalkyloxy, heterocyclyloxy, aralkyloxy, alkylamino, alkylaminoalkyl, cycloalkylamino, aralkylamino, alkylaminocarbonyl, alkylcarbonylamino, cycloalkylcarbonylamino, each of said heterocyclyl, heteroaryl, cycloalkyloxy, cycloalkylalkyloxy, heterocyclyloxy, aralkyloxy, cycloalkylamino, aralkylamino, cycloalkylcarbonylamino being optionally substituted by one or more further substituents selected from fluoro, chloro, oxo or methyl;
R is as defined above in respect to formula I; and

R² is as defined above in respect to formula I, preferably R² is H, linear or branched C₁-C₄ alkyl, C₁-C₂ hydroxyalkyl, allyl, propargyl, cyclopropyl, cyclopentyl, cyclopentylmethyl, cyclopropylmethyl, benzyl, benzyloxyethyl, methoxyethyl, 1,1,1-trifluoroethyl, —C₂H₄CO₂CH₃, —CH₂CO₂CH₃, or —CH₂CONH₂, more preferably R² is H, methyl, ethyl, allyl, cyclopropyl, hydroxyethyl, —C₂H₄CO₂CH₃, —CH₂CO₂CH₃, or —CH₂CONH₂, more preferably R² is methyl or cyclopropyl.

In still another embodiment, preferred compounds of Formula I are those of formula Ie:

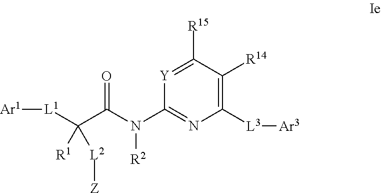

wherein
Y is CH or N; and
R¹⁴ and R¹⁵ are independently H, halo, cyano, hydroxyl, linear or branched C₁-C₃ alkyl, C₁-C₃ hydroxyalkyl, C₁-C₃ haloalkyl, preferably H, F, Cl, or CF₃, more preferably H;
Ar¹ and L¹ are as defined above in respect to formula I, preferably as defined in respect to formula Ib, more preferably Ar¹ is a 5- to 6-membered aryl or heteroaryl group, optionally substituted by one or more groups selected from halogen, trifluoromethyl, cyano, and methoxy, and L¹ is a methylene group, C₁-C₂ alkylene, or C₂ alkenylene; or Ar¹ is a linear or branched C₃-C₆ alkyl group, optionally substituted by one or more groups selected from halogen, trifluoromethyl, cyano, and methoxy, and L¹ is a methylene group;
Ar³ is as defined above in respect to formula I, preferably Ar³ is an aryl or heteroaryl group, optionally substituted by one or more substituents selected from halogen, C₁-C₄ alkyl, C₁-C₄ haloalkyl, C₁-C₄ alkoxy, cyano, 5 or 6 membered heteroaryl such as pyridinyl, phenyl, methylcarbonylamino, —NH—SO₂CF₃, and L³ is a single bond or C₁-C₂ alkylene; or Ar³ is a C₁-C₄ alkyl group and L³ is a single bond, more preferably Ar³ is an aryl, preferably phenyl, or heteroaryl group, preferably thiophenyl, more preferably thiophen-2-yl, furanyl, more preferably furan-2-yl, each of said aryl or heteroaryl being optionally substituted by one or more substituents selected from halo, C₁-C₄ alkyl, cyclopropyl, C₁-C₄ haloalkyl, C₁-C₄ alkoxy, C₁-C₄ haloalkoxy, cyano, ethoxycarbamoyl, methylenedioxy, 5 or 6 membered aryl, preferably phenyl, 5 or 6 membered heteroaryl, preferably furanyl, thiophenyl, pyridinyl, pyrimidinyl, pyrazinyl, and pyridazinyl, more preferably furan-3-yl, thiophen-3-yl, pyridinyl, still more preferably pyridin-3-yl each of said 5 or 6 membered aryl or 5 or 6 membered heteroaryl being optionally fused to one or more 5 or 6 membered cycloalkyl, aryl, heterocyclyl or heteroaryl moiety, thus forming a fused ring system, and the latter fused ring being optionally substituted by one or more further substituents selected from halo, hydroxyl, oxo, alkyl, and/or each of said 5 or 6 membered aryl or 5 or 6 membered heteroaryl groups being optionally substituted by one or more substituents selected from halo, cyano, hydroxyl, alkyl, cycloalkyl, heterocyclyl, aryl, heteroaryl, aralkyl, heteroarylalkyl, haloalkyl, alkoxy, haloalkoxy, alkoxyalkyl, alkoxyalkoxy, alkylaminoalkoxy, cycloalkyloxy, cycloalkylalkyloxy, heterocyclyloxy, aryloxy, aralkyloxy, alkylamino, alkylaminoalkyl, cycloalkylamino, arylamino, aralkylamino, alkylaminocarbonyl, heteroarylcarbonyl, alkylcarbonylamino, cycloalkylcarbonylamino, alkylsulfonyl, haloalkylsulfonyl, alkylsulfonylamino, each of said cycloalkyl, heterocyclyl, aryl, heteroaryl, aralkyl, heteroarylalkyl, cycloalkyloxy, cycloalkylalkyloxy, heterocyclyloxy, aryloxy, aralkyloxy, heteroarylcarbonyl, cycloalkylamino, arylamino, aralkylamino, cycloalkylcarbonylamino being optionally substituted by one or more further substituents selected from halo, preferably chloro or fluoro, oxo or alkyl, preferably methyl; still more preferably Ar³ is phenyl, thiophenyl, furanyl, preferably phenyl, thiophen-2-yl, furan-2-yl, each of said phenyl, thiophenyl, furanyl, being optionally substituted by one or more substituents selected from halo, C₁-C₄ alkyl, cyclopropyl, C₁-C₄ haloalkyl, C₁-C₄ alkoxy, C₁-C₄ haloalkoxy, cyano, ethoxycarbamoyl, methylenedioxy, phenyl, pyridin-3-yl, each of said phenyl or pyridin-3-yl being optionally fused to one or more 5 or 6 membered heterocyclyl, phenyl, or 5 or 6 membered heteroaryl moiety, preferably oxopyrrolidinyl, imidazolinyl, piperidinyl, morpholinyl, pyrrolyl, imidazolyl, or pyridyl, more preferably 2-oxopyrrolidinyl, 2-oxoimidazolinyl 2-oxopiperidinyl, or pyrrolyl, thus forming a fused ring system, and the latter fused ring being optionally substituted by one or more further substituents selected from halo, preferably chloro or fluoro, oxo, alkyl, preferably methyl, and/or each of said phenyl or pyridin-3-yl groups being optionally substituted by one or more substituents selected from halo, alkyl, heterocyclyl, heteroaryl, haloalkyl, alkoxy, haloalkoxy, alkoxyalkyl, alkoxyalkoxy, cycloalkyloxy, cycloalkylalkyloxy, heterocyclyloxy, aralkyloxy, alkylamino, alkylaminoalkyl, cycloalkylamino, aralkylamino, alkylaminocarbonyl, alkylcarbonylamino, cycloalkylcarbonylamino, each of said heterocyclyl, heteroaryl, cycloalkyloxy, cycloalkylalkyloxy, heterocyclyloxy, aralkyloxy, cycloalkylamino, aralkylamino, cycloalkylcarbonylamino being optionally substituted by one or more further substituents selected from fluoro, chloro, oxo or methyl;
R¹ is as defined above in respect to formula I, preferably R¹ is hydrogen, halogen, or a group selected from C₁₋₄ alkyl optionally substituted by one or more substituents selected from halogen or alkyl; more preferably R¹ is selected from hydrogen, fluoro, or methyl or ethyl, the methyl or ethyl group being optionally substituted with one or more substituents selected from fluoro or alkyl, even more preferably R¹ is hydrogen, fluoro or methyl, and most preferably R¹ is hydrogen, and L² is as defined above in respect to formula I, preferably L² is cyclopropylene, ethenylene, n-propylene, or —C(R′R″)—, wherein R′ and R″ are independently selected from H, halogen, methyl, and ethyl, more preferably L² is cyclopropylene, ethenylene, methylene, —CHMe-, —CHF—, even more preferably L² is methylene; or R¹ and L² together are ═CH-under the condition that L¹-Ar¹ is H;
Z is as defined above in respect to formula I, preferably Z is —COOR, wherein R is defined as above in respect to formula I; preferably Z is COOH and
R² is as defined above in respect to formula I, preferably R² is H, linear or branched C₁-C₄ alkyl, C₁-C₂ hydroxyalkyl, allyl, propargyl, cyclopropyl, cyclopentyl, cyclopentylmethyl, cyclopropylmethyl, benzyl, benzyloxyethyl, methoxyethyl, 1,1,1-trifluoroethyl, —C₂H₄CO₂CH₃, —CH₂CO₂CH₃, or —CH₂CONH₂, more preferably R² is H, methyl, ethyl, allyl, cyclopropyl, hydroxyethyl, —C₂H₄CO₂CH₃, —CH₂CO₂CH₃, or —CH₂CONH₂, most preferably R² is methyl or cyclopropyl.
Preferred compounds of formula Ie are those wherein Z is —COOR and R, Ar¹, Ar², Ar³, R¹, R², L¹, L² and L³ are as defined above in respect to formula I, preferably L¹ is a methylene group and Ar¹ is phenyl.
In still another embodiment, preferred compounds of Formula I are those of formula If

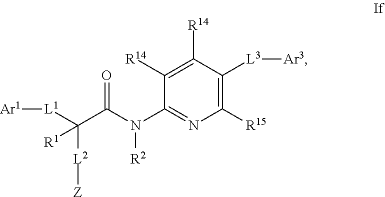

wherein
Ar¹, Ar³, L¹, L², L³, R¹, R², R¹⁴, R¹⁵, Y and Z are as defined above in respect to formula Ie.

In still another embodiment, preferred compounds of Formula I are those of formula Ig:

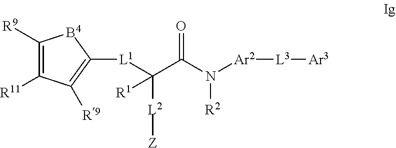

wherein
B⁴ is O or S or N—R^{b} where R^{b} is H or alkyl, preferably linear or branched C₁-C₄ alkyl; C₁-C₄ alkylcarbonyl, C₁-C₄ alkylsulfonyl, C₁-C₄ alkylaminocarbonyl, C₃-C₆ cycloalkyl; preferably O or S, more preferably O,
R⁹, R^{′9}, and R¹¹ are independently selected from H, halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or one of R⁹ or R′⁹ and R¹¹ together form an alkylenedioxy group or a haloalkylenedioxy group, or one of R⁹ or R′⁹ and R¹¹ together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, preferably R⁹, R^{′9}, and R¹¹ are independently selected from H, halo, cyano, alkyl, hydroxyalkyl, haloalkyl, cycloalkyl, cycloalkylalkyl, heteroalkyl, heterocyclyl, heterocyclylalkyl, aryl, heteroaryl, heteroarylalkyl, hydroxyl, alkoxy, haloalkoxy, cycloalkyloxy, heterocyclyloxy, aryloxy, carboxy, alkylcarbonylamino, haloalkylcarbonylamino, cycloalkylcarbonylamino, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl, alkylsulfonylamino, cycloalkylsulfonylamino, or one of R⁹ or R′⁹ and R¹¹ together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, more preferably R⁹, R^{′9}, and R¹¹ are independently selected from H, hydroxyl, C₁-C₃-alkyl, halo, preferably chloro or fluoro, haloalkyl, alkoxy, alkoxyalkyl preferably methoxyethyl, haloalkoxy, preferably —OCF₃, alkylsulfonyl, haloalkylsulfonyl and cyano, even more preferably from H, C₁-C₃-alkyl, halo, CF₃, C₁-C₂ alkoxy, and cyano, and still more preferably from H, F, Cl, methyl, CF₃, methoxy, and cyano, and most preferably H, F or methyl; and
Ar², Ar³, L¹, L², L³, R¹, R², and Z are as defined above in respect to formula I.

In still another embodiment, preferred compounds of Formula I are those of formula Ih:

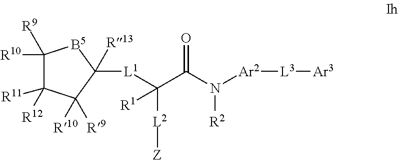

wherein
B⁵ is CH₂ or O preferably O;
R⁹, R¹⁰, R^{′9}, R^{′10}, R¹¹, R¹² and R″¹³ are independently selected from H, halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, alkoxyalkyl, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or one of R¹¹ or R¹² and one of R⁹, R¹⁰, R′⁹ or R′¹⁰, or R¹³ and one of R′⁹ or R′¹⁰ together form an alkylenedioxy group or a haloalkylenedioxy group, or one of R¹¹ or R¹² and one of R⁹, R¹⁰, R′⁹ or R′¹⁰, or R¹³ and one of R′⁹ or R′¹⁰ together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, preferably R⁹, R¹⁰, R¹¹, R¹², R′⁹, R′¹⁰, and R″¹³ are independently selected from H, halo, cyano, alkyl, hydroxyalkyl, haloalkyl, cycloalkyl, cycloalkylalkyl, heteroalkyl, heterocyclyl, heterocyclylalkyl, aryl, heteroaryl, heteroarylalkyl, hydroxyl, alkoxy, haloalkoxy, cycloalkyloxy, heterocyclyloxy, aryloxy, carboxy, alkylcarbonylamino, haloalkylcarbonylamino, cycloalkylcarbonylamino, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl, alkylsulfonylamino, cycloalkylsulfonylamino, or one of R¹¹ or R¹² and one of R⁹, R¹⁰, R′⁹ or R′¹⁰, or R¹³ and one of R′⁹ or R′¹⁰ together form an alkylenedioxy group or a haloalkylenedioxy group, or one of R¹¹ or R¹² and one of R⁹, R¹⁰, R′⁹ or R′¹⁰, or R¹³ and one of R′⁹ or R′¹⁰ together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, more preferably R⁹, R¹⁰, R¹¹, R¹², R¹³, R′⁹, R′¹⁰, R′¹¹, R′¹², R′¹³ and R″¹³ are independently selected from H, hydroxyl, C₁-C₃-alkyl, halo, preferably chloro or fluoro, haloalkyl, alkoxy, alkoxyalkyl preferably methoxyethyl, haloalkoxy, preferably —OCF₃, alkylsulfonyl, haloalkylsulfonyl and cyano, even more preferably from H, C₁-C₃-alkyl, halo, CF₃, C₁-C₂ alkoxy, preferably methoxy, and cyano, and still more preferably from H, F, Cl, methyl, CF₃, methoxy, and cyano, and most preferably H or methyl; and
Ar², Ar³, L¹, L², L³, R¹, R², and Z are as defined above in respect to formula I.

In still another embodiment, preferred compounds of Formula I are those of formula Ii

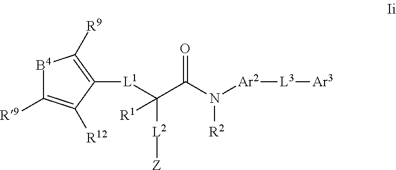

wherein
B⁴ is as defined above in respect to formula Ig,
R⁹, R^{′9} and R¹² independently selected from H, halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, alkoxyalkyl, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoyl alkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or R⁹ and R¹² together form an alkylenedioxy group or a haloalkylenedioxy group, or R⁹ and R¹² together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, preferably R⁹, R′⁹, and R¹², are independently selected from H, halo, cyano, alkyl, hydroxyalkyl, haloalkyl, cycloalkyl, cycloalkylalkyl, heteroalkyl, heterocyclyl, heterocyclylalkyl, aryl, heteroaryl, heteroarylalkyl, hydroxyl, alkoxy, haloalkoxy, haloalkoxy, cycloalkyloxy, heterocyclyloxy, aryloxy, carboxy, alkylcarbonylamino, haloalkylcarbonylamino, cycloalkylcarbonylamino, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl, alkylsulfonylamino, cycloalkylsulfonylamino, or R⁹ and R¹² together form an alkylenedioxy group or a haloalkylenedioxy group, or R⁹ and R¹² together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, more preferably R⁹, R′⁹, and R¹², are independently selected from H, hydroxyl, C₁-C₃-alkyl, halo, preferably chloro or fluoro, haloalkyl, alkoxy, alkoxyalkyl preferably methoxyethyl, haloalkoxy, preferably —OCF₃, alkylsulfonyl, haloalkylsulfonyl and cyano, even more preferably from H, C₁-C₃-alkyl, halo, CF₃, C₁-C₂ alkoxy, preferably methoxy, and cyano, and still more preferably from H, F, Cl, methyl, CF₃, methoxy, and cyano, and most preferably H or methyl; and
Ar², Ar³, L¹, L², L³, R¹, R², and Z are as defined above in respect to formula I.

In still another embodiment, preferred compounds of Formula I are those of formula Ij:

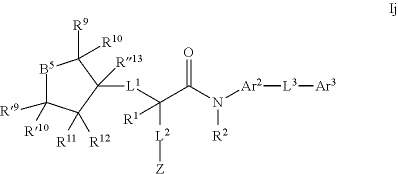

wherein
B⁵ is as defined above in respect to formula Ih,
R⁹, R^{′9}, R¹⁰, R^{′10}, R¹¹, R¹² and R″¹³ are independently selected from H, halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, alkoxyalkyl, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or one of R¹¹ or R¹² and one of R⁹, R¹⁰, R′⁹ or R′¹⁰, or one of R′⁹ or R′¹⁰ and R″¹³ together form an alkylenedioxy group or a haloalkylenedioxy group, or one of R¹¹ or R¹² and one of R⁹, R¹⁰, R′⁹ or R′¹⁰, or one of R′⁹ or R′¹⁰ and R″¹³ together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, preferably R⁹, R^{′9}, R¹⁰, R^{′10}, R¹¹, R¹² and R″¹³ are independently selected from H, halo, cyano, alkyl, hydroxyalkyl, haloalkyl, cycloalkyl, cycloalkylalkyl, heteroalkyl, heterocyclyl, heterocyclylalkyl, aryl, heteroaryl, heteroarylalkyl, hydroxyl, alkoxy, haloalkoxy, cycloalkyloxy, heterocyclyloxy, aryloxy, carboxy, alkylcarbonylamino, haloalkylcarbonylamino, cycloalkylcarbonylamino, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl, alkylsulfonylamino, cycloalkylsulfonylamino, or one of R¹¹ or R¹² and one of R⁹, R¹⁰, R′⁹ or R′¹⁰, or one of R′⁹ or R′¹⁰ and R″¹³ together form an alkylenedioxy group or a haloalkylenedioxy group, or one of R¹¹ or R¹² and one of R⁹, R¹⁰, R′⁹ or R′¹⁰, or one of R′⁹ or R′¹⁰ and R″¹³ together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, more preferably R⁹, R^{′9}, R¹⁰, R^{′10}, R¹¹, R¹² and R″¹³ are independently selected from H, hydroxyl, C₁-C₃-alkyl, halo, preferably chloro or fluoro, haloalkyl, alkoxy, alkoxyalkyl preferably methoxyethyl, haloalkoxy, preferably —OCF₃, alkylsulfonyl, haloalkylsulfonyl and cyano, even more preferably from H, C₁-C₃-alkyl, halo, CF₃, C₁-C₂ alkoxy, preferably methoxy, and cyano, and still more preferably from H, F, Cl, methyl, CF₃, methoxy, and cyano, and most preferably H or methyl; and
Ar², Ar³, L¹, L², L³, R¹, R², and Z are as defined above in respect to formula I.

In still another embodiment, preferred compounds of Formula I are those of formula Ik:

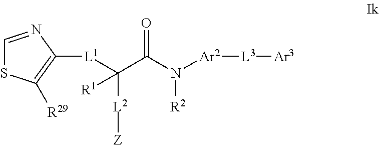

wherein
R²⁹ is H, halo, alkyl, haloalkyl preferably —CF₃ or —CF₂H, alkoxy, haloalkoxy preferably —OCF₃ or —OCF₂H, cyano, preferably R²⁹ is H, F, —CF₃, alkyl preferably methyl, more preferably R²⁹ is H, F or methyl; and
Ar², Ar³, L¹, L², L³, R¹, R², and Z are as defined above in respect to formula I.

In still another embodiment, preferred compounds of Formula I are those of formula Il:

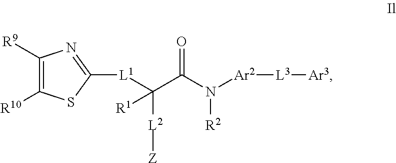

wherein
R⁹ and R¹⁰ are independently selected from H, halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, alkoxyalkyl, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or R⁹ and R¹⁰ together form an alkylenedioxy group or a haloalkylenedioxy group, or R⁹ and R¹⁰ together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, preferably R⁹ and R¹⁰ are independently selected from H, halo, cyano, alkyl, hydroxyalkyl, haloalkyl, cycloalkyl, cycloalkylalkyl, heteroalkyl, heterocyclyl, heterocyclylalkyl, aryl, heteroaryl, heteroarylalkyl, hydroxyl, alkoxy, haloalkoxy, cycloalkyloxy, heterocyclyloxy, aryloxy, carboxy, alkylcarbonylamino, haloalkylcarbonylamino, cycloalkylcarbonylamino, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl, alkylsulfonylamino, cycloalkylsulfonylamino, or R⁹ and R¹⁰ together form an alkylenedioxy group or a haloalkylenedioxy group, or one of R⁹ and R¹⁰ together form a cycloalkyl, aryl, heterocyclyl or heteroaryl moiety together with the cyclic group they are attached to, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl, more preferably R⁹ and R¹⁰ are independently selected from H, hydroxyl, C₁-C₃-alkyl, halo, preferably chloro or fluoro, haloalkyl, alkoxy, alkoxyalkyl preferably methoxyethyl, haloalkoxy, preferably —OCF₃, alkylsulfonyl, haloalkylsulfonyl and cyano, even more preferably from H, C₁-C₃-alkyl, halo, CF₃, C₁-C₂ alkoxy, preferably methoxy, and cyano, and still more preferably from H, F, Cl, methyl, CF₃, methoxy, and cyano, and most preferably H or methyl; and
Ar², Ar³, L¹, L², L³, R¹, R², and Z are as defined above in respect to formula I.

Particularly preferred compounds of the invention are those listed in Table 1 hereafter:

TABLE 1

| Com- | | |
|---|---|---|
| pound | | (M + |
| number | Compound name | H)+ |
| 1 | 6-((4-(2-chlorophenyl)thiazol-2- | 363.83 |
| | yl)carbamoyl)cyclohex-3-enecarboxylic acid | |
| 2 | (R)-3-benzyl-4-((4-(2-chlorophenyl)thiazol-2- | 401.88 |
| | yl)amino)-4-oxobutanoic acid | |
| 3 | (R)-3-benzyl-4-((4-(2,4-dichlorophenyl)thiazol-2- | 436.3 |
| | yl)amino)-4-oxobutanoic acid | |
| 4 | (R)-3-benzyl-4-((4-(2-fluorophenyl)thiazol-2- | 385.4 |
| | yl)amino)-4-oxobutanoic acid | |
| 5 | (R)-3-benzyl-4-((4-(3,4-dichlorophenyl)thiazol-2- | 436.3 |
| | yl)amino)-4-oxobutanoic acid | |
| 8 | (R)-3-benzyl-4-((4-(4-cyanophenyl)thiazol-2- | 392.4 |
| | yl)amino)-4-oxobutanoic acid | |
| 9 | (S)-4-((4-(2-chlorophenyl)thiazol-2-yl)amino)-4-oxo- | 387.9 |
| | 3-phenylbutanoic acid | |
| 10 | (Z)-4-((4-(2-chlorophenyl)thiazol-2-yl)amino)-4- | 309.7 |
| | oxobut-2-enoic acid | |
| 11 | (R)-3-benzyl-4-oxo-4-((3-phenyl-1,2,4-thiadiazol-5- | 368.4 |
| | yl)amino)butanoic acid | |
| 12 | (R)-3-benzyl-4-((4-(3-chlorophenyl)thiazol-2- | 401.9 |
| | yl)amino)-4-oxobutanoic acid | |
| 13 | (R)-3-benzyl-4-oxo-4-((4-(3- | 435.4 |
| | (trifluoromethyl)phenyl)thiazol-2-yl)amino)butanoic | |
| | acid | |
| 14 | (R)-3-benzyl-4-((4-(2-chlorophenyl)thiazol-2- | 415.9 |
| | yl)(methyl)amino)-4-oxobutanoic acid | |
| 15 | (R)-3-benzyl-4-((5-(2-chlorophenyl)pyridin-2- | 395.9 |
| | yl)amino)-4-oxobutanoic acid | |
| 16 | (R)-3-((4-(2-chlorophenyl)thiazol-2- | 367.9 |
| | yl)carbamoyl)heptanoic acid | |
| 17 | (R)-4-((4-(2-chlorophenyl)thiazol-2-yl)amino)-3-(4- | 419.9 |
| | fluorobenzyl)-4-oxobutanoic acid | |
| 18 | (R)-4-((4-(2-chlorophenyl)thiazol-2-yl)amino)-3- | 407.9 |
| | (cyclohexylmethyl)-4-oxobutanoic acid | |
| 19 | (R)-3-((4-(2-chlorophenyl)thiazol-2-yl)carbamoyl)-5- | 367.9 |
| | methylhexanoic acid | |
| 20 | (R)-3-benzyl-4-((4-(2-chlorophenyl)-5-fluorothiazol- | 419.9 |
| | 2-yl)amino)-4-oxobutanoic acid | |
| 21 | (R)-3-benzyl-4-((5-chloro-4-(2-chlorophenyl)thiazol- | 450.4 |
| | 2-yl)(methyl)amino)-4-oxobutanoic acid | |
| 22 | (R)-4-(allyl(4-(2-chlorophenyl)thiazol-2-yl)amino)-3- | 441.9 |
| | benzyl-4-oxobutanoic acid | |
| 23 | (R)-3-benzyl-4-((4-(2-chlorophenyl)thiazol-2-yl)(2- | 473.9 |
| | methoxy-2-oxoethyl)amino)-4-oxobutanoic acid | |
| 24 | (R)-methyl-3-benzyl-4-((4-(2-chlorophenyl)thiazol- | 415.9 |
| | 2-yl)amino)-4-oxobutanoate | |
| 26 | (R)-3-(4-(2-chlorophenyl)thiazol-2-ylcarbamoyl)-5- | 415.9 |
| | phenylpentanoic acid | |
| 27 | (S)-3-(4-(2-chlorophenyl)thiazol-2-ylcarbamoyl)-5- | 415.9 |
| | phenylpentanoic acid | |
| 28 | (R)-4-(4-(2-chlorophenyl)thiazol-2-ylamino)-4-oxo- | 469.9 |
| | 3-(4-(trifluoromethyl)benzyl)butanoic acid | |
| 29 | (R)-4-((4-(2-chlorophenyl)thiazol-2-yl)amino)-4- | 469.9 |
| | oxo-3-(3-(trifluoromethyl)benzyl)butanoic acid | |
| 30 | (R)-4-((4-(2-chlorophenyl)thiazol-2-yl)amino)-3-(2- | 426.9 |
| | cyanobenzyl)-4-oxobutanoic acid | |
| 31 | (R)-4-((4-(2-chlorophenyl)thiazol-2-yl)amino)-3-(3- | 426.9 |
| | cyanobenzyl)-4-oxobutanoic acid | |
| 32 | (R)-4-((4-(2-chlorophenyl)thiazol-2-yl)amino)-3-(4- | 426.9 |
| | cyanobenzyl)-4-oxobutanoic acid | |
| 33 | (R)-4-((4-(2-chlorophenyl)thiazol-2-yl)amino)-3-(4- | 431.9 |
| | methoxybenzyl)-4-oxobutanoic acid | |
| 34 | (R)-4-((4-(2-chlorophenyl)thiazol-2-yl)amino)-3-(3- | 431.9 |
| | methoxybenzyl)-4-oxobutanoic acid | |
| 35 | (R)-4-((4-(2-chlorophenyl)thiazol-2-yl)amino)-3-(2- | 431.9 |
| | methoxybenzyl)-4-oxobutanoic acid | |
| 36 | (R)-3-benzyl-4-((4-(2-methoxyphenyl)thiazol-2- | 397.5 |
| | yl)amino)-4-oxobutanoic acid | |
| 37 | (R)-3-benzyl-4-oxo-4-(4-(2,4,6- | 470.8 |
| | trichlorophenyl)thiazol-2-ylamino)butanoic acid | |
| 38 | (R)-4-benzyl-5-((4-(2-chlorophenyl)thiazol-2- | 429.9 |
| | yl)(methyl)amino)-5-oxopentanoic acid | |
| 39 | (S)-4-benzyl-5-((4-(2-chlorophenyl)thiazol-2- | 429.9 |
| | yl)(methyl)amino)-5-oxopentanoic acid | |
| 40 | (R)-methyl 4-benzyl-5-(4-(2-chlorophenyl)thiazol-2- | 429.9 |
| | ylamino)-5-oxopentanoate | |
| 41 | (S)-methyl 4-benzyl-5-(4-(2-chlorophenyl)thiazol-2- | 429.9 |
| | ylamino)-5-oxopentanoate | |
| 42 | (R)-3-benzyl-4-((4-(2-chlorophenyl)thiazol-2- | 456.0 |
| | yl)(cyclopropylmethyl)amino)-4-oxobutanoic acid | |
| 43 | (R)-3-benzyl-4-(benzyl(4-(2-chlorophenyl)thiazol-2- | 492.0 |
| | yl)amino)-4-oxobutanoic acid | |
| 44 | (R)-3-benzyl-4-((4-(2-chlorophenyl)thiazol-2- | 483.9 |
| | yl)(2,2,2-trifluoroethyl)amino)-4-oxobutanoic acid | |
| 45 | (R)-4-((4-(2-chlorophenyl)thiazol-2- | 445.9 |
| | yl)(methyl)amino)-3-(4-methoxybenzyl)-4- | |
| | oxobutanoic acid | |
| 46 | (R)-4-((4-(2-chlorophenyl)thiazol-2- | 445.9 |
| | yl)(methyl)amino)-3-(3-methoxybenzyl)-4- | |
| | oxobutanoic acid | |
| 47 | (R)-4-((4-(2-chlorophenyl)thiazol-2- | 445.9 |
| | yl)(methyl)amino)-3-(2-methoxybenzyl)-4- | |
| | oxobutanoic acid | |
| 48 | (R)-4-((4-(2-chlorophenyl)thiazol-2- | 440.9 |
| | yl)(methyl)amino)-3-(4-cyanobenzyl)-4-oxobutanoic | |
| | acid | |
| 49 | (R)-4-((4-(2-chlorophenyl)thiazol-2- | 440.9 |
| | yl)(methyl)amino)-3-(3-cyanobenzyl)-4-oxobutanoic | |
| | acid | |
| 50 | (R)-4-((4-(2-chlorophenyl)thiazol-2- | 440.9 |
| | yl)(methyl)amino)-3-(2-cyanobenzyl)-4-oxobutanoic | |
| | acid | |
| 51 | (R)-3-(4-chlorobenzyl)-4-((4-(2- | 450.4 |
| | chlorophenyl)thiazol-2-yl)(methyl)amino)-4- | |
| | oxobutanoic acid | |
| 52 | (R)-3-(3-chlorobenzyl)-4-((4-(2- | 450.4 |
| | chlorophenyl)thiazol-2-yl)(methyl)amino)-4- | |
| | oxobutanoic acid | |
| 53 | (R)-3-(2-chlorobenzyl)-4-((4-(2- | 450.4 |
| | chlorophenyl)thiazol-2-yl)(methyl)amino)-4- | |
| | oxobutanoic acid | |
| 54 | (3S)-4-((4-(2-chlorophenyl)thiazol-2- | 441.9 |
| | yl)(methyl)amino)-3-(2,3-dihydro-1H-inden-1-yl)-4- | |
| | oxobutanoic acid | |
| 55 | (S)-4-((4-(2-chlorophenyl)thiazol-2- | 441.9 |
| | yl)(methyl)amino)-3-(2,3-dihydro-1H-inden-2-yl)-4- | |
| | oxobutanoic acid | |
| 56 | (R)-4-(benzo[d]thiazol-2-yl(methyl)amino)-3-benzyl- | 355.4 |
| | 4-oxobutanoic acid | |
| 57 | (R)-4-(benzo[d]oxazol-2-yl(methyl)amino)-3-benzyl- | 339.4 |
| | 4-oxobutanoic acid | |
| 58 | (R)-2-((1H-tetrazol-5-yl)methyl)-N-(4-(2- | 439.9 |
| | chlorophenyl)thiazol-2-yl)-N-methyl-3- | |
| | phenylpropanamide | |
| 59 | (R)-2-benzyl-N-(4-(2-chlorophenyl)thiazol-2-yl)-N- | 455.9 |
| | methyl-3-(5-oxo-4,5-dihydro-1,2,4-oxadiazol-3- | |
| | yl)propanamide | |
| 60 | (R)-3-benzyl-4-((4-(2-chlorophenyl)-5-fluorothiazol- | 433.9 |
| | 2-yl)(methyl)amino)-4-oxobutanoic acid | |
| 61 | (S)-4-(4-(2-chlorophenyl)thiazol-2-ylamino)-3- | 393.9 |
| | cyclohexyl-4-oxobutanoic acid | |
| 62 | (S)-4-((4-(2-chlorophenyl)thiazol-2- | 407.9 |
| | yl)(methyl)amino)-3-cyclohexyl-4-oxobutanoic acid | |
| 63 | (S)-4-((4-(2-chlorophenyl)thiazol-2- | 401.9 |
| | yl)(methyl)amino)-4-oxo-3-phenylbutanoic acid | |
| 64 | (3R)-3-(4-(2-chlorophenyl)thiazol-2-ylcarbamoyl)-4- | 415.9 |
| | phenylpentanoic acid | |
| 65 | (R)-2-((1H-tetrazol-5-yl)methyl)-N-(4-(2- | 425.9 |
| | chlorophenyl)thiazol-2-yl)-3-phenylpropanamide | |
| 66 | (R)-2-benzyl-N-(4-(2-chlorophenyl)thiazol-2-yl)-3- | 441.9 |
| | (5-oxo-4,5-dihydro-1,2,4-oxadiazol-3- | |
| | yl)propanamide | |
| 68 | (3R)-3-benzyl-4-(4-(2-chlorophenyl)thiazol-2- | 415.9 |
| | ylamino)-2-methyl-4-oxobutanoic acid | |
| 69 | (R)-2-benzyl-N-(4-(2-chlorophenyl)thiazol-2-yl)-3- | 440.9 |
| | (3-hydroxyisoxazol-5-yl)propanamide | |
| 70 | (R)-3-benzyl-4-(4-(2-chlorophenyl)pyrimidin-2- | 396.8 |
| | ylamino)-4-oxobutanoic acid | |
| 71 | (R)-3-benzyl-4-(6-(2-chlorophenyl)pyridin-2- | 395.9 |
| | ylamino)-4-oxobutanoic acid | |
| 72 | (E)-3-(4-(2-chlorophenyl)thiazol-2-ylcarbamoyl)-4- | 399.9 |
| | phenylbut-3-enoic acid | |
| 74 | (Z)-4-((4-(2-chlorophenyl)thiazol-2- | 399.9 |
| | yl)(methyl)amino)-4-oxo-3-phenylbut-2-enoic acid | |
| 75 | (R)-3-(N-(4-(2-chlorophenyl)thiazol-2-yl)-N- | 452.0 |
| | methylsulfamoyl)-4-phenylbutanoic acid | |
| 76 | (S)-3-(N-(4-(2-chlorophenyl)thiazol-2-yl)-N- | 452.0 |
| | methylsulfamoyl)-4-phenylbutanoic acid | |
| 79 | (R)-3-benzyl-4-(4-(2-chlorophenyl)thiazol-2- | 419.9 |
| | ylamino)-3-fluoro-4-oxobutanoic acid | |
| 80 | (R)-3-benzyl-3-(4-(2-chlorophenyl)thiazol-2- | 441.9 |
| | ylcarbamoyl)hex-5-enoic acid | |
| 81 | (E)-3-((4-(2-chlorophenyl)thiazol-2- | 413.9 |
| | yl)(methyl)carbamoyl)-4-phenylbut-3-enoic acid | |
| 82 | (3S)-3-((4-(2-chlorophenyl)thiazol-2- | 429.9 |
| | yl)(methyl)carbamoyl)-4-phenylpentanoic acid | |
| 83 | (R)-3-benzyl-4-((3-(2-chlorophenyl)-1,2,4-thiadiazol- | 416.9 |
| | 5-yl)(methyl)amino)-4-oxobutanoic acid | |
| 84 | (R)-3-benzyl-4-((3-(2-chlorophenyl)-1,2,4-oxadiazol- | 400.8 |
| | 5-yl)(methyl)amino)-4-oxobutanoic acid | |
| 85 | (R)-3-benzyl-4-((1-(2-chlorophenyl)-1H-pyrazol-3- | 398.9 |
| | yl)(methyl)amino)-4-oxobutanoic acid | |
| 86 | (R)-2-benzyl-N-(4-(2-chlorophenyl)thiazol-2-yl)-3- | 454.9 |
| | (3-hydroxyisoxazol-5-yl)-N-methylpropanamide | |
| 89 | (R)-4-((4-(2-chlorophenyl)thiazol-2- | 422 |
| | yl)(methyl)amino)-3-(cyclohexylmethyl)-4- | |
| | oxobutanoic acid | |
| 90 | (R)-3-((4-(2-chlorophenyl)thiazol-2- | 381.9 |
| | yl)(methyl)carbamoyl)-5-methylhexanoic acid | |
| 91 | (R)-3-benzyl-4-((4-(2-cyanophenyl)thiazol-2- | 406.5 |
| | yl)(methyl)amino)-4-oxobutanoic acid | |
| 92 | (R)-4-(4-(2-chlorophenyl)thiazol-2-ylamino)-4-oxo- | 387.9 |
| | 3-phenylbutanoic acid | |
| 93 | (R)-4-(4-(2-chlorophenyl)thiazol-2-ylamino)-3-(3- | 419.9 |
| | fluorobenzyl)-4-oxobutanoic acid | |
| 94 | (S)-3-((4-(2-chlorophenyl)thiazol-2- | 367.9 |
| | yl)(methyl)carbamoyl)-4-methylpentanoic acid | |
| 95 | 4-((4-(2-chlorophenyl)thiazol-2-yl)(methyl)amino)-4- | 423.9 |
| | oxo-3-((tetrahydro-2H-pyran-4-yl)methyl)butanoic | |
| | acid | |
| 96 | (R)-3-benzyl-4-((4-(2-chlorophenyl)thiazol-2- | 429.9 |
| | yl)(ethyl)amino)-4-oxobutanoic acid | |
| 97 | (R)-3-benzyl-4-((4-(2-chlorophenyl)thiazol-2- | 441.9 |
| | yl)(cyclopropyl)amino)-4-oxobutanoic acid | |
| 98 | cis-6-(4-(2-chlorophenyl)thiazol-2- | 363.8 |
| | ylcarbamoyl)cyclohex-3-enecarboxylic acid | |
| 99 | 4-((4-(2-chlorophenyl)thiazol-2-yl)(methyl)amino)-3- | 445.9 |
| | (4-methoxybenzyl)-4-oxobutanoic acid | |
| 100 | cis-6-((4-(2-chlorophenyl)thiazol-2- | 377.9 |
| | yl)(methyl)carbamoyl)cyclohex-3-enecarboxylic acid | |
| 101 | cis-2-((4-(2-chlorophenyl)thiazol-2- | 379.9 |
| | yl)(methyl)carbamoyl)cyclohexanecarboxylic acid | |
| 102 | (R)-3-benzyl-4-(4-(2,5-dimethylthiophen-3- | 401.5 |
| | yl)thiazol-2-ylamino)-4-oxobutanoic acid | |
| 103 | 4-((4-(2-chlorophenyl)thiazol-2-yl)(methyl)amino)-3- | 422.0 |
| | (cyclohexylmethyl)-4-oxobutanoic acid | |
| 105 | 4-((4-(2-chlorophenyl)thiazol-2-yl)(methyl)amino)-3- | 407.9 |
| | (cyclopentylmethyl)-4-oxobutanoic acid | |
| 106 | (3S,4R)-3-((4-(2-chlorophenyl)thiazol-2- | 429.9 |
| | yl)(methyl)carbamoyl)-4-phenylpentanoic acid | |
| 107 | (R)-3-benzyl-4-(methyl(4-(2-(thiophen-3- | 463.6 |
| | yl)phenyl)thiazol-2-yl)amino)-4-oxobutanoic acid | |
| 108 | (R)-3-benzyl-4-((4-(2-(6-chloropyridin-3- | 493.0 |
| | yl)phenyl)thiazol-2-yl)(methyl)amino)-4-oxobutanoic | |
| | acid | |
| 109 | (R)-4-((4-(2-chlorophenyl)thiazol-2- | 416.9 |
| | yl)(methyl)amino)-4-oxo-3-(phenylamino)butanoic | |
| | acid | |
| 110 | 4-((4-(2-chlorophenyl)thiazol-2-yl)(methyl)amino)-3- | 429.9 |
| | (4-methylbenzyl)-4-oxobutanoic acid | |
| 111 | (R)-4-((4-([1,1′-biphenyl]-2-yl)thiazol-2- | 457.6 |
| | yl)(methyl)amino)-3-benzyl-4-oxobutanoic acid | |
| 112 | (R)-3-benzyl-4-(4-(2,5-dichlorothiophen-3-yl)thiazol- | 442.4 |
| | 2-ylamino)-4-oxobutanoic acid | |
| 113 | 4-((4-(2-chlorophenyl)thiazol-2-yl)(methyl)amino)-3- | 379.9 |
| | (cyclopropylmethyl)-4-oxobutanoic acid | |
| 114 | 4-((4-(2-chlorophenyl)thiazol-2-yl)(methyl)amino)-4- | 422.9 |
| | oxo-3-(thiazol-4-ylmethyl)butanoic acid | |
| 115 | (R)-3-benzyl-4-((4-(2-(6-(dimethylamino)pyridin-3- | 501.6 |
| | yl)phenyl)thiazol-2-yl)(methyl)amino)-4-oxobutanoic | |
| | acid | |
| 116 | (R)-3-benzyl-4-((4-(2-(6-methoxypyridin-3- | 488.6 |
| | yl)phenyl)thiazol-2-yl)(methyl)amino)-4-oxobutanoic | |
| | acid | |
| 117 | (R)-3-benzyl-4-((4-(2-(2-methoxypyridin-3- | 488.6 |
| | yl)phenyl)thiazol-2-yl)(methyl)amino)-4-oxobutanoic | |
| | acid | |
| 118 | (R)-3-benzyl-4-((4-(2- | 468.5 |
| | ((ethoxycarbonyl)amino)phenyl)thiazol-2- | |
| | yl)(methyl)amino)-4-oxobutanoic acid | |
| 119 | (R)-3-benzyl-4-((4-(2-(6-fluoropyridin-3- | 476.5 |
| | yl)phenyl)thiazol-2-yl)(methyl)amino)-4-oxobutanoic | |
| | acid | |
| 120 | (R)-3-benzyl-4-(methyl(4-(2-(6-methylpyridin-3- | 472.6 |
| | yl)phenyl)thiazol-2-yl)amino)-4-oxobutanoic acid | |
| 121 | (R)-4-((2-amino-2-oxoethyl)(4-(2- | 458.9 |
| | chlorophenyl)thiazol-2-yl)amino)-3-benzyl-4- | |
| | oxobutanoic acid | |
| 122 | (R)-3-benzyl-4-oxo-4-((4-(3- | 451.4 |
| | (trifluoromethoxy)phenyl)thiazol-2- | |
| | yl)amino)butanoic acid | |
| 123 | (R)-3-benzyl-4-((4-(2,5-dichlorophenyl)thiazol-2- | 436.3 |
| | yl)amino)-4-oxobutanoic acid | |
| 124 | (R)-3-benzyl-4-((4-(3-chloro-4-fluorophenyl)thiazol- | 419.9 |
| | 2-yl)amino)-4-oxobutanoic acid | |
| 125 | (R)-3-benzyl-4-((4-(3-chloro-4- | 431.9 |
| | methoxyphenyl)thiazol-2-yl)amino)-4-oxobutanoic | |
| | acid | |
| 126 | (R)-3-benzyl-4-((4-(2-chlorophenyl)thiazol-2-yl)(3- | 488.0 |
| | methoxy-3-oxopropyl)amino)-4-oxobutanoic acid | |
| 127 | 3-(bicyclo[2.2.1]heptan-2-ylmethyl)-4-((4-(2- | 434.0 |
| | chlorophenyl)thiazol-2-yl)(methyl)amino)-4- | |
| | oxobutanoic acid | |
| 128 | (R)-3-benzyl-4-((4-(2-(6-ethoxypyridin-3- | 502.6 |
| | yl)phenyl)thiazol-2-yl)(methyl)amino)-4-oxobutanoic | |
| | acid | |
| 129 | (R)-3-benzyl-4-((4-(4′-methoxy-[1,1′-biphenyl]-2- | 487.6 |
| | yl)thiazol-2-yl)(methyl)amino)-4-oxobutanoic acid | |
| 130 | (R)-3-benzyl-4-((4-(2,5-dichlorophenyl)thiazol-2- | 450.4 |
| | yl)(methyl)amino)-4-oxobutanoic acid | |
| 131 | (R)-1-(5-(2-(2-(2-benzyl-3-carboxy-N- | 641.7 |
| | methylpropanamido)thiazol-4-yl)phenyl)pyridin-2- | |
| | yl)pyrrolidin-1-ium 2,2,2-trifluoroacetate | |
| 132 | (R)-4-(2′-(2-(2-benzyl-3-carboxy-N- | 656.7 |
| | methylpropanamido)thiazol-4-yl)-[1,1′-biphenyl]-4- | |
| | yl)morpholin-4-ium 2,2,2-trifluoroacetate | |
| 133 | (R)-3-benzyl-4-(methyl(4-(2-(6-morpholinopyridin- | 543.6 |
| | 3-yl)phenyl)thiazol-2-yl)amino)-4-oxobutanoic acid | |
| 134 | (R)-3-benzyl-4-((4-(3′-chloro-[1,1′-biphenyl]-2- | 492.0 |
| | yl)thiazol-2-yl)(methyl)amino)-4-oxobutanoic acid | |
| 135 | (R)-3-benzyl-4-((4-(2-(furan-3-yl)phenyl)thiazol-2- | 447.5 |
| | yl)(methyl)amino)-4-oxobutanoic acid | |
| 136 | (R)-3-benzyl-4-((4-(2-(6-(2-methoxyethoxy)pyridin- | 532.6 |
| | 3-yl)phenyl)thiazol-2-yl)(methyl)amino)-4- | |
| | oxobutanoic acid | |
| 138 | (R)-3-benzyl-4-((4-(4′-isopropyl-[1,1′-biphenyl]-2- | 499.6 |
| | yl)thiazol-2-yl)(methyl)amino)-4-oxobutanoic acid | |
| 139 | (R)-3-(cyclopentylmethyl)-4-((4-(2-(6- | 480.6 |
| | methoxypyridin-3-yl)phenyl)thiazol-2- | |
| | yl)(methyl)amino)-4-oxobutanoic acid | |
| 140 | (R)-3-benzyl-4-((4-(2-(5-fluoro-6-methoxypyridin-3- | 506.6 |
| | yl)phenyl)thiazol-2-yl)(methyl)amino)-4-oxobutanoic | |
| | acid | |
| 141 | (R)-3-benzyl-4-(methyl(4-(2-(6-((tetrahydro-2H- | 558.7 |
| | pyran-4-yl)oxy)pyridin-3-yl)phenyl)thiazol-2- | |
| | yl)amino)-4-oxobutanoic acid | |
| 142 | (R)-3-benzyl-4-(cyclopropyl(4-(2,5- | 476.4 |
| | dichlorophenyl)thiazol-2-yl)amino)-4-oxobutanoic | |
| | acid | |
| 143 | 4-((4-(2-chlorophenyl)thiazol-2-yl)(methyl)amino)-3- | 405.9 |
| | (furan-2-ylmethyl)-4-oxobutanoic acid | |
| 144 | (R)-3-benzyl-4-((4-(2-cyclopropylphenyl)thiazol-2- | 421.5 |
| | yl)(methyl)amino)-4-oxobutanoic acid | |
| 145 | (R)-3-benzyl-4-((4-(4′-(dimethylamino)-[1,1′- | 500.6 |
| | biphenyl]-2-yl)thiazol-2-yl)(methyl)amino)-4- | |
| | oxobutanoic acid | |
| 146 | (R)-3-benzyl-4-((4-(3′-fluoro-[1,1′-biphenyl]-2- | 475.5 |
| | yl)thiazol-2-yl)(methyl)amino)-4-oxobutanoic acid | |
| 147 | (R)-3-benzyl-4-((4-(3′,5′-difluoro-[1,1′-biphenyl]-2- | 493.5 |
| | yl)thiazol-2-yl)(methyl)amino)-4-oxobutanoic acid | |
| 148 | (R)-3-benzyl-4-((4-(2-chloro-6-fluorophenyl)thiazol- | 419.9 |
| | 2-yl)amino)-4-oxobutanoic acid | |
| 149 | (R)-3-benzyl-4-((4-(4′-chloro-[1,1′-biphenyl]-2- | 492.0 |
| | yl)thiazol-2-yl)(methyl)amino)-4-oxobutanoic acid | |
| 150 | (R)-3-benzyl-4-(methyl(4-(2-(6-(2-oxopyrrolidin-1- | 541.6 |
| | yl)pyridin-3-yl)phenyl)thiazol-2-yl)amino)-4- | |
| | oxobutanoic acid | |
| 151 | (R)-3-benzyl-4-((4-(4-chloro-2-(6-methoxypyridin-3- | 523.0 |
| | yl)phenyl)thiazol-2-yl)(methyl)amino)-4-oxobutanoic | |
| | acid | |
| 152 | (R)-3-benzyl-4-((4-(5-chloro-2-(6-methoxypyridin-3- | 523.0 |
| | yl)phenyl)thiazol-2-yl)(methyl)amino)-4-oxobutanoic | |
| | acid | |
| 153 | (R)-3-benzyl-4-((4-(3-fluoro-2-(6-methoxypyridin-3- | 506.6 |
| | yl)phenyl)thiazol-2-yl)(methyl)amino)-4-oxobutanoic | |
| | acid | |
| 154 | (3R)-4-((4-(2-chlorophenyl)thiazol-2- | 409.9 |
| | yl)(methyl)amino)-4-oxo-3-((tetrahydrofuran-2- | |
| | yl)methyl)butanoic acid | |
| 155 | (R)-3-benzyl-4-((4-(2-chlorophenyl)thiazol-2-yl)(2- | 445.9 |
| | hydroxyethyl)amino)-4-oxobutanoic acid | |
| 156 | (R)-3-benzyl-4-((4-(2-chlorophenyl)thiazol-2-yl)(3- | 460.0 |
| | hydroxypropyl)amino)-4-oxobutanoic acid | |
| 157 | (R)-3-benzyl-4-((4-(2-(5-chloro-6-methoxypyridin-3- | 523.0 |
| | yl)phenyl)thiazol-2-yl)(methyl)amino)-4-oxobutanoic | |
| | acid | |
| 158 | (R)-3-benzyl-4-((4-(2-(6-(benzyloxy)pyridin-3- | 564.7 |
| | yl)phenyl)thiazol-2-yl)(methyl)amino)-4-oxobutanoic | |
| | acid | |
| 159 | (R)-3-(cyclopentylmethyl)-4-((4-(2,5- | 442.4 |
| | dichlorophenyl)thiazol-2-yl)(methyl)amino)-4- | |
| | oxobutanoic acid | |
| 160 | (R)-4-((4-(2-(6-methoxypyridin-3-yl)phenyl)thiazol- | 496.6 |
| | 2-yl)(methyl)amino)-4-oxo-3-((tetrahydro-2H-pyran- | |
| | 4-yl)methyl)butanoic acid | |
| 161 | (R)-3-benzyl-4-((4-(2-chloro-5- | 483.9 |
| | (trifluoromethyl)phenyl)thiazol-2-yl)(methyl)amino)- | |
| | 4-oxobutanoic acid | |
| 162 | (R)-3-benzyl-4-((4-(2-chloro-5-fluorophenyl)thiazol- | 433.9 |
| | 2-yl)(methyl)amino)-4-oxobutanoic acid | |
| 163 | (R)-3-benzyl-4-((4-(3,5-dichlorophenyl)thiazol-2- | 450.4 |
| | yl)(methyl)amino)-4-oxobutanoic acid | |
| 164 | (R)-3-benzyl-4-((4-(3- | 447.5 |
| | (difluoromethoxy)phenyl)thiazol-2- | |
| | yl)(methyl)amino)-4-oxobutanoic acid | |
| 165 | (R)-4-((4-(2-chlorophenyl)thiazol-2- | 407.9 |
| | yl)(methyl)amino)-3-(cyclopentylmethyl)-4- | |
| | oxobutanoic acid | |
| 166 | (R)-3-(cyclopentylmethyl)-4-(cyclopropyl(4-(2,5- | 468.4 |
| | dichlorophenyl)thiazol-2-yl)amino)-4-oxobutanoic | |
| | acid | |
| 167 | (R)-4-(cyclopropyl(4-(2,5-dichlorophenyl)thiazol-2- | 484.4 |
| | yl)amino)-4-oxo-3-((tetrahydro-2H-pyran-4- | |
| | yl)methyl)butanoic acid | |
| 168 | (R)-4-((4-(2,5-dichlorophenyl)thiazol-2- | 458.4 |
| | yl)(methyl)amino)-4-oxo-3-((tetrahydro-2H-pyran-4- | |
| | yl)methyl)butanoic acid | |
| 169 | (R)-3-(cyclopentylmethyl)-4-(cyclopropyl(4-(2-(6- | 506.6 |
| | methoxypyridin-3-yl)phenyl)thiazol-2-yl)amino)-4- | |
| | oxobutanoic acid | |
| 170 | (R)-3-benzyl-4-((2-hydroxyethyl)(4-(2-(6- | 518.6 |
| | methoxypyridin-3-yl)phenyl)thiazol-2-yl)amino)-4- | |
| | oxobutanoic acid | |
| 171 | (R)-3-(cyclopentylmethyl)-4-(methyl(4-(2-(6- | 535.7 |
| | morpholinopyridin-3-yl)phenyl)thiazol-2-yl)amino)- | |
| | 4-oxobutanoic acid | |
| 172 | (R)-3-(cyclopentylmethyl)-4-((4-(2,5- | 472.4 |
| | dichlorophenyl)thiazol-2-yl)(2-hydroxyethyl)amino)- | |
| | 4-oxobutanoic acid | |
| 173 | (R)-4-((4-(2-chlorophenyl)thiazol-2- | 423.9 |
| | yl)(methyl)amino)-4-oxo-3-((tetrahydro-2H-pyran-4- | |
| | yl)methyl)butanoic acid | |
| 174 | (R)-3-benzyl-4-((4-(5-chloro-2- | 483.9 |
| | (trifluoromethyl)phenyl)thiazol-2-yl)(methyl)amino)- | |
| | 4-oxobutanoic acid | |
| 175 | (R)-3-benzyl-4-(methyl(4-(2,3,5- | 484.8 |
| | trichlorophenyl)thiazol-2-yl)amino)-4-oxobutanoic | |
| | acid | |
| 176 | (R)-3-benzyl-4-((4-(4-chloro-[1,1′-biphenyl]-3- | 492.0 |
| | yl)thiazol-2-yl)(methyl)amino)-4-oxobutanoic acid | |
| 177 | (R)-3-benzyl-4-((4-(2-chloro-5-(6-methoxypyridin-3- | 523.0 |
| | yl)phenyl)thiazol-2-yl)(methyl)amino)-4-oxobutanoic | |
| | acid | |
| 178 | (R)-3-benzyl-4-(cyclopropyl(4-(2-(6- | 514.6 |
| | methoxypyridin-3-yl)phenyl)thiazol-2-yl)amino)-4- | |
| | oxobutanoic acid | |
| 179 | (R)-4-(cyclopropyl(4-(2-(6-methoxypyridin-3- | 522.6 |
| | yl)phenyl)thiazol-2-yl)amino)-4-oxo-3-((tetrahydro- | |
| | 2H-pyran-4-yl)methyl)butanoic acid | |
| 180 | (R)-3-benzyl-4-(cyclopropyl(4-(2-(6- | 569.7 |
| | morpholinopyridin-3-yl)phenyl)thiazol-2-yl)amino)- | |
| | 4-oxobutanoic acid | |
| 181 | (R)-3-(cyclopentylmethyl)-4-(cyclopropyl(4-(2-(6- | 561.7 |
| | morpholinopyridin-3-yl)phenyl)thiazol-2-yl)amino)- | |
| | 4-oxobutanoic acid | |
| 182 | (R)-3-benzyl-4-(methyl(4-(2-(4-methyl-3,4-dihydro- | 529.6 |
| | 2H-pyrido[3,2-b][1,4]oxazin-7-yl)phenyl)thiazol-2- | |
| | yl)amino)-4-oxobutanoic acid | |
| 183 | (R)-3-(cyclopentylmethyl)-4-(cyclopropyl(4-(2-(6-(2- | 559.7 |
| | oxopyrrolidin-1-yl)pyridin-3-yl)phenyl)thiazol-2- | |
| | yl)amino)-4-oxobutanoic acid | |
| 184 | (R)-4-(cyclopropyl(4-(2-(6-morpholinopyridin-3- | 577.7 |
| | yl)phenyl)thiazol-2-yl)amino)-4-oxo-3-((tetrahydro- | |
| | 2H-pyran-4-yl)methyl)butanoic acid | |
| 185 | (R)-3-benzyl-4-(methyl(4-(2- | 465.5 |
| | (trifluoromethoxy)phenyl)thiazol-2-yl)amino)-4- | |
| | oxobutanoic acid | |
| 186 | (R)-4-((4-(2-chloro-5-fluorophenyl)thiazol-2- | 452.0 |
| | yl)(cyclopropyl)amino)-3-(cyclopentylmethyl)-4- | |
| | oxobutanoic acid | |
| 187 | (R)-3-(cyclopentylmethyl)-4-(methyl(4-(2-(6-(2- | 533.7 |
| | oxopyrrolidin-1-yl)pyridin-3-yl)phenyl)thiazol-2- | |
| | yl)amino)-4-oxobutanoic acid | |
| 188 | (R)-3-benzyl-4-(cyclopropyl(4-(3- | 473.5 |
| | (difluoromethoxy)phenyl)thiazol-2-yl)amino)-4- | |
| | oxobutanoic acid | |
| 189 | (R)-3-benzyl-4-((4-(2-chloro-5-fluorophenyl)thiazol- | 459.9 |
| | 2-yl)(cyclopropyl)amino)-4-oxobutanoic acid | |
| 190 | (R)-4-((4-(2-chloro-5-fluorophenyl)thiazol-2- | 468.0 |
| | yl)(cyclopropyl)amino)-4-oxo-3-((tetrahydro-2H- | |
| | pyran-4-yl)methyl)butanoic acid | |
| 191 | (R)-3-benzyl-4-((4-(2-chloro-5- | 509.9 |
| | (trifluoromethyl)phenyl)thiazol-2- | |
| | yl)(cyclopropyl)amino)-4-oxobutanoic acid | |
| 192 | (R)-3-benzyl-4-((4-(2- | 447.5 |
| | (difluoromethoxy)phenyl)thiazol-2- | |
| | yl)(methyl)amino)-4-oxobutanoic acid | |
| 193 | (R)-4-((4-(2-chloro-5- | 518.0 |
| | (trifluoromethyl)phenyl)thiazol-2- | |
| | yl)(cyclopropyl)amino)-4-oxo-3-((tetrahydro-2H- | |
| | pyran-4-yl)methyl)butanoic acid | |
| 194 | (R)-3-(cyclopentylmethyl)-4-(cyclopropyl(4-(2-(4- | 547.7 |
| | methyl-3,4-dihydro-2H-pyrido[3,2-b][1,4]oxazin-7- | |
| | yl)phenyl)thiazol-2-yl)amino)-4-oxobutanoic acid | |
| 195 | (3R,4S)-3-((4-(2-chlorophenyl)thiazol-2- | 429.9 |
| | yl)(methyl)carbamoyl)-4-phenylpentanoic acid | |
| 196 | (R)-2-(2-benzyl-3-carboxypropanamido)-5-(2- | 411.9 |
| | chlorophenyl)pyridine 1-oxide | |
| 197 | (R)-3-benzyl-4-((5-(2-chlorophenyl)pyrazin-2- | 396.8 |
| | yl)amino)-4-oxobutanoic acid | |
| 198 | 4-((4-(2-chlorophenyl)thiazol-2-yl)(methyl)amino)-3- | 424.9 |
| | (morpholinomethyl)-4-oxobutanoic acid | |
| 199 | (R)-3-benzyl-4-((4-(2-chlorophenyl)thiazol-2-yl)(2- | 460.0 |
| | methoxyethyl)amino)-4-oxobutanoic acid | |
| 200 | (R)-4-((4-(2-chlorophenyl)thiazol-2- | 408.9 |
| | yl)(methyl)amino)-3-(cyclopentylamino)-4- | |
| | oxobutanoic acid | |
| 201 | (R)-3-benzyl-4-((2-(benzyloxy)ethyl)(4-(2- | 536.1 |
| | chlorophenyl)thiazol-2-yl)amino)-4-oxobutanoic acid | |
| 202 | (R)-3-benzyl-4-((4-(5-methylfuran-2-yl)thiazol-2- | 371.4 |
| | yl)amino)-4-oxobutanoic acid | |
| 203 | (R)-3-benzyl-4-oxo-4-((3-(3- | 418.4 |
| | (trifluoromethyl)phenyl)-1H-pyrazol-5- | |
| | yl)amino)butanoic acid | |
| 204 | (R)-3-benzyl-4-((4-(5-chloro-2- | 431.9 |
| | methoxyphenyl)thiazol-2-yl)amino)-4-oxobutanoic | |
| | acid | |
| 205 | 4-((4-(2-chlorophenyl)thiazol-2-yl)(methyl)amino)-3- | 431.9 |
| | (4-hydroxybenzyl)-4-oxobutanoic acid | |
| 206 | (R)-3-benzyl-4-((4-(4′-cyano-[1,1′-biphenyl]-2- | 482.6 |
| | yl)thiazol-2-yl)(methyl)amino)-4-oxobutanoic acid | |
| 207 | (3R)-3-benzyl-4-((3-carbamoyl-4-(2,4- | 492.4 |
| | dichlorophenyl)-5-methylthiophen-2-yl)amino)-4- | |
| | oxobutanoic acid | |
| 208 | (R)-3-benzyl-4-((4-(3′-methoxy-[1,1′-biphenyl]-2- | 487.6 |
| | yl)thiazol-2-yl)(methyl)amino)-4-oxobutanoic acid | |
| 209 | 4-((4-(2-chlorophenyl)thiazol-2-yl)(methyl)amino)-3- | 436.9 |
| | ((2-methylthiazol-4-yl)methyl)-4-oxobutanoic acid | |
| 210 | 4-((4-(2-chlorophenyl)thiazol-2-yl)(methyl)amino)-3- | 420.9 |
| | ((5-methylisoxazol-3-yl)methyl)-4-oxobutanoic acid | |
| 211 | (R)-3-benzyl-4-((4-(2′-chloro-[1,1′-biphenyl]-2- | 492.0 |
| | yl)thiazol-2-yl)(methyl)amino)-4-oxobutanoic acid | |
| 212 | (R)-3-benzyl-4-((4-(2-(2-methoxypyrimidin-5- | 489.6 |
| | yl)phenyl)thiazol-2-yl)(methyl)amino)-4-oxobutanoic | |
| | acid | |
| 213 | (R)-3-benzyl-4-((4-(2,5-difluorophenyl)thiazol-2- | 403.4 |
| | yl)amino)-4-oxobutanoic acid | |
| 214 | 4-((4-(2-chlorophenyl)thiazol-2-yl)(methyl)amino)-3- | 406.9 |
| | (oxazol-4-ylmethyl)-4-oxobutanoic acid | |
| 215 | (3R)-4-((4-(2-chlorophenyl)thiazol-2- | 409.9 |
| | yl)(methyl)amino)-4-oxo-3-((tetrahydrofuran-3- | |
| | yl)methyl)butanoic acid | |
| 216 | (R)-3-benzyl-4-(methyl(4-(2-(8-methyl-7-oxo- | 541.6 |
| | 5,6,7,8-tetrahydro-1,8-naphthyridin-3- | |
| | yl)phenyl)thiazol-2-yl)amino)-4-oxobutanoic acid | |
| 217 | (R)-3-benzyl-4-(methyl(4-(2-(1-methyl-1H- | 511.6 |
| | pyrrolo[2,3-b]pyridin-5-yl)phenyl)thiazol-2- | |
| | yl)amino)-4-oxobutanoic acid | |
| 218 | (R)-3-(cyclopentylmethyl)-4-(cyclopropyl(4-(2-(6- | 519.7 |
| | (dimethylamino)pyridin-3-yl)phenyl)thiazol-2- | |
| | yl)amino)-4-oxobutanoic acid | |
| 219 | (R)-4-((4-(2-(5-chloro-6-methoxypyridin-3- | 541.1 |
| | yl)phenyl)thiazol-2-yl)(cyclopropyl)amino)-3- | |
| | (cyclopentylmethyl)-4-oxobutanoic acid | |
| 220 | (R)-3-(cyclopentylmethyl)-4-(cyclopropyl(4-(2-(5- | 524.6 |
| | fluoro-6-methoxypyridin-3-yl)phenyl)thiazol-2- | |
| | yl)amino)-4-oxobutanoic acid | |
| 221 | (R)-3-benzyl-4-((4-(2-chloro-5- | 481.9 |
| | (difluoromethoxy)phenyl)thiazol-2- | |
| | yl)(methyl)amino)-4-oxobutanoic acid | |
| 222 | (R)-3-benzyl-4-((4-(5-chloro-2-(5-chloro-6- | 557.5 |
| | methoxypyridin-3-yl)phenyl)thiazol-2- | |
| | yl)(methyl)amino)-4-oxobutanoic acid | |
| 223 | (R)-4-((4-(5-chloro-2-(5-chloro-6-methoxypyridin-3- | 575.5 |
| | yl)phenyl)thiazol-2-yl)(cyclopropyl)amino)-3- | |
| | (cyclopentylmethyl)-4-oxobutanoic acid | |
| 224 | (R)-4-((4-(5-chloro-2-(5-fluoro-6-methoxypyridin-3- | 559.1 |
| | yl)phenyl)thiazol-2-yl)(cyclopropyl)amino)-3- | |
| | (cyclopentylmethyl)-4-oxobutanoic acid | |
| 225 | (S)-3-benzyl-4-((4-(2-chlorophenyl)thiazol-2- | 401.9 |
| | yl)amino)-4-oxobutanoic acid | |
| 227 | (R)-3-benzyl-4-((4-benzylthiazol-2-yl)amino)-4- | 381.5 |
| | oxobutanoic acid | |
| 229 | (R)-3-benzyl-4-oxo-4-((5-phenyl-4H-1,2,4-triazol-3- | 351.4 |
| | yl)amino)butanoic acid | |
| 230 | 3-([1,1′-biphenyl]-4-ylmethyl)-4-((4-(2- | 492.0 |
| | chlorophenyl)thiazol-2-yl)(methyl)amino)-4- | |
| | oxobutanoic acid | |
| 231 | (R)-3-benzyl-4-((4-(1-methyl-1H-pyrazol-4- | 371.4 |
| | yl)thiazol-2-yl)amino)-4-oxobutanoic acid | |
| 232 | (R)-3-benzyl-4-((4-(4-methyl-1,2,5-oxadiazol-3- | 373.4 |
| | yl)thiazol-2-yl)amino)-4-oxobutanoic acid | |
| 233 | (R)-3-benzyl-4-(methyl(4-(2-(1-methyl-1H-pyrazol- | 461.5 |
| | 4-yl)phenyl)thiazol-2-yl)amino)-4-oxobutanoic acid | |
| 234 | (3R)-3-benzyl-4-((4-(2-(3,5-dimethylisoxazol-4- | 476.6 |
| | yl)phenyl)thiazol-2-yl)(methyl)amino)-4-oxobutanoic | |
| | acid | |
| 235 | (R)-3-benzyl-4-((4-((2- | 444.9 |
| | chlorophenyl)carbamoyl)thiazol-2-yl)amino)-4- | |
| | oxobutanoic acid | |
| 236 | (R)-3-benzyl-4-((6-(2-chlorophenyl)pyridazin-3- | 396.8 |
| | yl)amino)-4-oxobutanoic acid | |
| 237 | (R)-3-benzyl-4-(methyl(4-(2-(2-oxopyrrolidin-1- | 464.5 |
| | yl)phenyl)thiazol-2-yl)amino)-4-oxobutanoic acid | |
| 238 | (S)-2-((1-((4-(2-chlorophenyl)thiazol-2- | 431.9 |
| | yl)(methyl)amino)-1-oxo-3-phenylpropan-2- | |
| | yl)oxy)acetic acid | |
| 239 | (R)-3-benzyl-4-((1-methyl-5-phenyl-1H-imidazol-2- | 364.4 |
| | yl)amino)-4-oxobutanoic acid | |
| 240 | (R)-3-benzyl-4-((4-(2-(1-(2-methoxyethyl)-6-oxo- | 532.6 |
| | 1,6-dihydropyridin-3-yl)phenyl)thiazol-2- | |
| | yl)(methyl)amino)-4-oxobutanoic acid | |
| 241 | (R)-3-benzyl-4-(methyl(4-(2-(1-methyl-6-oxo-1,6- | 488.6 |
| | dihydropyridin-3-yl)phenyl)thiazol-2-yl)amino)-4- | |
| | oxobutanoic acid | |
| 242 | 4-((4-(2-chlorophenyl)thiazol-2-yl)(methyl)amino)-3- | 434.9 |
| | ((2,5-dimethyloxazol-4-yl)methyl)-4-oxobutanoic | |
| | acid | |
| 243 | 4-((4-(2-chlorophenyl)thiazol-2-yl)(methyl)amino)-3- | 419.9 |
| | ((1-methyl-1H-pyrazol-5-yl)methyl)-4-oxobutanoic | |
| | acid | |
| 244 | (R)-3-benzyl-4-((4-(2-(6-hydroxypyridin-3- | 474.5 |
| | yl)phenyl)thiazol-2-yl)(methyl)amino)-4-oxobutanoic | |
| | acid | |
| 245 | (R)-3-benzyl-4-((4-(2-chlorophenyl)thiazol-2-yl)((S)- | 460.0 |
| | 2-hydroxypropyl)amino)-4-oxobutanoic acid | |
| 246 | (R)-3-benzyl-4-((4-(2-chlorophenyl)thiazol-2- | 460.0 |
| | yl)((R)-2-hydroxypropyl)amino)-4-oxobutanoic acid | |
| 247 | (R)-3-(cyclohexylmethyl)-4-(cyclopropyl(4-(2-(6- | 520.7 |
| | methoxypyridin-3-yl)phenyl)thiazol-2-yl)amino)-4- | |
| | oxobutanoic acid | |
| 248 | (R)-3-benzyl-4-((4-(5-fluoro-2-(6-methoxypyridin-3- | 506.6 |
| | yl)phenyl)thiazol-2-yl)(methyl)amino)-4-oxobutanoic | |
| | acid | |
| 250 | (R)-3-benzyl-4-((4-(4,5-difluoro-2-(6- | 524.6 |
| | methoxypyridin-3-yl)phenyl)thiazol-2- | |
| | yl)(methyl)amino)-4-oxobutanoic acid | |
| 251 | (R)-4-((4-(2,5-dichlorophenyl)thiazol-2- | 440.3 |
| | yl)(methyl)amino)-3-(furan-2-ylmethyl)-4- | |
| | oxobutanoic acid | |
| 252 | (R)-4-((4-(2-chloro-5-fluorophenyl)thiazol-2- | 423.9 |
| | yl)(methyl)amino)-3-(furan-2-ylmethyl)-4- | |
| | oxobutanoic acid | |
| 253 | (R)-3-(furan-2-ylmethyl)-4-((4-(2-(6- | 478.5 |
| | methoxypyridin-3-yl)phenyl)thiazol-2- | |
| | yl)(methyl)amino)-4-oxobutanoic acid | |
| 254 | (S)-4-((4-(2-chlorophenyl)thiazol-2- | 421.9 |
| | yl)(methyl)amino)-4-oxo-3-(thiophen-2- | |
| | ylmethyl)butanoic acid | |
| 255 | (R)-4-((4-(5-chloro-2-(6-methoxypyridin-3- | 541.1 |
| | yl)phenyl)thiazol-2-yl)(cyclopropyl)amino)-3- | |
| | (cyclopentylmethyl)-4-oxobutanoic acid | |
| 256 | (R)-3-benzyl-4-(cyclopropyl(4-(2-(6-(2- | 567.7 |
| | oxopyrrolidin-1-yl)pyridin-3-yl)phenyl)thiazol-2- | |
| | yl)amino)-4-oxobutanoic acid | |
| 257 | (R)-3-benzyl-4-((4-(2,3-dichlorophenyl)thiazol-2- | 450.4 |
| | yl)(methyl)amino)-4-oxobutanoic acid | |
| 258 | (R)-3-benzyl-4-(methyl(4-(3- | 465.5 |
| | (trifluoromethoxy)phenyl)thiazol-2-yl)amino)-4- | |
| | oxobutanoic acid | |
| 259 | (R)-4-(cyclopropyl(4-(3- | 481.5 |
| | (difluoromethoxy)phenyl)thiazol-2-yl)amino)-4-oxo- | |
| | 3-((tetrahydro-2H-pyran-4-yl)methyl)butanoic acid | |
| 260 | (R)-4-((4-(2-chlorophenyl)thiazol-2- | 405.9 |
| | yl)(methyl)amino)-3-(furan-2-ylmethyl)-4- | |
| | oxobutanoic acid | |
| 261 | (R)-4-(methyl(4-(3-(trifluoromethoxy)phenyl)thiazol- | 473.5 |
| | 2-yl)amino)-4-oxo-3-((tetrahydro-2H-pyran-4- | |
| | yl)methyl)butanoic acid | |
| 262 | (R)-3-benzyl-4-(cyclopropyl(4-(3- | 491.5 |
| | (trifluoromethoxy)phenyl)thiazol-2-yl)amino)-4- | |
| | oxobutanoic acid | |
| 263 | (R)-4-(cyclopropyl(4-(3- | 499.5 |
| | (trifluoromethoxy)phenyl)thiazol-2-yl)amino)-4-oxo- | |
| | 3-((tetrahydro-2H-pyran-4-yl)methyl)butanoic acid | |
| 264 | (R)-3-benzyl-4-((4-(2-(6-isopropoxypyridin-3- | 516.6 |
| | yl)phenyl)thiazol-2-yl)(methyl)amino)-4-oxobutanoic | |
| | acid | |
| 265 | (R)-3-benzyl-4-((4-(2-(6- | 528.6 |
| | (cyclopropylmethoxy)pyridin-3-yl)phenyl)thiazol-2- | |
| | yl)(methyl)amino)-4-oxobutanoic acid | |
| 266 | (R)-3-benzyl-4-((4-(2-(6-(methoxymethyl)pyridin-3- | 502.6 |
| | yl)phenyl)thiazol-2-yl)(methyl)amino)-4-oxobutanoic | |
| | acid | |
| 267 | (R)-3-benzyl-4-((4-(2-(6- | 515.6 |
| | ((dimethylamino)methyl)pyridin-3-yl)phenyl)thiazol- | |
| | 2-yl)(methyl)amino)-4-oxobutanoic acid | |
| 268 | (R)-3-benzyl-4-(methyl(4-(2-(6-(N- | 555.7 |
| | methylcyclopropanecarboxamido)pyridin-3- | |
| | yl)phenyl)thiazol-2-yl)amino)-4-oxobutanoic acid | |
| 269 | (R)-3-benzyl-4-((4-(2-(6- | 529.6 |
| | (dimethylcarbamoyl)pyridin-3-yl)phenyl)thiazol-2- | |
| | yl)(methyl)amino)-4-oxobutanoic acid | |
| 270 | (R)-4-((4-(2-(6-(4H-1,2,4-triazol-4-yl)pyridin-3- | 525.6 |
| | yl)phenyl)thiazol-2-yl)(methyl)amino)-3-benzyl-4- | |
| | oxobutanoic acid | |
| 271 | (R)-3-benzyl-4-(methyl(4-(2-(6-(3-methyl-2- | 556.6 |
| | oxoimidazolidin-1-yl)pyridin-3-yl)phenyl)thiazol-2- | |
| | yl)amino)-4-oxobutanoic acid | |
| 272 | (R)-3-benzyl-4-(methyl(4-(2-(1-methyl-2-oxo-2,3- | 527.6 |
| | dihydro-1H-pyrrolo[2,3-b]pyridin-5- | |
| | yl)phenyl)thiazol-2-yl)amino)-4-oxobutanoic acid | |
| 273 | (R)-3-benzyl-4-(methyl(4-(2-(3-methyl-3H- | 512.6 |
| | imidazo[4,5-b]pyridin-6-yl)phenyl)thiazol-2- | |
| | yl)amino)-4-oxobutanoic acid | |
| 274 | (R)-3-benzyl-4-((4-(2-(6- | 577.7 |
| | (benzyl(methyl)amino)pyridin-3-yl)phenyl)thiazol-2- | |
| | yl)(methyl)amino)-4-oxobutanoic acid | |
| 275 | (R)-3-benzyl-4-((4-(2-(6- | 569.7 |
| | (cyclohexyl(methyl)amino)pyridin-3- | |
| | yl)phenyl)thiazol-2-yl)(methyl)amino)-4-oxobutanoic | |
| | acid | |
| 276 | (R)-3-benzyl-4-(methyl(4-(2-(6-(4-methylpiperazin- | 556.7 |
| | 1-yl)pyridin-3-yl)phenyl)thiazol-2-yl)amino)-4- | |
| | oxobutanoic acid | |
| 277 | (R)-4-((4-(5-chloro-2-(6-methoxypyridin-3- | 541.1 |
| | yl)phenyl)thiazol-2-yl)(cyclopropyl)amino)-3- | |
| | (cyclopentylmethyl)-4-oxobutanoic acid | |
| 278 | (R)-3-(cyclopentylmethyl)-4-(cyclopropyl(4-(3- | 524.6 |
| | fluoro-2-(6-methoxypyridin-3-yl)phenyl)thiazol-2- | |
| | yl)amino)-4-oxobutanoic acid | |
| 279 | (R)-3-benzyl-4-((4-(2-(5-chloro-6-methoxypyridin-3- | 541.0 |
| | yl)-3-fluorophenyl)thiazol-2-yl)(methyl)amino)-4- | |
| | oxobutanoic acid | |
| 280 | (R)-3-benzyl-4-((4-(3-fluoro-2-(5-fluoro-6- | 524.6 |
| | methoxypyridin-3-yl)phenyl)thiazol-2- | |
| | yl)(methyl)amino)-4-oxobutanoic acid | |
| 281 | (R)-3-benzyl-4-((4-(5-chloro-2-(5-fluoro-6- | 541.0 |
| | methoxypyridin-3-yl)phenyl)thiazol-2- | |
| | yl)(methyl)amino)-4-oxobutanoic acid | |
| 282 | (R)-3-benzyl-4-((4-(3,5-difluoro-2-(6- | 524.6 |
| | methoxypyridin-3-yl)phenyl)thiazol-2- | |
| | yl)(methyl)amino)-4-oxobutanoic acid | |
| 283 | (R)-4-((4-(2-chlorophenyl)thiazol-2- | 409.9 |
| | yl)(methyl)amino)-4-oxo-3-(((S)-tetrahydrofuran-2- | |
| | yl)methyl)butanoic acid | |
| 284 | (R)-4-((4-(2-chlorophenyl)thiazol-2- | 409.9 |
| | yl)(methyl)amino)-4-oxo-3-(((R)-tetrahydrofuran-2- | |
| | yl)methyl)butanoic acid | |
| 285 | (R)-4-((4-(2-chloro-5- | 473.9 |
| | (trifluoromethyl)phenyl)thiazol-2-yl)(methyl)amino)- | |
| | 3-(furan-2-ylmethyl)-4-oxobutanoic acid | |
| 286 | (R)-4-((4-(2-chloro-5- | 489.9 |
| | (trifluoromethoxy)phenyl)thiazol-2- | |
| | yl)(methyl)amino)-3-(furan-2-ylmethyl)-4- | |
| | oxobutanoic acid | |
| 287 | (R)-4-((4-(2-chloro-5- | 471.9 |
| | (difluoromethoxy)phenyl)thiazol-2- | |
| | yl)(methyl)amino)-3-(furan-2-ylmethyl)-4- | |
| | oxobutanoic acid | |
| 288 | (R)-4-((4-(2-chlorophenyl)thiazol-2- | 431.9 |
| | yl)(cyclopropyl)amino)-3-(furan-2-ylmethyl)-4- | |
| | oxobutanoic acid | |
| 289 | (R)-4-((4-(5-chloro-2-(6-methoxypyridin-3- | 513.0 |
| | yl)phenyl)thiazol-2-yl)(methyl)amino)-3-(furan-2- | |
| | ylmethyl)-4-oxobutanoic acid | |
| 290 | (R)-4-((4-(2-chloro-5-(6-methoxypyridin-3- | 513.0 |
| | yl)phenyl)thiazol-2-yl)(methyl)amino)-3-(furan-2- | |
| | ylmethyl)-4-oxobutanoic acid | |
| 291 | (R)-3-(furan-2-ylmethyl)-4-((4-(2-(6- | 546.5 |
| | methoxypyridin-3-yl)-5- | |
| | (trifluoromethyl)phenyl)thiazol-2-yl)(methyl)amino)- | |
| | 4-oxobutanoic acid | |
| 292 | (R)-3-(furan-2-ylmethyl)-4-((4-(2-(6- | 562.5 |
| | methoxypyridin-3-yl)-5- | |
| | (trifluoromethoxy)phenyl)thiazol-2- | |
| | yl)(methyl)amino)-4-oxobutanoic acid | |
| 293 | (R)-4-((4-(5-(difluoromethoxy)-2-(6- | 544.5 |
| | methoxypyridin-3-yl)phenyl)thiazol-2- | |
| | yl)(methyl)amino)-3-(furan-2-ylmethyl)-4- | |
| | oxobutanoic acid | |
| 294 | (R)-4-(cyclopropyl(4-(2,5-dichlorophenyl)thiazol-2- | 466.4 |
| | yl)amino)-3-(furan-2-ylmethyl)-4-oxobutanoic acid | |
| 295 | (R)-4-((4-(2-chloro-5-fluorophenyl)thiazol-2- | 449.9 |
| | yl)(cyclopropyl)amino)-3-(furan-2-ylmethyl)-4- | |
| | oxobutanoic acid | |
| 296 | (R)-4-((4-(2-chloro-5- | 499.9 |
| | (trifluoromethyl)phenyl)thiazol-2- | |
| | yl)(cyclopropyl)amino)-3-(furan-2-ylmethyl)-4- | |
| | oxobutanoic acid | |
| 297 | (R)-4-((4-(2-chloro-5- | 515.9 |
| | (trifluoromethoxy)phenyl)thiazol-2- | |
| | yl)(cyclopropyl)amino)-3-(furan-2-ylmethyl)-4- | |
| | oxobutanoic acid | |
| 298 | (R)-4-((4-(2-chloro-5- | 497.9 |
| | (difluoromethoxy)phenyl)thiazol-2- | |
| | yl)(cyclopropyl)amino)-3-(furan-2-ylmethyl)-4- | |
| | oxobutanoic acid | |
| 299 | 4-((4-(2-chlorophenyl)thiazol-2-yl)(methyl)amino)-3- | 419.9 |
| | ((5-methylfuran-2-yl)methyl)-4-oxobutanoic acid | |
| 300 | 4-((4-(2-chlorophenyl)thiazol-2-yl)(methyl)amino)-3- | 433.9 |
| | ((4,5-dimethylfuran-2-yl)methyl)-4-oxobutanoic acid | |
| 301 | 3-(benzofuran-2-ylmethyl)-4-((4-(2- | 455.9 |
| | chlorophenyl)thiazol-2-yl)(methyl)amino)-4- | |
| | oxobutanoic acid | |
| 302 | (R)-4-((4-(2-chlorophenyl)thiazol-2- | 416.9 |
| | yl)(methyl)amino)-4-oxo-3-(pyridin-2- | |
| | ylmethyl)butanoic acid | |
| 303 | (R)-4-((4-(2-chlorophenyl)thiazol-2- | 417.9 |
| | yl)(methyl)amino)-4-oxo-3-(pyrimidin-2- | |
| | ylmethyl)butanoic acid | |
| 304 | (3R)-4-((4-(2,5-dichlorophenyl)thiazol-2- | 458.4 |
| | yl)(methyl)amino)-4-oxo-3-((tetrahydro-2H-pyran-2- | |
| | yl)methyl)butanoic acid | |
| 305 | (3R)-4-((4-(2,5-dichlorophenyl)thiazol-2- | 458.4 |
| | yl)(methyl)amino)-4-oxo-3-((tetrahydro-2H-pyran-3- | |
| | yl)methyl)butanoic acid | |
| 306 | (R)-4-((4-(2,5-dichlorophenyl)thiazol-2- | 472.4 |
| | yl)(methyl)amino)-3-(((2R,3R)-2-methyltetrahydro- | |
| | 2H-pyran-3-yl)methyl)-4-oxobutanoic acid | |
| 307 | (3R)-4-((4-(2,5-dichlorophenyl)thiazol-2- | 472.4 |
| | yl)(methyl)amino)-3-(((2R)-2-methyltetrahydro-2H- | |
| | pyran-4-yl)methyl)-4-oxobutanoic acid | |
| 308 | (3R)-4-((4-(2,5-dichlorophenyl)thiazol-2- | 486.4 |
| | yl)(methyl)amino)-3-(((2R,6S)-2,6- | |
| | dimethyltetrahydro-2H-pyran-4-yl)methyl)-4- | |
| | oxobutanoic acid | |
| 309 | (3R)-4-((4-(2,5-dichlorophenyl)thiazol-2- | 472.4 |
| | yl)(methyl)amino)-3-(((3S)-3-methyltetrahydro-2H- | |
| | pyran-4-yl)methyl)-4-oxobutanoic acid | |
| 310 | (3R)-4-((4-(2,5-dichlorophenyl)thiazol-2- | 486.4 |
| | yl)(methyl)amino)-3-(((3R,5S)-3,5- | |
| | dimethyltetrahydro-2H-pyran-4-yl)methyl)-4- | |
| | oxobutanoic acid | |
| 311 | (R)-2-benzyl-N-(4-(2-chlorophenyl)thiazol-2-yl)-3- | 472.0 |
| | (4-hydroxy-1,2,5-thiadiazol-3-yl)-N- | |
| | methylpropanamide | |
| 312 | (R)-2-benzyl-N-(4-(2-chlorophenyl)thiazol-2-yl)-3- | 469.0 |
| | (3-hydroxy-5-methylisoxazol-4-yl)-N- | |
| | methylpropanamide | |
| 313 | (R)-4-((4-(5-chloro-2-(6-(2-oxopiperidin-1- | 624.2 |
| | yl)pyridin-3-yl)phenyl)thiazol-2- | |
| | yl)(cyclopropyl)amino)-4-oxo-3-((tetrahydro-2H- | |
| | pyran-4-yl)methyl)butanoic acid | |
| 314 | (R)-4-((4-(5-chloro-2-(6-(2-oxopiperidin-1- | 642.2 |
| | yl)pyridin-3-yl)phenyl)-5-fluorothiazol-2- | |
| | yl)(cyclopropyl)amino)-4-oxo-3-((tetrahydro-2H- | |
| | pyran-4-yl)methyl)butanoic acid | |
| 315 | (R)-4-((4-(5-chloro-2-(6-methoxypyridin-3- | 557.1 |
| | yl)phenyl)thiazol-2-yl)(cyclopropyl)amino)-4-oxo-3- | |
| | ((tetrahydro-2H-pyran-4-yl)methyl)butanoic acid | |
| 316 | (R)-4-((4-(5-chloro-2-(6-methoxypyridin-3- | 575.1 |
| | yl)phenyl)-5-fluorothiazol-2-yl)(cyclopropyl)amino)- | |
| | 4-oxo-3-((tetrahydro-2H-pyran-4-yl)methyl)butanoic | |
| | acid | |
| 317 | (R)-4-((4-(5-chloro-2-(6-(2-oxopyrrolidin-1- | 610.1 |
| | yl)pyridin-3-yl)phenyl)thiazol-2- | |
| | yl)(cyclopropyl)amino)-4-oxo-3-((tetrahydro-2H- | |
| | pyran-4-yl)methyl)butanoic acid | |
| 318 | (R)-4-((4-(5-chloro-2-(6-(2-oxopyrrolidin-1- | 628.1 |
| | yl)pyridin-3-yl)phenyl)-5-fluorothiazol-2- | |
| | yl)(cyclopropyl)amino)-4-oxo-3-((tetrahydro-2H- | |
| | pyran-4-yl)methyl)butanoic acid | |
| 319 | (R)-4-(cyclopropyl(4-(5-(difluoromethoxy)-2-(6-(2- | 655.7 |
| | oxopiperidin-1-yl)pyridin-3-yl)phenyl)thiazol-2- | |
| | yl)amino)-4-oxo-3-((tetrahydro-2H-pyran-4- | |
| | yl)methyl)butanoic acid | |
| 320 | (R)-4-(cyclopropyl(4-(5-(difluoromethoxy)-2-(6-(2- | 673.7 |
| | oxopiperidin-1-yl)pyridin-3-yl)phenyl)-5- | |
| | fluorothiazol-2-yl)amino)-4-oxo-3-((tetrahydro-2H- | |
| | pyran-4-yl)methyl)butanoic acid | |
| 321 | (R)-4-(cyclopropyl(4-(5-(difluoromethoxy)-2-(6- | 588.6 |
| | methoxypyridin-3-yl)phenyl)thiazol-2-yl)amino)-4- | |
| | oxo-3-((tetrahydro-2H-pyran-4-yl)methyl)butanoic | |
| | acid | |
| 322 | (R)-4-(cyclopropyl(4-(5-(difluoromethoxy)-2-(6- | 606.6 |
| | methoxypyridin-3-yl)phenyl)-5-fluorothiazol-2- | |
| | yl)amino)-4-oxo-3-((tetrahydro-2H-pyran-4- | |
| | yl)methyl)butanoic acid | |
| 323 | (R)-4-(cyclopropyl(4-(5-(difluoromethoxy)-2-(6-(2- | 641.7 |
| | oxopyrrolidin-1-yl)pyridin-3-yl)phenyl)thiazol-2- | |
| | yl)amino)-4-oxo-3-((tetrahydro-2H-pyran-4- | |
| | yl)methyl)butanoic acid | |
| 324 | (R)-4-(cyclopropyl(4-(5-(difluoromethoxy)-2-(6-(2- | 659.7 |
| | oxopyrrolidin-1-yl)pyridin-3-yl)phenyl)-5- | |
| | fluorothiazol-2-yl)amino)-4-oxo-3-((tetrahydro-2H- | |
| | pyran-4-yl)methyl)butanoic acid | |
| 325 | (R)-4-(cyclopropyl(4-(2-(6-(2-oxopiperidin-1- | 589.7 |
| | yl)pyridin-3-yl)phenyl)thiazol-2-yl)amino)-4-oxo-3- | |
| | ((tetrahydro-2H-pyran-4-yl)methyl)butanoic acid | |
| 326 | (R)-4-(cyclopropyl(5-fluoro-4-(2-(6-(2-oxopiperidin- | 607.7 |
| | 1-yl)pyridin-3-yl)phenyl)thiazol-2-yl)amino)-4-oxo- | |
| | 3-((tetrahydro-2H-pyran-4-yl)methyl)butanoic acid | |
| 327 | (R)-4-(cyclopropyl(5-fluoro-4-(2-(6-methoxypyridin- | 540.6 |
| | 3-yl)phenyl)thiazol-2-yl)amino)-4-oxo-3- | |
| | ((tetrahydro-2H-pyran-4-yl)methyl)butanoic acid | |
| 328 | (R)-4-(cyclopropyl(4-(2-(6-(2-oxopyrrolidin-1- | 575.7 |
| | yl)pyridin-3-yl)phenyl)thiazol-2-yl)amino)-4-oxo-3- | |
| | ((tetrahydro-2H-pyran-4-yl)methyl)butanoic acid | |
| 329 | (R)-4-(cyclopropyl(5-fluoro-4-(2-(6-(2- | 593.7 |
| | oxopyrrolidin-1-yl)pyridin-3-yl)phenyl)thiazol-2- | |
| | yl)amino)-4-oxo-3-((tetrahydro-2H-pyran-4- | |
| | yl)methyl)butanoic acid | |
| 330 | (R)-4-(cyclopropyl(4-(5-fluoro-2-(6-(2-oxopiperidin- | 607.7 |
| | 1-yl)pyridin-3-yl)phenyl)thiazol-2-yl)amino)-4-oxo- | |
| | 3-((tetrahydro-2H-pyran-4-yl)methyl)butanoic acid | |
| 331 | (R)-4-(cyclopropyl(5-fluoro-4-(5-fluoro-2-(6-(2- | 625.7 |
| | oxopiperidin-1-yl)pyridin-3-yl)phenyl)thiazol-2- | |
| | yl)amino)-4-oxo-3-((tetrahydro-2H-pyran-4- | |
| | yl)methyl)butanoic acid | |
| 332 | (R)-4-(cyclopropyl(4-(5-fluoro-2-(6-methoxypyridin- | 540.6 |
| | 3-yl)phenyl)thiazol-2-yl)amino)-4-oxo-3- | |
| | ((tetrahydro-2H-pyran-4-yl)methyl)butanoic acid | |
| 333 | (R)-4-(cyclopropyl(5-fluoro-4-(5-fluoro-2-(6- | 558.6 |
| | methoxypyridin-3-yl)phenyl)thiazol-2-yl)amino)-4- | |
| | oxo-3-((tetrahydro-2H-pyran-4-yl)methyl)butanoic | |
| | acid | |
| 334 | (R)-4-(cyclopropyl(4-(5-fluoro-2-(6-(2- | 593.7 |
| | oxopyrrolidin-1-yl)pyridin-3-yl)phenyl)thiazol-2- | |
| | yl)amino)-4-oxo-3-((tetrahydro-2H-pyran-4- | |
| | yl)methyl)butanoic acid | |
| 335 | (R)-4-(cyclopropyl(5-fluoro-4-(5-fluoro-2-(6-(2- | 611.7 |
| | oxopyrrolidin-1-yl)pyridin-3-yl)phenyl)thiazol-2- | |
| | yl)amino)-4-oxo-3-((tetrahydro-2H-pyran-4- | |
| | yl)methyl)butanoic acid | |
| 336 | (R)-4-((4-(5-chloro-2-(6-(2-oxopiperidin-1- | 608.2 |
| | yl)pyridin-3-yl)phenyl)thiazol-2- | |
| | yl)(cyclopropyl)amino)-3-(cyclopentylmethyl)-4- | |
| | oxobutanoic acid | |
| 337 | (R)-4-((4-(5-chloro-2-(6-(2-oxopiperidin-1- | 626.2 |
| | yl)pyridin-3-yl)phenyl)-5-fluorothiazol-2- | |
| | yl)(cyclopropyl)amino)-3-(cyclopentylmethyl)-4- | |
| | oxobutanoic acid | |
| 338 | (R)-4-((4-(5-chloro-2-(6-methoxypyridin-3- | 559.1 |
| | yl)phenyl)-5-fluorothiazol-2-yl)(cyclopropyl)amino)- | |
| | 3-(cyclopentylmethyl)-4-oxobutanoic acid | |
| 339 | (R)-4-((4-(5-chloro-2-(6-(2-oxopyrrolidin-1- | 594.1 |
| | yl)pyridin-3-yl)phenyl)thiazol-2- | |
| | yl)(cyclopropyl)amino)-3-(cyclopentylmethyl)-4- | |
| | oxobutanoic acid | |
| 340 | (R)-4-((4-(5-chloro-2-(6-(2-oxopyrrolidin-1- | 612.1 |
| | yl)pyridin-3-yl)phenyl)-5-fluorothiazol-2- | |
| | yl)(cyclopropyl)amino)-3-(cyclopentylmethyl)-4- | |
| | oxobutanoic acid | |
| 341 | (R)-3-(cyclopentylmethyl)-4-(cyclopropyl(4-(5- | 639.7 |
| | (difluoromethoxy)-2-(6-(2-oxopiperidin-1-yl)pyridin- | |
| | 3-yl)phenyl)thiazol-2-yl)amino)-4-oxobutanoic acid | |
| 342 | (R)-3-(cyclopentylmethyl)-4-(cyclopropyl(4-(5- | 657.7 |
| | (difluoromethoxy)-2-(6-(2-oxopiperidin-1-yl)pyridin- | |
| | 3-yl)phenyl)-5-fluorothiazol-2-yl)amino)-4- | |
| | oxobutanoic acid | |
| 343 | (R)-3-(cyclopentylmethyl)-4-(cyclopropyl(4-(5- | 572.6 |
| | (difluoromethoxy)-2-(6-methoxypyridin-3- | |
| | yl)phenyl)thiazol-2-yl)amino)-4-oxobutanoic acid | |
| 344 | (R)-3-(cyclopentylmethyl)-4-(cyclopropyl(4-(5- | 590.6 |
| | (difluoromethoxy)-2-(6-methoxypyridin-3- | |
| | yl)phenyl)-5-fluorothiazol-2-yl)amino)-4- | |
| | oxobutanoic acid | |
| 345 | (R)-3-(cyclopentylmethyl)-4-(cyclopropyl(4-(5- | 625.7 |
| | (difluoromethoxy)-2-(6-(2-oxopyrrolidin-1- | |
| | yl)pyridin-3-yl)phenyl)thiazol-2-yl)amino)-4- | |
| | oxobutanoic acid | |
| 346 | (R)-3-(cyclopentylmethyl)-4-(cyclopropyl(4-(5- | 643.7 |
| | (difluoromethoxy)-2-(6-(2-oxopyrrolidin-1- | |
| | yl)pyridin-3-yl)phenyl)-5-fluorothiazol-2-yl)amino)- | |
| | 4-oxobutanoic acid | |
| 347 | (R)-3-(cyclopentylmethyl)-4-(cyclopropyl(4-(2-(6-(2- | 573.7 |
| | oxopiperidin-1-yl)pyridin-3-yl)phenyl)thiazol-2- | |
| | yl)amino)-4-oxobutanoic acid | |
| 348 | (R)-3-(cyclopentylmethyl)-4-(cyclopropyl(5-fluoro- | 591.7 |
| | 4-(2-(6-(2-oxopiperidin-1-yl)pyridin-3- | |
| | yl)phenyl)thiazol-2-yl)amino)-4-oxobutanoic acid | |
| 349 | (R)-3-(cyclopentylmethyl)-4-(cyclopropyl(5-fluoro- | 524.6 |
| | 4-(2-(6-methoxypyridin-3-yl)phenyl)thiazol-2- | |
| | yl)amino)-4-oxobutanoic acid | |
| 350 | (R)-3-(cyclopentylmethyl)-4-(cyclopropyl(5-fluoro- | 577.7 |
| | 4-(2-(6-(2-oxopyrrolidin-1-yl)pyridin-3- | |
| | yl)phenyl)thiazol-2-yl)amino)-4-oxobutanoic acid | |
| 351 | (R)-3-(cyclopentylmethyl)-4-(cyclopropyl(4-(5- | 591.7 |
| | fluoro-2-(6-(2-oxopiperidin-1-yl)pyridin-3- | |
| | yl)phenyl)thiazol-2-yl)amino)-4-oxobutanoic acid | |
| 352 | (R)-3-(cyclopentylmethyl)-4-(cyclopropyl(5-fluoro- | 609.7 |
| | 4-(5-fluoro-2-(6-(2-oxopiperidin-1-yl)pyridin-3- | |
| | yl)phenyl)thiazol-2-yl)amino)-4-oxobutanoic acid | |
| 353 | (R)-3-(cyclopentylmethyl)-4-(cyclopropyl(4-(5- | 524.6 |
| | fluoro-2-(6-methoxypyridin-3-yl)phenyl)thiazol-2- | |
| | yl)amino)-4-oxobutanoic acid | |
| 354 | (R)-3-(cyclopentylmethyl)-4-(cyclopropyl(5-fluoro- | 542.6 |
| | 4-(5-fluoro-2-(6-methoxypyridin-3-yl)phenyl)thiazol- | |
| | 2-yl)amino)-4-oxobutanoic acid | |
| 355 | (R)-3-(cyclopentylmethyl)-4-(cyclopropyl(4-(5- | 577.7 |
| | fluoro-2-(6-(2-oxopyrrolidin-1-yl)pyridin-3- | |
| | yl)phenyl)thiazol-2-yl)amino)-4-oxobutanoic acid | |
| 356 | (R)-3-(cyclopentylmethyl)-4-(cyclopropyl(5-fluoro- | 595.7 |
| | 4-(5-fluoro-2-(6-(2-oxopyrrolidin-1-yl)pyridin-3- | |
| | yl)phenyl)thiazol-2-yl)amino)-4-oxobutanoic acid | |
| 357 | (R)-3-benzyl-4-((4-(5-chloro-2-(6-(2-oxopiperidin-1- | 616.1 |
| | yl)pyridin-3-yl)phenyl)thiazol-2- | |
| | yl)(cyclopropyl)amino)-4-oxobutanoic acid | |
| 358 | (R)-3-benzyl-4-((4-(5-chloro-2-(6-(2-oxopiperidin-1- | 634.1 |
| | yl)pyridin-3-yl)phenyl)-5-fluorothiazol-2- | |
| | yl)(cyclopropyl)amino)-4-oxobutanoic acid | |
| 359 | (R)-3-benzyl-4-((4-(5-chloro-2-(6-methoxypyridin-3- | 549.1 |
| | yl)phenyl)thiazol-2-yl)(cyclopropyl)amino)-4- | |
| | oxobutanoic acid | |
| 360 | (R)-3-benzyl-4-((4-(5-chloro-2-(6-methoxypyridin-3- | 567.0 |
| | yl)phenyl)-5-fluorothiazol-2-yl)(cyclopropyl)amino)- | |
| | 4-oxobutanoic acid | |
| 361 | (R)-3-benzyl-4-((4-(5-chloro-2-(6-(2-oxopyrrolidin- | 602.1 |
| | 1-yl)pyridin-3-yl)phenyl)thiazol-2- | |
| | yl)(cyclopropyl)amino)-4-oxobutanoic acid | |
| 362 | (R)-3-benzyl-4-((4-(5-chloro-2-(6-(2-oxopyrrolidin- | 620.1 |
| | 1-yl)pyridin-3-yl)phenyl)-5-fluorothiazol-2- | |
| | yl)(cyclopropyl)amino)-4-oxobutanoic acid | |
| 363 | (R)-3-benzyl-4-(cyclopropyl(4-(5-(difluoromethoxy)- | 647.7 |
| | 2-(6-(2-oxopiperidin-1-yl)pyridin-3- | |
| | yl)phenyl)thiazol-2-yl)amino)-4-oxobutanoic acid | |
| 364 | (R)-3-benzyl-4-(cyclopropyl(4-(5-(difluoromethoxy)- | 665.7 |
| | 2-(6-(2-oxopiperidin-1-yl)pyridin-3-yl)phenyl)-5- | |
| | fluorothiazol-2-yl)amino)-4-oxobutanoic acid | |
| 365 | (R)-3-benzyl-4-(cyclopropyl(4-(5-(difluoromethoxy)- | 580.6 |
| | 2-(6-methoxypyridin-3-yl)phenyl)thiazol-2- | |
| | yl)amino)-4-oxobutanoic acid | |
| 366 | (R)-3-benzyl-4-(cyclopropyl(4-(5-(difluoromethoxy)- | 598.6 |
| | 2-(6-methoxypyridin-3-yl)phenyl)-5-fluorothiazol-2- | |
| | yl)amino)-4-oxobutanoic acid | |
| 367 | (R)-3-benzyl-4-(cyclopropyl(4-(5-(difluoromethoxy)- | 633.7 |
| | 2-(6-(2-oxopyrrolidin-1-yl)pyridin-3- | |
| | yl)phenyl)thiazol-2-yl)amino)-4-oxobutanoic acid | |
| 368 | (R)-3-benzyl-4-(cyclopropyl(4-(5-(difluoromethoxy)- | 651.7 |
| | 2-(6-(2-oxopyrrolidin-1-yl)pyridin-3-yl)phenyl)-5- | |
| | fluorothiazol-2-yl)amino)-4-oxobutanoic acid | |
| 369 | (R)-3-benzyl-4-(cyclopropyl(4-(2-(6-(2-oxopiperidin- | 581.7 |
| | 1-yl)pyridin-3-yl)phenyl)thiazol-2-yl)amino)-4- | |
| | oxobutanoic acid | |
| 370 | (R)-3-benzyl-4-(cyclopropyl(5-fluoro-4-(2-(6-(2- | 599.7 |
| | oxopiperidin-1-yl)pyridin-3-yl)phenyl)thiazol-2- | |
| | yl)amino)-4-oxobutanoic acid | |
| 371 | (R)-3-benzyl-4-(cyclopropyl(5-fluoro-4-(2-(6- | 532.6 |
| | methoxypyridin-3-yl)phenyl)thiazol-2-yl)amino)-4- | |
| | oxobutanoic acid | |
| 372 | (R)-3-benzyl-4-(cyclopropyl(5-fluoro-4-(2-(6-(2- | 585.7 |
| | oxopyrrolidin-1-yl)pyridin-3-yl)phenyl)thiazol-2- | |
| | yl)amino)-4-oxobutanoic acid | |
| 373 | (R)-3-benzyl-4-(cyclopropyl(4-(5-fluoro-2-(6-(2- | 599.7 |
| | oxopiperidin-1-yl)pyridin-3-yl)phenyl)thiazol-2- | |
| | yl)amino)-4-oxobutanoic acid | |
| 374 | (R)-3-benzyl-4-(cyclopropyl(5-fluoro-4-(5-fluoro-2- | 617.7 |
| | (6-(2-oxopiperidin-1-yl)pyridin-3-yl)phenyl)thiazol- | |
| | 2-yl)amino)-4-oxobutanoic acid | |
| 375 | (R)-3-benzyl-4-(cyclopropyl(4-(5-fluoro-2-(6- | 532.6 |
| | methoxypyridin-3-yl)phenyl)thiazol-2-yl)amino)-4- | |
| | oxobutanoic acid | |
| 376 | (R)-3-benzyl-4-(cyclopropyl(5-fluoro-4-(5-fluoro-2- | 550.6 |
| | (6-methoxypyridin-3-yl)phenyl)thiazol-2-yl)amino)- | |
| | 4-oxobutanoic acid | |
| 377 | (R)-3-benzyl-4-(cyclopropyl(4-(5-fluoro-2-(6-(2- | 585.7 |
| | oxopyrrolidin-1-yl)pyridin-3-yl)phenyl)thiazol-2- | |
| | yl)amino)-4-oxobutanoic acid | |
| 378 | (R)-3-benzyl-4-(cyclopropyl(5-fluoro-4-(5-fluoro-2- | 603.7 |
| | (6-(2-oxopyrrolidin-1-yl)pyridin-3-yl)phenyl)thiazol- | |
| | 2-yl)amino)-4-oxobutanoic acid | |
| 379 | (R)-4-((4-(5-chloro-2-(6-(2-oxopiperidin-1- | 598.1 |
| | yl)pyridin-3-yl)phenyl)thiazol-2-yl)(methyl)amino)- | |
| | 4-oxo-3-((tetrahydro-2H-pyran-4-yl)methyl)butanoic | |
| | acid | |
| 380 | (R)-4-((4-(5-chloro-2-(6-(2-oxopiperidin-1- | 616.1 |
| | yl)pyridin-3-yl)phenyl)-5-fluorothiazol-2- | |
| | yl)(methyl)amino)-4-oxo-3-((tetrahydro-2H-pyran-4- | |
| | yl)methyl)butanoic acid | |
| 381 | (R)-4-((4-(5-chloro-2-(6-methoxypyridin-3- | 531.0 |
| | yl)phenyl)thiazol-2-yl)(methyl)amino)-4-oxo-3- | |
| | ((tetrahydro-2H-pyran-4-yl)methyl)butanoic acid | |
| 382 | (R)-4-((4-(5-chloro-2-(6-methoxypyridin-3- | 549.0 |
| | yl)phenyl)-5-fluorothiazol-2-yl)(methyl)amino)-4- | |
| | oxo-3-((tetrahydro-2H-pyran-4-yl)methyl)butanoic | |
| | acid | |
| 383 | (R)-4-((4-(5-chloro-2-(6-(2-oxopyrrolidin-1- | 584.1 |
| | yl)pyridin-3-yl)phenyl)thiazol-2-yl)(methyl)amino)- | |
| | 4-oxo-3-((tetrahydro-2H-pyran-4-yl)methyl)butanoic | |
| | acid | |
| 384 | (R)-4-((4-(5-chloro-2-(6-(2-oxopyrrolidin-1- | 602.1 |
| | yl)pyridin-3-yl)phenyl)-5-fluorothiazol-2- | |
| | yl)(methyl)amino)-4-oxo-3-((tetrahydro-2H-pyran-4- | |
| | yl)methyl)butanoic acid | |
| 385 | (R)-4-((4-(5-(difluoromethoxy)-2-(6-(2-oxopiperidin- | 629.7 |
| | 1-yl)pyridin-3-yl)phenyl)thiazol-2- | |
| | yl)(methyl)amino)-4-oxo-3-((tetrahydro-2H-pyran-4- | |
| | yl)methyl)butanoic acid | |
| 386 | (R)-4-((4-(5-(difluoromethoxy)-2-(6-(2-oxopiperidin- | 647.7 |
| | 1-yl)pyridin-3-yl)phenyl)-5-fluorothiazol-2- | |
| | yl)(methyl)amino)-4-oxo-3-((tetrahydro-2H-pyran-4- | |
| | yl)methyl)butanoic acid | |
| 387 | (R)-4-((4-(5-(difluoromethoxy)-2-(6- | 562.6 |
| | methoxypyridin-3-yl)phenyl)thiazol-2- | |
| | yl)(methyl)amino)-4-oxo-3-((tetrahydro-2H-pyran-4- | |
| | yl)methyl)butanoic acid | |
| 388 | (R)-4-((4-(5-(difluoromethoxy)-2-(6- | 580.6 |
| | methoxypyridin-3-yl)phenyl)-5-fluorothiazol-2- | |
| | yl)(methyl)amino)-4-oxo-3-((tetrahydro-2H-pyran-4- | |
| | yl)methyl)butanoic acid | |
| 389 | (R)-4-((4-(5-(difluoromethoxy)-2-(6-(2- | 615.7 |
| | oxopyrrolidin-1-yl)pyridin-3-yl)phenyl)thiazol-2- | |
| | yl)(methyl)amino)-4-oxo-3-((tetrahydro-2H-pyran-4- | |
| | yl)methyl)butanoic acid | |
| 390 | (R)-4-((4-(5-(difluoromethoxy)-2-(6-(2- | 633.7 |
| | oxopyrrolidin-1-yl)pyridin-3-yl)phenyl)-5- | |
| | fluorothiazol-2-yl)(methyl)amino)-4-oxo-3- | |
| | ((tetrahydro-2H-pyran-4-yl)methyl)butanoic acid | |
| 391 | (R)-4-(methyl(4-(2-(6-(2-oxopiperidin-1-yl)pyridin- | 563.7 |
| | 3-yl)phenyl)thiazol-2-yl)amino)-4-oxo-3- | |
| | ((tetrahydro-2H-pyran-4-yl)methyl)butanoic acid | |
| 392 | (R)-4-((5-fluoro-4-(2-(6-(2-oxopiperidin-1- | 581.7 |
| | yl)pyridin-3-yl)phenyl)thiazol-2-yl)(methyl)amino)- | |
| | 4-oxo-3-((tetrahydro-2H-pyran-4-yl)methyl)butanoic | |
| | acid | |
| 393 | (R)-4-((5-fluoro-4-(2-(6-methoxypyridin-3- | 514.6 |
| | yl)phenyl)thiazol-2-yl)(methyl)amino)-4-oxo-3- | |
| | ((tetrahydro-2H-pyran-4-yl)methyl)butanoic acid | |
| 394 | (R)-4-(methyl(4-(2-(6-(2-oxopyrrolidin-1-yl)pyridin- | 549.7 |
| | 3-yl)phenyl)thiazol-2-yl)amino)-4-oxo-3- | |
| | ((tetrahydro-2H-pyran-4-yl)methyl)butanoic acid | |
| 395 | (R)-4-((5-fluoro-4-(2-(6-(2-oxopyrrolidin-1- | 567.6 |
| | yl)pyridin-3-yl)phenyl)thiazol-2-yl)(methyl)amino)- | |
| | 4-oxo-3-((tetrahydro-2H-pyran-4-yl)methyl)butanoic | |
| | acid | |
| 396 | (R)-4-((4-(5-fluoro-2-(6-(2-oxopiperidin-1- | 581.7 |
| | yl)pyridin-3-yl)phenyl)thiazol-2-yl)(methyl)amino)- | |
| | 4-oxo-3-((tetrahydro-2H-pyran-4-yl)methyl)butanoic | |
| | acid | |
| 397 | (R)-4-((5-fluoro-4-(5-fluoro-2-(6-(2-oxopiperidin-1- | 599.7 |
| | yl)pyridin-3-yl)phenyl)thiazol-2-yl)(methyl)amino)- | |
| | 4-oxo-3-((tetrahydro-2H-pyran-4-yl)methyl)butanoic | |
| | acid | |
| 398 | (R)-4-((4-(5-fluoro-2-(6-methoxypyridin-3- | 514.6 |
| | yl)phenyl)thiazol-2-yl)(methyl)amino)-4-oxo-3- | |
| | ((tetrahydro-2H-pyran-4-yl)methyl)butanoic acid | |
| 399 | (R)-4-((5-fluoro-4-(5-fluoro-2-(6-methoxypyridin-3- | 532.6 |
| | yl)phenyl)thiazol-2-yl)(methyl)amino)-4-oxo-3- | |
| | ((tetrahydro-2H-pyran-4-yl)methyl)butanoic acid | |
| 400 | (R)-4-((4-(5-fluoro-2-(6-(2-oxopyrrolidin-1- | 567.6 |
| | yl)pyridin-3-yl)phenyl)thiazol-2-yl)(methyl)amino)- | |
| | 4-oxo-3-((tetrahydro-2H-pyran-4-yl)methyl)butanoic | |
| | acid | |
| 401 | (R)-4-((5-fluoro-4-(5-fluoro-2-(6-(2-oxopyrrolidin-1- | 585.6 |
| | yl)pyridin-3-yl)phenyl)thiazol-2-yl)(methyl)amino)- | |
| | 4-oxo-3-((tetrahydro-2H-pyran-4-yl)methyl)butanoic | |
| | acid | |
| 402 | (R)-4-((4-(5-chloro-2-(6-(2-oxopiperidin-1- | 582.1 |
| | yl)pyridin-3-yl)phenyl)thiazol-2-yl)(methyl)amino)- | |
| | 3-(cyclopentylmethyl)-4-oxobutanoic acid | |
| 403 | (R)-4-((4-(5-chloro-2-(6-(2-oxopiperidin-1- | 600.1 |
| | yl)pyridin-3-yl)phenyl)-5-fluorothiazol-2- | |
| | yl)(methyl)amino)-3-(cyclopentylmethyl)-4- | |
| | oxobutanoic acid | |
| 404 | (R)-4-((4-(5-chloro-2-(6-methoxypyridin-3- | 515.0 |
| | yl)phenyl)thiazol-2-yl)(methyl)amino)-3- | |
| | (cyclopentylmethyl)-4-oxobutanoic acid | |
| 405 | (R)-4-((4-(5-chloro-2-(6-methoxypyridin-3- | 533.0 |
| | yl)phenyl)-5-fluorothiazol-2-yl)(methyl)amino)-3- | |
| | (cyclopentylmethyl)-4-oxobutanoic acid | |
| 406 | (R)-4-((4-(5-chloro-2-(6-(2-oxopyrrolidin-1- | 568.1 |
| | yl)pyridin-3-yl)phenyl)thiazol-2-yl)(methyl)amino)- | |
| | 3-(cyclopentylmethyl)-4-oxobutanoic acid | |
| 407 | (R)-4-((4-(5-chloro-2-(6-(2-oxopyrrolidin-1- | 586.1 |
| | yl)pyridin-3-yl)phenyl)-5-fluorothiazol-2- | |
| | yl)(methyl)amino)-3-(cyclopentylmethyl)-4- | |
| | oxobutanoic acid | |
| 408 | (R)-3-(cyclopentylmethyl)-4-((4-(5- | 613.7 |
| | (difluoromethoxy)-2-(6-(2-oxopiperidin-1-yl)pyridin- | |
| | 3-yl)phenyl)thiazol-2-yl)(methyl)amino)-4- | |
| | oxobutanoic acid | |
| 409 | (R)-3-(cyclopentylmethyl)-4-((4-(5- | 631.7 |
| | (difluoromethoxy)-2-(6-(2-oxopiperidin-1-yl)pyridin- | |
| | 3-yl)phenyl)-5-fluorothiazol-2-yl)(methyl)amino)-4- | |
| | oxobutanoic acid | |
| 410 | (R)-3-(cyclopentylmethyl)-4-((4-(5- | 546.6 |
| | (difluoromethoxy)-2-(6-methoxypyridin-3- | |
| | yl)phenyl)thiazol-2-yl)(methyl)amino)-4-oxobutanoic | |
| | acid | |
| 411 | (R)-3-(cyclopentylmethyl)-4-((4-(5- | 564.6 |
| | (difluoromethoxy)-2-(6-methoxypyridin-3- | |
| | yl)phenyl)-5-fluorothiazol-2-yl)(methyl)amino)-4- | |
| | oxobutanoic acid | |
| 412 | (R)-3-(cyclopentylmethyl)-4-((4-(5- | 599.7 |
| | (difluoromethoxy)-2-(6-(2-oxopyrrolidin-1- | |
| | yl)pyridin-3-yl)phenyl)thiazol-2-yl)(methyl)amino)- | |
| | 4-oxobutanoic acid | |
| 413 | (R)-3-(cyclopentylmethyl)-4-((4-(5- | 617.7 |
| | (difluoromethoxy)-2-(6-(2-oxopyrrolidin-1- | |
| | yl)pyridin-3-yl)phenyl)-5-fluorothiazol-2- | |
| | yl)(methyl)amino)-4-oxobutanoic acid | |
| 414 | (R)-3-(cyclopentylmethyl)-4-(methyl(4-(2-(6-(2- | 547.7 |
| | oxopiperidin-1-yl)pyridin-3-yl)phenyl)thiazol-2- | |
| | yl)amino)-4-oxobutanoic acid | |
| 415 | (R)-3-(cyclopentylmethyl)-4-((5-fluoro-4-(2-(6-(2- | 565.7 |
| | oxopiperidin-1-yl)pyridin-3-yl)phenyl)thiazol-2- | |
| | yl)(methyl)amino)-4-oxobutanoic acid | |
| 416 | (R)-3-(cyclopentylmethyl)-4-((4-(2-(6- | 480.6 |
| | methoxypyridin-3-yl)phenyl)thiazol-2- | |
| | yl)(methyl)amino)-4-oxobutanoic acid | |
| 417 | (R)-3-(cyclopentylmethyl)-4-((5-fluoro-4-(2-(6- | 498.6 |
| | methoxypyridin-3-yl)phenyl)thiazol-2- | |
| | yl)(methyl)amino)-4-oxobutanoic acid | |
| 418 | (R)-3-(cyclopentylmethyl)-4-((5-fluoro-4-(2-(6-(2- | 551.6 |
| | oxopyrrolidin-1-yl)pyridin-3-yl)phenyl)thiazol-2- | |
| | yl)(methyl)amino)-4-oxobutanoic acid | |
| 419 | (R)-3-(cyclopentylmethyl)-4-((4-(5-fluoro-2-(6-(2- | 565.7 |
| | oxopiperidin-1-yl)pyridin-3-yl)phenyl)thiazol-2- | |
| | yl)(methyl)amino)-4-oxobutanoic acid | |
| 420 | (R)-3-(cyclopentylmethyl)-4-((5-fluoro-4-(5-fluoro- | 583.7 |
| | 2-(6-(2-oxopiperidin-1-yl)pyridin-3- | |
| | yl)phenyl)thiazol-2-yl)(methyl)amino)-4-oxobutanoic | |
| | acid | |
| 421 | (R)-3-(cyclopentylmethyl)-4-((4-(5-fluoro-2-(6- | 498.6 |
| | methoxypyridin-3-yl)phenyl)thiazol-2- | |
| | yl)(methyl)amino)-4-oxobutanoic acid | |
| 422 | (R)-3-(cyclopentylmethyl)-4-((5-fluoro-4-(5-fluoro- | 516.6 |
| | 2-(6-methoxypyridin-3-yl)phenyl)thiazol-2- | |
| | yl)(methyl)amino)-4-oxobutanoic acid | |
| 423 | (R)-3-(cyclopentylmethyl)-4-((4-(5-fluoro-2-(6-(2- | 551.6 |
| | oxopyrrolidin-1-yl)pyridin-3-yl)phenyl)thiazol-2- | |
| | yl)(methyl)amino)-4-oxobutanoic acid | |
| 424 | (R)-3-(cyclopentylmethyl)-4-((5-fluoro-4-(5-fluoro- | 569.6 |
| | 2-(6-(2-oxopyrrolidin-1-yl)pyridin-3- | |
| | yl)phenyl)thiazol-2-yl)(methyl)amino)-4-oxobutanoic | |
| | acid | |
| 425 | (R)-3-benzyl-4-((4-(5-chloro-2-(6-(2-oxopiperidin-1- | 590.1 |
| | yl)pyridin-3-yl)phenyl)thiazol-2-yl)(methyl)amino)- | |
| | 4-oxobutanoic acid | |
| 426 | (R)-3-benzyl-4-((4-(5-chloro-2-(6-(2-oxopiperidin-1- | 608.1 |
| | yl)pyridin-3-yl)phenyl)-5-fluorothiazol-2- | |
| | yl)(methyl)amino)-4-oxobutanoic acid | |
| 427 | (R)-3-benzyl-4-((4-(5-chloro-2-(6-methoxypyridin-3- | 541.0 |
| | yl)phenyl)-5-fluorothiazol-2-yl)(methyl)amino)-4- | |
| | oxobutanoic acid | |
| 428 | (R)-3-benzyl-4-((4-(5-chloro-2-(6-(2-oxopyrrolidin- | 576.1 |
| | 1-yl)pyridin-3-yl)phenyl)thiazol-2- | |
| | yl)(methyl)amino)-4-oxobutanoic acid | |
| 429 | (R)-3-benzyl-4-((4-(5-chloro-2-(6-(2-oxopyrrolidin- | 594.1 |
| | 1-yl)pyridin-3-yl)phenyl)-5-fluorothiazol-2- | |
| | yl)(methyl)amino)-4-oxobutanoic acid | |
| 430 | (R)-3-benzyl-4-((4-(5-(difluoromethoxy)-2-(6-(2- | 621.7 |
| | oxopiperidin-1-yl)pyridin-3-yl)phenyl)thiazol-2- | |
| | yl)(methyl)amino)-4-oxobutanoic acid | |
| 431 | (R)-3-benzyl-4-((4-(5-(difluoromethoxy)-2-(6-(2- | 639.7 |
| | oxopiperidin-1-yl)pyridin-3-yl)phenyl)-5- | |
| | fluorothiazol-2-yl)(methyl)amino)-4-oxobutanoic | |
| | acid | |
| 432 | (R)-3-benzyl-4-((4-(5-(difluoromethoxy)-2-(6- | 554.6 |
| | methoxypyridin-3-yl)phenyl)thiazol-2- | |
| | yl)(methyl)amino)-4-oxobutanoic acid | |
| 433 | (R)-3-benzyl-4-((4-(5-(difluoromethoxy)-2-(6- | 572.6 |
| | methoxypyridin-3-yl)phenyl)-5-fluorothiazol-2- | |
| | yl)(methyl)amino)-4-oxobutanoic acid | |
| 434 | (R)-3-benzyl-4-((4-(5-(difluoromethoxy)-2-(6-(2- | 607.6 |
| | oxopyrrolidin-1-yl)pyridin-3-yl)phenyl)thiazol-2- | |
| | yl)(methyl)amino)-4-oxobutanoic acid | |
| 435 | (R)-3-benzyl-4-((4-(5-(difluoromethoxy)-2-(6-(2- | 625.6 |
| | oxopyrrolidin-1-yl)pyridin-3-yl)phenyl)-5- | |
| | fluorothiazol-2-yl)(methyl)amino)-4-oxobutanoic | |
| | acid | |
| 436 | (R)-3-benzyl-4-(methyl(4-(2-(6-(2-oxopiperidin-1- | 555.7 |
| | yl)pyridin-3-yl)phenyl)thiazol-2-yl)amino)-4- | |
| | oxobutanoic acid | |
| 437 | (R)-3-benzyl-4-((5-fluoro-4-(2-(6-(2-oxopiperidin-1- | 573.7 |
| | yl)pyridin-3-yl)phenyl)thiazol-2-yl)(methyl)amino)- | |
| | 4-oxobutanoic acid | |
| 438 | (R)-3-benzyl-4-((5-fluoro-4-(2-(6-methoxypyridin-3- | 506.6 |
| | yl)phenyl)thiazol-2-yl)(methyl)amino)-4-oxobutanoic | |
| | acid | |
| 439 | (R)-3-benzyl-4-((5-fluoro-4-(2-(6-(2-oxopyrrolidin- | 559.6 |
| | 1-yl)pyridin-3-yl)phenyl)thiazol-2- | |
| | yl)(methyl)amino)-4-oxobutanoic acid | |
| 440 | (R)-3-benzyl-4-((4-(5-fluoro-2-(6-(2-oxopiperidin-1- | 573.7 |
| | yl)pyridin-3-yl)phenyl)thiazol-2-yl)(methyl)amino)- | |
| | 4-oxobutanoic acid | |
| 441 | (R)-3-benzyl-4-((5-fluoro-4-(5-fluoro-2-(6-(2- | 591.6 |
| | oxopiperidin-1-yl)pyridin-3-yl)phenyl)thiazol-2- | |
| | yl)(methyl)amino)-4-oxobutanoic acid | |
| 442 | (R)-3-benzyl-4-((5-fluoro-4-(5-fluoro-2-(6- | 524.6 |
| | methoxypyridin-3-yl)phenyl)thiazol-2- | |
| | yl)(methyl)amino)-4-oxobutanoic acid | |
| 443 | (R)-3-benzyl-4-((4-(5-fluoro-2-(6-(2-oxopyrrolidin- | 559.6 |
| | 1-yl)pyridin-3-yl)phenyl)thiazol-2- | |
| | yl)(methyl)amino)-4-oxobutanoic acid | |
| 444 | (R)-3-benzyl-4-((5-fluoro-4-(5-fluoro-2-(6-(2- | 577.6 |
| | oxopyrrolidin-1-yl)pyridin-3-yl)phenyl)thiazol-2- | |
| | yl)(methyl)amino)-4-oxobutanoic acid | |
| 445 | (R)-3-(cyclopentylmethyl)-4-((4-(5-fluoro-2-(8- | 551.6 |
| | methyl-7-oxo-5,6,7,8-tetrahydro-1,8-naphthyridin-3- | |
| | yl)phenyl)thiazol-2-yl)(methyl)amino)-4-oxobutanoic | |
| | acid | |
| 446 | (R)-3-(cyclopentylmethyl)-4-(cyclopropyl(4-(5- | 577.7 |
| | fluoro-2-(8-methyl-7-oxo-5,6,7,8-tetrahydro-1,8- | |
| | naphthyridin-3-yl)phenyl)thiazol-2-yl)amino)-4- | |
| | oxobutanoic acid | |
| 447 | (R)-4-((4-(5-fluoro-2-(8-methyl-7-oxo-5,6,7,8- | 567.6 |
| | tetrahydro-1,8-naphthyridin-3-yl)phenyl)thiazol-2- | |
| | yl)(methyl)amino)-4-oxo-3-((tetrahydro-2H-pyran-4- | |
| | yl)methyl)butanoic acid | |
| 448 | (R)-4-(cyclopropyl(4-(5-fluoro-2-(8-methyl-7-oxo- | 593.7 |
| | 5,6,7,8-tetrahydro-1,8-naphthyridin-3- | |
| | yl)phenyl)thiazol-2-yl)amino)-4-oxo-3-((tetrahydro- | |
| | 2H-pyran-4-yl)methyl)butanoic acid | |
| 449 | (R)-3-benzyl-4-((4-(5-fluoro-2-(8-methyl-7-oxo- | 559.6 |
| | 5,6,7,8-tetrahydro-1,8-naphthyridin-3- | |
| | yl)phenyl)thiazol-2-yl)(methyl)amino)-4-oxobutanoic | |
| | acid | |
| 450 | (R)-3-benzyl-4-(cyclopropyl(4-(5-fluoro-2-(8- | 585.7 |
| | methyl-7-oxo-5,6,7,8-tetrahydro-1,8-naphthyridin-3- | |
| | yl)phenyl)thiazol-2-yl)amino)-4-oxobutanoic acid | |
| 451 | (R)-3-(cyclopentylmethyl)-4-((4-(5-fluoro-2-(1- | 537.6 |
| | methyl-2-oxo-2,3-dihydro-1H-pyrrolo[2,3-b]pyridin- | |
| | 5-yl)phenyl)thiazol-2-yl)(methyl)amino)-4- | |
| | oxobutanoic acid | |
| 452 | (R)-3-(cyclopentylmethyl)-4-(cyclopropyl(4-(5- | 563.7 |
| | fluoro-2-(1-methyl-2-oxo-2,3-dihydro-1H- | |
| | pyrrolo[2,3-b]pyridin-5-yl)phenyl)thiazol-2- | |
| | yl)amino)-4-oxobutanoic acid | |
| 453 | (R)-4-((4-(5-fluoro-2-(1-methyl-2-oxo-2,3-dihydro- | 553.6 |
| | 1H-pyrrolo[2,3-b]pyridin-5-yl)phenyl)thiazol-2- | |
| | yl)(methyl)amino)-4-oxo-3-((tetrahydro-2H-pyran-4- | |
| | yl)methyl)butanoic acid | |
| 454 | (R)-4-(cyclopropyl(4-(5-fluoro-2-(1-methyl-2-oxo- | 579.7 |
| | 2,3-dihydro-1H-pyrrolo[2,3-b]pyridin-5- | |
| | yl)phenyl)thiazol-2-yl)amino)-4-oxo-3-((tetrahydro- | |
| | 2H-pyran-4-yl)methyl)butanoic acid | |
| 455 | (R)-3-benzyl-4-((4-(5-fluoro-2-(1-methyl-2-oxo-2,3- | 545.6 |
| | dihydro-1H-pyrrolo[2,3-b]pyridin-5- | |
| | yl)phenyl)thiazol-2-yl)(methyl)amino)-4-oxobutanoic | |
| | acid | |
| 456 | (R)-3-benzyl-4-(cyclopropyl(4-(5-fluoro-2-(1- | 571.6 |
| | methyl-2-oxo-2,3-dihydro-1H-pyrrolo[2,3-b]pyridin- | |
| | 5-yl)phenyl)thiazol-2-yl)amino)-4-oxobutanoic acid | |
| 457 | (R)-3-(cyclopentylmethyl)-4-((4-(5-fluoro-2-(4- | 539.6 |
| | methyl-3,4-dihydro-2H-pyrido[3,2-b][1,4]oxazin-7- | |
| | yl)phenyl)thiazol-2-yl)(methyl)amino)-4-oxobutanoic | |
| | acid | |
| 458 | (R)-3-(cyclopentylmethyl)-4-(cyclopropyl(4-(5- | 565.7 |
| | fluoro-2-(4-methyl-3,4-dihydro-2H-pyrido[3,2- | |
| | b][1,4]oxazin-7-yl)phenyl)thiazol-2-yl)amino)-4- | |
| | oxobutanoic acid | |
| 459 | (R)-4-((4-(5-fluoro-2-(4-methyl-3,4-dihydro-2H- | 555.6 |
| | pyrido[3,2-b][1,4]oxazin-7-yl)phenyl)thiazol-2- | |
| | yl)(methyl)amino)-4-oxo-3-((tetrahydro-2H-pyran-4- | |
| | yl)methyl)butanoic acid | |
| 460 | (R)-4-(cyclopropyl(4-(5-fluoro-2-(4-methyl-3,4- | 581.7 |
| | dihydro-2H-pyrido[3,2-b][1,4]oxazin-7- | |
| | yl)phenyl)thiazol-2-yl)amino)-4-oxo-3-((tetrahydro- | |
| | 2H-pyran-4-yl)methyl)butanoic acid | |
| 461 | (R)-3-benzyl-4-((4-(5-fluoro-2-(4-methyl-3,4- | 547.6 |
| | dihydro-2H-pyrido[3,2-b][1,4]oxazin-7- | |
| | yl)phenyl)thiazol-2-yl)(methyl)amino)-4-oxobutanoic | |
| | acid | |
| 462 | (R)-3-benzyl-4-(cyclopropyl(4-(5-fluoro-2-(4- | 573.7 |
| | methyl-3,4-dihydro-2H-pyrido[3,2-b][1,4]oxazin-7- | |
| | yl)phenyl)thiazol-2-yl)amino)-4-oxobutanoic acid | |
| 463 | (R)-3-(cyclopentylmethyl)-4-((4-(5-fluoro-2-(1- | 521.6 |
| | methyl-1H-pyrrolo[2,3-b]pyridin-5- | |
| | yl)phenyl)thiazol-2-yl)(methyl)amino)-4-oxobutanoic | |
| | acid | |
| 464 | (R)-3-(cyclopentylmethyl)-4-(cyclopropyl(4-(5- | 547.7 |
| | fluoro-2-(1-methyl-1H-pyrrolo[2,3-b]pyridin-5- | |
| | yl)phenyl)thiazol-2-yl)amino)-4-oxobutanoic acid | |
| 465 | (R)-4-((4-(5-fluoro-2-(1-methyl-1H-pyrrolo[2,3- | 537.6 |
| | b]pyridin-5-yl)phenyl)thiazol-2-yl)(methyl)amino)-4- | |
| | oxo-3-((tetrahydro-2H-pyran-4-yl)methyl)butanoic | |
| | acid | |
| 466 | (R)-4-(cyclopropyl(4-(5-fluoro-2-(1-methyl-1H- | 563.7 |
| | pyrrolo[2,3-b]pyridin-5-yl)phenyl)thiazol-2- | |
| | yl)amino)-4-oxo-3-((tetrahydro-2H-pyran-4- | |
| | yl)methyl)butanoic acid | |
| 467 | (R)-3-benzyl-4-((4-(5-fluoro-2-(1-methyl-1H- | 529.6 |
| | pyrrolo[2,3-b]pyridin-5-yl)phenyl)thiazol-2- | |
| | yl)(methyl)amino)-4-oxobutanoic acid | |
| 468 | (R)-3-benzyl-4-(cyclopropyl(4-(5-fluoro-2-(1- | 555.6 |
| | methyl-1H-pyrrolo[2,3-b]pyridin-5- | |
| | yl)phenyl)thiazol-2-yl)amino)-4-oxobutanoic acid | |
| 469 | (R)-3-(cyclopentylmethyl)-4-((4-(5-(fluoromethoxy)- | 581.7 |
| | 2-(8-methyl-7-oxo-5,6,7,8-tetrahydro-1,8- | |
| | naphthyridin-3-yl)phenyl)thiazol-2- | |
| | yl)(methyl)amino)-4-oxobutanoic acid | |
| 470 | (R)-3-(cyclopentylmethyl)-4-(cyclopropyl(4-(5- | 607.7 |
| | (fluoromethoxy)-2-(8-methyl-7-oxo-5,6,7,8- | |
| | tetrahydro-1,8-naphthyridin-3-yl)phenyl)thiazol-2- | |
| | yl)amino)-4-oxobutanoic acid | |
| 471 | (R)-4-((4-(5-(fluoromethoxy)-2-(8-methyl-7-oxo- | 597.7 |
| | 5,6,7,8-tetrahydro-1,8-naphthyridin-3- | |
| | yl)phenyl)thiazol-2-yl)(methyl)amino)-4-oxo-3- | |
| | ((tetrahydro-2H-pyran-4-yl)methyl)butanoic acid | |
| 472 | (R)-4-(cyclopropyl(4-(5-(fluoromethoxy)-2-(8- | 623.7 |
| | methyl-7-oxo-5,6,7,8-tetrahydro-1,8-naphthyridin-3- | |
| | yl)phenyl)thiazol-2-yl)amino)-4-oxo-3-((tetrahydro- | |
| | 2H-pyran-4-yl)methyl)butanoic acid | |
| 473 | (R)-3-benzyl-4-((4-(5-(fluoromethoxy)-2-(8-methyl- | 589.6 |
| | 7-oxo-5,6,7,8-tetrahydro-1,8-naphthyridin-3- | |
| | yl)phenyl)thiazol-2-yl)(methyl)amino)-4-oxobutanoic | |
| | acid | |
| 474 | (R)-3-benzyl-4-(cyclopropyl(4-(5-(fluoromethoxy)-2- | 615.7 |
| | (8-methyl-7-oxo-5,6,7,8-tetrahydro-1,8-naphthyridin- | |
| | 3-yl)phenyl)thiazol-2-yl)amino)-4-oxobutanoic acid | |
| 475 | (R)-3-(cyclopentylmethyl)-4-((4-(5-(fluoromethoxy)- | 567.6 |
| | 2-(1-methyl-2-oxo-2,3-dihydro-1H-pyrrolo[2,3- | |
| | b]pyridin-5-yl)phenyl)thiazol-2-yl)(methyl)amino)-4- | |
| | oxobutanoic acid | |
| 476 | (R)-3-(cyclopentylmethyl)-4-(cyclopropyl(4-(5- | 593.7 |
| | (fluoromethoxy)-2-(1-methyl-2-oxo-2,3-dihydro-1H- | |
| | pyrrolo[2,3-b]pyridin-5-yl)phenyl)thiazol-2- | |
| | yl)amino)-4-oxobutanoic acid | |
| 477 | (R)-4-((4-(5-(fluoromethoxy)-2-(1-methyl-2-oxo-2,3- | 583.6 |
| | dihydro-1H-pyrrolo[2,3-b]pyridin-5- | |
| | yl)phenyl)thiazol-2-yl)(methyl)amino)-4-oxo-3- | |
| | ((tetrahydro-2H-pyran-4-yl)methyl)butanoic acid | |
| 478 | (R)-4-(cyclopropyl(4-(5-(fluoromethoxy)-2-(1- | 609.7 |
| | methyl-2-oxo-2,3-dihydro-1H-pyrrolo[2,3-b]pyridin- | |
| | 5-yl)phenyl)thiazol-2-yl)amino)-4-oxo-3- | |
| | ((tetrahydro-2H-pyran-4-yl)methyl)butanoic acid | |
| 479 | (R)-3-benzyl-4-((4-(5-(fluoromethoxy)-2-(1-methyl- | 575.6 |
| | 2-oxo-2,3-dihydro-1H-pyrrolo[2,3-b]pyridin-5- | |
| | yl)phenyl)thiazol-2-yl)(methyl)amino)-4-oxobutanoic | |
| | acid | |
| 480 | (R)-3-benzyl-4-(cyclopropyl(4-(5-(fluoromethoxy)-2- | 601.7 |
| | (1-methyl-2-oxo-2,3-dihydro-1H-pyrrolo[2,3- | |
| | b]pyridin-5-yl)phenyl)thiazol-2-yl)amino)-4- | |
| | oxobutanoic acid | |
| 481 | (R)-3-(cyclopentylmethyl)-4-((4-(5-(fluoromethoxy)- | 569.7 |
| | 2-(4-methyl-3,4-dihydro-2H-pyrido[3,2- | |
| | b][1,4]oxazin-7-yl)phenyl)thiazol-2- | |
| | yl)(methyl)amino)-4-oxobutanoic acid | |
| 482 | (R)-3-(cyclopentylmethyl)-4-(cyclopropyl(4-(5- | 595.7 |
| | (fluoromethoxy)-2-(4-methyl-3,4-dihydro-2H- | |
| | pyrido[3,2-b][1,4]oxazin-7-yl)phenyl)thiazol-2- | |
| | yl)amino)-4-oxobutanoic acid | |
| 483 | (R)-4-((4-(5-(fluoromethoxy)-2-(4-methyl-3,4- | 585.7 |
| | dihydro-2H-pyrido[3,2-b][1,4]oxazin-7- | |
| | yl)phenyl)thiazol-2-yl)(methyl)amino)-4-oxo-3- | |
| | ((tetrahydro-2H-pyran-4-yl)methyl)butanoic acid | |
| 484 | (R)-4-(cyclopropyl(4-(5-(fluoromethoxy)-2-(4- | 611.7 |
| | methyl-3,4-dihydro-2H-pyrido[3,2-b][1,4]oxazin-7- | |
| | yl)phenyl)thiazol-2-yl)amino)-4-oxo-3-((tetrahydro- | |
| | 2H-pyran-4-yl)methyl)butanoic acid | |
| 485 | (R)-3-benzyl-4-((4-(5-(fluoromethoxy)-2-(4-methyl- | 577.6 |
| | 3,4-dihydro-2H-pyrido[3,2-b][1,4]oxazin-7- | |
| | yl)phenyl)thiazol-2-yl)(methyl)amino)-4-oxobutanoic | |
| | acid | |
| 486 | (R)-3-benzyl-4-(cyclopropyl(4-(5-(fluoromethoxy)-2- | 603.7 |
| | (4-methyl-3,4-dihydro-2H-pyrido[3,2-b][1,4]oxazin- | |
| | 7-yl)phenyl)thiazol-2-yl)amino)-4-oxobutanoic acid | |
| 487 | (R)-3-(cyclopentylmethyl)-4-((4-(5-(fluoromethoxy)- | 551.6 |
| | 2-(1-methyl-1H-pyrrolo[2,3-b]pyridin-5- | |
| | yl)phenyl)thiazol-2-yl)(methyl)amino)-4-oxobutanoic | |
| | acid | |
| 488 | (R)-3-(cyclopentylmethyl)-4-(cyclopropyl(4-(5- | 577.7 |
| | (fluoromethoxy)-2-(1-methyl-1H-pyrrolo[2,3- | |
| | b]pyridin-5-yl)phenyl)thiazol-2-yl)amino)-4- | |
| | oxobutanoic acid | |
| 489 | (R)-4-((4-(5-(fluoromethoxy)-2-(1-methyl-1H- | 567.6 |
| | pyrrolo[2,3-b]pyridin-5-yl)phenyl)thiazol-2- | |
| | yl)(methyl)amino)-4-oxo-3-((tetrahydro-2H-pyran-4- | |
| | yl)methyl)butanoic acid | |
| 490 | (R)-4-(cyclopropyl(4-(5-(fluoromethoxy)-2-(1- | 593.7 |
| | methyl-1H-pyrrolo[2,3-b]pyridin-5- | |
| | yl)phenyl)thiazol-2-yl)amino)-4-oxo-3-((tetrahydro- | |
| | 2H-pyran-4-yl)methyl)butanoic acid | |
| 491 | (R)-3-benzyl-4-((4-(5-(fluoromethoxy)-2-(1-methyl- | 559.6 |
| | 1H-pyrrolo[2,3-b]pyridin-5-yl)phenyl)thiazol-2- | |
| | yl)(methyl)amino)-4-oxobutanoic acid | |
| 492 | (R)-3-benzyl-4-(cyclopropyl(4-(5-(fluoromethoxy)-2- | 585.7 |
| | (1-methyl-1H-pyrrolo[2,3-b]pyridin-5- | |
| | yl)phenyl)thiazol-2-yl)amino)-4-oxobutanoic acid | |
| 493 | (R)-4-((4-(5-chloro-2-(8-methyl-7-oxo-5,6,7,8- | 568.1 |
| | tetrahydro-1,8-naphthyridin-3-yl)phenyl)thiazol-2- | |
| | yl)(methyl)amino)-3-(cyclopentylmethyl)-4- | |
| | oxobutanoic acid | |
| 494 | (R)-4-((4-(5-chloro-2-(8-methyl-7-oxo-5,6,7,8- | 594.1 |
| | tetrahydro-1,8-naphthyridin-3-yl)phenyl)thiazol-2- | |
| | yl)(cyclopropyl)amino)-3-(cyclopentylmethyl)-4- | |
| | oxobutanoic acid | |
| 495 | (R)-4-((4-(5-chloro-2-(8-methyl-7-oxo-5,6,7,8- | 584.1 |
| | tetrahydro-1,8-naphthyridin-3-yl)phenyl)thiazol-2- | |
| | yl)(methyl)amino)-4-oxo-3-((tetrahydro-2H-pyran-4- | |
| | yl)methyl)butanoic acid | |
| 496 | (R)-4-((4-(5-chloro-2-(8-methyl-7-oxo-5,6,7,8- | 610.1 |
| | tetrahydro-1,8-naphthyridin-3-yl)phenyl)thiazol-2- | |
| | yl)(cyclopropyl)amino)-4-oxo-3-((tetrahydro-2H- | |
| | pyran-4-yl)methyl)butanoic acid | |
| 497 | (R)-3-benzyl-4-((4-(5-chloro-2-(8-methyl-7-oxo- | 576.1 |
| | 5,6,7,8-tetrahydro-1,8-naphthyridin-3- | |
| | yl)phenyl)thiazol-2-yl)(methyl)amino)-4-oxobutanoic | |
| | acid | |
| 498 | (R)-3-benzyl-4-((4-(5-chloro-2-(8-methyl-7-oxo- | 602.1 |
| | 5,6,7,8-tetrahydro-1,8-naphthyridin-3- | |
| | yl)phenyl)thiazol-2-yl)(cyclopropyl)amino)-4- | |
| | oxobutanoic acid | |
| 499 | (R)-4-((4-(5-chloro-2-(1-methyl-2-oxo-2,3-dihydro- | 554.1 |
| | 1H-pyrrolo[2,3-b]pyridin-5-yl)phenyl)thiazol-2- | |
| | yl)(methyl)amino)-3-(cyclopentylmethyl)-4- | |
| | oxobutanoic acid | |
| 500 | (R)-4-((4-(5-chloro-2-(1-methyl-2-oxo-2,3-dihydro- | 580.1 |
| | 1H-pyrrolo[2,3-b]pyridin-5-yl)phenyl)thiazol-2- | |
| | yl)(cyclopropyl)amino)-3-(cyclopentylmethyl)-4- | |
| | oxobutanoic acid | |
| 501 | (R)-4-((4-(5-chloro-2-(1-methyl-2-oxo-2,3-dihydro- | 570.1 |
| | 1H-pyrrolo[2,3-b]pyridin-5-yl)phenyl)thiazol-2- | |
| | yl)(methyl)amino)-4-oxo-3-((tetrahydro-2H-pyran-4- | |
| | yl)methyl)butanoic acid | |
| 502 | (R)-4-((4-(5-chloro-2-(1-methyl-2-oxo-2,3-dihydro- | 596.1 |
| | 1H-pyrrolo[2,3-b]pyridin-5-yl)phenyl)thiazol-2- | |
| | yl)(cyclopropyl)amino)-4-oxo-3-((tetrahydro-2H- | |
| | pyran-4-yl)methyl)butanoic acid | |
| 503 | (R)-3-benzyl-4-((4-(5-chloro-2-(1-methyl-2-oxo-2,3- | 562.1 |
| | dihydro-1H-pyrrolo[2,3-b]pyridin-5- | |
| | yl)phenyl)thiazol-2-yl)(methyl)amino)-4-oxobutanoic | |
| | acid | |
| 504 | (R)-3-benzyl-4-((4-(5-chloro-2-(1-methyl-2-oxo-2,3- | 588.1 |
| | dihydro-1H-pyrrolo[2,3-b]pyridin-5- | |
| | yl)phenyl)thiazol-2-yl)(cyclopropyl)amino)-4- | |
| | oxobutanoic acid | |
| 505 | (R)-4-((4-(5-chloro-2-(4-methyl-3,4-dihydro-2H- | 556.1 |
| | pyrido[3,2-b][1,4]oxazin-7-yl)phenyl)thiazol-2- | |
| | yl)(methyl)amino)-3-(cyclopentylmethyl)-4- | |
| | oxobutanoic acid | |
| 506 | (R)-4-((4-(5-chloro-2-(4-methyl-3,4-dihydro-2H- | 582.1 |
| | pyrido[3,2-b][1,4]oxazin-7-yl)phenyl)thiazol-2- | |
| | yl)(cyclopropyl)amino)-3-(cyclopentylmethyl)-4- | |
| | oxobutanoic acid | |
| 507 | (R)-4-((4-(5-chloro-2-(4-methyl-3,4-dihydro-2H- | 572.1 |
| | pyrido[3,2-b][1,4]oxazin-7-yl)phenyl)thiazol-2- | |
| | yl)(methyl)amino)-4-oxo-3-((tetrahydro-2H-pyran-4- | |
| | yl)methyl)butanoic acid | |
| 508 | (R)-4-((4-(5-chloro-2-(4-methyl-3,4-dihydro-2H- | 598.1 |
| | pyrido[3,2-b][1,4]oxazin-7-yl)phenyl)thiazol-2- | |
| | yl)(cyclopropyl)amino)-4-oxo-3-((tetrahydro-2H- | |
| | pyran-4-yl)methyl)butanoic acid | |
| 509 | (R)-3-benzyl-4-((4-(5-chloro-2-(4-methyl-3,4- | 564.1 |
| | dihydro-2H-pyrido[3,2-b][1,4]oxazin-7- | |
| | yl)phenyl)thiazol-2-yl)(methyl)amino)-4-oxobutanoic | |
| | acid | |
| 510 | (R)-3-benzyl-4-((4-(5-chloro-2-(4-methyl-3,4- | 590.1 |
| | dihydro-2H-pyrido[3,2-b][1,4]oxazin-7- | |
| | yl)phenyl)thiazol-2-yl)(cyclopropyl)amino)-4- | |
| | oxobutanoic acid | |
| 511 | (R)-4-((4-(5-chloro-2-(1-methyl-1H-pyrrolo[2,3- | 538.1 |
| | b]pyridin-5-yl)phenyl)thiazol-2-yl)(methyl)amino)-3- | |
| | (cyclopentylmethyl)-4-oxobutanoic acid | |
| 512 | (R)-4-((4-(5-chloro-2-(1-methyl-1H-pyrrolo[2,3- | 564.1 |
| | b]pyridin-5-yl)phenyl)thiazol-2- | |
| | yl)(cyclopropyl)amino)-3-(cyclopentylmethyl)-4- | |
| | oxobutanoic acid | |
| 513 | (R)-4-((4-(5-chloro-2-(1-methyl-1H-pyrrolo[2,3- | 554.1 |
| | b]pyridin-5-yl)phenyl)thiazol-2-yl)(methyl)amino)-4- | |
| | oxo-3-((tetrahydro-2H-pyran-4-yl)methyl)butanoic | |
| | acid | |
| 514 | (R)-4-((4-(5-chloro-2-(1-methyl-1H-pyrrolo[2,3- | 580.1 |
| | b]pyridin-5-yl)phenyl)thiazol-2- | |
| | yl)(cyclopropyl)amino)-4-oxo-3-((tetrahydro-2H- | |
| | pyran-4-yl)methyl)butanoic acid | |
| 515 | (R)-3-benzyl-4-((4-(5-chloro-2-(1-methyl-1H- | 546.1 |
| | pyrrolo[2,3-b]pyridin-5-yl)phenyl)thiazol-2- | |
| | yl)(methyl)amino)-4-oxobutanoic acid | |
| 516 | (R)-3-benzyl-4-((4-(5-chloro-2-(1-methyl-1H- | 572.1 |
| | pyrrolo[2,3-b]pyridin-5-yl)phenyl)thiazol-2- | |
| | yl)(cyclopropyl)amino)-4-oxobutanoic acid | |
| 517 | (R)-3-(cyclopentylmethyl)-4-(methyl(4-(2-(8-methyl- | 533.7 |
| | 7-oxo-5,6,7,8-tetrahydro-1,8-naphthyridin-3- | |
| | yl)phenyl)thiazol-2-yl)amino)-4-oxobutanoic acid | |
| 518 | (R)-3-(cyclopentylmethyl)-4-(cyclopropyl(4-(2-(8- | 559.7 |
| | methyl-7-oxo-5,6,7,8-tetrahydro-1,8-naphthyridin-3- | |
| | yl)phenyl)thiazol-2-yl)amino)-4-oxobutanoic acid | |
| 519 | (R)-4-(methyl(4-(2-(8-methyl-7-oxo-5,6,7,8- | 549.7 |
| | tetrahydro-1,8-naphthyridin-3-yl)phenyl)thiazol-2- | |
| | yl)amino)-4-oxo-3-((tetrahydro-2H-pyran-4- | |
| | yl)methyl)butanoic acid | |
| 520 | (R)-4-(cyclopropyl(4-(2-(8-methyl-7-oxo-5,6,7,8- | 575.7 |
| | tetrahydro-1,8-naphthyridin-3-yl)phenyl)thiazol-2- | |
| | yl)amino)-4-oxo-3-((tetrahydro-2H-pyran-4- | |
| | yl)methyl)butanoic acid | |
| 521 | (R)-3-benzyl-4-(cyclopropyl(4-(2-(8-methyl-7-oxo- | 567.7 |
| | 5,6,7,8-tetrahydro-1,8-naphthyridin-3- | |
| | yl)phenyl)thiazol-2-yl)amino)-4-oxobutanoic acid | |
| 522 | (R)-3-(cyclopentylmethyl)-4-(methyl(4-(2-(1-methyl- | 519.6 |
| | 2-oxo-2,3-dihydro-1H-pyrrolo[2,3-b]pyridin-5- | |
| | yl)phenyl)thiazol-2-yl)amino)-4-oxobutanoic acid | |
| 523 | (R)-3-(cyclopentylmethyl)-4-(cyclopropyl(4-(2-(1- | 545.7 |
| | methyl-2-oxo-2,3-dihydro-1H-pyrrolo[2,3-b]pyridin- | |
| | 5-yl)phenyl)thiazol-2-yl)amino)-4-oxobutanoic acid | |
| 524 | (R)-4-(methyl(4-(2-(1-methyl-2-oxo-2,3-dihydro-1H- | 535.6 |
| | pyrrolo[2,3-b]pyridin-5-yl)phenyl)thiazol-2- | |
| | yl)amino)-4-oxo-3-((tetrahydro-2H-pyran-4- | |
| | yl)methyl)butanoic acid | |
| 525 | (R)-4-(cyclopropyl(4-(2-(1-methyl-2-oxo-2,3- | 561.7 |
| | dihydro-1H-pyrrolo[2,3-b]pyridin-5- | |
| | yl)phenyl)thiazol-2-yl)amino)-4-oxo-3-((tetrahydro- | |
| | 2H-pyran-4-yl)methyl)butanoic acid | |
| 526 | (R)-3-benzyl-4-(cyclopropyl(4-(2-(1-methyl-2-oxo- | 553.6 |
| | 2,3-dihydro-1H-pyrrolo[2,3-b]pyridin-5- | |
| | yl)phenyl)thiazol-2-yl)amino)-4-oxobutanoic acid | |
| 527 | (R)-3-(cyclopentylmethyl)-4-(methyl(4-(2-(4-methyl- | 521.6 |
| | 3,4-dihydro-2H-pyrido[3,2-b][1,4]oxazin-7- | |
| | yl)phenyl)thiazol-2-yl)amino)-4-oxobutanoic acid | |
| 528 | (R)-4-(methyl(4-(2-(4-methyl-3,4-dihydro-2H- | 537.6 |
| | pyrido[3,2-b][1,4]oxazin-7-yl)phenyl)thiazol-2- | |
| | yl)amino)-4-oxo-3-((tetrahydro-2H-pyran-4- | |
| | yl)methyl)butanoic acid | |
| 529 | (R)-4-(cyclopropyl(4-(2-(4-methyl-3,4-dihydro-2H- | 563.7 |
| | pyrido[3,2-b][1,4]oxazin-7-yl)phenyl)thiazol-2- | |
| | yl)amino)-4-oxo-3-((tetrahydro-2H-pyran-4- | |
| | yl)methyl)butanoic acid | |
| 530 | (R)-3-benzyl-4-(cyclopropyl(4-(2-(4-methyl-3,4- | 555.7 |
| | dihydro-2H-pyrido[3,2-b][1,4]oxazin-7- | |
| | yl)phenyl)thiazol-2-yl)amino)-4-oxobutanoic acid | |
| 531 | (R)-3-(cyclopentylmethyl)-4-(methyl(4-(2-(1-methyl- | 503.6 |
| | 1H-pyrrolo[2,3-b]pyridin-5-yl)phenyl)thiazol-2- | |
| | yl)amino)-4-oxobutanoic acid | |
| 532 | (R)-3-(cyclopentylmethyl)-4-(cyclopropyl(4-(2-(1- | 529.7 |
| | methyl-1H-pyrrolo[2,3-b]pyridin-5- | |
| | yl)phenyl)thiazol-2-yl)amino)-4-oxobutanoic acid | |
| 533 | (R)-4-(methyl(4-(2-(1-methyl-1H-pyrrolo[2,3- | 519.6 |
| | b]pyridin-5-yl)phenyl)thiazol-2-yl)amino)-4-oxo-3- | |
| | ((tetrahydro-2H-pyran-4-yl)methyl)butanoic acid | |
| 534 | (R)-4-(cyclopropyl(4-(2-(1-methyl-1H-pyrrolo[2,3- | 545.7 |
| | b]pyridin-5-yl)phenyl)thiazol-2-yl)amino)-4-oxo-3- | |
| | ((tetrahydro-2H-pyran-4-yl)methyl)butanoic acid | |
| 535 | (R)-3-benzyl-4-(cyclopropyl(4-(2-(1-methyl-1H- | 537.6 |
| | pyrrolo[2,3-b]pyridin-5-yl)phenyl)thiazol-2- | |
| | yl)amino)-4-oxobutanoic acid | |
| 536 | (R)-3-(cyclopentylmethyl)-4-((5-fluoro-4-(5-fluoro- | 569.6 |
| | 2-(8-methyl-7-oxo-5,6,7,8-tetrahydro-1,8- | |
| | naphthyridin-3-yl)phenyl)thiazol-2- | |
| | yl)(methyl)amino)-4-oxobutanoic acid | |
| 537 | (R)-3-(cyclopentylmethyl)-4-(cyclopropyl(5-fluoro- | 595.7 |
| | 4-(5-fluoro-2-(8-methyl-7-oxo-5,6,7,8-tetrahydro- | |
| | 1,8-naphthyridin-3-yl)phenyl)thiazol-2-yl)amino)-4- | |
| | oxobutanoic acid | |
| 538 | (R)-4-((5-fluoro-4-(5-fluoro-2-(8-methyl-7-oxo- | 585.6 |
| | 5,6,7,8-tetrahydro-1,8-naphthyridin-3- | |
| | yl)phenyl)thiazol-2-yl)(methyl)amino)-4-oxo-3- | |
| | ((tetrahydro-2H-pyran-4-yl)methyl)butanoic acid | |
| 539 | (R)-4-(cyclopropyl(5-fluoro-4-(5-fluoro-2-(8-methyl- | 611.7 |
| | 7-oxo-5,6,7,8-tetrahydro-1,8-naphthyridin-3- | |
| | yl)phenyl)thiazol-2-yl)amino)-4-oxo-3-((tetrahydro- | |
| | 2H-pyran-4-yl)methyl)butanoic acid | |
| 540 | (R)-3-benzyl-4-((5-fluoro-4-(5-fluoro-2-(8-methyl-7- | 577.6 |
| | oxo-5,6,7,8-tetrahydro-1,8-naphthyridin-3- | |
| | yl)phenyl)thiazol-2-yl)(methyl)amino)-4-oxobutanoic | |
| | acid | |
| 541 | (R)-3-benzyl-4-(cyclopropyl(5-fluoro-4-(5-fluoro-2- | 603.7 |
| | (8-methyl-7-oxo-5,6,7,8-tetrahydro-1,8-naphthyridin- | |
| | 3-yl)phenyl)thiazol-2-yl)amino)-4-oxobutanoic acid | |
| 542 | (R)-3-(cyclopentylmethyl)-4-((5-fluoro-4-(5-fluoro- | 555.6 |
| | 2-(1-methyl-2-oxo-2,3-dihydro-1H-pyrrolo[2,3- | |
| | b]pyridin-5-yl)phenyl)thiazol-2-yl)(methyl)amino)-4- | |
| | oxobutanoic acid | |
| 543 | (R)-3-(cyclopentylmethyl)-4-(cyclopropyl(5-fluoro- | 581.6 |
| | 4-(5-fluoro-2-(1-methyl-2-oxo-2,3-dihydro-1H- | |
| | pyrrolo[2,3-b]pyridin-5-yl)phenyl)thiazol-2- | |
| | yl)amino)-4-oxobutanoic acid | |
| 544 | (R)-4-((5-fluoro-4-(5-fluoro-2-(1-methyl-2-oxo-2,3- | 571.6 |
| | dihydro-1H-pyrrolo[2,3-b]pyridin-5- | |
| | yl)phenyl)thiazol-2-yl)(methyl)amino)-4-oxo-3- | |
| | ((tetrahydro-2H-pyran-4-yl)methyl)butanoic acid | |
| 545 | (R)-4-(cyclopropyl(5-fluoro-4-(5-fluoro-2-(1-methyl- | 597.6 |
| | 2-oxo-2,3-dihydro-1H-pyrrolo[2,3-b]pyridin-5- | |
| | yl)phenyl)thiazol-2-yl)amino)-4-oxo-3-((tetrahydro- | |
| | 2H-pyran-4-yl)methyl)butanoic acid | |
| 546 | (R)-3-benzyl-4-((5-fluoro-4-(5-fluoro-2-(1-methyl-2- | 563.6 |
| | oxo-2,3-dihydro-1H-pyrrolo[2,3-b]pyridin-5- | |
| | yl)phenyl)thiazol-2-yl)(methyl)amino)-4-oxobutanoic | |
| | acid | |
| 547 | (R)-3-benzyl-4-(cyclopropyl(5-fluoro-4-(5-fluoro-2- | 589.6 |
| | (1-methyl-2-oxo-2,3-dihydro-1H-pyrrolo[2,3- | |
| | b]pyridin-5-yl)phenyl)thiazol-2-yl)amino)-4- | |
| | oxobutanoic acid | |
| 548 | (R)-3-(cyclopentylmethyl)-4-((5-fluoro-4-(5-fluoro- | 557.6 |
| | 2-(4-methyl-3,4-dihydro-2H-pyrido[3,2- | |
| | b][1,4]oxazin-7-yl)phenyl)thiazol-2- | |
| | yl)(methyl)amino)-4-oxobutanoic acid | |
| 549 | (R)-3-(cyclopentylmethyl)-4-(cyclopropyl(5-fluoro- | 583.7 |
| | 4-(5-fluoro-2-(4-methyl-3,4-dihydro-2H-pyrido[3,2- | |
| | b][1,4]oxazin-7-yl)phenyl)thiazol-2-yl)amino)-4- | |
| | oxobutanoic acid | |
| 550 | (R)-4-((5-fluoro-4-(5-fluoro-2-(4-methyl-3,4- | 573.6 |
| | dihydro-2H-pyrido[3,2-b][1,4]oxazin-7- | |
| | yl)phenyl)thiazol-2-yl)(methyl)amino)-4-oxo-3- | |
| | ((tetrahydro-2H-pyran-4-yl)methyl)butanoic acid | |
| 551 | (R)-4-(cyclopropyl(5-fluoro-4-(5-fluoro-2-(4-methyl- | 599.7 |
| | 3,4-dihydro-2H-pyrido[3,2-b][1,4]oxazin-7- | |
| | yl)phenyl)thiazol-2-yl)amino)-4-oxo-3-((tetrahydro- | |
| | 2H-pyran-4-yl)methyl)butanoic acid | |
| 552 | (R)-3-benzyl-4-((5-fluoro-4-(5-fluoro-2-(4-methyl- | 565.6 |
| | 3,4-dihydro-2H-pyrido[3,2-b][1,4]oxazin-7- | |
| | yl)phenyl)thiazol-2-yl)(methyl)amino)-4-oxobutanoic | |
| | acid | |
| 553 | (R)-3-benzyl-4-(cyclopropyl(5-fluoro-4-(5-fluoro-2- | 591.6 |
| | (4-methyl-3,4-dihydro-2H-pyrido[3,2-b][1,4]oxazin- | |
| | 7-yl)phenyl)thiazol-2-yl)amino)-4-oxobutanoic acid | |
| 554 | (R)-3-(cyclopentylmethyl)-4-((5-fluoro-4-(5-fluoro- | 539.6 |
| | 2-(1-methyl-1H-pyrrolo[2,3-b]pyridin-5- | |
| | yl)phenyl)thiazol-2-yl)(methyl)amino)-4-oxobutanoic | |
| | acid | |
| 555 | (R)-3-(cyclopentylmethyl)-4-(cyclopropyl(5-fluoro- | 565.6 |
| | 4-(5-fluoro-2-(1-methyl-1H-pyrrolo[2,3-b]pyridin-5- | |
| | yl)phenyl)thiazol-2-yl)amino)-4-oxobutanoic acid | |
| 556 | (R)-4-((5-fluoro-4-(5-fluoro-2-(1-methyl-1H- | 555.6 |
| | pyrrolo[2,3-b]pyridin-5-yl)phenyl)thiazol-2- | |
| | yl)(methyl)amino)-4-oxo-3-((tetrahydro-2H-pyran-4- | |
| | yl)methyl)butanoic acid | |
| 557 | (R)-4-(cyclopropyl(5-fluoro-4-(5-fluoro-2-(1-methyl- | 581.6 |
| | 1H-pyrrolo[2,3-b]pyridin-5-yl)phenyl)thiazol-2- | |
| | yl)amino)-4-oxo-3-((tetrahydro-2H-pyran-4- | |
| | yl)methyl)butanoic acid | |
| 558 | (R)-3-benzyl-4-((5-fluoro-4-(5-fluoro-2-(1-methyl- | 547.6 |
| | 1H-pyrrolo[2,3-b]pyridin-5-yl)phenyl)thiazol-2- | |
| | yl)(methyl)amino)-4-oxobutanoic acid | |
| 559 | (R)-3-benzyl-4-(cyclopropyl(5-fluoro-4-(5-fluoro-2- | 573.6 |
| | (1-methyl-1H-pyrrolo[2,3-b]pyridin-5- | |
| | yl)phenyl)thiazol-2-yl)amino)-4-oxobutanoic acid | |
| 560 | (R)-3-(cyclopentylmethyl)-4-((5-fluoro-4-(5- | 599.7 |
| | (fluoromethoxy)-2-(8-methyl-7-oxo-5,6,7,8- | |
| | tetrahydro-1,8-naphthyridin-3-yl)phenyl)thiazol-2- | |
| | yl)(methyl)amino)-4-oxobutanoic acid | |
| 561 | (R)-3-(cyclopentylmethyl)-4-(cyclopropyl(5-fluoro- | 625.7 |
| | 4-(5-(fluoromethoxy)-2-(8-methyl-7-oxo-5,6,7,8- | |
| | tetrahydro-1,8-naphthyridin-3-yl)phenyl)thiazol-2- | |
| | yl)amino)-4-oxobutanoic acid | |
| 562 | (R)-4-((5-fluoro-4-(5-(fluoromethoxy)-2-(8-methyl- | 615.7 |
| | 7-oxo-5,6,7,8-tetrahydro-1,8-naphthyridin-3- | |
| | yl)phenyl)thiazol-2-yl)(methyl)amino)-4-oxo-3- | |
| | ((tetrahydro-2H-pyran-4-yl)methyl)butanoic acid | |
| 563 | (R)-4-(cyclopropyl(5-fluoro-4-(5-(fluoromethoxy)-2- | 641.7 |
| | (8-methyl-7-oxo-5,6,7,8-tetrahydro-1,8-naphthyridin- | |
| | 3-yl)phenyl)thiazol-2-yl)amino)-4-oxo-3- | |
| | ((tetrahydro-2H-pyran-4-yl)methyl)butanoic acid | |
| 564 | (R)-3-benzyl-4-((5-fluoro-4-(5-(fluoromethoxy)-2-(8- | 607.6 |
| | methyl-7-oxo-5,6,7,8-tetrahydro-1,8-naphthyridin-3- | |
| | yl)phenyl)thiazol-2-yl)(methyl)amino)-4-oxobutanoic | |
| | acid | |
| 565 | (R)-3-benzyl-4-(cyclopropyl(5-fluoro-4-(5- | 633.7 |
| | (fluoromethoxy)-2-(8-methyl-7-oxo-5,6,7,8- | |
| | tetrahydro-1,8-naphthyridin-3-yl)phenyl)thiazol-2- | |
| | yl)amino)-4-oxobutanoic acid | |
| 566 | (R)-3-(cyclopentylmethyl)-4-((5-fluoro-4-(5- | 585.6 |
| | (fluoromethoxy)-2-(1-methyl-2-oxo-2,3-dihydro-1H- | |
| | pyrrolo[2,3-b]pyridin-5-yl)phenyl)thiazol-2- | |
| | yl)(methyl)amino)-4-oxobutanoic acid | |
| 567 | (R)-3-(cyclopentylmethyl)-4-(cyclopropyl(5-fluoro- | 611.7 |
| | 4-(5-(fluoromethoxy)-2-(1-methyl-2-oxo-2,3- | |
| | dihydro-1H-pyrrolo[2,3-b]pyridin-5- | |
| | yl)phenyl)thiazol-2-yl)amino)-4-oxobutanoic acid | |
| 568 | (R)-4-((5-fluoro-4-(5-(fluoromethoxy)-2-(1-methyl- | 601.6 |
| | 2-oxo-2,3-dihydro-1H-pyrrolo[2,3-b]pyridin-5- | |
| | yl)phenyl)thiazol-2-yl)(methyl)amino)-4-oxo-3- | |
| | ((tetrahydro-2H-pyran-4-yl)methyl)butanoic acid | |
| 569 | (R)-4-(cyclopropyl(5-fluoro-4-(5-(fluoromethoxy)-2- | 627.7 |
| | (1-methyl-2-oxo-2,3-dihydro-1H-pyrrolo[2,3- | |
| | b]pyridin-5-yl)phenyl)thiazol-2-yl)amino)-4-oxo-3- | |
| | ((tetrahydro-2H-pyran-4-yl)methyl)butanoic acid | |
| 570 | (R)-3-benzyl-4-((5-fluoro-4-(5-(fluoromethoxy)-2-(1- | 593.6 |
| | methyl-2-oxo-2,3-dihydro-1H-pyrrolo[2,3-b]pyridin- | |
| | 5-yl)phenyl)thiazol-2-yl)(methyl)amino)-4- | |
| | oxobutanoic acid | |
| 571 | (R)-3-benzyl-4-(cyclopropyl(5-fluoro-4-(5- | 619.7 |
| | (fluoromethoxy)-2-(1-methyl-2-oxo-2,3-dihydro-1H- | |
| | pyrrolo[2,3-b]pyridin-5-yl)phenyl)thiazol-2- | |
| | yl)amino)-4-oxobutanoic acid | |
| 572 | (R)-3-(cyclopentylmethyl)-4-((5-fluoro-4-(5- | 587.7 |
| | (fluoromethoxy)-2-(4-methyl-3,4-dihydro-2H- | |
| | pyrido[3,2-b][1,4]oxazin-7-yl)phenyl)thiazol-2- | |
| | yl)(methyl)amino)-4-oxobutanoic acid | |
| 573 | (R)-3-(cyclopentylmethyl)-4-(cyclopropyl(5-fluoro- | 613.7 |
| | 4-(5-(fluoromethoxy)-2-(4-methyl-3,4-dihydro-2H- | |
| | pyrido[3,2-b][1,4]oxazin-7-yl)phenyl)thiazol-2- | |
| | yl)amino)-4-oxobutanoic acid | |
| 574 | (R)-4-((5-fluoro-4-(5-(fluoromethoxy)-2-(4-methyl- | 603.6 |
| | 3,4-dihydro-2H-pyrido[3,2-b][1,4]oxazin-7- | |
| | yl)phenyl)thiazol-2-yl)(methyl)amino)-4-oxo-3- | |
| | ((tetrahydro-2H-pyran-4-yl)methyl)butanoic acid | |
| 575 | (R)-4-(cyclopropyl(5-fluoro-4-(5-(fluoromethoxy)-2- | 629.7 |
| | (4-methyl-3,4-dihydro-2H-pyrido[3,2-b][1,4]oxazin- | |
| | 7-yl)phenyl)thiazol-2-yl)amino)-4-oxo-3- | |
| | ((tetrahydro-2H-pyran-4-yl)methyl)butanoic acid | |
| 576 | (R)-3-benzyl-4-((5-fluoro-4-(5-(fluoromethoxy)-2-(4- | 595.6 |
| | methyl-3,4-dihydro-2H-pyrido[3,2-b][1,4]oxazin-7- | |
| | yl)phenyl)thiazol-2-yl)(methyl)amino)-4-oxobutanoic | |
| | acid | |
| 577 | (R)-3-benzyl-4-(cyclopropyl(5-fluoro-4-(5- | 621.7 |
| | (fluoromethoxy)-2-(4-methyl-3,4-dihydro-2H- | |
| | pyrido[3,2-b][1,4]oxazin-7-yl)phenyl)thiazol-2- | |
| | yl)amino)-4-oxobutanoic acid | |
| 578 | (R)-3-(cyclopentylmethyl)-4-((5-fluoro-4-(5- | 569.6 |
| | (fluoromethoxy)-2-(1-methyl-1H-pyrrolo[2,3- | |
| | b]pyridin-5-yl)phenyl)thiazol-2-yl)(methyl)amino)-4- | |
| | oxobutanoic acid | |
| 579 | (R)-3-(cyclopentylmethyl)-4-(cyclopropyl(5-fluoro- | 595.7 |
| | 4-(5-(fluoromethoxy)-2-(1-methyl-1H-pyrrolo[2,3- | |
| | b]pyridin-5-yl)phenyl)thiazol-2-yl)amino)-4- | |
| | oxobutanoic acid | |
| 580 | (R)-4-((5-fluoro-4-(5-(fluoromethoxy)-2-(1-methyl- | 585.6 |
| | 1H-pyrrolo[2,3-b]pyridin-5-yl)phenyl)thiazol-2- | |
| | yl)(methyl)amino)-4-oxo-3-((tetrahydro-2H-pyran-4- | |
| | yl)methyl)butanoic acid | |
| 581 | (R)-4-(cyclopropyl(5-fluoro-4-(5-(fluoromethoxy)-2- | 611.7 |
| | (1-methyl-1H-pyrrolo[2,3-b]pyridin-5- | |
| | yl)phenyl)thiazol-2-yl)amino)-4-oxo-3-((tetrahydro- | |
| | 2H-pyran-4-yl)methyl)butanoic acid | |
| 582 | (R)-3-benzyl-4-((5-fluoro-4-(5-(fluoromethoxy)-2-(1- | 577.6 |
| | methyl-1H-pyrrolo[2,3-b]pyridin-5- | |
| | yl)phenyl)thiazol-2-yl)(methyl)amino)-4-oxobutanoic | |
| | acid | |
| 583 | (R)-3-benzyl-4-(cyclopropyl(5-fluoro-4-(5- | 603.7 |
| | (fluoromethoxy)-2-(1-methyl-1H-pyrrolo[2,3- | |
| | b]pyridin-5-yl)phenyl)thiazol-2-yl)amino)-4- | |
| | oxobutanoic acid | |
| 584 | (R)-4-((4-(5-chloro-2-(8-methyl-7-oxo-5,6,7,8- | 586.1 |
| | tetrahydro-1,8-naphthyridin-3-yl)phenyl)-5- | |
| | fluorothiazol-2-yl)(methyl)amino)-3- | |
| | (cyclopentylmethyl)-4-oxobutanoic acid | |
| 585 | (R)-4-((4-(5-chloro-2-(8-methyl-7-oxo-5,6,7,8- | 612.1 |
| | tetrahydro-1,8-naphthyridin-3-yl)phenyl)-5- | |
| | fluorothiazol-2-yl)(cyclopropyl)amino)-3- | |
| | (cyclopentylmethyl)-4-oxobutanoic acid | |
| 586 | (R)-4-((4-(5-chloro-2-(8-methyl-7-oxo-5,6,7,8- | 602.1 |
| | tetrahydro-1,8-naphthyridin-3-yl)phenyl)-5- | |
| | fluorothiazol-2-yl)(methyl)amino)-4-oxo-3- | |
| | ((tetrahydro-2H-pyran-4-yl)methyl)butanoic acid | |
| 587 | (R)-4-((4-(5-chloro-2-(8-methyl-7-oxo-5,6,7,8- | 628.1 |
| | tetrahydro-1,8-naphthyridin-3-yl)phenyl)-5- | |
| | fluorothiazol-2-yl)(cyclopropyl)amino)-4-oxo-3- | |
| | ((tetrahydro-2H-pyran-4-yl)methyl)butanoic acid | |
| 588 | (R)-3-benzyl-4-((4-(5-chloro-2-(8-methyl-7-oxo- | 594.1 |
| | 5,6,7,8-tetrahydro-1,8-naphthyridin-3-yl)phenyl)-5- | |
| | fluorothiazol-2-yl)(methyl)amino)-4-oxobutanoic | |
| | acid | |
| 589 | (R)-3-benzyl-4-((4-(5-chloro-2-(8-methyl-7-oxo- | 620.1 |
| | 5,6,7,8-tetrahydro-1,8-naphthyridin-3-yl)phenyl)-5- | |
| | fluorothiazol-2-yl)(cyclopropyl)amino)-4- | |
| | oxobutanoic acid | |
| 590 | (R)-4-((4-(5-chloro-2-(1-methyl-2-oxo-2,3-dihydro- | 572.1 |
| | 1H-pyrrolo[2,3-b]pyridin-5-yl)phenyl)-5- | |
| | fluorothiazol-2-yl)(methyl)amino)-3- | |
| | (cyclopentylmethyl)-4-oxobutanoic acid | |
| 591 | (R)-4-((4-(5-chloro-2-(1-methyl-2-oxo-2,3-dihydro- | 598.1 |
| | 1H-pyrrolo[2,3-b]pyridin-5-yl)phenyl)-5- | |
| | fluorothiazol-2-yl)(cyclopropyl)amino)-3- | |
| | (cyclopentylmethyl)-4-oxobutanoic acid | |
| 592 | (R)-4-((4-(5-chloro-2-(1-methyl-2-oxo-2,3-dihydro- | 588.1 |
| | 1H-pyrrolo[2,3-b]pyridin-5-yl)phenyl)-5- | |
| | fluorothiazol-2-yl)(methyl)amino)-4-oxo-3- | |
| | ((tetrahydro-2H-pyran-4-yl)methyl)butanoic acid | |
| 593 | (R)-4-((4-(5-chloro-2-(1-methyl-2-oxo-2,3-dihydro- | 614.1 |
| | 1H-pyrrolo[2,3-b]pyridin-5-yl)phenyl)-5- | |
| | fluorothiazol-2-yl)(cyclopropyl)amino)-4-oxo-3- | |
| | ((tetrahydro-2H-pyran-4-yl)methyl)butanoic acid | |
| 594 | (R)-3-benzyl-4-((4-(5-chloro-2-(1-methyl-2-oxo-2,3- | 580.0 |
| | dihydro-1H-pyrrolo[2,3-b]pyridin-5-yl)phenyl)-5- | |
| | fluorothiazol-2-yl)(methyl)amino)-4-oxobutanoic | |
| | acid | |
| 595 | (R)-3-benzyl-4-((4-(5-chloro-2-(1-methyl-2-oxo-2,3- | 606.1 |
| | dihydro-1H-pyrrolo[2,3-b]pyridin-5-yl)phenyl)-5- | |
| | fluorothiazol-2-yl)(cyclopropyl)amino)-4- | |
| | oxobutanoic acid | |
| 596 | (R)-4-((4-(5-chloro-2-(4-methyl-3,4-dihydro-2H- | 574.1 |
| | pyrido[3,2-b][1,4]oxazin-7-yl)phenyl)-5- | |
| | fluorothiazol-2-yl)(methyl)amino)-3- | |
| | (cyclopentylmethyl)-4-oxobutanoic acid | |
| 597 | (R)-4-((4-(5-chloro-2-(4-methyl-3,4-dihydro-2H- | 600.1 |
| | pyrido[3,2-b][1,4]oxazin-7-yl)phenyl)-5- | |
| | fluorothiazol-2-yl)(cyclopropyl)amino)-3- | |
| | (cyclopentylmethyl)-4-oxobutanoic acid | |
| 598 | (R)-4-((4-(5-chloro-2-(4-methyl-3,4-dihydro-2H- | 590.1 |
| | pyrido[3,2-b][1,4]oxazin-7-yl)phenyl)-5- | |
| | fluorothiazol-2-yl)(methyl)amino)-4-oxo-3- | |
| | ((tetrahydro-2H-pyran-4-yl)methyl)butanoic acid | |
| 599 | (R)-4-((4-(5-chloro-2-(4-methyl-3,4-dihydro-2H- | 616.1 |
| | pyrido[3,2-b][1,4]oxazin-7-yl)phenyl)-5- | |
| | fluorothiazol-2-yl)(cyclopropyl)amino)-4-oxo-3- | |
| | ((tetrahydro-2H-pyran-4-yl)methyl)butanoic acid | |
| 600 | (R)-3-benzyl-4-((4-(5-chloro-2-(4-methyl-3,4- | 582.1 |
| | dihydro-2H-pyrido[3,2-b][1,4]oxazin-7-yl)phenyl)-5- | |
| | fluorothiazol-2-yl)(methyl)amino)-4-oxobutanoic | |
| | acid | |
| 601 | (R)-3-benzyl-4-((4-(5-chloro-2-(4-methyl-3,4- | 608.1 |
| | dihydro-2H-pyrido[3,2-b][1,4]oxazin-7-yl)phenyl)-5- | |
| | fluorothiazol-2-yl)(cyclopropyl)amino)-4- | |
| | oxobutanoic acid | |
| 602 | (R)-4-((4-(5-chloro-2-(1-methyl-1H-pyrrolo[2,3- | 556.1 |
| | b]pyridin-5-yl)phenyl)-5-fluorothiazol-2- | |
| | yl)(methyl)amino)-3-(cyclopentylmethyl)-4- | |
| | oxobutanoic acid | |
| 603 | (R)-4-((4-(5-chloro-2-(1-methyl-1H-pyrrolo[2,3- | 582.1 |
| | b]pyridin-5-yl)phenyl)-5-fluorothiazol-2- | |
| | yl)(cyclopropyl)amino)-3-(cyclopentylmethyl)-4- | |
| | oxobutanoic acid | |
| 604 | (R)-4-((4-(5-chloro-2-(1-methyl-1H-pyrrolo[2,3- | 572.1 |
| | b]pyridin-5-yl)phenyl)-5-fluorothiazol-2- | |
| | yl)(methyl)amino)-4-oxo-3-((tetrahydro-2H-pyran-4- | |
| | yl)methyl)butanoic acid | |
| 605 | (R)-4-((4-(5-chloro-2-(1-methyl-1H-pyrrolo[2,3- | 598.1 |
| | b]pyridin-5-yl)phenyl)-5-fluorothiazol-2- | |
| | yl)(cyclopropyl)amino)-4-oxo-3-((tetrahydro-2H- | |
| | pyran-4-yl)methyl)butanoic acid | |
| 606 | (R)-3-benzyl-4-((4-(5-chloro-2-(1-methyl-1H- | 564.0 |
| | pyrrolo[2,3-b]pyridin-5-yl)phenyl)-5-fluorothiazol-2- | |
| | yl)(methyl)amino)-4-oxobutanoic acid | |
| 607 | (R)-3-benzyl-4-((4-(5-chloro-2-(1-methyl-1H- | 590.1 |
| | pyrrolo[2,3-b]pyridin-5-yl)phenyl)-5-fluorothiazol-2- | |
| | yl)(cyclopropyl)amino)-4-oxobutanoic acid | |
| 608 | (R)-3-(cyclopentylmethyl)-4-((5-fluoro-4-(2-(8- | 551.6 |
| | methyl-7-oxo-5,6,7,8-tetrahydro-1,8-naphthyridin-3- | |
| | yl)phenyl)thiazol-2-yl)(methyl)amino)-4-oxobutanoic | |
| | acid | |
| 609 | (R)-3-(cyclopentylmethyl)-4-(cyclopropyl(5-fluoro- | 577.7 |
| | 4-(2-(8-methyl-7-oxo-5,6,7,8-tetrahydro-1,8- | |
| | naphthyridin-3-yl)phenyl)thiazol-2-yl)amino)-4- | |
| | oxobutanoic acid | |
| 610 | (R)-4-((5-fluoro-4-(2-(8-methyl-7-oxo-5,6,7,8- | 567.6 |
| | tetrahydro-1,8-naphthyridin-3-yl)phenyl)thiazol-2- | |
| | yl)(methyl)amino)-4-oxo-3-((tetrahydro-2H-pyran-4- | |
| | yl)methyl)butanoic acid | |
| 611 | (R)-4-(cyclopropyl(5-fluoro-4-(2-(8-methyl-7-oxo- | 593.7 |
| | 5,6,7,8-tetrahydro-1,8-naphthyridin-3- | |
| | yl)phenyl)thiazol-2-yl)amino)-4-oxo-3-((tetrahydro- | |
| | 2H-pyran-4-yl)methyl)butanoic acid | |
| 612 | (R)-3-benzyl-4-((5-fluoro-4-(2-(8-methyl-7-oxo- | 559.6 |
| | 5,6,7,8-tetrahydro-1,8-naphthyridin-3- | |
| | yl)phenyl)thiazol-2-yl)(methyl)amino)-4-oxobutanoic | |
| | acid | |
| 613 | (R)-3-benzyl-4-(cyclopropyl(5-fluoro-4-(2-(8- | 585.7 |
| | methyl-7-oxo-5,6,7,8-tetrahydro-1,8-naphthyridin-3- | |
| | yl)phenyl)thiazol-2-yl)amino)-4-oxobutanoic acid | |
| 614 | (R)-3-(cyclopentylmethyl)-4-((5-fluoro-4-(2-(1- | 537.6 |
| | methyl-2-oxo-2,3-dihydro-1H-pyrrolo[2,3-b]pyridin- | |
| | 5-yl)phenyl)thiazol-2-yl)(methyl)amino)-4- | |
| | oxobutanoic acid | |
| 615 | (R)-3-(cyclopentylmethyl)-4-(cyclopropyl(5-fluoro- | 563.7 |
| | 4-(2-(1-methyl-2-oxo-2,3-dihydro-1H-pyrrolo[2,3- | |
| | b]pyridin-5-yl)phenyl)thiazol-2-yl)amino)-4- | |
| | oxobutanoic acid | |
| 616 | (R)-4-((5-fluoro-4-(2-(1-methyl-2-oxo-2,3-dihydro- | 553.6 |
| | 1H-pyrrolo[2,3-b]pyridin-5-yl)phenyl)thiazol-2- | |
| | yl)(methyl)amino)-4-oxo-3-((tetrahydro-2H-pyran-4- | |
| | yl)methyl)butanoic acid | |
| 617 | (R)-4-(cyclopropyl(5-fluoro-4-(2-(1-methyl-2-oxo- | 579.7 |
| | 2,3-dihydro-1H-pyrrolo[2,3-b]pyridin-5- | |
| | yl)phenyl)thiazol-2-yl)amino)-4-oxo-3-((tetrahydro- | |
| | 2H-pyran-4-yl)methyl)butanoic acid | |
| 618 | (R)-3-benzyl-4-((5-fluoro-4-(2-(1-methyl-2-oxo-2,3- | 545.6 |
| | dihydro-1H-pyrrolo[2,3-b]pyridin-5- | |
| | yl)phenyl)thiazol-2-yl)(methyl)amino)-4-oxobutanoic | |
| | acid | |
| 619 | (R)-3-benzyl-4-(cyclopropyl(5-fluoro-4-(2-(1- | 571.6 |
| | methyl-2-oxo-2,3-dihydro-1H-pyrrolo[2,3-b]pyridin- | |
| | 5-yl)phenyl)thiazol-2-yl)amino)-4-oxobutanoic acid | |
| 620 | (R)-3-(cyclopentylmethyl)-4-((5-fluoro-4-(2-(4- | 539.6 |
| | methyl-3,4-dihydro-2H-pyrido[3,2-b][1,4]oxazin-7- | |
| | yl)phenyl)thiazol-2-yl)(methyl)amino)-4-oxobutanoic | |
| | acid | |
| 621 | (R)-3-(cyclopentylmethyl)-4-(cyclopropyl(5-fluoro- | 565.7 |
| | 4-(2-(4-methyl-3,4-dihydro-2H-pyrido[3,2- | |
| | b][1,4]oxazin-7-yl)phenyl)thiazol-2-yl)amino)-4- | |
| | oxobutanoic acid | |
| 622 | (R)-4-((5-fluoro-4-(2-(4-methyl-3,4-dihydro-2H- | 555.6 |
| | pyrido[3,2-b][1,4]oxazin-7-yl)phenyl)thiazol-2- | |
| | yl)(methyl)amino)-4-oxo-3-((tetrahydro-2H-pyran-4- | |
| | yl)methyl)butanoic acid | |
| 623 | (R)-4-(cyclopropyl(5-fluoro-4-(2-(4-methyl-3,4- | 581.7 |
| | dihydro-2H-pyrido[3,2-b][1,4]oxazin-7- | |
| | yl)phenyl)thiazol-2-yl)amino)-4-oxo-3-((tetrahydro- | |
| | 2H-pyran-4-yl)methyl)butanoic acid | |
| 624 | (R)-3-benzyl-4-((5-fluoro-4-(2-(4-methyl-3,4- | 547.6 |
| | dihydro-2H-pyrido[3,2-b][1,4]oxazin-7- | |
| | yl)phenyl)thiazol-2-yl)(methyl)amino)-4-oxobutanoic | |
| | acid | |
| 625 | (R)-3-benzyl-4-(cyclopropyl(5-fluoro-4-(2-(4- | 573.7 |
| | methyl-3,4-dihydro-2H-pyrido[3,2-b][1,4]oxazin-7- | |
| | yl)phenyl)thiazol-2-yl)amino)-4-oxobutanoic acid | |
| 626 | (R)-3-(cyclopentylmethyl)-4-((5-fluoro-4-(2-(1- | 521.6 |
| | methyl-1H-pyrrolo[2,3-b]pyridin-5- | |
| | yl)phenyl)thiazol-2-yl)(methyl)amino)-4-oxobutanoic | |
| | acid | |
| 627 | (R)-3-(cyclopentylmethyl)-4-(cyclopropyl(5-fluoro- | 547.7 |
| | 4-(2-(1-methyl-1H-pyrrolo[2,3-b]pyridin-5- | |
| | yl)phenyl)thiazol-2-yl)amino)-4-oxobutanoic acid | |
| 628 | (R)-4-((5-fluoro-4-(2-(1-methyl-1H-pyrrolo[2,3- | 537.6 |
| | b]pyridin-5-yl)phenyl)thiazol-2-yl)(methyl)amino)-4- | |
| | oxo-3-((tetrahydro-2H-pyran-4-yl)methyl)butanoic | |
| | acid | |
| 629 | (R)-4-(cyclopropyl(5-fluoro-4-(2-(1-methyl-1H- | 563.7 |
| | pyrrolo[2,3-b]pyridin-5-yl)phenyl)thiazol-2- | |
| | yl)amino)-4-oxo-3-((tetrahydro-2H-pyran-4- | |
| | yl)methyl)butanoic acid | |
| 630 | (R)-3-benzyl-4-((5-fluoro-4-(2-(1-methyl-1H- | 529.6 |
| | pyrrolo[2,3-b]pyridin-5-yl)phenyl)thiazol-2- | |
| | yl)(methyl)amino)-4-oxobutanoic acid | |
| 631 | (R)-3-benzyl-4-(cyclopropyl(5-fluoro-4-(2-(1- | 555.6 |
| | methyl-1H-pyrrolo[2,3-b]pyridin-5- | |
| | yl)phenyl)thiazol-2-yl)amino)-4-oxobutanoic acid | |
| 632 | (R)-3-(cyclopentylmethyl)-4-(cyclopropyl(4-(2-(1- | 529.7 |
| | methyl-1H-pyrrolo[3,2-b]pyridin-6- | |
| | yl)phenyl)thiazol-2-yl)amino)-4-oxobutanoic acid | |
| 633 | (R)-3-(cyclopentylmethyl)-4-(cyclopropyl(4-(2-(1- | 546.7 |
| | methyl-2-oxo-2,3-dihydro-1H-imidazo[4,5-b]pyridin- | |
| | 6-yl)phenyl)thiazol-2-yl)amino)-4-oxobutanoic acid | |
| 634 | (R)-3-(cyclopentylmethyl)-4-(cyclopropyl(4-(2-(1- | 560.7 |
| | methyl-2-oxo-1,2,3,4-tetrahydropyrido[3,2- | |
| | d]pyrimidin-7-yl)phenyl)thiazol-2-yl)amino)-4- | |
| | oxobutanoic acid | |
| 635 | (R)-3-(cyclopentylmethyl)-4-(cyclopropyl(4-(2-(1- | 545.7 |
| | methyl-2-oxo-2,3-dihydro-1H-pyrrolo[3,2-b]pyridin- | |
| | 6-yl)phenyl)thiazol-2-yl)amino)-4-oxobutanoic acid | |
| 636 | (R)-3-(cyclopentylmethyl)-4-(cyclopropyl(4-(2-(6- | 559.7 |
| | methyl-5-oxo-5,6,7,8-tetrahydro-1,6-naphthyridin-3- | |
| | yl)phenyl)thiazol-2-yl)amino)-4-oxobutanoic acid | |
| 637 | (R)-3-(cyclopentylmethyl)-4-(cyclopropyl(4-(2-(1,3- | 574.7 |
| | dimethyl-2-oxo-1,2,3,4-tetrahydropyrido[3,2- | |
| | d]pyrimidin-7-yl)phenyl)thiazol-2-yl)amino)-4- | |
| | oxobutanoic acid | |
| 638 | (R)-3-(cyclopentylmethyl)-4-(cyclopropyl(4-(2-(7- | 559.7 |
| | methyl-8-oxo-5,6,7,8-tetrahydro-1,7-naphthyridin-3- | |
| | yl)phenyl)thiazol-2-yl)amino)-4-oxobutanoic acid | |
| 639 | (R)-3-(cyclopentylmethyl)-4-(cyclopropyl(4-(2-(6- | 545.7 |
| | methyl-5-oxo-6,7-dihydro-5H-pyrrolo[3,4-b]pyridin- | |
| | 3-yl)phenyl)thiazol-2-yl)amino)-4-oxobutanoic acid | |
| 640 | (R)-4-((4-(2-(5-chloro-6-(2-oxopyrrolidin-1- | 594.1 |
| | yl)pyridin-3-yl)phenyl)thiazol-2- | |
| | yl)(cyclopropyl)amino)-3-(cyclopentylmethyl)-4- | |
| | oxobutanoic acid | |
| 641 | (R)-3-(cyclopentylmethyl)-4-(cyclopropyl(4-(2-(3- | 530.7 |
| | methyl-3H-imidazo[4,5-b]pyridin-6- | |
| | yl)phenyl)thiazol-2-yl)amino)-4-oxobutanoic acid | |
| 642 | (R)-3-(cyclopentylmethyl)-4-(cyclopropyl(4-(2-(1- | 547.7 |
| | methyl-2,3-dihydro-1H-pyrido[2,3-b][1,4]oxazin-7- | |
| | yl)phenyl)thiazol-2-yl)amino)-4-oxobutanoic acid | |
| 643 | (R)-3-(cyclopentylmethyl)-4-(cyclopropyl(4-(2-(3- | 546.7 |
| | methyl-2-oxo-2,3-dihydro-1H-imidazo[4,5-b]pyridin- | |
| | 6-yl)phenyl)thiazol-2-yl)amino)-4-oxobutanoic acid | |
| 644 | (R)-3-(cyclopentylmethyl)-4-(cyclopropyl(4-(2-(7- | 559.7 |
| | methyl-6-oxo-5,6,7,8-tetrahydro-1,7-naphthyridin-3- | |
| | yl)phenyl)thiazol-2-yl)amino)-4-oxobutanoic acid | |
| 645 | (R)-3-(cyclopentylmethyl)-4-(cyclopropyl(4-(2-(6- | 545.7 |
| | methyl-7-oxo-6,7-dihydro-5H-pyrrolo[3,4-b]pyridin- | |
| | 3-yl)phenyl)thiazol-2-yl)amino)-4-oxobutanoic acid | |
| 646 | (R)-3-(cyclopentylmethyl)-4-(cyclopropyl(4-(2-(1,3- | 560.7 |
| | dimethyl-2-oxo-2,3-dihydro-1H-imidazo[4,5- | |
| | b]pyridin-6-yl)phenyl)thiazol-2-yl)amino)-4- | |
| | oxobutanoic acid | |
| 647 | (R)-3-(cyclopentylmethyl)-4-(cyclopropyl(4-(2-(1- | 530.7 |
| | methyl-1H-imidazo[4,5-b]pyridin-6- | |
| | yl)phenyl)thiazol-2-yl)amino)-4-oxobutanoic acid | |
| 648 | (R)-3-(cyclopentylmethyl)-4-(cyclopropyl(4-(2-(5- | 577.7 |
| | fluoro-6-(2-oxopyrrolidin-1-yl)pyridin-3- | |
| | yl)phenyl)thiazol-2-yl)amino)-4-oxobutanoic acid | |
| 649 | (R)-3-(cyclopentylmethyl)-4-(cyclopropyl(4-(2-(1- | 559.7 |
| | methyl-2-oxo-1,2,3,4-tetrahydro-1,5-naphthyridin-7- | |
| | yl)phenyl)thiazol-2-yl)amino)-4-oxobutanoic acid | |
| 650 | (R)-3-(cyclopentylmethyl)-4-(cyclopropyl(4-(2-(3- | 560.7 |
| | methyl-2-oxo-1,2,3,4-tetrahydropyrido[3,2- | |
| | d]pyrimidin-7-yl)phenyl)thiazol-2-yl)amino)-4- | |
| | oxobutanoic acid | |
| 651 | (R)-4-(cyclopropyl(5-fluoro-4-(5-methyl-2-(6-(2- | 597.7 |
| | oxopyrrolidin-1-yl)pyridin-3-yl)furan-3-yl)thiazol-2- | |
| | yl)amino)-4-oxo-3-((tetrahydro-2H-pyran-4- | |
| | yl)methyl)butanoic acid | |
| 652 | (R)-4-(cyclopropyl(4-(5-methyl-2-(6-(2- | 579.7 |
| | oxopyrrolidin-1-yl)pyridin-3-yl)furan-3-yl)thiazol-2- | |
| | yl)amino)-4-oxo-3-((tetrahydro-2H-pyran-4- | |
| | yl)methyl)butanoic acid | |
| 653 | (R)-4-((5-fluoro-4-(5-methyl-2-(6-(2-oxopyrrolidin- | 571.6 |
| | 1-yl)pyridin-3-yl)furan-3-yl)thiazol-2- | |
| | yl)(methyl)amino)-4-oxo-3-((tetrahydro-2H-pyran-4- | |
| | yl)methyl)butanoic acid | |
| 654 | (R)-4-(methyl(4-(5-methyl-2-(6-(2-oxopyrrolidin-1- | 553.6 |
| | yl)pyridin-3-yl)furan-3-yl)thiazol-2-yl)amino)-4-oxo- | |
| | 3-((tetrahydro-2H-pyran-4-yl)methyl)butanoic acid | |
| 655 | (R)-3-(cyclopentylmethyl)-4-(cyclopropyl(5-fluoro- | 581.7 |
| | 4-(5-methyl-2-(6-(2-oxopyrrolidin-1-yl)pyridin-3- | |
| | yl)furan-3-yl)thiazol-2-yl)amino)-4-oxobutanoic acid | |
| 656 | (R)-3-(cyclopentylmethyl)-4-(cyclopropyl(4-(5- | 563.7 |
| | methyl-2-(6-(2-oxopyrrolidin-1-yl)pyridin-3- | |
| | yl)furan-3-yl)thiazol-2-yl)amino)-4-oxobutanoic acid | |
| 657 | (R)-3-(cyclopentylmethyl)-4-((5-fluoro-4-(5-methyl- | 555.6 |
| | 2-(6-(2-oxopyrrolidin-1-yl)pyridin-3-yl)furan-3- | |
| | yl)thiazol-2-yl)(methyl)amino)-4-oxobutanoic acid | |
| 658 | (R)-3-(cyclopentylmethyl)-4-(methyl(4-(5-methyl-2- | 537.6 |
| | (6-(2-oxopyrrolidin-1-yl)pyridin-3-yl)furan-3- | |
| | yl)thiazol-2-yl)amino)-4-oxobutanoic acid | |
| 659 | (R)-3-benzyl-4-(cyclopropyl(5-fluoro-4-(5-methyl-2- | 589.6 |
| | (6-(2-oxopyrrolidin-1-yl)pyridin-3-yl)furan-3- | |
| | yl)thiazol-2-yl)amino)-4-oxobutanoic acid | |
| 660 | (R)-3-benzyl-4-(cyclopropyl(4-(5-methyl-2-(6-(2- | 571.7 |
| | oxopyrrolidin-1-yl)pyridin-3-yl)furan-3-yl)thiazol-2- | |
| | yl)amino)-4-oxobutanoic acid | |
| 661 | (R)-3-benzyl-4-((5-fluoro-4-(5-methyl-2-(6-(2- | 563.6 |
| | oxopyrrolidin-1-yl)pyridin-3-yl)furan-3-yl)thiazol-2- | |
| | yl)(methyl)amino)-4-oxobutanoic acid | |
| 662 | (R)-3-benzyl-4-(methyl(4-(5-methyl-2-(6-(2- | 545.6 |
| | oxopyrrolidin-1-yl)pyridin-3-yl)furan-3-yl)thiazol-2- | |
| | yl)amino)-4-oxobutanoic acid | |
| 663 | (R)-3-benzyl-4-(methyl(3-(2-(6-(2-oxopyrrolidin-1- | 542.6 |
| | yl)pyridin-3-yl)phenyl)-1,2,4-thiadiazol-5-yl)amino)- | |
| | 4-oxobutanoic acid | |
| 664 | (R)-3-benzyl-4-(cyclopropyl(3-(2-(6-(2- | 568.7 |
| | oxopyrrolidin-1-yl)pyridin-3-yl)phenyl)-1,2,4- | |
| | thiadiazol-5-yl)amino)-4-oxobutanoic acid | |
| 665 | (R)-3-(cyclopentylmethyl)-4-(methyl(3-(2-(6-(2- | 534.6 |
| | oxopyrrolidin-1-yl)pyridin-3-yl)phenyl)-1,2,4- | |
| | thiadiazol-5-yl)amino)-4-oxobutanoic acid | |
| 666 | (R)-3-(cyclopentylmethyl)-4-(cyclopropyl(3-(2-(6-(2- | 560.7 |
| | oxopyrrolidin-1-yl)pyridin-3-yl)phenyl)-1,2,4- | |
| | thiadiazol-5-yl)amino)-4-oxobutanoic acid | |
| 667 | (R)-4-(methyl(3-(2-(6-(2-oxopyrrolidin-1-yl)pyridin- | 550.6 |
| | 3-yl)phenyl)-1,2,4-thiadiazol-5-yl)amino)-4-oxo-3- | |
| | ((tetrahydro-2H-pyran-4-yl)methyl)butanoic acid | |
| 668 | (R)-4-(cyclopropyl(3-(2-(6-(2-oxopyrrolidin-1- | 576.7 |
| | yl)pyridin-3-yl)phenyl)-1,2,4-thiadiazol-5-yl)amino)- | |
| | 4-oxo-3-((tetrahydro-2H-pyran-4-yl)methyl)butanoic | |
| | acid | |
| 669 | (3R)-3-(cyclopentylmethyl)-4-(cyclopropyl(4-(2,5- | 595.7 |
| | dimethyl-4-(6-(2-oxopyrrolidin-1-yl)pyridin-3- | |
| | yl)furan-3-yl)-5-fluorothiazol-2-yl)amino)-4- | |
| | oxobutanoic acid | |
| 670 | (3R)-3-(cyclopentylmethyl)-4-((4-(2,5-dimethyl-4-(6- | 569.7 |
| | (2-oxopyrrolidin-1-yl)pyridin-3-yl)furan-3-yl)-5- | |
| | fluorothiazol-2-yl)(methyl)amino)-4-oxobutanoic | |
| | acid | |
| 671 | (R)-3-(cyclopentylmethyl)-4-(cyclopropyl(4-(2,5- | 577.7 |
| | dimethyl-4-(6-(2-oxopyrrolidin-1-yl)pyridin-3- | |
| | yl)furan-3-yl)thiazol-2-yl)amino)-4-oxobutanoic acid | |
| 672 | (R)-3-(cyclopentylmethyl)-4-((4-(2,5-dimethyl-4-(6- | 551.7 |
| | (2-oxopyrrolidin-1-yl)pyridin-3-yl)furan-3-yl)thiazol- | |
| | 2-yl)(methyl)amino)-4-oxobutanoic acid | |
| 673 | (3R)-4-(cyclopropyl(4-(2,5-dimethyl-4-(6-(2- | 611.7 |
| | oxopyrrolidin-1-yl)pyridin-3-yl)furan-3-yl)-5- | |
| | fluorothiazol-2-yl)amino)-4-oxo-3-((tetrahydro-2H- | |
| | pyran-4-yl)methyl)butanoic acid | |
| 674 | (3R)-4-((4-(2,5-dimethyl-4-(6-(2-oxopyrrolidin-1- | 585.7 |
| | yl)pyridin-3-yl)furan-3-yl)-5-fluorothiazol-2- | |
| | yl)(methyl)amino)-4-oxo-3-((tetrahydro-2H-pyran-4- | |
| | yl)methyl)butanoic acid | |
| 675 | (R)-4-(cyclopropyl(4-(2,5-dimethyl-4-(6-(2- | 593.7 |
| | oxopyrrolidin-1-yl)pyridin-3-yl)furan-3-yl)thiazol-2- | |
| | yl)amino)-4-oxo-3-((tetrahydro-2H-pyran-4- | |
| | yl)methyl)butanoic acid | |
| 676 | (R)-4-((4-(2,5-dimethyl-4-(6-(2-oxopyrrolidin-1- | 567.7 |
| | yl)pyridin-3-yl)furan-3-yl)thiazol-2- | |
| | yl)(methyl)amino)-4-oxo-3-((tetrahydro-2H-pyran-4- | |
| | yl)methyl)butanoic acid | |
| 677 | (3R)-3-benzyl-4-(cyclopropyl(4-(2,5-dimethyl-4-(6- | 603.7 |
| | (2-oxopyrrolidin-1-yl)pyridin-3-yl)furan-3-yl)-5- | |
| | fluorothiazol-2-yl)amino)-4-oxobutanoic acid | |
| 678 | (3R)-3-benzyl-4-((4-(2,5-dimethyl-4-(6-(2- | 577.6 |
| | oxopyrrolidin-1-yl)pyridin-3-yl)furan-3-yl)-5- | |
| | fluorothiazol-2-yl)(methyl)amino)-4-oxobutanoic | |
| | acid | |
| 679 | (R)-3-benzyl-4-(cyclopropyl(4-(2,5-dimethyl-4-(6-(2- | 585.7 |
| | oxopyrrolidin-1-yl)pyridin-3-yl)furan-3-yl)thiazol-2- | |
| | yl)amino)-4-oxobutanoic acid | |
| 680 | (R)-3-benzyl-4-((4-(2,5-dimethyl-4-(6-(2- | 559.6 |
| | oxopyrrolidin-1-yl)pyridin-3-yl)furan-3-yl)thiazol-2- | |
| | yl)(methyl)amino)-4-oxobutanoic acid | |
| 681 | (3R)-3-(cyclopentylmethyl)-4-(cyclopropyl(4-(2-(6- | 587.7 |
| | (1-methyl-6-oxopiperidin-3-yl)pyridin-3- | |
| | yl)phenyl)thiazol-2-yl)amino)-4-oxobutanoic acid | |
| 682 | (3R)-3-(cyclopentylmethyl)-4-(cyclopropyl(4-(2-(6- | 587.7 |
| | (1-methyl-2-oxopiperidin-4-yl)pyridin-3- | |
| | yl)phenyl)thiazol-2-yl)amino)-4-oxobutanoic acid | |
| 683 | (3R)-3-(cyclopentylmethyl)-4-(cyclopropyl(4-(2-(6- | 574.7 |
| | (3-methyl-2-oxoimidazolidin-4-yl)pyridin-3- | |
| | yl)phenyl)thiazol-2-yl)amino)-4-oxobutanoic acid | |
| 684 | (R)-3-(cyclopentylmethyl)-4-(cyclopropyl(4-(2-(6- | 547.7 |
| | (N-methylacetamido)pyridin-3-yl)phenyl)thiazol-2- | |
| | yl)amino)-4-oxobutanoic acid | |
| 685 | (R)-3-(cyclopentylmethyl)-4-(cyclopropyl(4-(2-(6- | 602.8 |
| | (1,3-dimethyl-2-oxohexahydropyrimidin-5- | |
| | yl)pyridin-3-yl)phenyl)thiazol-2-yl)amino)-4- | |
| | oxobutanoic acid | |
| 686 | (3R)-3-(cyclopentylmethyl)-4-(cyclopropyl(4-(2-(6- | 559.7 |
| | (5-oxopyrrolidin-3-yl)pyridin-3-yl)phenyl)thiazol-2- | |
| | yl)amino)-4-oxobutanoic acid | |
| 687 | (3R)-3-(cyclopentylmethyl)-4-(cyclopropyl(4-(2-(6- | 574.7 |
| | (1-methyl-2-oxoimidazolidin-4-yl)pyridin-3- | |
| | yl)phenyl)thiazol-2-yl)amino)-4-oxobutanoic acid | |
| 688 | (R)-3-(cyclopentylmethyl)-4-(cyclopropyl(4-(2-(6- | 545.7 |
| | (pyrrolidin-1-yl)pyridin-3-yl)phenyl)thiazol-2- | |
| | yl)amino)-4-oxobutanoic acid | |
| 689 | (3R)-3-(cyclopentylmethyl)-4-(cyclopropyl(4-(2-(6- | 560.7 |
| | (2-oxoimidazolidin-4-yl)pyridin-3-yl)phenyl)thiazol- | |
| | 2-yl)amino)-4-oxobutanoic acid | |
| 690 | (3R)-3-(cyclopentylmethyl)-4-(cyclopropyl(4-(2-(6- | 573.7 |
| | (1-methyl-5-oxopyrrolidin-2-yl)pyridin-3- | |
| | yl)phenyl)thiazol-2-yl)amino)-4-oxobutanoic acid | |
| 691 | (R)-3-(cyclopentylmethyl)-4-(cyclopropyl(4-(2-(6-(4- | 588.7 |
| | methyl-3-oxopiperazin-1-yl)pyridin-3- | |
| | yl)phenyl)thiazol-2-yl)amino)-4-oxobutanoic acid | |
| 692 | (R)-3-(cyclopentylmethyl)-4-(cyclopropyl(4-(2-(6-(4- | 602.7 |
| | methyl-2,5-dioxopiperazin-1-yl)pyridin-3- | |
| | yl)phenyl)thiazol-2-yl)amino)-4-oxobutanoic acid | |
| 693 | (R)-3-(cyclopentylmethyl)-4-(cyclopropyl(4-(2-(6- | 547.7 |
| | (dimethylcarbamoyl)pyridin-3-yl)phenyl)thiazol-2- | |
| | yl)amino)-4-oxobutanoic acid | |
| 694 | (3R)-3-(cyclopentylmethyl)-4-(cyclopropyl(4-(2-(6- | 588.7 |
| | (3-methyl-2-oxohexahydropyrimidin-4-yl)pyridin-3- | |
| | yl)phenyl)thiazol-2-yl)amino)-4-oxobutanoic acid | |
| 695 | (R)-3-(cyclopentylmethyl)-4-(cyclopropyl(4-(2-(6- | 534.7 |
| | isopropoxypyridin-3-yl)phenyl)thiazol-2-yl)amino)- | |
| | 4-oxobutanoic acid | |
| 696 | (3R)-3-(cyclopentylmethyl)-4-(cyclopropyl(4-(2-(6- | 587.7 |
| | (1-methyl-6-oxopiperidin-2-yl)pyridin-3- | |
| | yl)phenyl)thiazol-2-yl)amino)-4-oxobutanoic acid | |
| 697 | (3R)-3-(cyclopentylmethyl)-4-(cyclopropyl(4-(2-(6- | 573.7 |
| | (1-methyl-5-oxopyrrolidin-3-yl)pyridin-3- | |
| | yl)phenyl)thiazol-2-yl)amino)-4-oxobutanoic acid | |
| 698 | (R)-3-(cyclopentylmethyl)-4-(cyclopropyl(4-(2-(6-(3- | 588.7 |
| | methyl-2-oxotetrahydropyrimidin-1(2H)-yl)pyridin- | |
| | 3-yl)phenyl)thiazol-2-yl)amino)-4-oxobutanoic acid | |
| 699 | (R)-3-(cyclopentylmethyl)-4-(cyclopropyl(4-(2-(6-(2- | 574.7 |
| | oxotetrahydropyrimidin-1(2H)-yl)pyridin-3- | |
| | yl)phenyl)thiazol-2-yl)amino)-4-oxobutanoic acid | |
| 700 | (3R)-3-(cyclopentylmethyl)-4-(cyclopropyl(4-(2-(6- | 588.7 |
| | (1,3-dimethyl-2-oxoimidazolidin-4-yl)pyridin-3- | |
| | yl)phenyl)thiazol-2-yl)amino)-4-oxobutanoic acid | |
| 701 | (3R)-3-(cyclopentylmethyl)-4-(cyclopropyl(4-(2-(6- | 602.8 |
| | (1,3-dimethyl-2-oxohexahydropyrimidin-4- | |
| | yl)pyridin-3-yl)phenyl)thiazol-2-yl)amino)-4- | |
| | oxobutanoic acid | |
| 702 | (3R)-3-(cyclopentylmethyl)-4-(cyclopropyl(4-(2-(6- | 588.7 |
| | (1-methyl-2-oxohexahydropyrimidin-4-yl)pyridin-3- | |
| | yl)phenyl)thiazol-2-yl)amino)-4-oxobutanoic acid | |
| 703 | (R)-3-(cyclopentylmethyl)-4-(cyclopropyl(4-(2-(6- | 573.7 |
| | (N-methylcyclopropanecarboxamido)pyridin-3- | |
| | yl)phenyl)thiazol-2-yl)amino)-4-oxobutanoic acid | |
| 704 | (3R)-3-(cyclopentylmethyl)-4-(cyclopropyl(4-(2-(6- | 573.7 |
| | (1-methyl-2-oxopyrrolidin-3-yl)pyridin-3- | |
| | yl)phenyl)thiazol-2-yl)amino)-4-oxobutanoic acid | |
| 705 | (R)-3-(cyclopentylmethyl)-4-(cyclopropyl(4-(2-(6- | 546.7 |
| | (cyclopropylmethoxy)pyridin-3-yl)phenyl)thiazol-2- | |
| | yl)amino)-4-oxobutanoic acid | |
| 706 | (3R)-3-(cyclopentylmethyl)-4-(cyclopropyl(4-(2-(6- | 559.7 |
| | (2-oxopyrrolidin-3-yl)pyridin-3-yl)phenyl)thiazol-2- | |
| | yl)amino)-4-oxobutanoic acid | |
| 707 | (3R)-3-(cyclopentylmethyl)-4-(cyclopropyl(4-(2-(6- | 587.7 |
| | (1-methyl-2-oxopiperidin-3-yl)pyridin-3- | |
| | yl)phenyl)thiazol-2-yl)amino)-4-oxobutanoic acid | |
| 708 | (R)-3-(cyclopentylmethyl)-4-(cyclopropyl(4-(2-(6- | 520.7 |
| | (methoxymethyl)pyridin-3-yl)phenyl)thiazol-2- | |
| | yl)amino)-4-oxobutanoic acid | |
| 709 | (3R)-3-(cyclopentylmethyl)-4-(cyclopropyl(4-(2-(6- | 588.7 |
| | (1-methyl-2-oxohexahydropyrimidin-5-yl)pyridin-3- | |
| | yl)phenyl)thiazol-2-yl)amino)-4-oxobutanoic acid | |
| 710 | (3R)-3-(cyclopentylmethyl)-4-(cyclopropyl(4-(2-(6- | 559.7 |
| | (5-oxopyrrolidin-2-yl)pyridin-3-yl)phenyl)thiazol-2- | |
| | yl)amino)-4-oxobutanoic acid | |
| 711 | (R)-3-(cyclopentylmethyl)-4-(cyclopropyl(4-(2-(6-(2- | 627.7 |
| | oxopyrrolidin-1-yl)pyridin-3-yl)-5- | |
| | (trifluoromethyl)phenyl)thiazol-2-yl)amino)-4- | |
| | oxobutanoic acid | |
| 712 | (R)-3-(cyclopentylmethyl)-4-(cyclopropyl(4-(5- | 607.7 |
| | (fluoromethoxy)-2-(6-(2-oxopyrrolidin-1-yl)pyridin- | |
| | 3-yl)phenyl)thiazol-2-yl)amino)-4-oxobutanoic acid | |
| 713 | (R)-3-(cyclopentylmethyl)-4-(cyclopropyl(4-(2-(6-(2- | 643.7 |
| | oxopyrrolidin-1-yl)pyridin-3-yl)-5- | |
| | (trifluoromethoxy)phenyl)thiazol-2-yl)amino)-4- | |
| | oxobutanoic acid | |
| 714 | (R)-4-(cyclopropyl(4-(2-(6-(2-oxopyrrolidin-1- | 568.7 |
| | yl)pyridin-3-yl)phenyl)thiazol-2-yl)amino)-4-oxo-3- | |
| | (pyridin-2-ylmethyl)butanoic acid | |
| 715 | (R)-2-(2-(carboxymethyl)-3-(cyclopropyl(4-(2-(6-(2- | 584.7 |
| | oxopyrrolidin-1-yl)pyridin-3-yl)phenyl)thiazol-2- | |
| | yl)amino)-3-oxopropyl)pyridine 1-oxide | |
| 716 | (R)-4-(cyclopropyl(4-(2-(6-(2-oxopyrrolidin-1- | 575.7 |
| | yl)pyridin-3-yl)phenyl)thiazol-2-yl)amino)-4-oxo-3- | |
| | (((R)-tetrahydro-2H-pyran-2-yl)methyl)butanoic acid | |
| 717 | (R)-4-(cyclopropyl(4-(2-(6-(2-oxopyrrolidin-1- | 569.6 |
| | yl)pyridin-3-yl)phenyl)thiazol-2-yl)amino)-4-oxo-3- | |
| | (pyrimidin-2-ylmethyl)butanoic acid | |
| 718 | (S)-4-(cyclopropyl(4-(2-(6-(2-oxopyrrolidin-1- | 573.7 |
| | yl)pyridin-3-yl)phenyl)thiazol-2-yl)amino)-4-oxo-3- | |
| | (thiophen-2-ylmethyl)butanoic acid | |
| 719 | (R)-4-(cyclopropyl(4-(2-(6-(2-oxopyrrolidin-1- | 561.7 |
| | yl)pyridin-3-yl)phenyl)thiazol-2-yl)amino)-4-oxo-3- | |
| | (((S)-tetrahydrofuran-2-yl)methyl)butanoic acid | |
| 720 | (R)-4-(cyclopropyl(4-(2-(6-(2-oxopyrrolidin-1- | 575.7 |
| | yl)pyridin-3-yl)phenyl)thiazol-2-yl)amino)-4-oxo-3- | |
| | (((S)-tetrahydro-2H-pyran-3-yl)methyl)butanoic acid | |
| 721 | (3R)-4-(cyclopropyl(4-(2-(6-(2-oxopyrrolidin-1- | 589.7 |
| | yl)pyridin-3-yl)phenyl)thiazol-2-yl)amino)-3-(((2S)- | |
| | 2-methyltetrahydro-2H-pyran-4-yl)methyl)-4- | |
| | oxobutanoic acid | |
| 722 | (R)-4-(cyclopropyl(4-(2-(6-(2-oxopyrrolidin-1- | 575.7 |
| | yl)pyridin-3-yl)phenyl)thiazol-2-yl)amino)-4-oxo-3- | |
| | (((R)-tetrahydro-2H-pyran-3-yl)methyl)butanoic acid | |
| 723 | (3R)-4-(cyclopropyl(4-(2-(6-(2-oxopyrrolidin-1- | 603.7 |
| | yl)pyridin-3-yl)phenyl)thiazol-2-yl)amino)-3- | |
| | (((3R,5S)-3,5-dimethyltetrahydro-2H-pyran-4- | |
| | yl)methyl)-4-oxobutanoic acid | |
| 724 | (R)-4-(cyclopropyl(4-(2-(6-(2-oxopyrrolidin-1- | 589.7 |
| | yl)pyridin-3-yl)phenyl)thiazol-2-yl)amino)-3- | |
| | (((2R,3R)-2-methyltetrahydro-2H-pyran-3- | |
| | yl)methyl)-4-oxobutanoic acid | |
| 725 | (3R)-4-(cyclopropyl(4-(2-(6-(2-oxopyrrolidin-1- | 589.7 |
| | yl)pyridin-3-yl)phenyl)thiazol-2-yl)amino)-3-(((3S)- | |
| | 3-methyltetrahydro-2H-pyran-4-yl)methyl)-4- | |
| | oxobutanoic acid | |
| 726 | (R)-4-(cyclopropyl(4-(2-(6-(2-oxopyrrolidin-1- | 575.7 |
| | yl)pyridin-3-yl)phenyl)thiazol-2-yl)amino)-4-oxo-3- | |
| | (((S)-tetrahydro-2H-pyran-2-yl)methyl)butanoic acid | |
| 727 | (R)-3-(benzofuran-2-ylmethyl)-4-(cyclopropyl(4-(2- | 607.7 |
| | (6-(2-oxopyrrolidin-1-yl)pyridin-3-yl)phenyl)thiazol- | |
| | 2-yl)amino)-4-oxobutanoic acid | |
| 728 | (R)-4-(cyclopropyl(4-(2-(6-(2-oxopyrrolidin-1- | 561.7 |
| | yl)pyridin-3-yl)phenyl)thiazol-2-yl)amino)-4-oxo-3- | |
| | (((R)-tetrahydrofuran-2-yl)methyl)butanoic acid | |
| 729 | (R)-4-(cyclopropyl(4-(2-(6-(2-oxopyrrolidin-1- | 571.7 |
| | yl)pyridin-3-yl)phenyl)thiazol-2-yl)amino)-3-((5- | |
| | methylfuran-2-yl)methyl)-4-oxobutanoic acid | |
| 730 | (3R)-4-(cyclopropyl(4-(2-(6-(2-oxopyrrolidin-1- | 575.7 |
| | yl)pyridin-3-yl)phenyl)thiazol-2-yl)amino)-4-oxo-3- | |
| | ((tetrahydro-2H-pyran-3-yl)methyl)butanoic acid | |
| 731 | (3R)-4-(cyclopropyl(4-(2-(6-(2-oxopyrrolidin-1- | 603.7 |
| | yl)pyridin-3-yl)phenyl)thiazol-2-yl)amino)-3- | |
| | (((2R,6S)-2,6-dimethyltetrahydro-2H-pyran-4- | |
| | yl)methyl)-4-oxobutanoic acid | |
| 732 | (R)-4-(cyclopropyl(4-(2-(6-(2-oxopyrrolidin-1- | 557.6 |
| | yl)pyridin-3-yl)phenyl)thiazol-2-yl)amino)-3-(furan- | |
| | 2-ylmethyl)-4-oxobutanoic acid | |
| 733 | (3R)-4-(cyclopropyl(4-(2-(6-(2-oxopyrrolidin-1- | 575.7 |
| | yl)pyridin-3-yl)phenyl)thiazol-2-yl)amino)-4-oxo-3- | |
| | ((tetrahydro-2H-pyran-2-yl)methyl)butanoic acid | |
| 734 | (R)-3-(cyclopentylmethyl)-4-(cyclopropyl(4-(2-(6- | 506.6 |
| | methoxypyridin-3-yl)phenyl)thiazol-2-yl)amino)-4- | |
| | oxobutanoic acid | |
| 735 | (R)-4-((4-(5-chloro-2-(6-methoxypyridin-3- | 541.1 |
| | yl)phenyl)thiazol-2-yl)(cyclopropyl)amino)-3- | |
| | (cyclopentylmethyl)-4-oxobutanoic acid | |
| 736 | (R)-4-(cyclopropyl(4-(2-(6-(2-oxopyrrolidin-1- | 547.6 |
| | yl)pyridin-3-yl)phenyl)thiazol-2-yl)amino)-3- | |
| | (oxetan-3-ylmethyl)-4-oxobutanoic acid | |
| 737 | (R)-3-(cyclopentylmethyl)-4-(cyclopropyl(4-(2-(6- | 532.7 |
| | (oxetan-3-yl)pyridin-3-yl)phenyl)thiazol-2- | |
| | yl)amino)-4-oxobutanoic acid | |
| 738 | (R)-4-(cyclopropyl(4-(2-(6-(2-oxopyrrolidin-1- | 547.6 |
| | yl)pyridin-3-yl)phenyl)thiazol-2-yl)amino)-3- | |
| | (oxetan-3-ylmethyl)-4-oxobutanoic acid | |
| 739 | (R)-3-(cyclopentylmethyl)-4-(cyclopropyl(4-(2-(6-(3- | 546.7 |
| | methyloxetan-3-yl)pyridin-3-yl)phenyl)thiazol-2- | |
| | yl)amino)-4-oxobutanoic acid | |
| 740 | (R)-4-(cyclopropyl(4-(2-(6-(2-oxopyrrolidin-1- | 547.6 |
| | yl)pyridin-3-yl)phenyl)thiazol-2-yl)amino)-3- | |
| | (oxetan-3-ylmethyl)-4-oxobutanoic acid | |
| 741 | (R)-3-(cyclopentylmethyl)-4-(cyclopropyl(4-(2-(6-(3- | 550.7 |
| | fluorooxetan-3-yl)pyridin-3-yl)phenyl)thiazol-2- | |
| | yl)amino)-4-oxobutanoic acid | |
| 742 | (R)-4-(cyclopropyl(4-(2-(6-(2-oxopyrrolidin-1- | 561.7 |
| | yl)pyridin-3-yl)phenyl)thiazol-2-yl)amino)-3-((3- | |
| | methyloxetan-3-yl)methyl)-4-oxobutanoic acid | |
| 743 | (R)-3-(cyclopentylmethyl)-4-(cyclopropyl(4-(2-(6- | 532.7 |
| | (oxetan-3-yl)pyridin-3-yl)phenyl)thiazol-2- | |
| | yl)amino)-4-oxobutanoic acid | |
| 744 | (R)-4-(cyclopropyl(4-(2-(6-(2-oxopyrrolidin-1- | 561.7 |
| | yl)pyridin-3-yl)phenyl)thiazol-2-yl)amino)-3-((3- | |
| | methyloxetan-3-yl)methyl)-4-oxobutanoic acid | |
| 745 | (R)-3-(cyclopentylmethyl)-4-(cyclopropyl(4-(2-(6-(3- | 546.7 |
| | methyloxetan-3-yl)pyridin-3-yl)phenyl)thiazol-2- | |
| | yl)amino)-4-oxobutanoic acid | |
| 746 | (R)-4-(cyclopropyl(4-(2-(6-(2-oxopyrrolidin-1- | 561.7 |
| | yl)pyridin-3-yl)phenyl)thiazol-2-yl)amino)-3-((3- | |
| | methyloxetan-3-yl)methyl)-4-oxobutanoic acid | |
| 747 | (R)-3-(cyclopentylmethyl)-4-(cyclopropyl(4-(2-(6-(3- | 550.7 |
| | fluorooxetan-3-yl)pyridin-3-yl)phenyl)thiazol-2- | |
| | yl)amino)-4-oxobutanoic acid | |
| 748 | (S)-4-(cyclopropyl(4-(2-(6-(2-oxopyrrolidin-1- | 565.6 |
| | yl)pyridin-3-yl)phenyl)thiazol-2-yl)amino)-3-((3- | |
| | fluorooxetan-3-yl)methyl)-4-oxobutanoic acid | |
| 749 | (R)-3-(cyclopentylmethyl)-4-(cyclopropyl(4-(2-(6- | 532.7 |
| | (oxetan-3-yl)pyridin-3-yl)phenyl)thiazol-2- | |
| | yl)amino)-4-oxobutanoic acid | |
| 750 | (S)-4-(cyclopropyl(4-(2-(6-(2-oxopyrrolidin-1- | 565.6 |
| | yl)pyridin-3-yl)phenyl)thiazol-2-yl)amino)-3-((3- | |
| | fluorooxetan-3-yl)methyl)-4-oxobutanoic acid | |
| 751 | (R)-3-(cyclopentylmethyl)-4-(cyclopropyl(4-(2-(6-(3- | 546.7 |
| | methyloxetan-3-yl)pyridin-3-yl)phenyl)thiazol-2- | |
| | yl)amino)-4-oxobutanoic acid | |
| 752 | (S)-4-(cyclopropyl(4-(2-(6-(2-oxopyrrolidin-1- | 565.6 |
| | yl)pyridin-3-yl)phenyl)thiazol-2-yl)amino)-3-((3- | |
| | fluorooxetan-3-yl)methyl)-4-oxobutanoic acid | |
| 753 | (R)-3-(cyclopentylmethyl)-4-(cyclopropyl(4-(2-(6-(3- | 550.7 |
| | fluorooxetan-3-yl)pyridin-3-yl)phenyl)thiazol-2- | |
| | yl)amino)-4-oxobutanoic acid | |

The compounds of formula I can be prepared by different ways with reactions known by the person skilled in the art. Reaction schemes as described in the example section illustrate by way of example different possible approaches.

The invention further provides the use of the compounds of the invention or pharmaceutically acceptable salts, or solvates thereof as agonists or partial agonists of G-protein coupled receptor 43 (GPR43).

Accordingly, in a particularly preferred embodiment, the invention relates to the use of compounds of formula I and subformulae in particular those of table 1 above, or pharmaceutically acceptable salts and solvates thereof, as GPR43 agonists or partial agonists.

### [Applications]

The compounds of the invention are therefore useful in the prevention or in the prevention and/or treatment of type II diabetes, obesity, dyslipidemia such as mixed or diabetic dyslipidemia, hypercholesterolemia, low HDL cholesterol, high LDL cholesterol, hyperlipidemia, hypertriglyceridemia, hypoglycemia, hyperglycemia, glucose intolerance, insulin resistance, hyperinsulinemia hypertension, hyperlipoproteinemia, metabolic syndrome, syndrome X, thrombotic disorders, cardiovascular disease, atherosclerosis and its sequelae including angina, claudication, heart attack, stroke and others, kidney diseases, ketoacidosis, nephropathy, diabetic neuropathy, diabetic retinopathy, nonalcoholic fatty liver diseases such as steatosis or nonalcoholic steatohepatitis (NASH).

Preferred diseases are type II diabetes, lipid disorders such as dyslipidemia, hypertension, obesity, atherosclerosis and its sequelae.

In a particular preferred embodiment the diseases are type II diabetes and a lipid disorder such as dyslipidemia.

The invention also provides for a method for delaying in patient the onset of type II diabetes, obesity, dyslipidemia such as mixed or diabetic dyslipidemia, hypercholesterolemia, low HDL cholesterol, high LDL cholesterol, hyperlipidemia, hypertriglyceridemia, hypoglycemia, hyperglycemia, glucose intolerance, insulin resistance, hyperinsulinemia hypertension, hyperlipoproteinemia, metabolic syndrome, syndrome X, thrombotic disorders, cardiovascular disease, atherosclerosis and its sequelae including angina, claudication, heart attack, stroke and others, kidney diseases, ketoacidosis, nephropathy, diabetic neuropathy, diabetic retinopathy, nonalcoholic fatty liver diseases such as steatosis or nonalcoholic steatohepatitis (NASH)comprising the administration of a pharmaceutically effective amount of a compound of formula (I) or pharmaceutically acceptable salt thereof to a patient in need thereof.

Preferably, the patient is a warm-blooded animal, more preferably a human.

The invention further provides the use of a compound of formula (I) or a pharmaceutically acceptable salt or solvates thereof for the manufacture of a medicament for use in treating a patient and/or preventing a patient from developing a disease selected from the group consisting of type II diabetes, obesity, dyslipidemia such as mixed or diabetic dyslipidemia, hypercholesterolemia, low HDL cholesterol, high LDL cholesterol, hyperlipidemia, hypertriglyceridemia, hypoglycemia, hyperglycemia, glucose intolerance, insulin resistance, hyperinsulinemia hypertension, hyperlipoproteinemia, metabolic syndrome, syndrome X, thrombotic disorders, cardiovascular disease, atherosclerosis and its sequelae including angina, claudication, heart attack, stroke and others, kidney diseases, ketoacidosis, nephropathy, diabetic neuropathy, diabetic retinopathy, nonalcoholic fatty liver diseases such as steatosis or nonalcoholic steatohepatitis (NASH).

Preferably, the patient is a warm-blooded animal, more preferably a human.

According to a further feature of the present invention there is provided a method for modulating GPR43 receptor activity, in a patient, preferably a warm blooded animal, and even more preferably a human, in need of such treatment, which comprises administering to said animal an effective amount of compound of the present invention, or a pharmaceutically acceptable salt or solvate thereof.

According to one embodiment, the compounds of the invention, their pharmaceutical acceptable salts or solvates may be administered as part of a combination therapy. Thus, are included within the scope of the present invention embodiments comprising coadministration of, and compositions and medicaments which contain, in addition to a compound of the present invention, a pharmaceutically acceptable salt or solvate thereof as active ingredient, additional therapeutic agents and/or active ingredients. Such multiple drug regimens, often referred to as combination therapy, may be used in the treatment and/or prevention of any of the diseases or conditions mediated by or associated with GPR43 receptor modulation, particularly type II diabetes, obesity, dyslipidemia such as mixed or diabetic dyslipidemia, hypercholesterolemia, low HDL cholesterol, high LDL cholesterol, hyperlipidemia, hypertriglyceridemia, hypoglycemia, hyperglycemia, glucose intolerance, insulin resistance, hyperinsulinemia hypertension, hyperlipoproteinemia, metabolic syndrome, syndrome X, thrombotic disorders, cardiovascular disease, atherosclerosis and its sequelae including angina, claudication, heart attack, stroke and others, kidney diseases, ketoacidosis, nephropathy, diabetic neuropathy, diabetic retinopathy, nonalcoholic fatty liver diseases such as steatosis or nonalcoholic steatohepatitis (NASH). The use of such combinations of therapeutic agents is especially pertinent with respect to the treatment of the above-mentioned list of diseases within a patient in need of treatment or one at risk of becoming such a patient.

In addition to the requirement of therapeutic efficacy, which may necessitate the use of active agents in addition to the GPR43 agonist or partial agonist compounds of Formula I or their pharmaceutical acceptable salts or solvates thereof, there may be additional rationales which compel or highly recommend the use of combinations of drugs involving active ingredients which represent adjunct therapy, i.e., which complement and supplement the function performed by the GPR43 receptor agonist or partial agonist compounds of the present invention. Suitable supplementary therapeutic agents used for the purpose of auxiliary treatment include drugs which, instead of directly treating or preventing a disease or condition mediated by or associated with GPR43 receptor modulation, treat diseases or conditions which directly result from or indirectly accompany the basic or underlying GPR43 receptor modulated disease or condition.

Thus, the methods of treatment and pharmaceutical compositions of the present invention may employ the compounds of Formula I or their pharmaceutical acceptable salts or solvates thereof in the form of monotherapy, but said methods and compositions may also be used in the form of multiple therapy in which one or more compounds of Formula I or their pharmaceutically acceptable salts or solvates are coadministered in combination with one or more other therapeutic agents such as those described in detail further herein.

Examples of other active ingredients that may be administered in combination with a compound of Formula I or a pharmaceutically acceptable salt or solvate thereof, and either administered separately or in the same pharmaceutical composition, include but are not limited to:
(a) PPARγ agonists and partial agonists, including both glitazones and non-glitazones (e.g. troglitazone, pioglitazone, englitazone, MCC-555, rosiglitazone, balaglitazone, netoglitazone, T-131, LY-300512 and LY-818;
(b) Biguanides such as metformin and phenformin;
(c) Protein tyrosine phosphatase-1B (PTP-1B) inhibitors,
(d) Dipeptidyl peptidase IV (DP-IV) inhibitor, such as MK-0431 and LAF-237;
(e) Insulin or insulin mimetic s;
(f) Sulfonylureas such as tolbutamide and glipizide or related materials;
(g) α-glucosidase inhibitors (such as acarbose);
(h) agents which improve a patient's lipid profile such as (i) HMG-CoA reductase inhibitors (lovastatin, simvastatin, rosuvastatin, pravastatin, fluvastatin, atorvastatin, rivastatin, itavastatin, ZD-4522 and other statins), (ii) bile acid sequestrants (cholestyramine, colestipol and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) cholesterol absorption inhibitors such as for example ezetimibe, (vi) acyl CoA:cholesterol acyltransferase (ACAT) inhibitors such as avasimibe, (vii) CETP inhibitors such as torcetrapib and (viii) phenolic anti-oxidants such as probucol;
(i) PPARα/γ dual agonists such as muraglitazar, tesaglitazar, farglitazar and JT-501;
(j) PPARδ agonists such those disclosed in WO97/28149;
(k) Antiobesity compounds such as fenfluramine, dextenfluramine, phentiramine, subitramine, orlistat, neuropeptide Y5 inhibitors, MC4R agonists, cannabinoid receptor 1 antagonists/inverse agonists and β3 adrenergic receptor agonists;
(l) Ileal bile acid transporter inhibitors;
(m) Agents intended for use in inflammatory conditions such as aspirin, non-steroidal, anti-inflammatory drugs, glucocorticoids, azulfidine and cyclo-oxygenase 2 selective inhibitors;
(n) Glucagon receptor antagonists;
(o) GLP-1;
(p) GIP-1;
(q) GLP-1 analogs, such as exendins, for example exenitide, and
(r) Hydroxysterol dehydrogenase-1 (HSD-1) inhibitors.

The above combinations include combinations of a compound of the present invention or a pharmaceutically acceptable salt or solvate not only with one other active compound but also with two or more active compounds. Non limiting examples include combinations of compounds having Formula I with two or more active compounds selected from biguanides, sulfonylureas, HMG-CoA reductase inhibitors, other PPAR agonists, PTP-1B inhibitors, DP-IV inhibitors and anti-obesity compounds.

In the above-described embodiment combinations of the present invention, the compound of Formula I, a pharmaceutically acceptable salt or solvate thereof and other therapeutic active agents may be administered in terms of dosage forms either separately or in conjunction with each other, and in terms of their time of administration, either serially or simultaneously. Thus, the administration of one component agent may be prior to, concurrent with, or subsequent to the administration of the other component agent(s).

The invention also provides pharmaceutical compositions comprising a compound of formula I or a pharmaceutically acceptable salt or solvate thereof and at least one pharmaceutically acceptable carrier, diluent, excipient and/or adjuvant. As indicated above, the invention also covers pharmaceutical compositions which contain, in addition to a compound of the present invention, a pharmaceutically acceptable salt or solvate thereof as active ingredient, additional therapeutic agents and/or active ingredients.

Another object of this invention is a medicament comprising at least one compound of the invention, or a pharmaceutically acceptable salt or solvate thereof, as active ingredient.

The invention also provides the use of a compound of formula I or a pharmaceutically acceptable salt or solvate thereof for the manufacture of a medicament. Preferably, the medicament is used for the treatment and/or prevention of type II diabetes, obesity, dyslipidemia such as mixed or diabetic dyslipidemia, hypercholesterolemia, low HDL cholesterol, high LDL cholesterol, hyperlipidemia, hypertriglyceridemia, hypoglycemia, hyperglycemia, glucose intolerance, insulin resistance, hyperinsulinemia hypertension, hyperlipoproteinemia, metabolic syndrome, syndrome X, thrombotic disorders, cardiovascular disease, atherosclerosis and its sequelae including angina, claudication, heart attack, stroke and others, kidney diseases, ketoacidosis, nephropathy, diabetic neuropathy, diabetic retinopathy, nonalcoholic fatty liver diseases such as steatosis or nonalcoholic steatohepatitis (NASH).

Preferred diseases are type II diabetes, lipid disorders such as dyslipidemia, hypertension, obesity, atherosclerosis and its sequelae.

In a particular preferred embodiment the disease are type II diabetes and a lipid disorder such as dyslipidemia.

According to a further feature of the present invention there is provided the use of a compound of formula I or a pharmaceutically acceptable salt or solvate thereof for the manufacture of a medicament for modulating GPR43 receptor activity, in a patient, in need of such treatment, which comprises administering to said patient an effective amount of compound of the present invention, or a pharmaceutically acceptable salt or solvate thereof.

Preferably, the patient is a warm-blooded animal, more preferably a human.

As set forth above, the compounds of the invention, their pharmaceutically acceptable salts or solvates may be used in monotherapy or in combination therapy. Thus, according to one embodiment, the invention provides the use of a compound of the invention for the manufacture of a medicament for at least one of the purposes described above, wherein said medicament is administered to a patient in need thereof, preferably a warm-blooded animal, and even more preferably a human, in combination with at least one additional therapeutic agent and/or active ingredient. The benefits and advantages of such a multiple drug regimen, possible administration regimens as well as suitable additional therapeutic agents and/or active ingredients are those described above.

Generally, for pharmaceutical use, the compounds of the inventions may be formulated as a pharmaceutical preparation comprising at least one compound of the invention and at least one pharmaceutically acceptable carrier, diluent, excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, creams, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 0.05 and 1000 mg, and usually between 1 and 500 mg, of the at least one compound of the invention, e.g. about 10, 25, 50, 100, 200, 300 or 400 mg per unit dosage.

Usually, depending on the condition to be prevented or treated and the route of administration, the active compound of the invention will usually be administered between 0.01 to 100 mg per kilogram, more often between 0.1 and 50 mg, such as between 1 and 25 mg, for example about 0.5, 1, 5, 10, 15, 20 or 25 mg, per kilogram body weight day of the patient per day, which may be administered as a single daily dose, divided over one or more daily doses, or essentially continuously, e.g. using a drip infusion.

### [Definitions]

The definitions and explanations below are for the terms as used throughout the entire application, including both the specification and the claims.

When describing the compounds of the invention, the terms used are to be construed in accordance with the following definitions, unless indicated otherwise.

Where groups may be substituted, such groups may be substituted with one or more substituents, and preferably with one, two or three substituents. Substituents may be selected from but not limited to, for example, the group comprising halogen, hydroxyl, oxo, nitro, amido, carboxy, amino, cyano haloalkoxy, and haloalkyl.

As used herein the terms such as “alkyl, aryl, or cycloalkyl, each being optionally substituted with . . . ” or “alkyl, aryl, or cycloalkyl, optionally substituted with . . . ” encompasses “alkyl optionally substituted with . . . ”, “aryl optionally substituted with . . . ” and “cycloalkyl optionally substituted with . . . ”.

The term “halo” or “halogen” means fluoro, chloro, bromo, or iodo. Preferred halo groups are fluoro and chloro.

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 6 carbon atoms, preferably from 1 to 4 carbon atoms, more preferably from 1 to 3 carbon atoms, still more preferably 1 to 2 carbon atoms. Alkyl groups may be linear or branched and may be substituted as indicated herein.

Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl). Preferred alkyl groups include methyl, ethyl, n-propyl, i-propyl, n-butyl, i-butyl, s-butyl and t-butyl.

When the suffix “ene” (“alkylene”) is used in conjunction with an alkyl group, this is intended to mean the alkyl group as defined herein having two single bonds as points of attachment to other groups. The term “alkylene” includes methylene, ethylene, methylmethylene, propylene, ethylethylene, and 1,2-dimethylethylene.

The term “alkenyl” as used herein refers to an unsaturated hydrocarbyl group, which may be linear or branched, comprising one or more carbon-carbon double bonds. Suitable alkenyl groups comprise between 2 and 6 carbon atoms, preferably between 2 and 4 carbon atoms, still more preferably between 2 and 3 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like.

The term “alkynyl” as used herein refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkenyl groups. Non limiting examples of alkynyl groups are ethynyl, 2-propynyl, 2-butynyl, 3-butynyl, 2-pentynyl and its isomers, 2-hexynyl and its isomers-and the like. The terms “alkenylene” and “alkynylene” respectively mean an alkenyl group or an alkinyl group as defined above having two single bonds as points of attachment to other groups.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl and the like.

The term “cycloalkyl” as used herein is a cyclic alkyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. Examples of cycloalkyl groups include but are not limited to cyclopropyl, cyclobutyl, cyclopentyl, cyclohexyl, with cyclopropyl being particularly preferred.

When the suffix “ene” is used in conjunction with a cyclic group, this is intended to mean the cyclic group as defined herein having two single bonds as points of attachment to other groups.

Therefore, “cycloalkylene” herein refers to a saturated homocyclic hydrocarbyl biradical of Formula CₙH₂ₙ₋₂. Suitable cycloalkylene groups are C₃₋₆ cycloalkylene group, preferably a C₃₋₅ cycloalkylene (i.e. 1,3-cyclopropylene, 1,1-cyclopropylene, 1,1-cyclobutylene, 1,2-cyclobutylene, 1,3-cyclopentylene, or 1,1-cyclopentylene), more preferably a C₃₋₄ cycloalkylene (i.e. 1,3-cyclopropylene, 1,1-cyclopropylene, 1,1-cyclobutylene, 1,2-cyclobutylene).

Where at least one carbon atom in a cycloalkyl group is replaced with a heteroatom, the resultant ring is referred to herein as “heterocycloalkyl” or “heterocyclyl”.

The terms “heterocyclyl”, “heterocycloalkyl” or “heterocyclo” as used herein by itself or as part of another group refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen, oxygen and/or sulfur atoms, where the nitrogen and sulfur heteroatoms may optionally be oxidized and the nitrogen heteroatoms may optionally be quaternized. Any of the carbon atoms of the heterocyclic group may be substituted by oxo (for example piperidone, pyrrolidinone).The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include oxetanyl, piperidinyl, azetidinyl, 2-imidazolinyl, pyrazolidinyl imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1H-pyrrolizinyl, tetrahydro-1,1-dioxothiophenyl, N-formylpiperazinyl, and morpholin-4-yl.

The ring atoms of heterocyclyl and heterocyclylene moieties are numbered based on scheme below

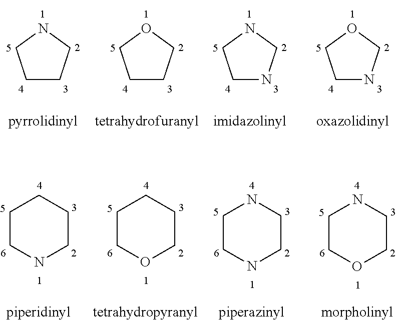

The term “aryl” as used herein refers to a polyunsaturated, aromatic hydrocarbyl group having a single ring (i.e. phenyl) or multiple aromatic rings fused together (e.g. naphthyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, naphthalen-1- or -2-yl, 4-, 5-, 6 or 7-indenyl, 1- 2-, 3-, 4- or 5-acenaphtylenyl, 3-, 4- or 5-acenaphtenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl.

The term “arylene” as used herein is intended to include divalent carbocyclic aromatic ring systems such as phenylene, biphenylylene, naphthylene, indenylene, pentalenylene, azulenylene and the like. Arylene is also intended to include the partially hydrogenated derivatives of the carbocyclic systems enumerated above. Non-limiting examples of such partially hydrogenated derivatives are 1,2,3,4-tetrahydronaphthylene, 1,4-dihydronaphthylene and the like.

Where at least one carbon atom in an aryl group is replaced with a heteroatom, the resultant ring is referred to herein as a heteroaryl ring.

The term “heteroaryl” as used herein by itself or as part of another group refers but is not limited to 5 to 12 carbon-atom aromatic rings or ring systems containing 1 to 2 rings which are fused together or linked covalently, typically containing 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

The term “heteroarylene” as used herein means divalent carbocyclic aromatic ring systems including pyridinylene and the like.

The ring atoms of heteroaryl or heteroarylene moieties are numbered on scheme below:

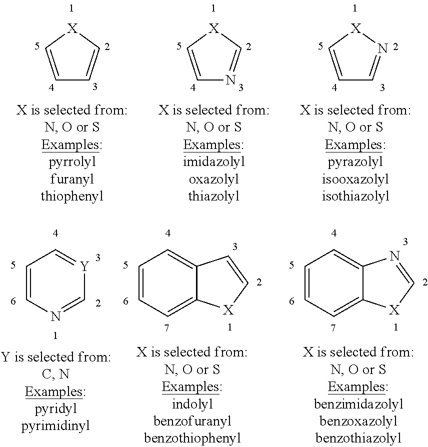

The term “biaryl” as used herein designates two aryl moieties as defined herein linked via a single bond. Non-limiting examples of such biaryl moieties include biphenyl.

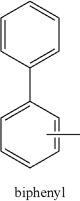

The term “heterobiaryl” as used herein designates two heteroaryl moieties as defined herein or a heteroaryl moiety and an aryl moiety as defined herein linked via a single bond. Non-limiting examples of such heterobiaryl moieties include pyridinylphenyl which is meant to include (2-pyridinyl)phenyl, (3-pyridinyl)phenyl and (4-pyridinyl)phenyl, bipyridinyl.

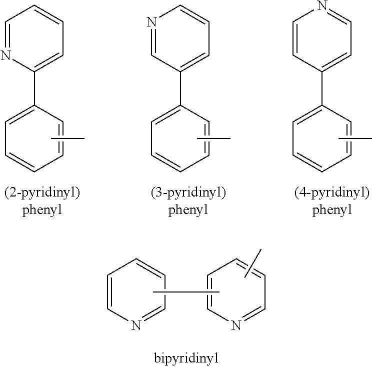

The term “alkylamino” as used herein means an amino group substituted with one or two alkyl groups. This includes monoalkylamino and dialkylamino groups.

The compounds of Formula I and subformulae thereof contain at least one asymmetric center and thus may exist as different stereoisomeric forms. Accordingly, the present invention includes all possible stereoisomers and includes not only racemic compounds but the individual enantiomers and their non racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

The bonds from an asymmetric carbon in compounds of the present invention may be depicted herein using a solid line ( [symbol] ), a zigzag line ( [symbol] ), a solid wedge ( [symbol] ), or a dotted wedge ( [symbol] ). The use of a solid line to depict bonds from an asymmetric carbon atom is meant to indicate that all possible stereoisomers are meant to be included, unless it is clear from the context that a specific stereoisomer is intended. The use of either a solid or dotted wedge to depict bonds from an asymmetric carbon atom is meant to indicate that only the stereoisomer shown is meant to be included.

The compounds of the invention may also contain more than one asymmetric carbon atom. In those compounds, the use of a solid line to depict bonds from asymmetric carbon atoms is meant to indicate that all possible stereoisomers are meant to be included, unless it is clear from the context that a specific stereoisomer is intended.

The compounds of the invention may be in the form of pharmaceutically acceptable salts. Pharmaceutically acceptable salts of the compounds of formula I include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine and zinc salts. Hemisalts of acids and bases may also be formed, for example, hemisulphate and hemicalcium salts. Preferred, pharmaceutically acceptable salts include hydrochloride/chloride, hydrobromide/bromide, bisulphate/sulphate, nitrate, citrate, and acetate.

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of compounds of Formula I may be prepared by one or more of these methods:

(i) by reacting the compound of Formula I with the desired acid;

(ii) by reacting the compound of Formula I with the desired base;

(iii) by removing an acid- or base-labile protecting group from a suitable precursor of the compound of Formula I or by ring-opening a suitable cyclic precursor, for example, a lactone or lactam, using the desired acid; or

(iv) by converting one salt of the compound of Formula I to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The term “solvate” is used herein to describe a molecular complex comprising the compound of the invention and one or more pharmaceutically acceptable solvent molecules, for example, ethanol. The term ‘hydrate’ is employed when said solvent is water.

All references to compounds of formula I include references to salts, solvates, multi-component complexes and liquid crystals thereof.

The compounds of the invention include compounds of formula I as hereinbefore defined, including all polymorphs and crystal habits thereof, prodrugs and isomers thereof (including optical, geometric and tautomeric isomers) and isotopically-labeled compounds of formula I.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula I above.

The invention also generally covers all pharmaceutically acceptable predrugs and prodrugs of the compounds of Formula I.

The term “prodrug” as used herein means the pharmacologically acceptable derivatives of compounds of formula I such as esters whose in vivo biotransformation product is the active drug. Prodrugs are characterized by increased bio-availability and are readily metabolized into the active compounds in vivo. Suitable prodrugs for the purpose of the invention include carboxylic esters, in particular alkyl esters, aryl esters, acyloxyalkyl esters, and dioxolene carboxylic esters; ascorbic acid esters as well as compounds of formula I in which Z is a substituent selected from the table 2 below.

TABLE 2

| Z | Q |
|---|---|
| —C(O)SQ | Alkyl or aryl |
| —C(O)NQ¹Q² | H, alkyl, aryl, OH or NH₂ |
| —C(O)OCHQ¹(O)CQ² | Q¹ = H or phenyl |
| | Q² = alkyl or aryl |
| —C(O)OCHQCl | H or aryl |
| —C(OQ)₃ | Alkyl |
| —C(O)OC(O)OQ | Alkyl or aryl |
| —C(O)CH₂Q | SMe, SOMe, SO₂Me |

The term “predrug”, as used herein, means any compound that will be modified to form a drug species, wherein the modification may take place either inside or outside of the body, and either before or after the predrug reaches the area of the body where administration of the drug is indicated.

The term “patient” refers to a warm-blooded animal, more preferably a human, who/which is awaiting or receiving medical care or is or will be the object of a medical procedure.

The term “human” refers to subject of both genders and at any stage of development (i.e. neonate, infant, juvenile, adolescent, adult).

The terms “treat”, “treating” and “treatment, as used herein, are meant to include alleviating or abrogating a condition or disease and/or its attendant symptoms.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “therapeutically effective amount” (or more simply an “effective amount”) as used herein means the amount of active agent or active ingredient (e. g. GPR43 agonist or partial agonist) which is sufficient to achieve the desired therapeutic or prophylactic effect in the individual to which it is administered.

The term “administration”, or a variant thereof (e.g., “administering”), means providing the active agent or active ingredient (e. g. a GPR43 agonist or partial agonist), alone or as part of a pharmaceutically acceptable composition, to the patient in whom/which the condition, symptom, or disease is to be treated or prevented.

By “pharmaceutically acceptable” is meant that the ingredients of a pharmaceutical composition are compatible with each other and not deleterious to the patient thereof.

The term “agonist” as used herein means a ligand that activates an intracellular response when it binds to a receptor. An agonist according to the invention may promote internalization of a cell surface receptor such that the cell surface concentration of a receptor is decreased or remove.

The term “partial agonist” as used herein means an agonist which is unable to induce maximal activation of a receptor, regardless of the amount of compound applied on the receptor.

The term “pharmaceutical vehicle” as used herein means a carrier or inert medium used as solvent or diluent in which the pharmaceutically active agent is formulated and/or administered. Non-limiting examples of pharmaceutical vehicles include creams, gels, lotions, solutions, and liposomes.

The term “lipid disorder” as used herein means any plasma lipid disorder including but not limited to dyslipidemia such as mixed or diabetic dyslipidemia, hypercholesterolemia, low HDL cholesterol, high LDL cholesterol, hyperlipidemia and hypertriglyceridemia.

The present invention will be better understood with reference to the following examples. These examples are intended to representative of specific embodiments of the invention, and are not intended as limiting the scope of the invention.

### CHEMISTRY EXAMPLES

All temperatures are expressed in ° C. and all reactions were carried out at room temperature (RT) unless otherwise stated.

Analytical thin layer chromatography (TLC) was used to monitor reactions, establish flash chromatography conditions and verify purity of intermediates or final products. TLC plates used were Merck TLC aluminium sheet silica gel 60 F₂₅₄ purchased from VWR International. TLC plates were revealed using ultraviolet irradiation (wavelength=254 nm) at room temperature or bromocresol green spray reagent at 0.1% in propan-2-ol purchased from VWR International upon heating at 160° C. or KMnO₄ revelator upon heating at 160° C. The KMnO₄ revelator was prepared by dissolving 3 g of potassium permanganate, 20 g of sodium carbonate, 0.5 g of sodium hydroxide in 100 mL of distilled water.

HPLC-MS spectra were obtained on Agilent LCMS using Electrospray ionization (ESI). The Agilent instrument includes an Autosampler 1200, a binary pump 1100, a 5 wave length detector 1100 and a 6100 Single Quad. The column used was an XBridge C18, 4.6×50 mm, 3.5 μm.

Eluent was a mixture of solution A (0.1% TFA in H₂O) and solution B (0.1% TFA in ACN). Gradient was applied at a flow rate of 2 mL min⁻¹ as follows: gradient A: held the initial conditions of 5% solution B for 1 min, increased linearly to 95% solution B in 4 min, held at 95% during 1 min, returned to initial conditions in 0.5 min and maintained for 1 min; gradient B: held the initial conditions of 5% solution B for 1 min, increased linearly to 60% in 10 min, increased linearly to 95% in 0.5 min, held at 95% during 3 min, returned to initial conditions in 0.5 min and maintained for 1 min.

Determination of ee was performed on an Agilent 1100 (binary pump and 5 wavelengths detector) with manual or automatic (Autosampler 1100) injection. Columns used were CHIRALPAK IA CHIRALPAK IB or CHIRALPAK IC in isocratic mode. Mixtures of eluents were selected depending on the separation obtained of enantiomers or diastereosiomers. Usual mixtures were:

Hexane and Ethanol (0.1% TFA)

Hexane and Propanol (0.1% TFA)

Hexane and Ethyl acetate (0.1% TFA)

Hexane and Dichloromethane (0.1% TFA)

Hexane and tert-butyl methyl ether (0.1% TFA)

Selected specific methods A, B and C are reported below. Method A: compound was characterized on a CHIRALPAK IA column (isocratic mode) using a mixture of hexane and dichloromethane (65/35) acidified by 0.4% of TFA at a flow rate of 1.2 mL/min, and confirmed on a CHIRALPAK IC column (isocratic mode) using a mixture of heptane and Ethyl acetate (75/25) acidified by 0.1% of TFA at 1 ml/min. Method B: compound was characterized on a CHIRALPAK IC column (isocratic mode) using a mixture of heptane and ethyl acetate (70/30) acidified by 0.1% of TFA at a flow rate of lml/min. Method C: compound was characterized on a CHIRALPAK IC column (isocratic mode) using a mixture of heptane and ethanol (95/5) acidified by 0.1% of TFA at a flow rate of 1.5 ml/min.

Preparative HPLC purifications were carried out on Fractionlynx instrument, from Waters. This instrument consists of a Fraction Collector, a 2767 Sample Manager, a pump control a module II, a 515 HPLC Pump, a 2525 Binary Gradient Module, a Switching Valve, a 2996 Photodiode Array Detector and a Micromass ZQ. The column used was a Waters Sunfire C18 Eluent was a mixture of solution A (0.1% TFA in H₂O) and solution B (0.1% TFA in ACN). The gradient was adapted depending on impurities present in samples, to allow sufficient separation between impurities and target compound.

Chiral preparative HPLC purification were performed on an Agilent 1100 instrument (binary pump and 5 wavelengths detector) with manual injection using a CHIRALPAK IA or a CHIRALPAK IB column in isocratic mode. Mixtures of eluents were selected depending on the separation of enantiomers or diastereosiomers obtained with the analytical method. Usual mixtures were the same as those used for the determination of ee.

¹H and ¹³C NMR spectra were recorded on a Bruker ARX 300 MHz. Chemical shifts are expressed in parts per million, (ppm, δ units). Coupling constants are expressed in Hertz units (Hz). Splitting patterns describe apparent multiplicities and are described as s (singlet), d (doublet), t (triplet), q (quintet), m (multiplet), or br (broad).

Solvents, reagents and starting materials were purchased from well known chemical suppliers such as for example Sigma Aldrich, Acros Organics, Fluorochem, Eurisotop, VWR International, Sopachem and Polymer labs and the following abbreviations are used:
ACN: Acetonitrile,
DCM: Dichloromethane,
DMF: N,N-dimethylformamide,
EtOAc: Ethyl acetate,
EtOH: Ethanol,
MeOH: Methanol,
RT: Room temperature,
DIEA: N,N-diisopropylethylamine,
HATU: O-(7-azabenzotriazol-1-yl)-N,N,N′,N′-tretramethyluronium hexafluorophosphate,
Y: Yield,
g: Grams,
mg: Milligrams,
L: Liters,
mL: Milliliters,
μL: Microliters,
mol: Moles,
mmol: Millimoles,
h: Hours,
min: Minutes,
TLC: Thin layer chromatography,
MW: Molecular weight,
eq: Equivalent,
μW: Microwave,
THF: Tetrahydrofuran,
TFA: Trifluoroacetic acid,
Ac: Acetyl,
NaHMDS: Sodium hexamethyldisilazane,
DCA: Dicyclohexylamine,
TCA: Trichloroacetimidate,
CDI: Carbonyl diimidazole,
ee: Enantiomeric excess,
DPP: Diphenylphosphino,
BINAP: 1,1′-Binaphtyl,
tBu: tert-Butyl
P: UV purity at 254 nm determined by HPLC-MS,
SPE: Sold phase extraction,
Rt: Retention time,
TMSCl: Chlorotrimethylsilane,
BuLi: Butyllithium,
MCPBA: 3-Chloroperbenzoic acid,
MOM: Methoxymethyl,
NCS: N-chlorosuccinimide,
NBS: N-bromosuccinimide.

### General Synthetic Scheme

Most compounds of the invention are synthesized according to Scheme 1.

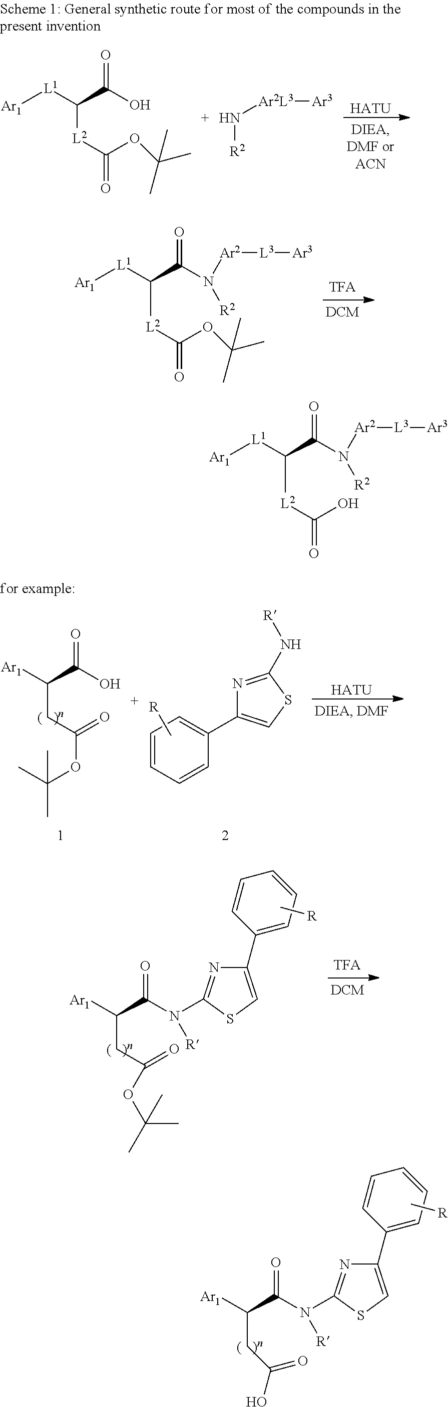

### Synthesis of Intermediates 1

Chiral syntheses of intermediates 1 were carried out using Evans' chiral auxiliary approach (Evans et al. J. Org. Chem. 1999, 64, 6411-6417; Tararov et al. J. Chem. Soc. Perkin Trans. 1, 1997, 3101-3106) (Scheme 2).

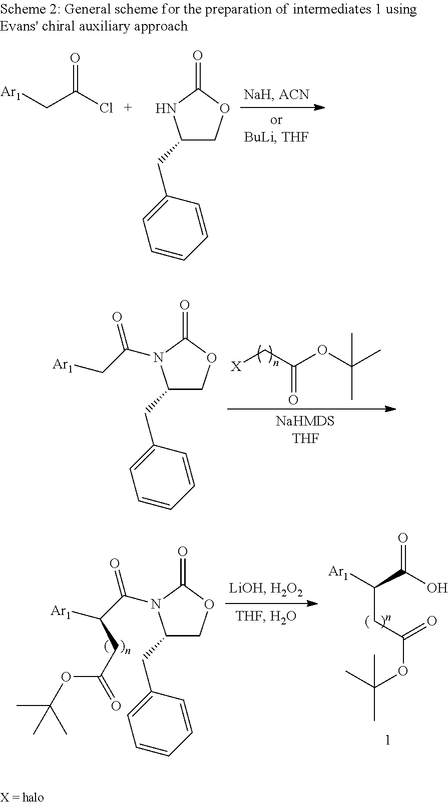

This methodology was also used for the synthesis of (R)-cycloalkylalkylsuccinic acid, (R)-heterocyclylalkylsuccinic acid, (R)-arylalkylsuccinic acid and (R)-heteroarylalkylsuccinic acid monoester intermediates 1.

As depicted on Scheme 3, (R)-benzylsuccinic acid monoester intermediates 1 can also be made starting from maleic anhydride followed by the application of Wittig reaction, asymmetric hydrogenation (Wallace et al. Org. Proc. Res. & Dev. 2004, 8, 738-743), tBu ester protection and selective saponification of the methyl ester (Atkinson et al. J. Org. Chem. 1999, 64, 3467).

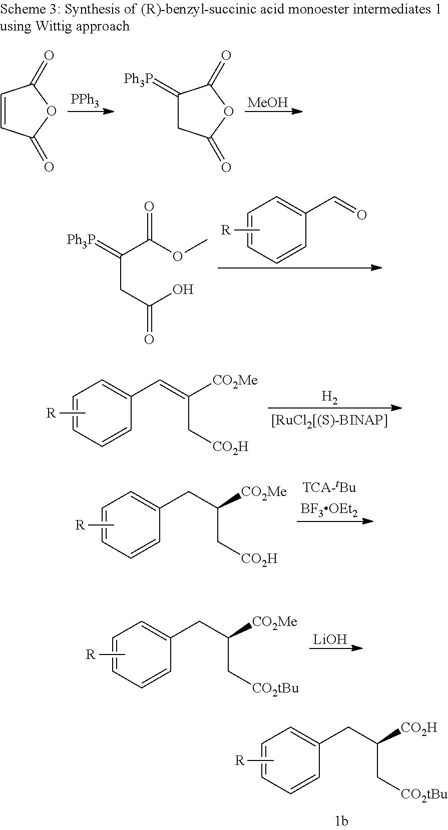

This methodology was also used for the synthesis of (R)-cycloalkylalkylsuccinic acid, (R)-heterocyclylalkylsuccinic acid, (R)-arylalkylsuccinic acid and (R)-heteroarylalkylsuccinic acid monoester intermediates 1.

### Synthesis of Intermediates 2

4-aryl-2-amino-thiazoles can be made using Hantzsch-type synthetic methodology as shown in Scheme 4. Thus, halogenation of substituted acetophenones (Larock, R. C. Comprehensive Org Transf 2^{nd} Ed., Wiley, 1999, pp 709-719; White et al. J. Med. Chem. 1996, 39, 4382-95) and subsequent condensation with thiourea (Swain et al. J. Med. Chem. 1991, 34, 140-151; Barton et al. J. Med. Chem. 1998, 41, 1855-68) will furnish 4-aryl-2-amino-thiazoles.

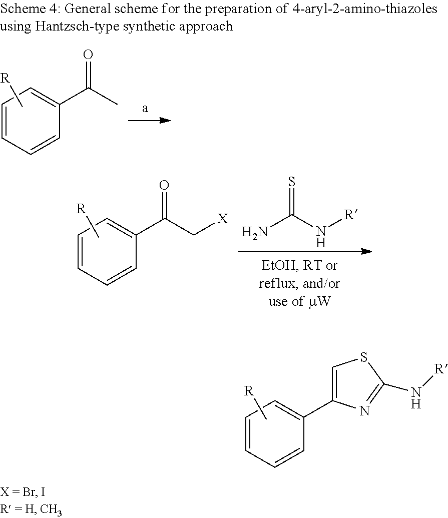

Alternatively, synthesis of N-substituted-4-aryl-2-amino-thiazoles can be achieved through the method described by Rudolph (Rudolph, J. Tetrahedron 2000, 56, 3161)

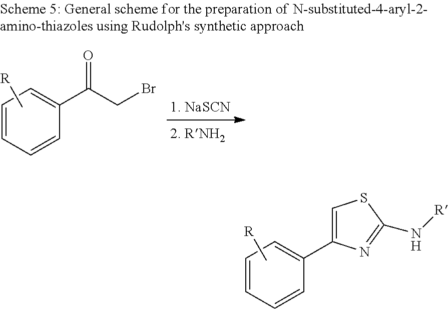

### Synthetic Schemes for the Preparation of the Carboxylic Acid Bioisosteres

Synthetic routes for the preparation of selected bioisosteres of the carboxylic acid moiety are given hereunder. Isosterism is a concept defined by I. Langmuir in J. Am. Chem. Soc. 1919, 41, 1549 and developed by H. L. Friedman in Symposium on Chemical-Biological correlations, National Council Publication, Washington, D.C. (1951). As used herein the term “bioisosteres” refers to “groups or molecules which have chemical and physical similarities producing similar biological effects” (as defined in Chem. Soc. Rev. 1979, 8, 563). Suitable well-known bioisosteric replacements of carboxylic acid groups and synthetic routes are reported in The Practice of Medicinal Chemistry, 2^{nd} edition, by C. G. Wermuth. It is obvious to the person skilled in the art to synthesize carboxylic acid isosteres, selected useful references are Drysdale et al. J. Med. Chem. 1992, 35, 2573-2581, Liljebris et al. J. Med. Chem. 2002, 45, 1785-1798.

### Synthesis of Tetrazole and Hydroxy-Oxadiazole Isosteres

The tetrazole and hydroxy-oxadiazole isosteres can be synthesized using a common nitrile intermediate (see Scheme below). (Arienti et al. J. Med. Chem. 2005, 48, 6, 1882; Rodriguez et al. Tetrahedron 1997, 38, 24, 4221; Claremon et al. Tet. Lett. 1988, 28, 2155).

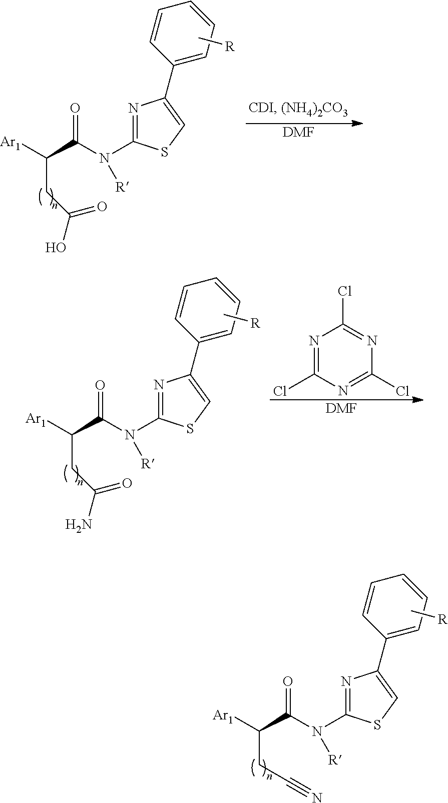

Treatment of the aforesaid nitrile intermediate with sodium azide can be used to afford the tetrazole isostere (see Scheme below). (Matthews et al. J. Comb. Chem. 2000, 2, 19-23)

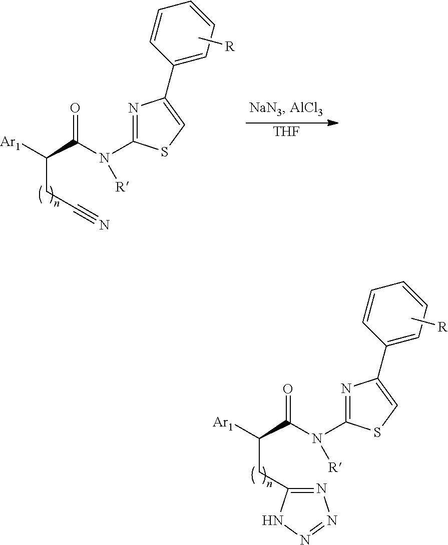

Treatment of the aforesaid nitrile intermediate with hydroxylamine, followed by dehydrative cyclization can be used to yield the hydroxy-oxadiazole isostere (see Scheme below) (Peretto et al. J. Med. Chem. 2005, 48, 5705-5720).

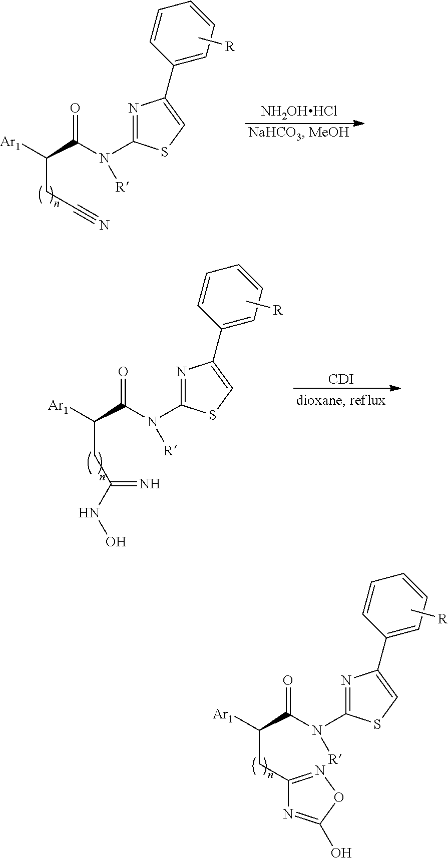

In addition, synthetic approaches to the preparation of other well-recognized carboxylic acid isosteres are outlined below.

### A Suggested Synthetic Approach for the Preparation of Hydroxy-Thiadiazole Isosteres

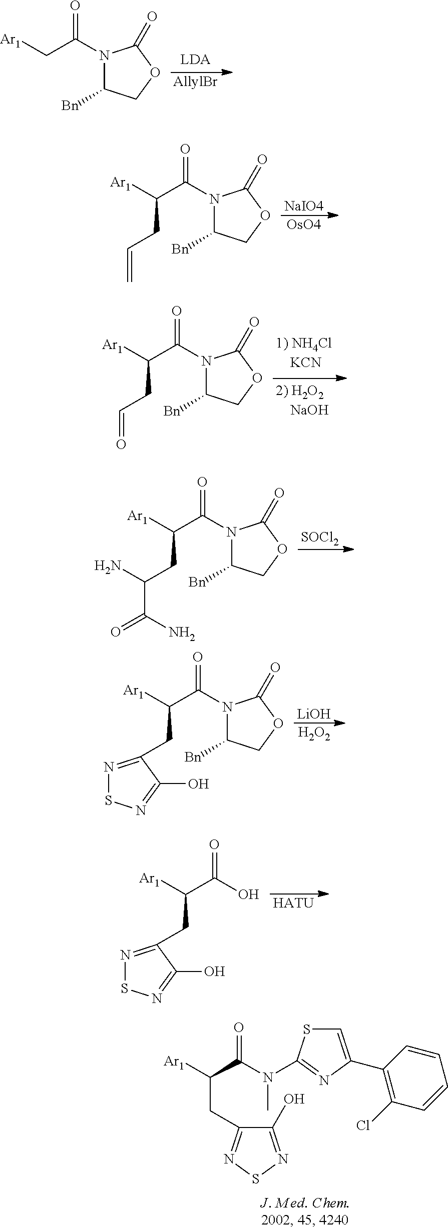

### Synthesis of Hydroxy-Isoxazole Isosteres

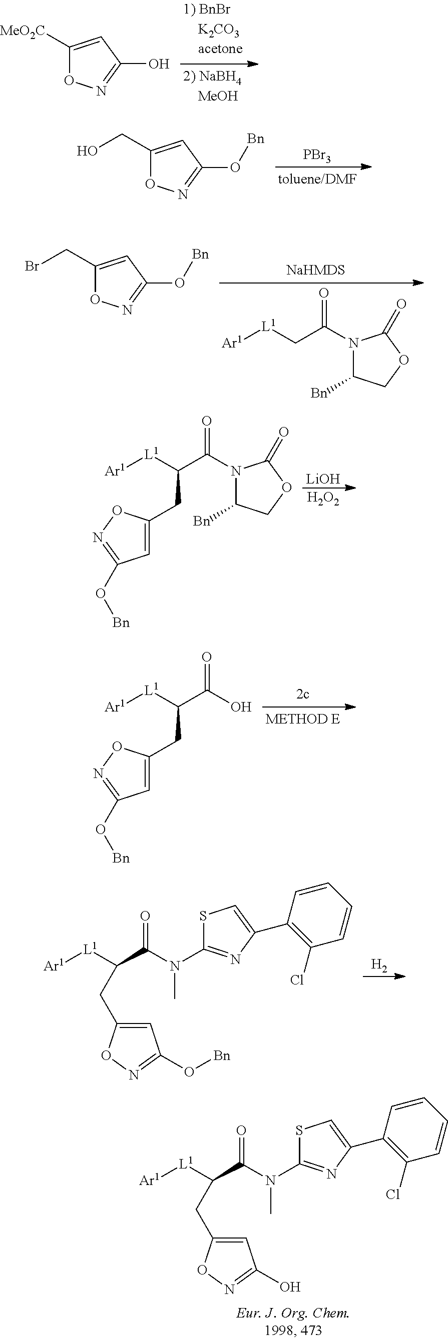

### An Alternative Suggested Synthetic Approach for the Preparation of Hydroxy-Isoxazole Isosteres

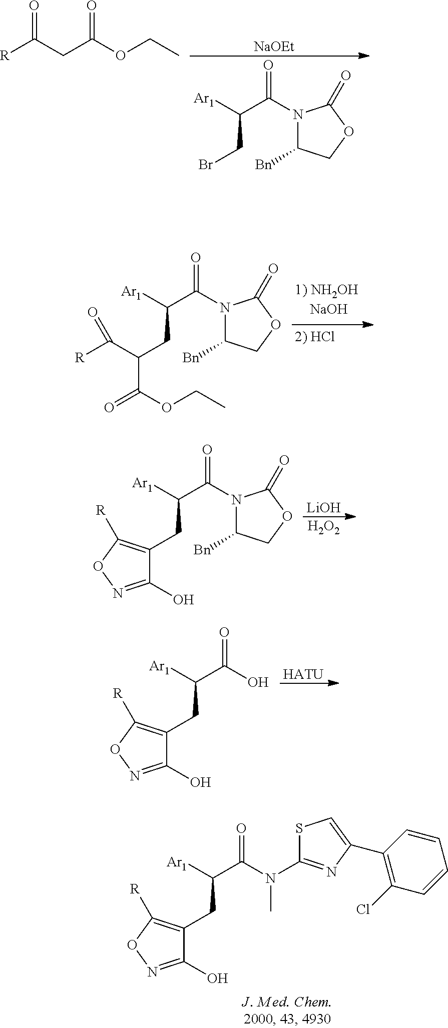

### Additional Synthetic Schemes

An alternative approach towards synthesis of intermediates 1 (see Scheme 2) can be envisioned through Stobbe condensation as depicted in Scheme 6.

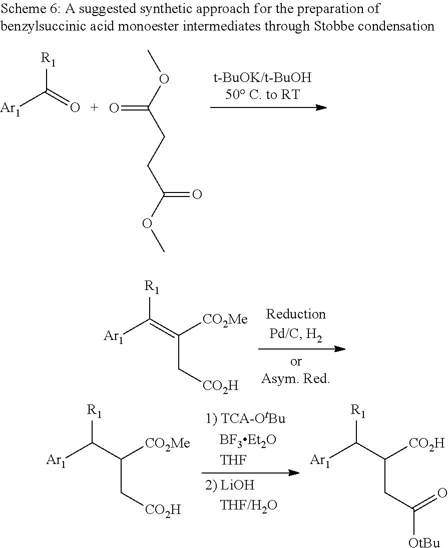

Synthesis of Compound n^{o}68 (Scheme 7):

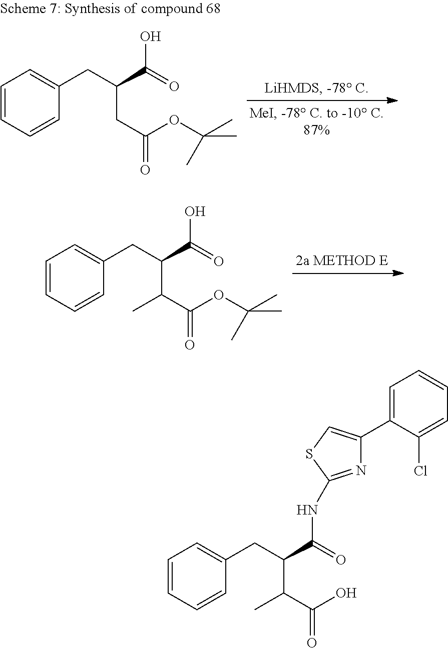

As shown in Scheme 7, upon treatment of (R)-benzylsuccinic acid t-butyl ester with excess LiHMDS in the presence of MeI, the desired monomethylated intermediate was isolated as an epimeric mixture, which was used in turn to furnish the final target structure (as epimeric mixture), as per the general procedure outlined on Scheme 1.

### Synthesis of Aryl-Pyridine and Aryl-Pyrimidines Intermediates 2 (Scheme 8):

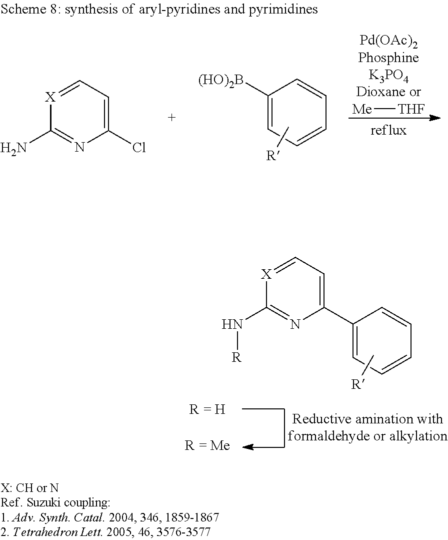

Suzuki coupling between pyridinyl or pyrimidinyl chloride and phenylboronic acid reagents allowed synthesizing the aryl-pyridine and aryl-pyrimidines intermediates 2.

A Suggested Synthesis of Compound n^{o}74 (Scheme 9):

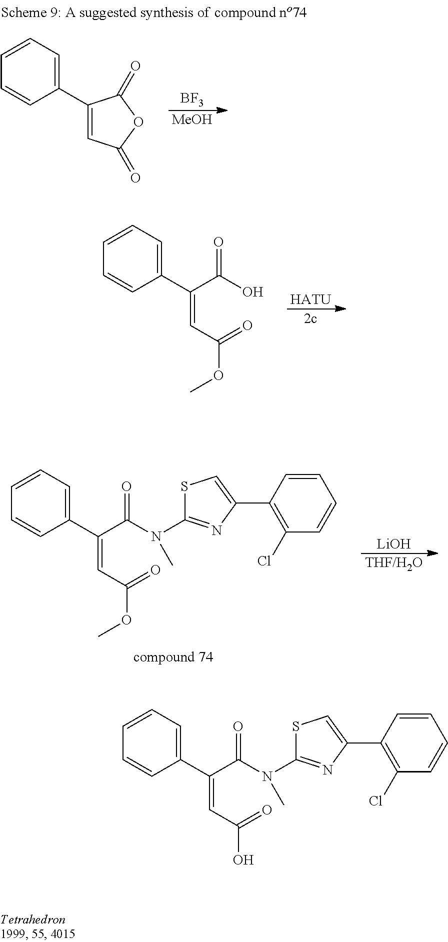

Suggested Syntheses of Compounds n^{o}75 and n^{o}76 (Scheme 10):

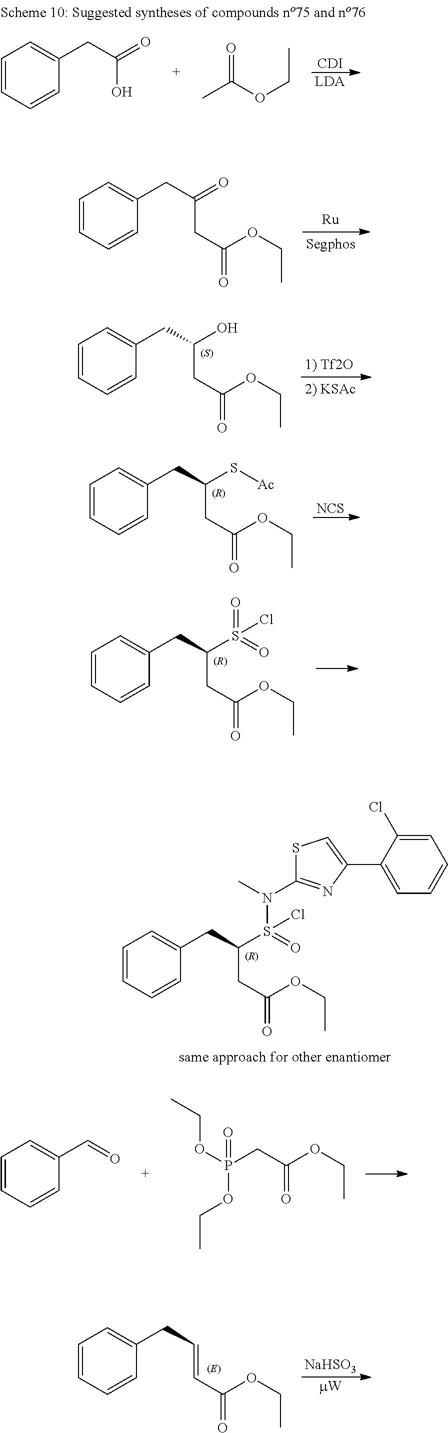

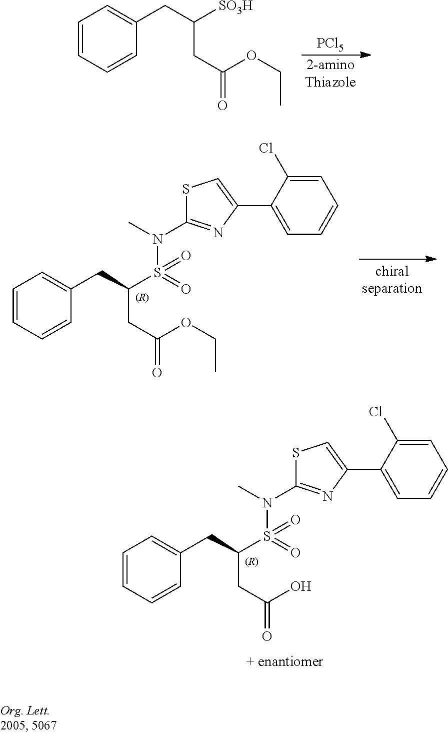

Suggested Syntheses of Compounds n^{o}79 and n^{o}80 (Scheme 11):

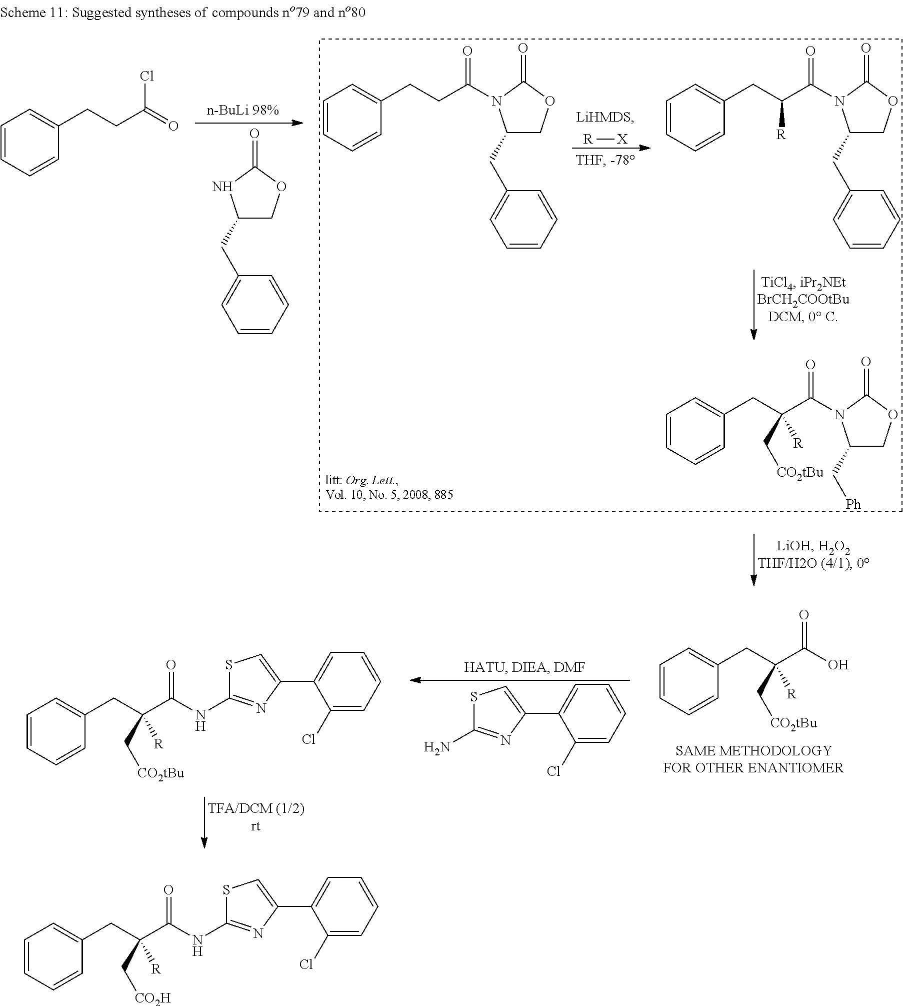

Suggested Syntheses of Compounds n^{o}83 to n^{o}85 (Scheme 12):

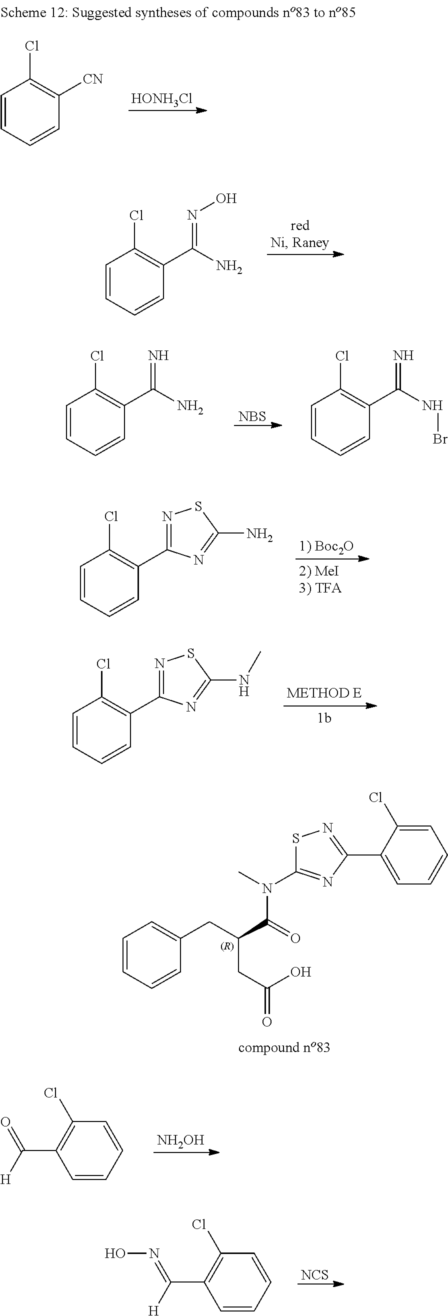

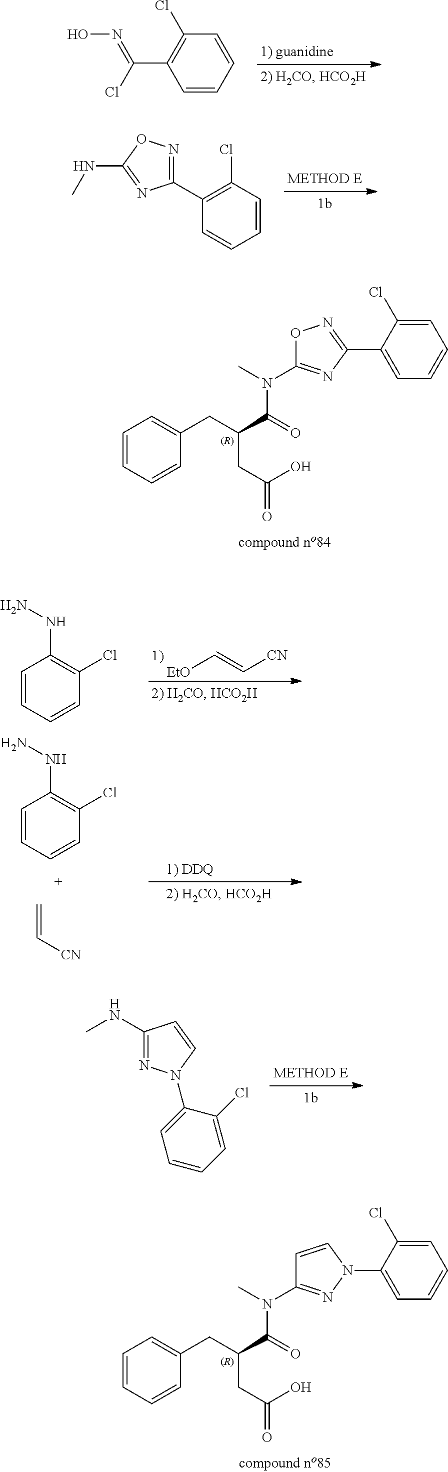

### Synthesis of Intermediates 1 Using Horner-Wadsworth Emmons Approach (HWE) (Scheme 13):

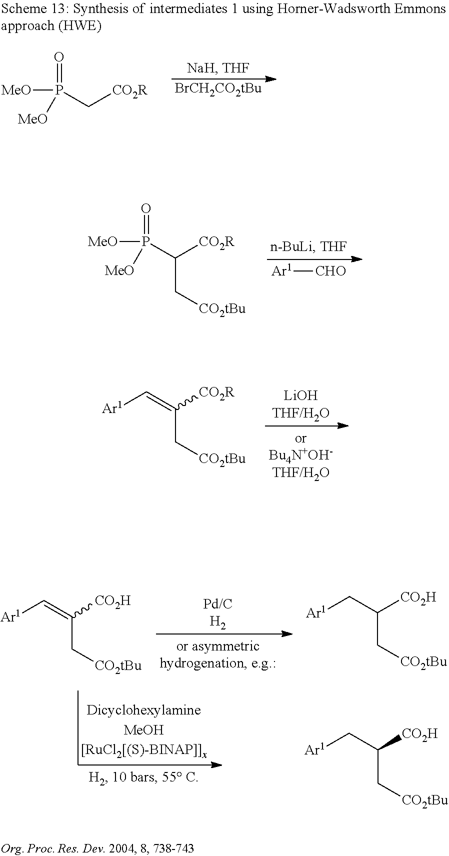

The HWE methodology as depicted in Scheme 13 is the preferred methodology of the invention for the synthesis of intermediates 1.

### Synthesis of Compounds 98, 100 and 101 (Scheme 14):

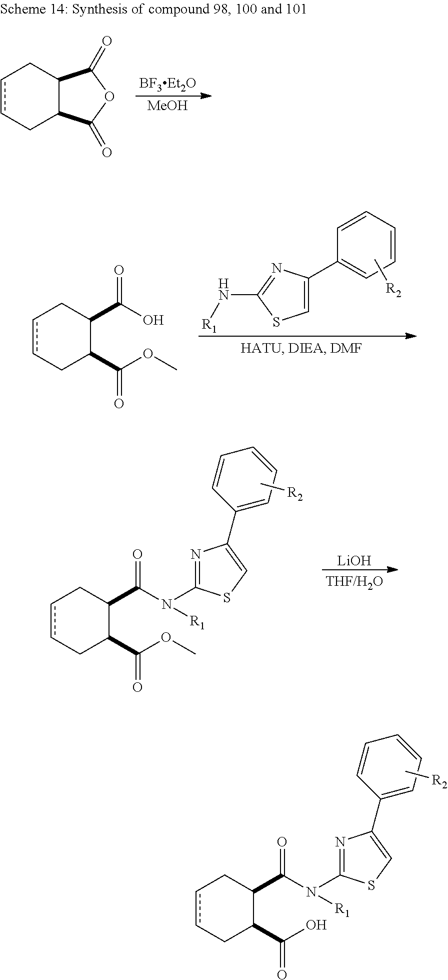

### General Scheme for the Preparation of Biaryl- or Heterobiaryl-Thiazole Amine Intermediates Using Suzuki Approach (Scheme 15):

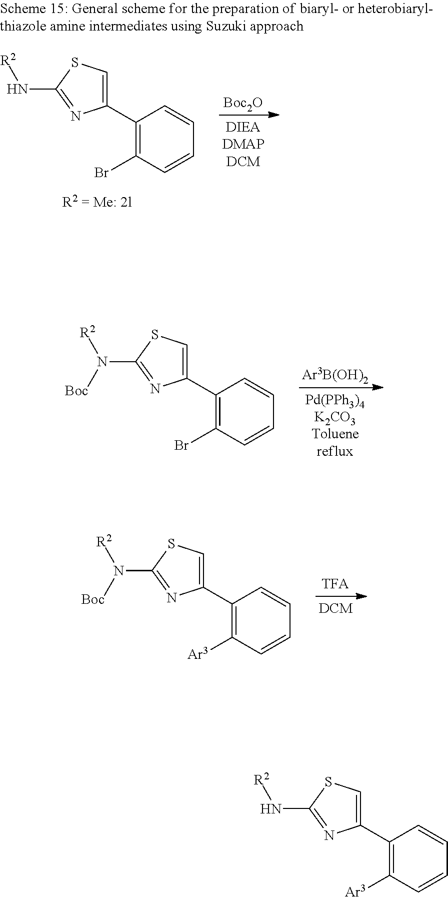

### Synthesis of Intermediates 2n and 2r3 (Scheme 16):

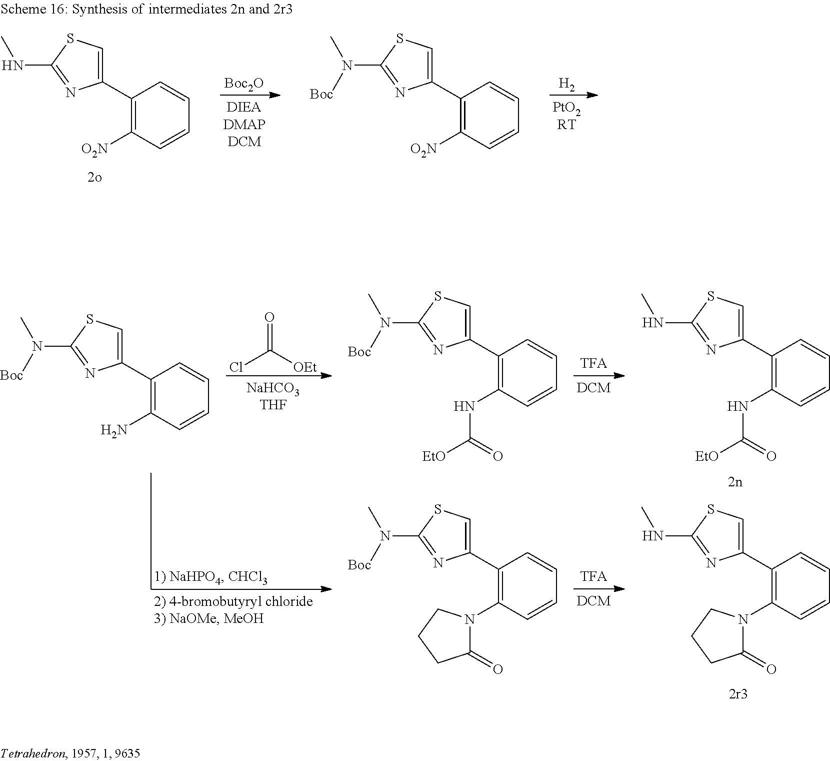

### Alternative General Scheme for the Preparation of Biaryl- or Heterobiaryl-Thiazole Amine Intermediates Using Suzuki Approach (Scheme 17):

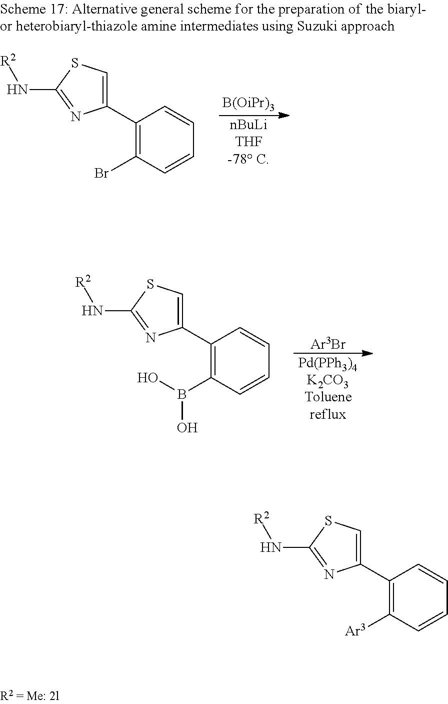

Synthesis of Compound n^{o}198 (Scheme 18):

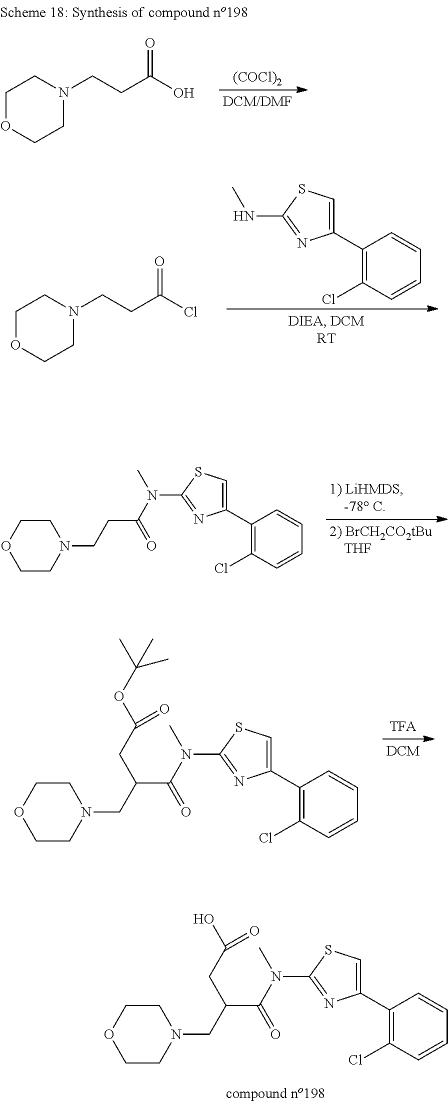

General Synthetic Scheme for the Preparation of Substituted Acetophenone Reagents through Weinreb Amide Approach (Scheme 20):

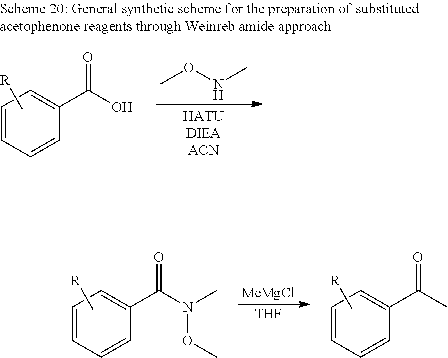

### Synthesis of Intermediate 2p3 (Scheme 21):

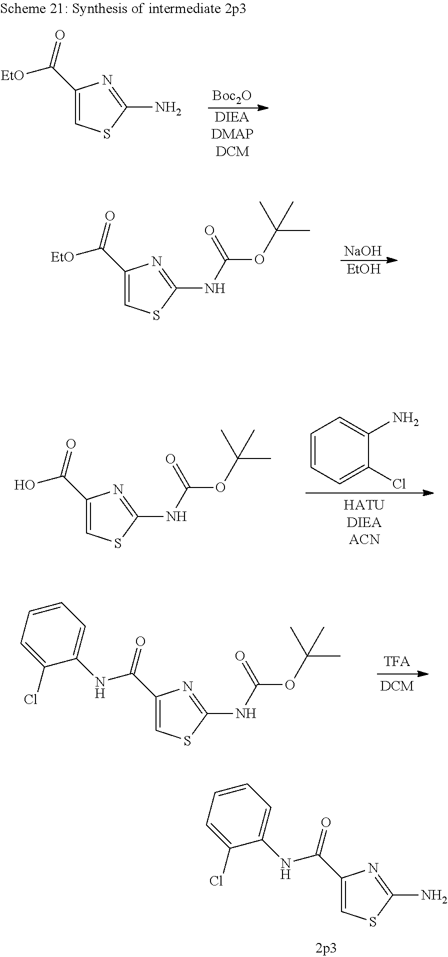

Synthesis of Compound n^{o}238 (Scheme 22):

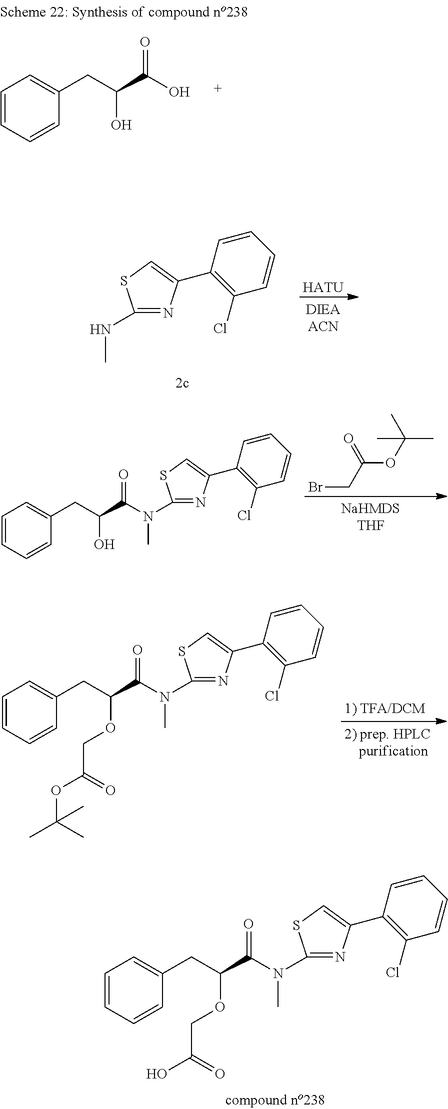

### General Synthetic Scheme for the Preparation of Substituted Thiourea Reagents (Scheme 23):

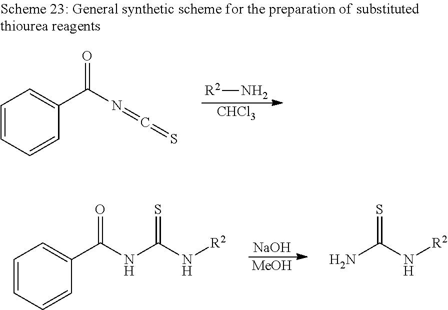

### General Method A: Synthesis of Intermediate 1a (S)-4-tert-butoxy-4-oxo-2-phenylbutanoic acid

### Step 1: Synthesis of (S)-4-benzyl-3-(2-phenylacetyl)oxazolidin-2-one

(S)-4-benzyloxazolidin-2-one (0.011 mol) was dissolved in THF (50 mL). A 1.6 M solution of n-BuLi (0.0124 mol) was added dropwise at −78° C. A solution of 2-phenylacetyl chloride (0.011 mol) in THF (20 mL) was added dropwise to the obtained dark solution at the same temperature. The reaction mixture was stirred for 1 h at −78° C. Then a saturated solution of NH₄Cl (2 mL) and a solution of NaHCO₃ (4 mL) were added dropwise, and the reaction mixture was warmed to RT. The organic layer was separated, and the aqueous one was extracted with diethyl ether (3×25 mL). The combined extracts were washed with water, brine, dried over Na₂SO₄, and evaporated. The residue was purified by chromatography (silica gel, hexane/ether, 2/1) to yield title compound. Y: 2.1 g (64.7%).

### Step 2: Synthesis of (S)-tert-butyl 4-((S)-4-benzyl-2-oxooxazolidin-3-yl)-4-oxo-3-phenylbutanoate

A 1M solution of NaHMDS (7.8 mmol) in THF was added to a solution of (S)-4-benzyl-3-(2-phenylacetyl)oxazolidin-2-one (7.1 mmol) in THF at −78° C. in a flow of argon. After keeping for 1.5 h at the same temperature tert-butyl bromoacetate (21.3 mmol) was added. The reaction mixture was stirred for 2 h at −78° C. and warmed to RT. A saturated solution of NH₄Cl (15 mL) and ethyl acetate (12 mL) were added. The organic layer was separated, and the aqueous one was extracted with ethyl acetate (3×30 mL). The combined extracts were washed with brine, dried over Na₂SO₄, and evaporated to give title compound. Y: 1.64 g (57%).

### Step 3: Synthesis of Intermediate 1a (S)-4-tert-butoxy-4-oxo-2-phenylbutanoic acid

(S)-Tert-butyl 4-((S)-4-benzyl-2-oxooxazolidin-3-yl)-4-oxo-3-phenylbutanoate (4 mmol) was dissolved in THF, and a 35% solution of H₂O₂ in water (16 mmol) was added dropwise at 0° C. Then a solution of LiOH (8 mmol) in H₂O (19 mL) was added. The reaction mixture was stirred for 1.5 h at 0° C. (TLC: CCl₄/ethyl acetate=7/3) indicated reaction was complete. A solution of Na₂SO₃ (15 mL) and NaHCO₃ (15 mL) were added at 0° C. The reaction mixture was evaporated in a rotary evaporator by one half. Water (50 mL) was added to the residue, and the mixture was extracted with CH₂Cl₂ (3×45 mL). The aqueous layer was acidified with 6M HCl to pH=2 at 0° C. The product was extracted with ethyl acetate (3×50 mL). Combined extracts were washed with brine, dried over Na₂SO₄, and evaporated. The residue was recrystallized from hexane to give title compound. Y: 0.75 g (75%).

The following intermediates were synthesized or may be synthesized using general method B adapting the oxazolidinone chirality and starting materials to targeted intermediate:
intermediate 1e: (R)-4-tert-butoxy-4-oxo-2-phenethylbutanoic acid,
intermediate 1f: (S)-4-tert-butoxy-4-oxo-2-phenethylbutanoic acid,
intermediate 1o: (R)-2-benzyl-5-methoxy-5-oxopentanoic acid; step 2 being replaced by a Michael addition on methyl acrylate using Ti(OiPr)₂Cl₂ and DIEA in DCM at 0° C. as described in WO1996/33176,
intermediate 1p: (S)-2-benzyl-5-methoxy-5-oxopentanoic acid; step 2 being replaced by a Michael addition on methyl acrylate using Ti(OiPr)₂Cl₂ and DIEA in DCM at 0° C. as described in WO1996/33176,
intermediate 1t: (2S)-4-tert-butoxy-2-(2,3-dihydro-1H-inden-1-yl)-4-oxobutanoic acid,
intermediate 1u: (S)-4-tert-butoxy-2-(2,3-dihydro-1H-inden-2-yl)-4-oxobutanoic acid,
intermediate 1v: (S)-4-tert-butoxy-2-cyclohexyl-4-oxobutanoic acid
intermediate 1w: (R)-4-tert-butoxy-2-(cyclohexylmethyl)-4-oxobutanoic acid,
intermediate 1x: (R)-4-tert-butoxy-4-oxo-2-phenylbutanoic acid,
intermediate 1z: (S)-4-tert-butoxy-4-oxo-2-((R)-1-phenylethyl)butanoic acid,
intermediate 1e1: (2R)-4-(tert-butoxy)-4-oxo-2-((tetrahydrofuran-2-yl)methyl)butanoic acid,
intermediate 1f1: (R)-4-(tert-butoxy)-2-(cyclopentylmethyl)-4-oxobutanoic acid,
intermediate 1g1: (R)-4-(tert-butoxy)-4-oxo-2-((tetrahydro-2H-pyran-4-yl)methyl)butanoic acid,
intermediate 1h1: (R)-4-(tert-butoxy)-4-oxo-2-((S)-1-phenylethyl)butanoic acid
intermediate 1o1: (2R)-4-(tert-butoxy)-4-oxo-2-((tetrahydrofuran-3-yl)methyl)butanoic acid
intermediate 1t1: (R)-4-(tert-butoxy)-2-(furan-2-ylmethyl)-4-oxobutanoic acid
intermediate 1u1: (S)-4-(tert-butoxy)-4-oxo-2-(thiophen-2-ylmethyl)butanoic acid.

### General Method B: Synthesis of Intermediate 1b (R)-2-benzyl-4-tert-butoxy-4-oxobutanoic acid

### Step 1: Synthesis of 3-(triphenylphosphoranylidene)dihydrofuran-2,5-dione

A solution of maleic anhydride (105 g, 1.07 mol) was added dropwise to a solution of triphenylphosphine (270 g, 1.03 mol) in acetone (1.2 L). The reaction mixture was stirred overnight at room temperature, cooled to 5° C., and filtered. The product was washed with acetone (2×100 mL), diethyl ether (100 mL), and dried under vacuum to give title compound. Y: 360 g (97%), rt=3.21 min (gradient A), (M+H)⁺=379.

### Step 2: Synthesis of 4-methoxy-4-oxo-3-(triphenylphosphoranylidene)butanoic acid

A solution of 3-(triphenylphosphoranylidene)dihydrofuran-2,5-dione (110 g, 0.305 mol) in methanol (600 mL) was stirred overnight at room temperature and evaporated. The residue was recrystallized from ethyl acetate (500 mL) to give title compound. Y: 98 g (81%) rt=3.32 min (gradient A), (M+H)⁺=393.

### Step 3: Synthesis of (3E)-3-(methoxycarbonyl)-4-phenylbut-3-enoic acid

4-methoxy-4-oxo-3-(triphenylphosphoranylidene)butanoic acid (50 g, 0.127 mol) was suspended in benzene (100 mL). A solution of benzaldehyde (14.8 g, 0.14 mol) in a mixture of dichloromethane (30 mL) and benzene (7.5 mL) was added dropwise. The reaction mixture was stirred at RT for 20 h, diluted with diethyl ether (200 mL), and extracted with a solution of potassium bicarbonate (0.23 mol) in water (300 mL). The organic layer was discarded and the aqueous one was washed with a mixture of benzene (200 mL) and ether (100 mL). The aqueous solution was acidified with HCl (30 mL) under cooling and extracted with an ethyl acetate/benzene mixture, 1:2 (2×400 mL). The organic layer was washed with water (50 mL) and brine (50 mL), dried over sodium sulfate, and evaporated. The obtained crude product (28 g) was purified by column chromatography (silica gel, CCl₄/ethyl acetate, 1:0→9:1) to give title compound. Y: 18.9 g (67.5%) rt=3.49 min (gradient A), (M+H)⁺=221.

### Step 4: Synthesis of (3R)-3-benzyl-4-methoxy-4-oxobutanoic acid

A mixture of (3E)-3-(methoxycarbonyl)-4-phenylbut-3-enoic acid (10.75 g, 48.8 mmol), dicyclohexylamine (18.62 g, 102.6 mmol), water (10 mL), and dichloro((S)-(−)-2,2-bis(diphenylphosphino)-1,1-binaphthyl)ruthenium(I) (40 mg) in methanol (90 mL) was hydrogenated in a Parr apparatus at 60° C. and 60 psi for 30 h. The resulting mixture was evaporated in a rotary evaporator by ½. Acetonitrile (90 mL) was added to the residue, and the mixture was evaporated again by ½. This operation was repeated once more, and the solution was left at RT overnight. The formed precipitate was filtered off and washed with cold acetonitrile. The product (9 g) was dissolved in water (150 mL) and acidified with concentrated HCl to pH=3 under cooling. The product was extracted with an ethyl acetate/benzene 1:2 mixture (300 mL). The organic layer was washed with water, brine, dried over Na₂SO₄, and evaporated to give title compound. Y: 6.35 g (58.6%) P>95%, rt=3.54 min (gradient A), (M+H)⁺=222, ee: 96% (method C).

### Step 5: Synthesis of (R)-4-tert-butyl 1-methyl 2-benzylsuccinate

Tert-butyl-2,2,2-trichloroacetimidate (9 mmol, 1.61 mL) and boron trifluoride diethyl etherate (0.675 mmol, 85 μL) was added to a solution of (3R)-3-Benzyl-4-methoxy-4-oxobutanoic acid (4.5 mmol, 1 g) in anhydrous THF (10 mL) at RT. The mixture stirred at RT under nitrogen for 3 h. TLC (cyclohexane/AcOEt=1/1) indicated reaction was complete. Reaction mixture was diluted with sat. aq. NaHCO₃ (10 mL) and extracted with AcOEt (2×20 mL). Combined organic layers were washed with brine, dried over MgSO₄, evaporated. Crude was purified by flash chromatography (cyclohexane/AcOEt=9/1) to give title compound as a very light yellow oil. Y: 1.25 g (62%), P>90% rt=4.65 mn (gradient A), (M+H)⁺=222 (−tBu) by ¹H NMR.

### Step 6: Synthesis of Intermediate 1b (R)-2-benzyl-4-tert-butoxy-4-oxobutanoic acid

To a solution of (R)-4-tert-butyl 1-methyl 2-benzylsuccinate (308 mg, 1.11 mmol) in THF (3 mL) was added a solution of lithium hydroxide (107 mg, 4.44 mmol) in water (3 mL). The mixture was stirred at RT overnight. TLC (cyclohexane/AcOEt=7/3) indicated reaction was complete. Reaction mixture was acidified to pH=1 with 2M HCl and extracted with DCM (2×20 mL). Combined organic layers were passed through a phase separator and evaporated. Crude was purified by flash chromatography (cyclohexane/AcOEt=9/1→7/3) (loading as solution in starting eluent) to yield title compound as a colorless oil. Y: 274 mg (94%), P>95%, rt=4.17 mn (gradient A), (M+H)⁺=209 (−tBu).

The following intermediates were or may be synthesized using general method B:
intermediate 1c: (R)-4-tert-butoxy-2-(4-fluorobenzyl)-4-oxobutanoic acid,
intermediate 1d: (R)-4-tert-butoxy-2-(cyclohexylmethyl)-4-oxobutanoic acid,
intermediate 1g: (R)-4-tert-butoxy-4-oxo-2-(4-(trifluoromethyl)benzyl)butanoic acid,
intermediate 1h: (R)-4-tert-butoxy-4-oxo-2-(3-(trifluoromethyl)benzyl)butanoic acid,
intermediate 1i: (R)-4-tert-butoxy-2-(2-cyanobenzyl)-4-oxobutanoic acid
intermediate 1j: (R)-4-tert-butoxy-2-(3-cyanobenzyl)-4-oxobutanoic acid,
intermediate 1k: (R)-4-tert-butoxy-2-(4-cyanobenzyl)-4-oxobutanoic acid,
intermediate 1l: (R)-4-tert-butoxy-2-(4-methoxybenzyl)-4-oxobutanoic acid,
intermediate 1m: (R)-4-tert-butoxy-2-(3-methoxybenzyl)-4-oxobutanoic acid,
intermediate 1n: (R)-4-tert-butoxy-2-(2-methoxybenzyl)-4-oxobutanoic acid,
intermediate 1q: (R)-4-tert-butoxy-2-(4-chlorobenzyl)-4-oxobutanoic acid,
intermediate 1r: (R)-4-tert-butoxy-2-(3-chlorobenzyl)-4-oxobutanoic acid,
intermediate 1s: (R)-4-tert-butoxy-2-(2-chlorobenzyl)-4-oxobutanoic acid,
intermediate 1y: (R)-4-tert-butoxy-2-(3-fluorobenzyl)-4-oxobutanoic acid.

### General Method C: Synthesis of Intermediate 2a 4-(2-chlorophenyl)thiazol-2-amine

Thiourea (2.1 g, 27.45 mmol) was added to a solution 2-bromo-1-(2-chlorophenyl)ethanone (7 g, 27.45 mmol) in ethanol (10 mL) and reaction mixture was stirred at RT for 18 h. The solvent was evaporated and refluxed for 5 minutes in DCM. Suspension was filtered to yield 7.84 g of 4-(2-chlorophenyl)thiazol-2-amine hydrobromide as a white powder. This powder was stirred in a mixture of aq. sat. Na₂CO₃ and AcOEt. Phases are separated and organic layer dried over MgSO₄, concentrated in vacuo to yield title compound as a yellow oil which solidifies spontaneously. Y: 5.37 g (93%), P=100%, rt=2.84 mn (gradient A), (M+H)⁺=211.

The following intermediates were or may be synthesized from the appropriate bromoketone (for which synthesis is described in Scheme 20) and thiourea (for which synthesis is described in Scheme 23) using general method C:
intermediate 2c: 4-(2-chlorophenyl)-N-methylthiazol-2-amine, using N-methylthiourea instead of thiourea,
intermediate 2f: 4-(2,4,6-trichlorophenyl)thiazol-2-amine,
intermediate 2g: N-benzyl-4-(2-chlorophenyl)thiazol-2-amine,
intermediate 2i: 2-(2-(methylamino)thiazol-4-yl)benzonitrile
intermediate 2j: 4-(2-chlorophenyl)-N-ethylthiazol-2-amine,
intermediate 2l: 4-(2-bromophenyl)-N-methylthiazol-2-amine,
intermediate 2o: N-methyl-4-(2-nitrophenyl)thiazol-2-amine,
intermediate 2s: 4-(3-(trifluoromethoxy)phenyl)thiazol-2-amine
intermediate 2w: N-cyclopropyl-4-(2,5-dichlorophenyl)thiazol-2-amine,
intermediate 2a1: 4-(2,5-dichlorophenyl)-N-methylthiazol-2-amine,
intermediate 2y1: 4-(2-chloro-5-(trifluoromethyl)phenyl)-N-methylthiazol-2-amine,
intermediate 2z1: 4-(2-chloro-5-fluorophenyl)-N-methylthiazol-2-amine
intermediate 2a2: 4-(3,5-dichlorophenyl)-N-methylthiazol-2-amine,
intermediate 2b2: 4-(3-(difluoromethoxy)phenyl)-N-methylthiazol-2-amine,
intermediate 2e2: 4-(5-chloro-2-(trifluoromethyl)phenyl)-N-methylthiazol-2-amine,
intermediate 2f2: N-methyl-4-(2,3,5-trichlorophenyl)thiazol-2-amine,
intermediate 2l2: N-methyl-4-(2-(trifluoromethoxy)phenyl)thiazol-2-amine,
intermediate 2m2: 4-(2-chloro-5-fluorophenyl)-N-cyclopropylthiazol-2-amine,
intermediate 2n2: N-cyclopropyl-4-(3-(difluoromethoxy)phenyl)thiazol-2-amine,
intermediate 2o2: 4-(2-chloro-5-(trifluoromethyl)phenyl)-N-cyclopropylthiazol-2-amine,
intermediate 2d3: N-(2-(benzyloxy)ethyl)-4-(2,5-dichlorophenyl)thiazol-2-amine,
intermediate 2e3: 4-(2-chloro-5-(difluoromethyl)phenyl)-N-methylthiazol-2-amine,
intermediate 2j3: (4-(2-chloro-5-(difluoromethoxy)phenyl)-N-methylthiazol-2-amine),
intermediate 2v3: (S)-1-((4-(2-chlorophenyl)thiazol-2-yl)amino)propan-2-ol,
intermediate 2w3: (R)-1-((4-(2-chlorophenyl)thiazol-2-yl)amino)propan-2-ol,
intermediate 2z3: 4-(2,3-dichlorophenyl)-N-methylthiazol-2-amine,
intermediate 2a4: N-methyl-4-(3-(trifluoromethoxy)phenyl)thiazol-2-amine,
intermediate 2b4: N-cyclopropyl-4-(3-(trifluoromethoxy)phenyl)thiazol-2-amine
intermediate 2c4: (4-(2-(difluoromethoxy)phenyl)-N-methylthiazol-2-amine).

### General Method D: Synthesis of Intermediate 2b 4-(2-chlorophenyl)-N-(cyclopropylmethyl)thiazol-2-amine

2-bromo-1-(2-chlorophenyl)ethanone (0.5 mmol, 116 mg) and dry sodium thiocyanate (0.55 mmol, 45 mg) were stirred in 1 mL ethanol for 3 h at 50° C. A solution of cyclopropane methyl amine (0.55 mmol, 39 mg) in 0.5 mL of ethanol was added at once and the reaction mixture was stirred for 12 h. The ethanol was distilled off, and ethyl acetate and water were added. The aqueous phase was extracted twice with ethyl acetate, the combined organic phases were dried over Na₂SO₄, and the solvent was removed in vacuo. Crude was purified by flash chromatography (cyclohexane/DCM=6/4) to give title compound as a dark yellow oil. Y: 40 mg (30%), P=100%, rt=3.5 mn (gradient A), (M+H)⁺=264.8.

The following intermediates were or may be synthesized from the appropriate bromoketone (for which synthesis is described in Scheme 20) and amine reagents using general method D:
intermediate 2d: N-allyl-4-(2-chlorophenyl)thiazol-2-amine,
intermediate 2e: methyl 2-(4-(2-chlorophenyl)thiazol-2-ylamino)acetate,
intermediate 2h: 4-(2-chlorophenyl)-N-(2,2,2-trifluoroethyl)thiazol-2-amine,
intermediate 2k: 4-(2-chlorophenyl)-N-cyclopropylthiazol-2-amine,
intermediate 2r: 2-((4-(2-chlorophenyl)thiazol-2-yl)amino)acetamide,
intermediate 2x: methyl 3-((4-(2-chlorophenyl)thiazol-2-yl)amino)propanoate,
intermediate 2u1: N-(2-(benzyloxy)ethyl)-4-(2-chlorophenyl)thiazol-2-amine,
intermediate 2v1: N-(3-(benzyloxy)propyl)-4-(2-chlorophenyl)thiazol-2-amine,
intermediate 2s2: 4-(2-chlorophenyl)-N-(2-methoxyethyl)thiazol-2-amine,
intermediate 2t2: N-(2-(benzyloxy)ethyl)-4-(2-chlorophenyl)thiazol-2-amine.

### General Method E: Synthesis of Example 1 compound n^{o}2: (R)-3-benzyl-4-(4-(2-chlorophenyl)thiazol-2-ylamino)-4-oxobutanoic acid

### Step 1: Synthesis of (R)-tert-butyl 3-benzyl-4-(4-(2-chlorophenyl)thiazol-2-ylamino)-4-oxobutanoate

To a solution of (R)-2-benzyl-4-tert-butoxy-4-oxobutanoic acid 1b (1.21 mmol, 320 mg) in anhydrous DMF (5 mL) was added HATU (1.33 mmol, 505 mg). After 5 min was added 4-(2-chlorophenyl)thiazol-2-amine 2a (1.33 mmol, 279 mg) and DIEA (1.815 mmol, 300 μL). Reaction mixture was stirred at RT for 4 days. TLC (cyclohexane/AcOEt=8/2) indicated reaction was complete. Reaction mixture (rm) was diluted with AcOEt (20 mL) and washed with sat. aq. NaHCO₃ (10 mL) and water (3×10 mL). The organic phase was dried over MgSO₄ and evaporated. Crude was purified by flash chromatography (cyclohexane/AcOEt=9/1) (loading onto silica) to yield title compound as a yellow gum. Y: 370 mg (67%), P>95%, rt=5.24 mn (gradient A), (M+H)⁺=457.1.

ACN was also used instead of DMF.

### Step 2: Synthesis of Example 1 Compound n^{o}2: (R)-3-benzyl-4-(4-(2-chlorophenyl)thiazol-2-ylamino)-4-oxobutanoic acid

To a solution of (R)-tert-butyl 3-benzyl-4-(4-(2-chlorophenyl)thiazol-2-ylamino)-4-oxobutanoate (0.7 mmol, 320 mg) in DCM (8 mL) was added TFA (2 mL). Rm was stirred at RT overnight. TLC (cyclohexane/AcOEt=7/3) indicated reaction was complete. Reaction mixture was evaporated and residue purified using a Biotage PEAX SPE cartridge. The oil obtained was triturated in diethyl ether/pentane=2/8 to yield title compound as a colorless solid. Y: 280 mg (99%), P>99% rt=9.32 mn (gradient B), (M+H)⁺=401.1, ee=96% (method B), ¹HNMR (CDCl₃): δ=12.2 (br s, 1H), 7.39-7.33 (m, 9H), 7.14 (s, 1H), 3.36 (q, 1H), 3.14 (m, 1H), 2.89-2.77 (m, 2H), 2.57 (dd, 1H).

Examples 2 to 18 were synthesized using general method E and intermediates described above or commercially available.

### Example 2

compound n^{o}9: (S)-4-(4-(2-chlorophenyl)thiazol-2-ylamino)-4-oxo-3-phenylbutanoic acid was synthesized using intermediates 1a and 2a. P=99%, (M+H)⁺=387, ee=98% (method A), ¹HNMR (DMSO-d₆): δ=7.78 (d, J=2.8Hz, 1H), 7.5 (m, 2H), 7.4-7.1 (m, 9H), 4.3 (q, 1H), 3.18 (dd, J=17Hz, J=27Hz, 1H), 2.66 (dd, J=4.8Hz, J=22Hz, 1H).

### Example 3

compound n^{o}3: (R)-3-benzyl-4-(4-(2,4-dichlorophenyl)thiazol-2-ylamino)-4-oxobutanoic acid was synthesized using intermediate 1b and 4-(2,4-dichlorophenyl)thiazol-2-amine.

### Example 4

compound n^{o}4: (R)-3-benzyl-4-(4-(2-fluorophenyl)thiazol-2-ylamino)-4-oxobutanoic acid was synthesized using intermediate 1b and 4-(2-fluorophenyl)thiazol-2-amine.

### Example 5

compound n^{o}5: (R)-3-benzyl-4-(4-(3,4-dichlorophenyl)thiazol-2-ylamino)-4-oxobutanoic acid was synthesized using intermediate 1b and 4-(3,4-dichlorophenyl)thiazol-2-amine.

### Example 8

compound n^{o}8: (R)-3-benzyl-4-(4-(4-cyanophenyl)thiazol-2-ylamino)-4-oxobutanoic acid was synthesized using intermediate 1b and 4-(2-aminothiazol-4-yl)benzonitrile.

### Example 9

compound n^{o}12: (R)-3-benzyl-4-(4-(3-chlorophenyl)thiazol-2-ylamino)-4-oxobutanoic acid was synthesized using intermediate 1b and 4-(3-chlorophenyl)thiazol-2-amine.

### Example 10

compound n^{o}13: (R)-3-benzyl-4-oxo-4-(4-(3-(trifluoromethyl)phenyl)thiazol-2-ylamino)butanoic acid was synthesized using intermediate 1b and 4-(3-(trifluoromethyl)phenyl)thiazol-2-amine.

### Example 11

compound n^{o}14: (R)-3-benzyl-4-((4-(2-chlorophenyl)thiazol-2-yl)(methyl)amino)-4-oxobutanoic acid was synthesized using intermediates 1b and 2c. Y: 142 mg (80%), P>99% rt=10.5 mn (gradient B), (M+H)⁺=414.8, ee=96% (method B), ¹HNMR (CDCl₃): δ=7.92 (d, 1H), 7.54 (s, 1H), 7.45 (d, 1H), 7.34-7.13 (m, 7H), 3.62 (s, 3H), 3.47 (m, 1H), 3.15-3.01 (m, 2H), 2.61-2.54 (m, 1H), 2.53-2.51 (dd, 1H).

### Example 12

compound n^{o}17: (R)-4-(4-(2-chlorophenyl)thiazol-2-ylamino)-3-(4-fluorobenzyl)-4-oxobutanoic acid was synthesized using intermediates 1c and 2a.

### Example 13

compound n^{o}18: (R)-4-(4-(2-chlorophenyl)thiazol-2-ylamino)-3-(cyclohexylmethyl)-4-oxobutanoic acid was synthesized using intermediates 1d and 2a. Y: 15 mg (30%), P>90% rt=10.76 mn (gradient B), (M+H)⁺=401.1, ee=96% (method B), ¹HNMR (CDCl₃): δ=12.26 (br s, 1H), 7.35-7.45 (m, 2H), 7.15-7.30 (m, 4H), 3.15-3.25 (m, 1H), 2.7 (dd, 1H), 2.5 (dd, 1H), 1.45-1.8 (m, 6H), 1.1-1.4 (m, 5H), 0.8-1.0 (m, 2H).

### Example 14

compound n^{o}22: (R)-4-(allyl(4-(2-chlorophenyl)thiazol-2-yl)amino)-3-benzyl-4-oxobutanoic acid was synthesized using intermediates 1b and 2d.

### Example 15

compound n^{o}23: (R)-3-benzyl-4-((4-(2-chlorophenyl)thiazol-2-yl)(2-methoxy-2-oxoethyl)amino)-4-oxobutanoic acid was synthesized using intermediate 1b and 2e.

### Example 16

compound n^{o}11: (R)-3-benzyl-4-oxo-4-(3-phenyl-1,2,4-thiadiazol-5-ylamino)butanoic acid was synthesized using intermediate 1b and 3-phenyl-1,2,4-thiadiazol-5-amine.

### Example 17

compound n^{o}20: (R)-3-benzyl-4-(4-(2-chlorophenyl)-5-fluorothiazol-2-ylamino)-4-oxobutanoic acid was synthesized using intermediate 1b and 4-(2-chlorophenyl)-5-fluorothiazol-2-amine which was prepared in one step from intermediate 2a as described in Chem. Res. Toxicol. 2007, 1954-1965.

### Example 18

compound n^{o}21: (R)-3-benzyl-4-((5-chloro-4-(2-chlorophenyl)thiazol-2-yl)(methyl)amino)-4-oxobutanoic acid was synthesized using intermediate 1b and 5-chloro-4-(2-chlorophenyl)-N-methylthiazol-2-amine which was prepared by reacting intermediate 2c with N-chlorosuccinimide and triethylamine in chloroform.

### Example 19

compound n^{o}15: (R)-3-benzyl-4-(5-(2-chlorophenyl)pyridin-2-ylamino)-4-oxobutanoic acid was synthesized using intermediate 1b and 5-iodopyridin-2-amine. Amide coupling such as in general method E, subsequent Suzuki coupling with 2-chlorophenylboronic acid using PdCl₂(PPh₃)₄ catalyst and K₂CO₃ in dioxane/H₂O followed by tBu deprotection as described in general method E provided title compound.

### Example 20

compound n^{o}10: (Z)-4-(4-(2-chlorophenyl)thiazol-2-ylamino)-4-oxobut-2-enoic acid was synthesized using intermediate 2a and (Z)-4-methoxy-4-oxobut-2-enoic acid. Amide coupling such as in general method E followed by saponification such as in step 6 of general method B provided title compound.

### Example 21

compound n^{o}16: (R)-3-(4-(2-chlorophenyl)thiazol-2-ylcarbamoyl)heptanoic acid was synthesized using intermediate 2a and (R)-2-(2-tert-butoxy-2-oxoethyl)hexanoic acid. (R)-2-(2-tert-butoxy-2-oxoethyl)hexanoic acid was prepared from (R)-3-(methoxycarbonyl)heptanoic acid as done in steps 5 and 6 of general method B.

### Example 22

compound n^{o}19: (R)-3-(4-(2-chlorophenyl)thiazol-2-ylcarbamoyl)-5-methylhexanoic acid was synthesized using intermediate 2a and (R)-2-(2-tert-butoxy-2-oxoethyl)-4-methylpentanoic acid. (R)-2-(2-tert-butoxy-2-oxoethyl)-4-methylpentanoic acid was prepared from (R)-3-(methoxycarbonyl)-5-methylhexanoic acid as done in steps 5 and 6 of general method B.

### Example 23

compound n^{o}1: 6-(4-(2-chlorophenyl)thiazol-2-ylcarbamoyl)cyclohex-3-enecarboxylic acid was synthesized using intermediate 2a and 6-(methoxycarbonyl)cyclohex-3-enecarboxylic acid. Amide coupling such as in general method E followed by saponification such as in step 6 of general method B provided title compound.

### Example 24

compound n^{o}24: (R)-methyl 3-benzyl-4-(4-(2-chlorophenyl)thiazol-2-ylamino)-4-oxobutanoate may be synthesized by treating compound n^{o}2 with TMSCl in MeOH.

### Example 26

compound n^{o}26: (R)-3-(4-(2-chlorophenyl)thiazol-2-ylcarbamoyl)-5-phenylpentanoic acid may be synthesized from intermediates 1e and 2a using general method E.

### Example 27

compound n^{o}27: (S)-3-(4-(2-chlorophenyl)thiazol-2-ylcarbamoyl)-5-phenylpentanoic acid may be synthesized from intermediates 1f and 2a using general method E.

### Example 28

compound n^{o}28: (R)-4-(4-(2-chlorophenyl)thiazol-2-ylamino)-4-oxo-3-(4-(trifluoromethyl)benzyl)butanoic acid was synthesized from intermediates 1g and 2a using general method E.

### Example 29

compound n^{o}29: (R)-4-(4-(2-chlorophenyl)thiazol-2-ylamino)-4-oxo-3-(3-(trifluoromethyl)benzyl)butanoic acid was synthesized from intermediates 1h and 2a using general method E.

### Example 30

compound n^{o}30: (R)-4-(4-(2-chlorophenyl)thiazol-2-ylamino)-3-(2-cyanobenzyl)-4-oxobutanoic acid may be synthesized from intermediates 1i and 2a using general method E.

### Example 31

compound n^{o}31: (R)-4-(4-(2-chlorophenyl)thiazol-2-ylamino)-3-(3-cyanobenzyl)-4-oxobutanoic acid may be synthesized from intermediates 1j and 2a using general method E.

### Example 32

compound n^{o}32: (R)-4-(4-(2-chlorophenyl)thiazol-2-ylamino)-3-(4-cyanobenzyl)-4-oxobutanoic acid may be synthesized from intermediates 1k and 2a using general method E.

### Example 33

compound n^{o}33: (R)-4-(4-(2-chlorophenyl)thiazol-2-ylamino)-3-(4-methoxybenzyl)-4-oxobutanoic acid may be synthesized from intermediates 1l and 2a using general method E.

### Example 34

compound n^{o}34: (R)-4-(4-(2-chlorophenyl)thiazol-2-ylamino)-3-(3-methoxybenzyl)-4-oxobutanoic acid may be synthesized from intermediates 1m and 2a using general method E.

### Example 35

compound n^{o}35: (R)-4-(4-(2-chlorophenyl)thiazol-2-ylamino)-3-(2-methoxybenzyl)-4-oxobutanoic acid may be synthesized from intermediates 1n and 2a using general method E.

### Example 36

compound n^{o}36: (R)-3-benzyl-4-(4-(2-methoxyphenyl)thiazol-2-ylamino)-4-oxobutanoic acid was synthesized from intermediate 1b and 4-(2-methoxyphenyl)thiazol-2-amine using general method E.

### Example 37

compound n^{o}37: ((R)-3-benzyl-4-oxo-4-(4-(2,4,6-trichlorophenyl)thiazol-2-ylamino)butanoic acid may be synthesized from intermediates 1b and 2f using general method E.

### Example 38

compound n^{o}38: (R)-4-benzyl-5-((4-(2-chlorophenyl)thiazol-2-yl)(methyl)amino)-5-oxopentanoic acid may be synthesized from intermediates 1o and 2c using general method E, replacing the TFA tBu ester deprotection by a methyl ester saponification using LiOH in THF/H₂O.

### Example 39

compound n^{o}39: (S)-4-benzyl-5-((4-(2-chlorophenyl)thiazol-2-yl)(methyl)amino)-5-oxopentanoic acid was synthesized from intermediates 1p and 2c using general method E, replacing the TFA tBu ester deprotection by a methyl ester saponification using LiOH in THF/H₂O.

### Example 40

compound n^{o}40: (R)-methyl 4-benzyl-5-(4-(2-chlorophenyl)thiazol-2-ylamino)-5-oxopentanoate may be synthesized from intermediates 1o and 2a using general method E.

### Example 41

compound n^{o}41: (S)-methyl 4-benzyl-5-(4-(2-chlorophenyl)thiazol-2-ylamino)-5-oxopentanoate may be synthesized from intermediates 1p and 2a using general method E.

### Example 42

compound n^{o}42: (R)-3-benzyl-4-((4-(2-chlorophenyl)thiazol-2-yl)(cyclopropylmethyl)amino)-4-oxobutanoic acid was synthesized from intermediates 1b and 2b using general method E.

### Example 43

compound n^{o}43:(R)-3-benzyl-4-(benzyl(4-(2-chlorophenyl)thiazol-2-yl)amino)-4-oxobutanoic acid was synthesized from intermediates 1b and 2g using general method E.

### Example 44

compound n^{o}44: (R)-3-benzyl-4-((4-(2-chlorophenyl)thiazol-2-yl)(2,2,2-trifluoroethyl)amino)-4-oxobutanoic acid may be synthesized from intermediates 1b and 2h using general method E.

### Example 45

compound n^{o}45: (R)-4-((4-(2-chlorophenyl)thiazol-2-yl)(methyl)amino)-3-(4-methoxybenzyl)-4-oxobutanoic acid was synthesized from 4-(tert-butoxy)-2-(4-methoxybenzyl)-4-oxobutanoic acid and intermediate 2c using general method E and chiral preparative HPLC purification. 4-(tert-butoxy)-2-(4-methoxybenzyl)-4-oxobutanoic acid was synthesized from commercially available 4-methoxybenzaldehyde using the HWE methodology (Scheme 13).

### Example 46

compound n^{o}46: (R)-4-((4-(2-chlorophenyl)thiazol-2-yl)(methyl)amino)-3-(3-methoxybenzyl)-4-oxobutanoic acid may be synthesized from intermediates 1m and 2c using general method E.

### Example 47

compound n^{o}47: (R)-4-((4-(2-chlorophenyl)thiazol-2-yl)(methyl)amino)-3-(2-methoxybenzyl)-4-oxobutanoic acid may be synthesized from intermediates 1n and 2c using general method E.

### Example 48

compound n^{o}48: (R)-4-((4-(2-chlorophenyl)thiazol-2-yl)(methyl)amino)-3-(4-cyanobenzyl)-4-oxobutanoic acid was synthesized from 4-(tert-butoxy)-2-(4-cyanobenzyl)-4-oxobutanoic acid and intermediate 2c using general method E and chiral preparative HPLC purification. 4-(tert-butoxy)-2-(4-cyanobenzyl)-4-oxobutanoic acid was synthesized from commercially available 4-cyanobenzaldehyde using the HWE methodology (Scheme 13).

### Example 49

compound n^{o}49: (R)-4-((4-(2-chlorophenyl)thiazol-2-yl)(methyl)amino)-3-(3-cyanobenzyl)-4-oxobutanoic acid may be synthesized from intermediates 1j and 2c using general method E.

### Example 50

compound n^{o}50: (R)-4-((4-(2-chlorophenyl)thiazol-2-yl)(methyl)amino)-3-(2-cyanobenzyl)-4-oxobutanoic acid may be synthesized from intermediates 1i and 2c using general method E.

### Example 51

compound n^{o}51: (R)-3-(4-chlorobenzyl)-4-((4-(2-chlorophenyl)thiazol-2-yl)(methyl)amino)-4-oxobutanoic acid was synthesized from 4-(tert-butoxy)-2-(4-chlorobenzyl)-4-oxobutanoic acid and intermediate 2c using general method E and chiral preparative HPLC purification. 4-(tert-butoxy)-2-(4-chlorobenzyl)-4-oxobutanoic acid was synthesized from commercially available 4-chlorobenzaldehyde using the HWE methodology (Scheme 13).

### Example 52

compound n^{o}52: (R)-3-(3-chlorobenzyl)-4-((4-(2-chlorophenyl)thiazol-2-yl)(methyl)amino)-4-oxobutanoic acid may be synthesized from intermediates 1r and 2c using general method E.

### Example 53

compound n^{o}53: (R)-3-(2-chlorobenzyl)-4-((4-(2-chlorophenyl)thiazol-2-yl)(methyl)amino)-4-oxobutanoic acid may be synthesized from intermediates 1s and 2c using general method E.

### Example 54

compound n^{o}54: (3S)-4-((4-(2-chlorophenyl)thiazol-2-yl)(methyl)amino)-3-(2,3-dihydro-1H-inden-1-yl)-4-oxobutanoic acid may be synthesized from intermediates 1t and 2c using general method E. 1t may be synthesized using Stobbe's condensation (Scheme 6).

### Example 55

compound n^{o}55: (S)-4-((4-(2-chlorophenyl)thiazol-2-yl)(methyl)amino)-3-(2,3-dihydro-1H-inden-2-yl)-4-oxobutanoic acid may be synthesized from intermediates 1u and 2c using general method E. 1u may be synthesized using Stobbe's condensation (Scheme 6).

### Example 56

compound n^{o}56: (R)-4-(benzo[d]thiazol-2-yl(methyl)amino)-3-benzyl-4-oxobutanoic acid may be synthesized from intermediate 1b and N-methylbenzo[d]thiazol-2-amine using general method E. N-methylbenzo[d]thiazol-2-amine may be prepared by Eischweiler-Clarke methylation of benzo[d]thiazol-2-amine.

### Example 57

compound n^{o}57: (R)-4-(benzo[d]oxazol-2-yl(methyl)amino)-3-benzyl-4-oxobutanoic acid may be synthesized from intermediate 1b and N-methylbenzo[d]oxazol-2-amine using general method E. N-methylbenzo[d]oxazol-2-amine may be prepared by Eischweiler-Clarke methylation of benzo[d]oxazol-2-amine.

### Example 58

compound n^{o}58: (R)-2-((1H-tetrazol-5-yl)methyl)-N-(4-(2-chlorophenyl)thiazol-2-yl)-N-methyl-3-phenylpropanamide may be synthesized from compound n^{o}14 using methodologies described in the isosteres synthetic schemes section.

### Example 59

compound n^{o}59: (R)-2-benzyl-N-(4-(2-chlorophenyl)thiazol-2-yl)-N-methyl-3-(5-oxo-4,5-dihydro-1,2,4-oxadiazol-3-yl)propanamide may be synthesized from compound n^{o}14 using methodologies described in the isosteres synthetic schemes section.

### Example 60

compound n^{o}60: (R)-3-benzyl-4-((4-(2-chlorophenyl)-5-fluorothiazol-2-yl)(methyl)amino)-4-oxobutanoic acid was synthesized using intermediate 1b and 4-(2-chlorophenyl)-5-fluoro-N-methylthiazol-2-amine which was prepared in one step from intermediate 2c as described in Chem. Res. Toxicol. 2007, 1954-1965.

### Example 61

compound n^{o}61: (S)-4-(4-(2-chlorophenyl)thiazol-2-ylamino)-3-cyclohexyl-4-oxobutanoic acid may be synthesized from intermediates 1v and 2a using general method E.

### Example 62

compound n^{o}62: (S)-4-((4-(2-chlorophenyl)thiazol-2-yl)(methyl)amino)-3-cyclohexyl-4-oxobutanoic acid was synthesized from (S)-4-(tert-butoxy)-2-cyclohexyl-4-oxobutanoic acid and intermediate 2c using general method E. (S)-4-(tert-butoxy)-2-cyclohexyl-4-oxobutanoic acid was synthesized from commercially available (S)-3-cyclohexyl-4-methoxy-4-oxobutanoic acid as described in steps 5 and 6 of general method B.

### Example 63

compound n^{o}63: (S)-4-((4-(2-chlorophenyl)thiazol-2-yl)(methyl)amino)-4-oxo-3-phenylbutanoic acid may be synthesized from intermediates 1a and 2c using general method E.

### Example 64

compound n^{o}64: (3R)-3-(4-(2-chlorophenyl)thiazol-2-ylcarbamoyl)-4-phenylpentanoic acid may be synthesized from intermediate 2a and (2R)-4-tert-butoxy-4-oxo-2-(1-phenylethyl)butanoic acid using general method E. (2R)-4-tert-butoxy-4-oxo-2-(1-phenylethyl)butanoic acid may be obtained by Stobbe condensation (Scheme 6).

### Example 65

compound n^{o}65: (R)-2-((1H-tetrazol-5-yl)methyl)-N-(4-(2-chlorophenyl)thiazol-2-yl)-3-phenylpropanamide was synthesized from compound n^{o}2 using methodologies described in the isosteres synthetic schemes section.

### Example 66

compound n^{o}66: (R)-2-benzyl-N-(4-(2-chlorophenyl)thiazol-2-yl)-3-(5-oxo-4,5-dihydro-1,2,4-oxadiazol-3-yl)propanamide was synthesized from compound n^{o}2 using methodologies described in the isosteres synthetic schemes section.

### Example 68

compound n^{o}68: (3R)-3-benzyl-4-(4-(2-chlorophenyl)thiazol-2-ylamino)-2-methyl-4-oxobutanoic acid was synthesized as described in Scheme 7.

### Example 69

compound n^{o}69: (R)-2-benzyl-N-(4-(2-chlorophenyl)thiazol-2-yl)-3-(3-hydroxyisoxazol-5-yl)propanamide may be synthesized using methodologies described in the isosteres synthetic schemes section.

### Example 70

compound n^{o}70: (R)-3-benzyl-4-(4-(2-chlorophenyl)pyrimidin-2-ylamino)-4-oxobutanoic acid was synthesized from intermediate 1b and 4-(2-chlorophenyl)pyrimidin-2-amine using general method E. 4-(2-chlorophenyl)pyrimidin-2-amine was synthesized as described in Scheme 8.

### Example 71

compound n^{o}71: (R)-3-benzyl-4-(6-(2-chlorophenyl)pyridin-2-ylamino)-4-oxobutanoic acid was synthesized from intermediate 1b and 6-(2-chlorophenyl)pyridin-2-amine using general method E. 6-(2-chlorophenyl)pyridin-2-amine was synthesized as described in Scheme 8.

### Example 72

compound n^{o}72: (E)-3-(4-(2-chlorophenyl)thiazol-2-ylcarbamoyl)-4-phenylbut-3-enoic acid may be synthesized from (E)-2-benzylidene-4-tert-butoxy-4-oxobutanoic acid and intermediate 2a using general method E. (E)-2-benzylidene-4-tert-butoxy-4-oxobutanoic acid was synthesized from maleic anhydride following steps 1, 2, 3, 5 and 6 of general method B.

### Example 74

compound n^{o}74: (Z)-4-((4-(2-chlorophenyl)thiazol-2-yl)(methyl)amino)-4-oxo-3-phenylbut-2-enoic acid may be synthesized as described in Scheme 9.

### Example 75

compound n^{o}75: (R)-3-(N-(4-(2-chlorophenyl)thiazol-2-yl)-N-methylsulfamoyl)-4-phenylbutanoic acid may be synthesized as described in Scheme 10.

### Example 76

compound n^{o}76: (S)-3-(N-(4-(2-chlorophenyl)thiazol-2-yl)-N-methylsulfamoyl)-4-phenylbutanoic acid may be synthesized as described in Scheme 10.

### Example 79

compound n^{o}79: (R)-3-benzyl-4-(4-(2-chlorophenyl)thiazol-2-ylamino)-3-fluoro-4-oxobutanoic acid may be synthesized as described in Scheme 11.

### Example 80

compound n^{o}80: (R)-3-benzyl-3-(4-(2-chlorophenyl)thiazol-2-ylcarbamoyl)hex-5-enoic acid may be synthesized as described in Scheme 11.

### Example 81

compound n^{o}81: (E)-3-(4-(2-chlorophenyl)thiazol-2-yl)(methyl)carbamoyl)-4-phenylbut-3-enoic acid was synthesized from (E)-2-benzylidene-4-tert-butoxy-4-oxobutanoic acid and intermediate 2c using general method E. (E)-2-benzylidene-4-tert-butoxy-4-oxobutanoic acid was synthesized from maleic anhydride following steps 1, 2, 3, 5 and 6 of general method B.

### Example 82

compound n^{o}82: (3S)-3-((4-(2-chlorophenyl)thiazol-2-yl)(methyl)carbamoyl)-4-phenylpentanoic acid may be synthesized from intermediate 2c and (2R)-4-tert-butoxy-4-oxo-2-(1-phenylethyl)butanoic acid using general method E. (2R)-4-tert-butoxy-4-oxo-2-(1-phenylethyl)butanoic acid may be obtained by Stobbe condensation (Scheme 6).

### Example 83

compound n^{o}83: (R)-3-benzyl-4-((3-(2-chlorophenyl)-1,2,4-thiadiazol-5-yl)(methyl)amino)-4-oxobutanoic acid was synthesized as described in Scheme 12.

### Example 84

compound n^{o}84: (R)-3-benzyl-4-((3-(2-chlorophenyl)-1,2,4-oxadiazol-5-yl)(methyl)amino)-4-oxobutanoic acid may be synthesized as described in Scheme 12.

### Example 85

compound n^{o}85: (R)-3-benzyl-4-((1-(2-chlorophenyl)-1H-pyrazol-3-yl(methyl)amino)-4-oxobutanoic acid may be synthesized as described in Scheme 12.

### Example 86

compound n^{o}86: (R)-2-benzyl-N-(4-(2-chlorophenyl)thiazol-2-yl)-3-(3-hydroxyisoxazol-5-yl)-N-methylpropanamide was synthesized using methodologies described in the isosteres synthetic schemes section.

### Example 89

compound n^{o}89: (R)-4-((4-(2-chlorophenyl)thiazol-2-yl)(methyl)amino)-3-(cyclohexylmethyl)-4-oxobutanoic acid was synthesized from intermediates 1w and 2c using general method E. Intermediate 1w was synthesized by hydrogenation of intermediate 1b using PtO₂ in MeOH.

### Example 90

compound n^{o}90: (R)-3-((4-(2-chlorophenyl)thiazol-2-yl)(methyl)carbamoyl)-5-methylhexanoic acid was synthesized from intermediate 2c and (R)-2-(2-tert-butoxy-2-oxoethyl)-4-methylpentanoic acid using general method E. (R)-2-(2-tert-butoxy-2-oxoethyl)-4-methylpentanoic acid was synthesized from (R)-3-(methoxycarbonyl)-5-methylhexanoic acid using methodology described in steps 5 and 6 of general method B.

### Example 91

compound n^{o}91: (R)-3-benzyl-4-((4-(2-cyanophenyl)thiazol-2-yl)(methyl)amino)-4-oxobutanoic acid was synthesized from intermediates 1b and 2i using general method E.

### Example 92

compound n^{o}92: (R)-4-(4-(2-chlorophenyl)thiazol-2-ylamino)-4-oxo-3-phenylbutanoic. Phenylacetic acid was converted to its tBu ester using tBu-TCA. Treatment of this tBu ester with LiHMDS followed by the addition of t-butyl bromoacetate provided 1-tert-butyl 4-methyl 2-phenylsuccinate. tBu deprotection with TFA yielded 4-methoxy-4-oxo-2-phenylbutanoic acid. HATU coupling of this acid with intermediate 2a and subsequent methyl ester saponification using LiOH yielded 4-(4-(2-chlorophenyl)thiazol-2-ylamino)-4-oxo-3-phenylbutanoic. Chiral preparative HPLC purification of this racemic mixture allowed isolating compound n^{o}92.

### Example 93

compound n^{o}93: (R)-4-(4-(2-chlorophenyl)thiazol-2-ylamino)-3-(3-fluorobenzyl)-4-oxobutanoic acid was synthesized from intermediates 1y and 2a using general method E and preparative HPLC purification.

### Example 94

compound n^{o}94: (S)-3-((4-(2-chlorophenyl)thiazol-2-yl)(methyl)carbamoyl)-4-methylpentanoic acid was synthesized from (S)-4-tert-butoxy-2-isopropyl-4-oxobutanoic acid and intermediate 2c using general method E. (S)-4-tert-butoxy-2-isopropyl-4-oxobutanoic acid was synthesized from commercially available (S)-3-(methoxycarbonyl)-4-methylpentanoic acid using reactions described in steps 5 and 6 of general method B.

### Example 95

compound n^{o}95: 4-((4-(2-chlorophenyl)thiazol-2-yl)(methyl)amino)-4-oxo-3-((tetrahydro-2H-pyran-4-yl)methyl)butanoic acid was synthesized from (R)-4-tert-butoxy-4-oxo-2-((tetrahydro-2H-pyran-4-yl)methyl)butanoic acid and intermediate 2c using general method E. (R)-4-tert-butoxy-4-oxo-2-((tetrahydro-2H-pyran-4-yl)methyl)butanoic acid was synthesized from commercially available tetrahydro-2H-pyran-4-carbaldehyde using the HWE methodology (Scheme 13).

### Example 96

compound n^{o}96: (R)-3-benzyl-4-((4-(2-chlorophenyl)thiazol-2-yl)(ethyl)amino)-4-oxobutanoic acid was synthesized from intermediates 1b and 2j using general method E.

### Example 97

compound n^{o}97: (R)-3-benzyl-4-((4-(2-chlorophenyl)thiazol-2-yl)(cyclopropyl)amino)-4-oxobutanoic acid was synthesized from intermediates 1b and 2k using general method E.

### Example 98

compound n^{o}98: cis-6-(4-(2-chlorophenyl)thiazol-2-ylcarbamoyl)cyclohex-3-enecarboxylic acid was synthesized from cis-3a,4,7,7a-tetrahydroisobenzofuran-1,3-dione and intermediate 2a as described in Scheme 14.

### Example 99

compound n^{o}99: 4-((4-(2-chlorophenyl)thiazol-2-yl)(methyl)amino)-3-(4-methoxybenzyl)-4-oxobutanoic acid was synthesized from 4-tert-butoxy-2-(4-methoxybenzyl)-4-oxobutanoic acid and intermediate 2c using general method E. 4-tert-butoxy-2-(4-methoxybenzyl)-4-oxobutanoic acid was synthesized from 4-methoxybenzaldehyde using the HWE methodology (Scheme 13).

### Example 100

compound n^{o}100: cis-6-((4-(2-chlorophenyl)thiazol-2-yl)(methyl)carbamoyl)cyclohex-3-enecarboxylic acid was synthesized from cis-3a,4,7,7a-tetrahydroisobenzofuran-1,3-dione and intermediate 2c as described in Scheme 14.

### Example 101

compound n^{o}101: cis-2-((4-(2-chlorophenyl)thiazol-2-yl)(methyl)carbamoyl)cyclohexanecarboxylic acid was synthesized from cis-hexahydroisobenzofuran-1,3-dione and intermediate 2c as described in Scheme 14.

### Example 102

compound n^{o}102: (R)-3-benzyl-4-(4-(2,5-dimethylthiophen-3-yl)thiazol-2-ylamino)-4-oxobutanoic acid was synthesized from intermediate 1b and commercially available 4-(2,5-dimethylthiophen-3-yl)thiazol-2-amine using general method E.

### Example 103

compound n^{o}103: 4-((4-(2-chlorophenyl)thiazol-2-yl)(methyl)amino)-3-(cyclohexylmethyl)-4-oxobutanoic acid was synthesized from 4-tert-butoxy-2-(cyclohexylmethyl)-4-oxobutanoic acid and intermediate 2c using general method E. 4-tert-butoxy-2-(cyclohexylmethyl)-4-oxobutanoic acid was synthesized by hydrogenation of (E)-4-tert-butyl 1-methyl 2-benzylidenesuccinate using PtO₂ in MeOH.

### Example 105

compound n^{o}105: 4-((4-(2-chlorophenyl)thiazol-2-yl)(methyl)amino)-3-(cyclopentylmethyl)-4-oxobutanoic acid was synthesized from 4-tert-butoxy-2-(cyclopentylmethyl)-4-oxobutanoic acid and intermediate 2c using general method E. 4-tert-butoxy-2-(cyclopentylmethyl)-4-oxobutanoic acid was synthesized from commercially available cyclopentanecarbaldehyde using the HWE methodology (Scheme 13).

### Example 106

compound n^{o}106: (3S ,4R)-3-((4-(2-chlorophenyl)thiazol-2-yl)(methyl)carbamoyl)-4-phenylpentanoic acid from intermediates 1z and 2c using general method E.

### Example 107

compound n^{o}107: (R)-3-benzyl-4-(methyl(4-(2-(thiophen-3-yl)phenyl)thiazol-2-yl)amino)-4-oxobutanoic acid was synthesized from intermediate 1b and N-methyl-4-(2-(thiophen-3-yl)phenyl)thiazol-2-amine using general method E. N-methyl-4-(2-(thiophen-3-yl)phenyl)thiazol-2-amine was synthesized from commercially available thiophen-3-ylboronic acid using the methodology shown in Scheme 15.

### Example 108

compound n^{o}108: (R)-3-benzyl-4-((4-(2-(6-chloropyridin-3-yl)phenyl)thiazol-2-yl)(methyl)amino)-4-oxobutanoic acid was synthesized from intermediate 1b and 4-(2-(6-chloropyridin-3-yl)phenyl)-N-methylthiazol-2-amine using general method E. 4-(2-(6-chloropyridin-3-yl)phenyl)-N-methylthiazol-2-amine was synthesized from commercially available 6-chloropyridin-3-ylboronic acid using the methodology shown in Scheme 15.

### Example 109

compound n^{o}109: (R)-4-((4-(2-chlorophenyl)thiazol-2-yl)methyl)amino)-4-oxo-3-(phenylamino)butanoic acid was synthesized from (R)-4-tert-butoxy-4-oxo-2-(phenylamino)butanoic acid and intermediate 2c using general method E. (R)-4-tert-butoxy-4-oxo-2-(phenylamino)butanoic acid was synthesized from commercially available (R)-2-amino-4-tert-butoxy-4-oxobutanoic acid and iodobenzene using CuI catalyzed coupling as described in J. Am. Chem. Soc. 1998, 120, 12459.

### Example 110

compound n^{o}110: 4-((4-(2-chlorophenyl)thiazol-2-yl)(methyl)amino)-3-(4-methylbenzyl)-4-oxobutanoic acid was synthesized from 4-tert-butoxy-2-(4-methylbenzyl)-4-oxobutanoic acid and intermediate 2c using general method E. 4-tert-butoxy-2-(4-methylbenzyl)-4-oxobutanoic acid was synthesized from 4-methylbenzaldehyde using the HWE methodology (Scheme 13).

### Example 111

compound n^{o}111: (R)-4-((4-([1,1′-biphenyl]-2-yl)thiazol-2-yl)(methyl)amino)-3-benzyl-4-oxobutanoic acid was synthesized from intermediate 1b and 4-([1,1′-biphenyl]-2-yl)-N-methylthiazol-2-amine using general method E. 4-([1,1′-biphenyl]-2-yl)-N-methylthiazol-2-amine was synthesized from commercially available phenylboronic acid using the methodology shown in Scheme 15.

### Example 112

compound n^{o}112: (R)-3-benzyl-4-(4-(2,5-dichlorothiophen-3-yl)thiazol-2-ylamino)-4-oxobutanoic acid was synthesized from intermediate 1b and commercially available 4-(2,5-dichlorothiophen-3-yl)thiazol-2-amine using general method E.

### Example 113

compound n^{o}113: 4-((4-(2-chlorophenyl)thiazol-2-yl)(methyl)amino)-3-(cyclopropylmethyl)-4-oxobutanoic acid was synthesized from intermediates 1a1 (4-(tert-butoxy)-2-(cyclopropylmethyl)-4-oxobutanoic acid) and 2c using general method E. 1a1 was synthesized from cyclopropanecarbaldehyde using the HWE methodology (Scheme 13).

### Example 114

compound n^{o}114: 4-((4-(2-chlorophenyl)thiazol-2-yl)(methyl)amino)-4-oxo-3-(thiazol-4-ylmethyl)butanoic acid was synthesized from intermediates 1b1 (4-(tert-butoxy)-4-oxo-2-(thiazol-4-ylmethyl)butanoic acid) and 2c using general method E. 1b1 was synthesized from thiazole-4-carbaldehyde using the HWE methodology (Scheme 13).

### Example 115

compound n^{o}115: (R)-3-benzyl-4-((4-(2-(6-(dimethylamino)pyridin-3-yl)phenyl)thiazol-2-yl)(methyl)amino)-4-oxobutanoic acid was synthesized from intermediate 1b and 4-(2-(6-(dimethylamino)pyridin-3-yl)phenyl)-N-methylthiazol-2-amine using general method E. 4-(2-(6-(dimethylamino)pyridin-3-yl)phenyl)-N-methylthiazol-2-amine was synthesized from (6-(dimethylamino)pyridin-3-yl)boronic acid and 2l using the methodology shown in Scheme 15.

### Example 116

compound n^{o}116: (R)-3-benzyl-4-((4-(2-(6-methoxypyridin-3-yl)phenyl)thiazol-2-yl)(methyl)amino)-4-oxobutanoic acid was synthesized from intermediate 1b and intermediate 2m (4-(2-(6-methoxypyridin-3-yl)phenyl)-N-methylthiazol-2-amine) using general method E. Intermediate 2m was synthesized from (6-methoxypyridin-3-yl)boronic acid and 2l using the methodology shown in Scheme 15.

### Example 117

compound n^{o}117: (R)-3-benzyl-4-((4-(2-(2-methoxypyridin-3-yl)phenyl)thiazol-2-yl)(methyl)amino)-4-oxobutanoic acid was synthesized from intermediate 1b and (4-(2-(2-methoxypyridin-3-yl)phenyl)-N-methylthiazol-2-amine) using general method E. (4-(2-(2-methoxypyridin-3-yl)phenyl)-N-methylthiazol-2-amine) was synthesized from (2-methoxypyridin-3-yl)boronic acid and 2l using the methodology shown in Scheme 15.

### Example 118

compound n^{o}118: (R)-3-benzyl-4-((4-(2-((ethoxycarbonyl)amino)phenyl)thiazol-2-yl)(methyl)amino)-4-oxobutanoic acid was synthesized from intermediates 1b and 2n (ethyl (2-(2-(methylamino)thiazol-4-yl)phenyl)carbamate) using general method E. Intermediate 2n was synthesized using the methodology described in Scheme 16.

### Example 119

compound n^{o}119: (R)-3-benzyl-4-((4-(2-(6-fluoropyridin-3-yl)phenyl)thiazol-2-yl)(methyl)amino)-4-oxobutanoic acid was synthesized from intermediates 1b and 2p (4-(2-(6-fluoropyridin-3-yl)phenyl)-N-methylthiazol-2-amine) using general method E and preparative HPLC purification. Intermediate 2p was synthesized from (6-fluoropyridin-3-yl)boronic acid and 2l using the methodology described in Scheme 15.

### Example 120

compound n^{o}120: (R)-3-benzyl-4-(methyl(4-(2-(6-methylpyridin-3-yl)phenyl)thiazol-2-yl)amino)-4-oxobutanoic acid was synthesized from intermediates 1b and 2q (N-methyl-4-(2-(6-methylpyridin-3-yl)phenyl)thiazol-2-amine) using general method E and preparative HPLC purification. Intermediate 2q was synthesized from (6-methylpyridin-3-yl)boronic acid and 2l using the methodology described in Scheme 15.

### Example 121

compound n^{o}121: (R)-4-((2-amino-2-oxoethyl)(4-(2-chlorophenyl)thiazol-2-yl)amino)-3-benzyl-4-oxobutanoic acid was synthesized from intermediates 1b and 2r using general method E.

### Example 122

compound n^{o}122: (R)-3-benzyl-4-oxo-4-((4-(3-(trifluoromethoxy)phenyl)thiazol-2-yl)amino)butanoic acid was synthesized from intermediates 1b and commercially available 2s (4-(3-(trifluoromethoxy)phenyl)thiazol-2-amine) using general method E.

### Example 123

compound n^{o}123: (R)-3-benzyl-4-((4-(2,5-dichlorophenyl)thiazol-2-yl)amino)-4-oxobutanoic acid was synthesized from intermediates 1b and commercially available 2t (4-(2,5-dichlorophenyl)thiazol-2-amine) using general method E.

### Example 124

compound n^{o}124: (R)-3-benzyl-4-((4-(3-chloro-4-fluorophenyl)thiazol-2-yl)amino)-4-oxobutanoic acid was synthesized from intermediates 1b and commercially available 2u (4-(3-chloro-4-fluorophenyl)thiazol-2-amine) using general method E.

### Example 125

compound n^{o}125: (R)-3-benzyl-4-((4-(3-chloro-4-methoxyphenyl)thiazol-2-yl)amino)-4-oxobutanoic acid was synthesized from intermediates 1b and commercially available 2v (4-(3-chloro-4-methoxyphenyl)thiazol-2-amine) using general method E.

### Example 126

compound n^{o}126: (R)-3-benzyl-4-((4-(2-chlorophenyl)thiazol-2-yl)(3-methoxy-3-oxopropyl)amino)-4-oxobutanoic acid was synthesized from intermediates 1b and 2x using general method E.

### Example 127

compound n^{o}127: 3-(bicyclo[2.2.1]heptan-2-ylmethyl)-4-((4-(2-chlorophenyl)thiazol-2-yl)(methyl)amino)-4-oxobutanoic acid was synthesized from intermediates 1c1 (2-(bicyclo[2.2.1]heptan-2-ylmethyl)-4-(tert-butoxy)-4-oxobutanoic acid) and 2c using general method E. 1c1 was synthesized from bicyclo[2.2.1]heptane-2-carbaldehyde using the HWE methodology (Scheme 13).

### Example 128

compound n^{o}128: (R)-3-benzyl-4-((4-(2-(6-ethoxypyridin-3-yl)phenyl)thiazol-2-yl)(methyl)amino)-4-oxobutanoic acid was synthesized from intermediates 1b and 2y (4-(2-(6-ethoxypyridin-3-yl)phenyl)-N-methylthiazol-2-amine) using general method E. Intermediate 2y was synthesized from (6-ethoxypyridin-3-yl)boronic acid and 2l using the methodology described in Scheme 15.

### Example 129

compound n^{o}129: (R)-3-benzyl-4-((4-(4′-methoxy-[1,1′-biphenyl]-2-yl)thiazol-2-yl)(methyl)amino)-4-oxobutanoic acid was synthesized from intermediates 1b and 2z (4-(4′-methoxy-[1,1′-biphenyl]-2-yl)-N-methylthiazol-2-amine) using general method E and preparative HPLC purification. Intermediate 2z was synthesized from (4-methoxyphenyl)boronic acid and 2l using the methodology described in Scheme 15.

### Example 130

compound n^{o}130: (R)-3-benzyl-4-((4-(2,5-dichlorophenyl)thiazol-2-yl)(methyl)amino)-4-oxobutanoic acid was synthesized from intermediates 1b and 2a1 using general method E.

### Example 131

compound n^{o}131: (R)-1-(5-(2-(2-(2-benzyl-3-carboxy-N-methylpropanamido)thiazol-4-yl)phenyl)pyridin-2-yl)pyrrolidin-1-ium 2,2,2-trifluoroacetate was synthesized from intermediates 1b and 2b1 (N-methyl-4-(2-(6-(pyrrolidin-1-yl)pyridin-3-yl)phenyl)thiazol-2-amine) using general method E. Intermediate 2b1 was synthesized from 2-(pyrrolidin-1-yl)-5-(4,4,5,5-tetramethyl-1,3,2-dioxaborolan-2-yl)pyridine and 2l by Suzuki coupling with the conditions described in Scheme 15.

### Example 132

compound n^{o}132: (R)-4-(2′-(2-(2-benzyl-3-carboxy-N-methylpropanamido)thiazol-4-yl)-[1,1′-biphenyl]-4-yl)morpholin-4-ium 2,2,2-trifluoroacetate was synthesized from intermediates 1b and 2c1 (N-methyl-4-(4′-morpholino-[1,1′-biphenyl]-2-yl)thiazol-2-amine) using general method E. Intermediate 2c1 was synthesized from 4-(4-(4,4,5,5-tetramethyl-1,3,2-dioxaborolan-2-yl)phenyl)morpholine and 2l by Suzuki coupling with the conditions described in Scheme 15.

### Example 133

compound n^{o}133: (R)-3-benzyl-4-(methyl(4-(2-(6-morpholinopyridin-3-yl)phenyl)thiazol-2-yl)amino)-4-oxobutanoic acid was synthesized from intermediates 1b and 2d1 (N-methyl-4-(2-(6-morpholinopyridin-3-yl)phenyl)thiazol-2-amine) using general method E. Intermediate 2d1 was synthesized from 4-(5-(4,4,5,5-tetramethyl-1,3,2-dioxaborolan-2-yl)pyridin-2-yl)morpholine and 2l using the methodology described in Scheme 15.

### Example 134

compound n^{o}134: (R)-3-benzyl-4-((4-(3′-chloro-[1,1′-biphenyl]-2-yl)thiazol-2-yl)(methyl)amino)-4-oxobutanoic acid was synthesized from intermediates 1b and 2e1 (4-(3′-chloro-[1,1′-biphenyl]-2-yl)-N-methylthiazol-2-amine) using general method E and preparative HPLC purification. Intermediate 2e1 was synthesized from (3-chlorophenyl)boronic acid and 2l using the methodology described in Scheme 15.

### Example 135

compound n^{o}135: (R)-3-benzyl-4-((4-(2-(furan-3-yl)phenyl)thiazol-2-yl)(methyl)amino)-4-oxobutanoic acid was synthesized from intermediates 1b and 2f1 (4-(2-(furan-3-yl)phenyl)-N-methylthiazol-2-amine) using general method E. Intermediate 2f1 was synthesized from furan-3-ylboronic acid and 2l by Suzuki coupling with the conditions described in Scheme 15.

### Example 136

compound n^{o}136: (R)-3-benzyl-4-((4-(2-(6-(2-methoxyethoxy)pyridin-3-yl)phenyl)thiazol-2-yl)(methyl)amino)-4-oxobutanoic acid was synthesized from intermediates 1b and 2g1 (4-(2-(6-(2-methoxyethoxy)pyridin-3-yl)phenyl)-N-methylthiazol-2-amine) using general method E. Intermediate 2g1 was synthesized from 5-bromo-2-(2-methoxyethoxy)pyridine and 2l using the methodology described in Scheme 17.

### Example 138

compound n^{o}138: (R)-3-benzyl-4-((4-(4′-isopropyl[1,1′-biphenyl]-2-yl)thiazol-2-yl)(methyl)amino)-4-oxobutanoic acid was synthesized from intermediates 1b and 2h1 (4-(4′-isopropyl-[1,1′-biphenyl]-2-yl)-N-methylthiazol-2-amine) using general method E and preparative HPLC purification. Intermediate 2h1 was synthesized from (4-isopropylphenyl)boronic acid and 2l by Suzuki coupling with the conditions described in Scheme 15.

### Example 139

compound n^{o}139: (R)-3-(cyclopentylmethyl)-4-((4-(2-(6-methoxypyridin-3-yl)phenyl)thiazol-2-yl)(methyl)amino)-4-oxobutanoic acid was synthesized from (R)-4-tert-butoxy-2-(cyclopentylmethyl)-4-oxobutanoic acid (ee=50%) and intermediate 2m using general method E and chiral preparative HPLC purification. (R)-4-tert-butoxy-2-(cyclopentylmethyl)-4-oxobutanoic acid (ee=50%) was synthesized from commercially available cyclopentanecarbaldehyde using the HWE methodology as described in Scheme 13.

### Example 140

compound n^{o}140: (R)-3-benzyl-4-((4-(2-(5-fluoro-6-methoxypyridin-3-yl)phenyl)thiazol-2-yl)(methyl)amino)-4-oxobutanoic acid was synthesized from intermediates 1b and 2i1 (4-(2-(5-fluoro-6-methoxypyridin-3-yl)phenyl)-N-methylthiazol-2-amine) using general method E. Intermediate 2i1 was synthesized from 5-bromo-3-fluoro-2-methoxypyridine using the methodology described in Scheme 17.

### Example 141

compound n^{o}141: (R)-3-benzyl-4-(methyl(4-(2-(6-((tetrahydro-2H-pyran-4-yl)oxy)pyridin-3-yl)phenyl)thiazol-2-yl)amino)-4-oxobutanoic acid was synthesized from intermediates 1b and 2j1 (N-methyl-4-(2-(6-((tetrahydro-2H-pyran-4-yl)oxy)pyridin-3-yl)phenyl)thiazol-2-amine) using general method E. Intermediate 2j1 was synthesized from 5-bromo-2-((tetrahydro-2H-pyran-4-yl)oxy)pyridine and 2l using the methodology described in Scheme 17.

### Example 142

compound n^{o}142: (R)-3-benzyl-4-(cyclopropyl(4-(2,5-dichlorophenyl)thiazol-2-yl)amino)-4-oxobutanoic acid from intermediates 1b and 2w using general method E.

### Example 143

compound n^{o}143: 4-((4-(2-chlorophenyl)thiazol-2-yl)methyl)amino)-3-(furan-2-ylmethyl)-4-oxobutanoic acid was synthesized from intermediates 1d1 (4-(tert-butoxy)-2-(furan-2-ylmethyl)-4-oxobutanoic acid) and 2c using general method E. 1d1 was synthesized from furan-2-carbaldehyde using the HWE methodology described Scheme 13.

### Example 144

compound n^{o}144: (R)-3-benzyl-4-((4-(2-cyclopropylphenyl)thiazol-2-yl)(methyl)amino)-4-oxobutanoic acid was synthesized from intermediates 1b and 2k1 (4-(2-cyclopropylphenyl)-N-methylthiazol-2-amine) using general method E and preparative HPLC purification. Intermediate 2k1 was synthesized from cyclopropylboronic acid and 2l using the methodology described in Scheme 15.

### Example 145

compound n^{o}145: (R)-3-benzyl-4-((4-(4′-(dimethylamino)-[1,1′-biphenyl]-2-yl)thiazol-2-yl)(methyl)amino)-4-oxobutanoic acid was synthesized from intermediates 1b and 2l1 (4-(4′-(dimethylamino)-[1,1′-biphenyl]-2-yl)-N-methylthiazol-2-amine) using general method E. Intermediate 2l1 was synthesized from N,N-dimethyl-4-(4,4,5,5-tetramethyl-1,3,2-dioxaborolan-2-yl)aniline using the methodology described in Scheme 15.

### Example 146

compound n^{o}146: (R)-3-benzyl-4-((4-(3′-fluoro-[1,1′-biphenyl]-2-yl)thiazol-2-yl)(methyl)amino)-4-oxobutanoic acid was synthesized from intermediates 1b and 2m1 (4-(3′-fluoro-[1,1′-biphenyl]-2-yl)-N-methylthiazol-2-amine) using general method E and preparative HPLC purification. Intermediate 2m1 was synthesized from (3-fluorophenyl)boronic acid and 4-(2-bromophenyl)-N-methylthiazol-2-amine by Suzuki coupling with the conditions described in Scheme 15.

### Example 147

compound n^{o}147: (R)-3-benzyl-4-((4-(3′,5′-difluoro-[1,1′-biphenyl]-2-yl)thiazol-2-yl)(methyl)amino)-4-oxobutanoic acid was synthesized from intermediates 1b and 2n1 (4-(3′,5′-difluoro-[1,1′-biphenyl]-2-yl)-N-methylthiazol-2-amine) using general method E. Intermediate 2n1 was synthesized from (3,5-difluorophenyl)boronic acid and 2l by Suzuki coupling with the conditions described in Scheme 15.

### Example 148

compound n^{o}148: (R)-3-benzyl-4-((4-(2-chloro-6-fluorophenyl)thiazol-2-yl)amino)-4-oxobutanoic acid was synthesized from intermediates 1b and commercially available 2o1 (4-(2-chloro-6-fluorophenyl)thiazol-2-amine) using general method E.

### Example 149

compound n^{o}149: (R)-3-benzyl-4-((4-(4′-chloro-[1,1′-biphenyl]-2-yl)thiazol-2-yl)(methyl)amino)-4-oxobutanoic acid was synthesized from intermediates 1b and 2p1 (4-(4′-chloro-[1,1′-biphenyl]-2-yl)-N-methylthiazol-2-amine) using general method E and preparative HPLC purification. Intermediate 2p1 was synthesized from (4-chlorophenyl)boronic acid and 2l by Suzuki coupling with the conditions described in Scheme 15.

### Example 150

compound n^{o}150: (R)-3-benzyl-4-(methyl(4-(2-(6-(2-oxopyrrolidin-1-yl)pyridin-3-yl)phenyl)thiazol-2-yl)amino)-4-oxobutanoic acid was synthesized from intermediates 1b and 2q1 (1-(5-(2-(2-(methylamino)thiazol-4-yl)phenyl)pyridin-2-yl)pyrrolidin-2-one) using general method E. Intermediate 2q1 was synthesized from 1-(5-bromopyridin-2-yl)pyrrolidin-2-one and 2l using the methodology described in Scheme 17.

### Example 151

compound n^{o}151: (R)-3-benzyl-4-((4-(4-chloro-2-(6-methoxypyridin-3-yl)phenyl)thiazol-2-yl)(methyl)amino)-4-oxobutanoic acid was synthesized from intermediates 1b and 2r1 (4-(4-chloro-2-(6-methoxypyridin-3-yl)phenyl)-N-methylthiazol-2-amine) using general method E. Intermediate 2r1 was synthesized from (6-methoxypyridin-3-yl)boronic acid and 4-(2-bromo-4-chlorophenyl)-N-methylthiazol-2-amine by Suzuki coupling with the conditions described in Scheme 15. 4-(2-bromo-4-chlorophenyl)-N-methylthiazol-2-amine was synthesized using general method C.

### Example 152

compound n^{o}152: (R)-3-benzyl-4-((4-(5-chloro-2-(6-methoxypyridin-3-yl)phenyl)thiazol-2-yl)(methyl)amino)-4-oxobutanoic acid was synthesized from intermediates 1b and 2s1 (4-(5-chloro-2-(6-methoxypyridin-3-yl)phenyl)-N-methylthiazol-2-amine) using general method E. Intermediate 2s1 was synthesized from (6-methoxypyridin-3-yl)boronic acid and 4-(2-bromo-5-chlorophenyl)-N-methylthiazol-2-amine by Suzuki coupling with the conditions described in Scheme 15. 4-(2-bromo-5-chlorophenyl)-N-methylthiazol-2-amine was synthesized using general method C.

### Example 153

compound n^{o}153: (R)-3-benzyl-4-((4-(3-fluoro-2-(6-methoxypyridin-3-yl)phenyl)thiazol-2-yl)(methyl)amino)-4-oxobutanoic acid was synthesized from intermediates 1b and 2t1 (4-(3-fluoro-2-(6-methoxypyridin-3-yl)phenyl)-N-methylthiazol-2-amine) using general method E and preparative HPLC purification. Intermediate 2t1 was synthesized from (6-methoxypyridin-3-yl)boronic acid and 4-(2-bromo-3-fluorophenyl)-N-methylthiazol-2-amine by Suzuki coupling with the conditions described in Scheme 15. 4-(2-bromo-3-fluorophenyl)-N-methylthiazol-2-amine was synthesized using general method C.

### Example 154

compound n^{o}154: (3R)-4-((4-(2-chlorophenyl)thiazol-2-yl)(methyl)amino)-4-oxo-3-((tetrahydrofuran-2-yl)methyl)butanoic acid was synthesized from intermediates 1e1 and 2c using general method E.

### Example 155

compound n^{o}155: (3R)-4-((4-(2-chlorophenyl)thiazol-2-yl)(methyl)amino)-4-oxo-3-((tetrahydrofuran-2-yl)methyl)butanoic acid was synthesized from intermediates 1b and 2u1 using general method E followed by debenzylation with FeCl₃ in DCM.

### Example 156

compound n^{o}156: (R)-3-benzyl-4-((4-(2-chlorophenyl)thiazol-2-yl)(3-hydroxypropyl)amino)-4-oxobutanoic acid was synthesized from intermediates 1b and 2v1 using general method E followed by debenzylation with FeCl₃ in DCM.

### Example 157

compound n^{o}157: (R)-3-benzyl-4-((4-(2-(5-chloro-6-methoxypyridin-3-yl)phenyl)thiazol-2-yl)(methyl)amino)-4-oxobutanoic acid was synthesized from intermediates 1b and 2w1 (4-(2-(5-chloro-6-methoxypyridin-3-yl)phenyl)-N-methylthiazol-2-amine) using general method E. Intermediate 2w1 was synthesized from 5-bromo-3-chloro-2-methoxypyridine and 2l using the methodology described in Scheme 17.

### Example 158

compound n^{o}158: (R)-3-benzyl-4-((4-(2-(6-(benzyloxy)pyridin-3-yl)phenyl)thiazol-2-yl)(methyl)amino)-4-oxobutanoic acid was synthesized from intermediates 1b and 2x1 (4-(2-(6-(benzyloxy)pyridin-3-yl)phenyl)-N-methylthiazol-2-amine) using general method E and preparative HPLC purification. Intermediate 2x1 was synthesized from (6-benzyloxypyridin-3-yl)boronic acid and 2l by Suzuki coupling with the conditions described in Scheme 15.

### Example 159

compound n^{o}159: (R)-3-(cyclopentylmethyl)-4-((4-(2,5-dichlorophenyl)thiazol-2-yl)(methyl)amino)-4-oxobutanoic acid was synthesized from intermediates 1f1 and 2a1 using general method E.

### Example 160

compound n^{o}160: (R)-4-((4-(2-(6-methoxypyridin-3-yl)phenyl)thiazol-2-yl)(methyl)amino)-4-oxo-3-((tetrahydro-2H-pyran-4-yl)methyl)butanoic acid was synthesized from intermediates 1g1 and 2c using general method E.

### Example 161

compound n^{o}161: (R)-3-benzyl-4-((4-(2-chloro-5-(trifluoromethyl)phenyl)thiazol-2-yl)(methyl)amino)-4-oxobutanoic acid was synthesized from intermediates 1b and 2y1 using general method E.

### Example 162

compound n^{o}162: (R)-3-benzyl-4-((4-(2-chloro-5-fluorophenyl)thiazol-2-yl)(methyl)amino)-4-oxobutanoic acid was synthesized from intermediates 1b and 2z1 using general method E.

### Example 163

compound n^{o}163: (R)-3-benzyl-4-((4-(3,5-dichlorophenyl)thiazol-2-yl)(methyl)amino)-4-oxobutanoic acid was synthesized from intermediates 1b and 2a2 using general method E.

### Example 164

compound n^{o}164: (R)-3-benzyl-4-((4-(3-(difluoromethoxy)phenyl)thiazol-2-yl)(methyl)amino)-4-oxobutanoic acid was synthesized from intermediates 1b and 2b2 using general method E.

### Example 165

compound n^{o}165: (R)-4-((4-(2-chlorophenyl)thiazol-2-yl)(methyl)amino)-3-(cyclopentylmethyl)-4-oxobutanoic acid was synthesized from intermediates 1f1 and 2c using general method E.

### Example 166

compound n^{o}166: (R)-3-(cyclopentylmethyl)-4-(cyclopropyl(4-(2,5-dichlorophenyl)thiazol-2-yl)amino)-4-oxobutanoic acid was synthesized from intermediates 1f1 and 2w using general method E.

### Example 167

compound n^{o}167: (R)-4-(cyclopropyl(4-(2,5-dichlorophenyl)thiazol-2-yl)amino)-4-oxo-3-((tetrahydro-2H-pyran-4-yl)methyl)butanoic acid was synthesized from intermediates 1g1 and 2w using general method E.

### Example 168

compound n^{o}168: (R)-4-((4-(2,5-dichlorophenyl)thiazol-2-yl)(methyl)amino)-4-oxo-3-((tetrahydro-2H-pyran-4-yl)methyl)butanoic acid was synthesized from intermediates 1g1 and 2a1 using general method E.

### Example 169

compound n^{o}169: (R)-3-(cyclopentylmethyl)-4-(cyclopropyl(4-(2-(6-methoxypyridin-3-yl)phenyl)thiazol-2-yl)amino)-4-oxobutanoic acid was synthesized from intermediates 1f1 and 2c2 (N-cyclopropyl-4-(2-(6-methoxypyridin-3-yl)phenyl)thiazol-2-amine) using general method E. Intermediate 2c2 was synthesized from (6-methoxypyridin-3-yl)boronic acid and 4-(2-bromo-4-chlorophenyl)-N-cyclopropylthiazol-2-amine by Suzuki coupling with the conditions described_in Scheme 15. 4-(2-bromo-4-chlorophenyl)-N-cyclopropylthiazol-2-amine was synthesized using general method C.

### Example 170

compound n^{o}170: (R)-3-benzyl-4-((2-hydroxyethyl)(4-(2-(6-methoxypyridin-3-yl)phenyl)thiazol-2-yl)amino)-4-oxobutanoic acid was synthesized from intermediates 1b and 2d2 (N-(2-(benzyloxy)ethyl)-4-(2-(6-methoxypyridin-3-yl)phenyl)thiazol-2-amine) using general method E followed by debenzylation with FeCl₃ in DCM. Intermediate 2d2 was synthesized from (6-methoxypyridin-3-yl)boronic acid and N-(2-(benzyloxy)ethyl)-4-(2-bromophenyl)thiazol-2-amine by Suzuki coupling with the conditions described in Scheme 15. N-(2-(benzyloxy)ethyl)-4-(2-bromophenyl)thiazol-2-amine was synthesized using general method C.

### Example 171

compound n^{o}171: (R)-3-(cyclopentylmethyl)-4-(methyl(4-(2-(6-morpholinopyridin-3-yl)phenyl)thiazol-2-yl)amino)-4-oxobutanoic acid was synthesized from intermediates 1f1 and 2d1 using general method E.

### Example 172

compound n^{o}172: (R)-3-(cyclopentylmethyl)-4-((4-(2,5-dichlorophenyl)thiazol-2-yl)(2-hydroxyethyl)amino)-4-oxobutanoic acid was synthesized from intermediates 1f1 and 2d3 using general method E followed by debenzylation with FeCl₃ in DCM.

### Example 173

compound n^{o}173: (R)-4-((4-(2-chlorophenyl)thiazol-2-yl)(methyl)amino)-4-oxo-3-((tetrahydro-2H-pyran-4-yl)methyl)butanoic acid was synthesized from intermediates 1g1 and 2c using general method E.

### Example 174

compound n^{o}174: (R)-3-benzyl-4-((4-(2-chloro-5-(trifluoromethyl)phenyl)thiazol-2-yl)(methyl)amino)-4-oxobutanoic acid was synthesized from intermediates 1b and 2e2 using general method E.

### Example 175

compound n^{o}175: (R)-3-benzyl-4-(methyl(4-(2,3,5-trichlorophenyl)thiazol-2-yl)amino)-4-oxobutanoic acid was synthesized from intermediates 1b and 2f2 using general method E.

### Example 176

compound n^{o}176: (R)-3-benzyl-4-((4-(4-chloro-[1,1′-biphenyl]-3-yl)thiazol-2-yl)(methyl)amino)-4-oxobutanoic acid was synthesized from intermediates 1b and 2g2 (4-(4-chloro-[1,1′-biphenyl]-3-yl)-N-methylthiazol-2-amine) using general method E. Intermediate 2g2 was synthesized from phenylboronic acid and 4-(5-bromo-2-chlorophenyl)-N-methylthiazol-2-amine by Suzuki coupling with the conditions described in Scheme 15. 4-(5-bromo-2-chlorophenyl)-N-methylthiazol-2-amine was synthesized using general method C.

### Example 177

compound n^{o}177: (R)-3-benzyl-4-((4-(2-chloro-5-(6-methoxypyridin-3-yl)phenyl)thiazol-2-yl)(methyl)amino)-4-oxobutanoic acid was synthesized from intermediates 1b and 2h2 (4-(2-chloro-5-(6-methoxypyridin-3-yl)phenyl)-N-methylthiazol-2-amine) using general method E. Intermediate 2h2 was synthesized from (6-methoxypyridin-3-yl)boronic acid and 4-(5-bromo-2-chlorophenyl)-N-methylthiazol-2-amine by Suzuki coupling with the conditions described in Scheme 15. 4-(5-bromo-2-chlorophenyl)-N-methylthiazol-2-amine was synthesized using general method C.

### Example 178

compound n^{o}178: (R)-3-benzyl-4-(cyclopropyl(4-(2-(6-methoxypyridin-3-yl)phenyl)thiazol-2-yl)amino)-4-oxobutanoic acid was synthesized from intermediates 1b and 2c2 using general method E.

### Example 179

compound n^{o}179: (R)-4-(cyclopropyl(4-(2-(6-methoxypyridin-3-yl)phenyl)thiazol-2-yl)amino)-4-oxo-3-((tetrahydro-2H-pyran-4-yl)methyl)butanoic acid was synthesized from intermediates 1g1 and 2c2 using general method E.

### Example 180

compound n^{o}180: (R)-3-benzyl-4-(cyclopropyl(4-(2-(6-morpholinopyridin-3-yl)phenyl)thiazol-2-yl)amino)-4-oxobutanoic acid was synthesized from intermediates 1b and 2i2 (N-cyclopropyl-4-(2-(6-morpholinopyridin-3-yl)phenyl)thiazol-2-amine) using general method E. Intermediate 2i2 was synthesized from 4-(4-(4,4,5,5-tetramethyl-1,3,2-dioxaborolan-2-yl)phenyl)morpholine and 4-(2-bromophenyl)-N-cyclopropylthiazol-2-amine by Suzuki coupling with the conditions described in Scheme 15. 4-(2-bromophenyl)-N-cyclopropylthiazol-2-amine was synthesized using general method C.

### Example 181

compound n^{o}181: (R)-3-(cyclopentylmethyl)-4-(cyclopropyl(4-(2-(6-morpholinopyridin-3-yl)phenyl)thiazol-2-yl)amino)-4-oxobutanoic acid was synthesized from intermediates 1f1 and 2i2 using general method E.

### Example 182

compound n^{o}182: (R)-3-benzyl-4-(methyl(4-(2-(4-methyl-3,4-dihydro-2H-pyrido[3,2-b][1,4]oxazin-7-yl)phenyl)thiazol-2-yl)amino)-4-oxobutanoic acid was synthesized from intermediates 1b and 2j2 (N-methyl-4-(2-(4-methyl-3,4-dihydro-2H-pyrido[3,2-b][1,4]oxazin-7-yl)phenyl)thiazol-2-amine) using general method E and preparative HPLC purification. Intermediate 2j2 was synthesized from commercially available 7-bromo-4-methyl-3,4-dihydro-2H-pyrido[3,2-b][1,4]oxazine and 2l using the methodology described in Scheme 17.

### Example 183

compound n^{o}183: (R)-3-(cyclopentylmethyl)-4-(cyclopropyl(4-(2-(6-(2-oxopyrrolidin-1-yl)pyridin-3-yl)phenyl)thiazol-2-yl)amino)-4-oxobutanoic acid was synthesized from intermediates 1b and 2k2 (1-(5-(2-(2-(cyclopropylamino)thiazol-4-yl)phenyl)pyridin-2-yl)pyrrolidin-2-one) using general method E. Intermediate 2k2 was synthesized from 1-(5-bromopyridin-2-yl)pyrrolidin-2-one and 4-(2-bromo-4-chlorophenyl)-N-cyclopropylthiazol-2-amine using the methodology described in Scheme 17. 1-(5-bromopyridin-2-yl)pyrrolidin-2-one was synthesized by reacting 5-bromopyridin-2-amine with Na₂HPO₄ in CHCl₃, 4-bromobutyryl chloride and NaOMe in MeOH as described in Tetrahedron 1957, 1, 9635. 4-(2-bromo-4-chlorophenyl)-N-cyclopropylthiazol-2-amine was synthesized using general method C.

### Example 184

compound n^{o}184: (R)-4-(cyclopropyl(4-(2-(6-morpholinopyridin-3-yl)phenyl)thiazol-2-yl)amino)-4-oxo-3-((tetrahydro-2H-pyran-4-yl)methyl)butanoic acid was synthesized from intermediates 1g1 and 2i2 using general method E.

### Example 185

compound n^{o}185: (R)-3-benzyl-4-(methyl(4-(2-(trifluoromethoxy)phenyl)thiazol-2-yl)amino)-4-oxobutanoic acid was synthesized from intermediates 1b and 2l2 using general method E.

### Example 186

compound n^{o}186: (R)-4-((4-(2-chloro-5-fluorophenyl)thiazol-2-yl)(cyclopropyl)amino)-3-(cyclopentylmethyl)-4-oxobutanoic acid was synthesized from intermediates 1b and 2m2 using general method E and preparative HPLC purification.

### Example 187

compound n^{o}187: (R)-3-(cyclopentylmethyl)-4-(methyl(4-(2-(6-(2-oxopyrrolidin-1-yl)pyridin-3-yl)phenyl)thiazol-2-yl)amino)-4-oxobutanoic acid was synthesized from intermediates 1f1 and 2q1 using general method E.

### Example 188

compound n^{o}188: (R)-3-benzyl-4-(cyclopropyl(4-(3-(difluoromethoxy)phenyl)thiazol-2-yl)amino)-4-oxobutanoic acid was synthesized from intermediates 1b and 2n2 using general method E and preparative HPLC purification.

### Example 189

compound n^{o}189: (R)-3-benzyl-4-((4-(2-chloro-5-fluorophenyl)thiazol-2-yl)(cyclopropyl)amino)-4-oxobutanoic acid was synthesized from intermediates 1b and 2m2 using general method E and preparative HPLC purification.

### Example 190

compound n^{o}190: (R)-4-((4-(2-chloro-5-fluorophenyl)thiazol-2-yl)(cyclopropyl)amino)-4-oxo-3-((tetrahydro-2H-pyran-4-yl)methyl)butanoic acid was synthesized from intermediates 1g1 and 2m2 using general method E and preparative HPLC purification.

### Example 191

compound n^{o}191: (R)-3-benzyl-4-((4-(2-chloro-5-(trifluoromethyl)phenyl)thiazol-2-yl)(cyclopropyl)amino)-4-oxobutanoic acid was synthesized from intermediates 1b and 2o2 using general method E and preparative HPLC purification.

### Example 192

compound n^{o}192: (R)-3-benzyl-4-((4-(2-(difluoromethoxy)phenyl)thiazol-2-yl)(methyl)amino)-4-oxobutanoic acid was synthesized from intermediates 1b and 2c4 using general method E

### Example 193

compound n^{o}193: (R)-4-((4-(2-chloro-5-(trifluoromethyl)phenyl)thiazol-2-yl)(cyclopropyl)amino)-4-oxo-3-((tetrahydro-2H-pyran-4-yl)methyl)butanoic acid was synthesized from intermediates 1g1 and 2o2 using general method E.

### Example 194

compound n^{o}194: (R)-3-(cyclopentylmethyl)-4-(cyclopropyl(4-(2-(4-methyl-3,4-dihydro-2H-pyrido[3,2-b][1,4]oxazin-7-yl)phenyl)thiazol-2-yl)amino)-4-oxobutanoic acid was synthesized from intermediates 1f1 and 2p2 (N-cyclopropyl-4-(2-(4-methyl-3,4-dihydro-2H-pyrido[3,2-b][1,4]oxazin-7-yl)phenyl)thiazol-2-amine) using general method E. Intermediate 2p2 was synthesized from commercially available 7-bromo-4-methyl-3,4-dihydro-2H-pyrido[3,2-b][1,4]oxazine and 2l using the methodology described in Scheme 17. 4-(2-bromo-4-chlorophenyl)-N-cyclopropylthiazol-2-amine was synthesized using general method C.

### Example 195

compound n^{o}195: (3R,4S)-3-((4-(2-chlorophenyl)thiazol-2-yl)(methyl)carbamoyl)-4-phenylpentanoic acid was synthesized from intermediates 1h1 and 2c using general method E.

### Example 196

compound n^{o}196: (R)-2-(2-benzyl-3-carboxypropanamido)-5-(2-chlorophenyl)pyridine 1-oxide was synthesized from intermediates 1b and 2q2 (2-amino-5-(2-chlorophenyl)pyridine) using general method E followed by oxidation with MCPBA. 2q2 was made from commercially available 5-bromopyridin-2-amine and (2-chlorophenyl)boronic acid using Suzuki coupling.

### Example 197

compound n^{o}197: (R)-3-benzyl-4-((5-(2-chlorophenyl)pyrazin-2-yl)amino)-4-oxobutanoic acid was synthesized from intermediates 1b and 2r2 (5-(2-chlorophenyl)pyrazin-2-amine) using general method E. 2r2 was made from commercially available 5-bromopyrazin-2-amine and (2-chlorophenyl)boronic acid using Suzuki coupling.

### Example 198

compound n^{o}198: 4-((4-(2-chlorophenyl)thiazol-2-yl)(methyl)amino)-3-(morpholinomethyl)-4-oxobutanoic acid was synthesized as described in Scheme 18.

### Example 199

compound n^{o}199: (R)-3-benzyl-4-((4-(2-chlorophenyl)thiazol-2-yl)(2-methoxyethyl)amino)-4-oxobutanoic acid was synthesized from intermediates 1b and 2s2 using general method E.

### Example 200

compound n^{o}200: (R)-4-((4-(2-chlorophenyl)thiazol-2-yl)(methyl)amino)-3-(cyclopentylamino)-4-oxobutanoic acid was synthesized from intermediates 1j1 ((R)-4-(tert-butoxy)-2-(cyclopentylamino)-4-oxobutanoic acid) and 2c using general method E. 1j1 was made from (R)-2-amino-4-(tert-butoxy)-4-oxobutanoic acid and cyclopentanone by reductive amination using sodium cyanoborohydride in methanol.

### Example 201

compound n^{o}201: (R)-3-benzyl-4-((2-(benzyloxy)ethyl)(4-(2-chlorophenyl)thiazol-2-yl)amino)-4-oxobutanoic acid was synthesized from intermediates 1b and 2u1 using general method E.

### Example 202

compound n^{o}202: (R)-3-benzyl-4-((4-(5-methylfuran-2-yl)thiazol-2-yl)amino)-4-oxobutanoic acid was synthesized from intermediates 1b and commercially available 2u2 (4-(5-methylfuran-2-yl)thiazol-2-amine) using general method E.

### Example 203

compound n^{o}203: (R)-3-benzyl-4-oxo-4-((3-(3-(trifluoromethyl)phenyl)-1H-pyrazol-5-yl)amino)butanoic acid was synthesized from intermediates 1b and commercially available 2v2 (3-(3-(trifluoromethyl)phenyl)-1H-pyrazol-5-amine) using general method E.

### Example 204

compound n^{o}204: (R)-3-benzyl-4-((4-(5-chloro-2-methoxyphenyl)thiazol-2-yl)amino)-4-oxobutanoic acid was synthesized from intermediates 1b and commercially available 2w2 (4-(5-chloro-2-methoxyphenyl)thiazol-2-amine) using general method E.

### Example 205

compound n^{o}205: 4-((4-(2-chlorophenyl)thiazol-2-yl)(methyl)amino)-3-(4-hydroxybenzyl)-4-oxobutanoic acid was synthesized from from intermediates 1k1 (4-(tert-butoxy)-2-(4-(methoxymethoxy)benzyl)-4-oxobutanoic acid) and 2c using general method E, the MOM group was deprotected with TFA in DCM. 1k1 was synthesized from 4-(methoxymethoxy)benzaldehyde using the HWE methodology (Scheme 13).

### Example 206

compound n^{o}206: (R)-3-benzyl-4-((4-(4′-cyano-[1,1′-biphenyl]-2-yl)thiazol-2-yl)(methyl)amino)-4-oxobutanoic acid was synthesized from intermediates 1b and 2x2 (2′-(2-(methylamino)thiazol-4-yl)-[1,1′-biphenyl]-4-carbonitrile) using general method E. Intermediate 2x2 was synthesized from (4-cyanophenyl)boronic acid and 2l by Suzuki coupling with the conditions described in Scheme 15.

### Example 207

compound n^{o}207: (3R)-3-benzyl-4-((3-carbamoyl-4-(2,4-dichlorophenyl)-5-methylthiophen-2-yl)amino)-4-oxobutanoic acid was synthesized from intermediates 1b and commercially available 2y2 (2-amino-4-(2,4-dichlorophenyl)-5-methylthiophene-3-carbonitrile) using general method E.

### Example 208

compound n^{o}208: (R)-3-benzyl-4-((4-(3′-methoxy-[1,1′-biphenyl]-2-yl)thiazol-2-yl)(methyl)amino)-4-oxobutanoic acid was synthesized from intermediates 1b and 2z2 (4-(3′-methoxy-[1,1′-biphenyl]-2-yl)-N-methylthiazol-2-amine) using general method E. Intermediate 2z2 was synthesized from (3-methoxyphenyl)boronic acid and 2l by Suzuki coupling with the conditions described in Scheme 15.

### Example 209

compound n^{o}209: 4-((4-(2-chlorophenyl)thiazol-2-yl)(methyl)amino)-3-((2-methylthiazol-4-yl)methyl)-4-oxobutanoic acid was synthesized from intermediates 1l1 (4-(tert-butoxy)-2-((2-methylthiazol-4-yl)methyl)-4-oxobutanoic acid) and 2c using general method E. 1l1 was synthesized from 2-methylthiazole-5-carbaldehyde using the HWE methodology (Scheme 13).

### Example 210

compound n^{o}210: 4-((4-(2-chlorophenyl)thiazol-2-yl)(methyl)amino)-3-((5-methylisoxazol-3-yl)methyl)-4-oxobutanoic acid was synthesized from intermediates 1m1 (4-(tert-butoxy)-2-((5-methylisoxazol-3-yl)methyl)-4-oxobutanoic acid) and 2c using general method E. 1m1 was synthesized from 5-methylisoxazole-3-carbaldehyde using the HWE methodology (Scheme 13).

### Example 211

compound n^{o}211: (R)-3-benzyl-4-((4-(2′-chloro-[1,1′-biphenyl]-2-yl)thiazol-2-yl)(methyl)amino)-4-oxobutanoic acid was synthesized from intermediates 1b and 2a3 (4-(2′-chloro-[1,1′-biphenyl]-2-yl)-N-methylthiazol-2-amine) using general method E and preparative HPLC purification. Intermediate 2a3 was synthesized from (2-chlorophenyl)boronic acid and 2l by Suzuki coupling with the conditions described in Scheme 15.

### Example 212

compound n^{o}212: (R)-3-benzyl-4-((4-(2-(2-methoxypyrimidin-5-yl)phenyl)thiazol-2-yl)(methyl)amino)-4-oxobutanoic acid was synthesized from intermediates 1b and 2b3 (4-(2-(2-methoxypyrimidin-5-yl)phenyl)-N-methylthiazol-2-amine) using general method E. Intermediate 2b3 was synthesized from 5-bromo-2-methoxypyrimidine and 2l using the methodology described in Scheme 17.

### Example 213

compound n^{o}213: (R)-3-benzyl-4-((4-(2,5-difluorophenyl)thiazol-2-yl)amino)-4-oxobutanoic acid was synthesized from intermediates 1b and commercially available 2c3 (4-(2,5-difluorophenyl)thiazol-2-amine) using general method E.

### Example 214

compound n^{o}214: 4-((4-(2-chlorophenyl)thiazol-2-yl)(methyl)amino)-3-(oxazol-4-ylmethyl)-4-oxobutanoic acid was synthesized from intermediates 1n1 (4-(tert-butoxy)-2-(oxazol-4-ylmethyl)-4-oxobutanoic acid) and 2c using general method E. 1n1 was synthesized from oxazole-4-carbaldehyde using the HWE methodology (Scheme 13).

### Example 215

compound n^{o}215: (3R)-4-((4-(2-chlorophenyl)thiazol-2-yl)(methyl)amino)-4-oxo-3-((tetrahydrofuran-3-yl)methyl)butanoic acid was synthesized from intermediates 1o1 and 2c using general method E.

### Example 216

compound n^{o}216: (R)-3-benzyl-4-(methyl(4-(2-(8-methyl-7-oxo-5,6,7,8-tetrahydro-1,8-naphthyridin-3-yl)phenyl)thiazol-2-yl)amino)-4-oxobutanoic acid was synthesized from intermediates 1b and 2e3 (1-methyl-6-(2-(2-(methylamino)thiazol-4-yl)phenyl)-3,4-dihydro-1,8-naphthyridin-2(1H)-one) using general method E.

Intermediate 2e3 was synthesized from 6-bromo-1-methyl-3,4-dihydro-1,8-naphthyridin-2(1H)-one (which was obtained by treatment of 6-bromo-3,4-dihydro-1,8-naphthyridin-2(1H)-one with NaH in DMF and MeI) and 2l using the methodology described in Scheme 17. Intermediate 1b was synthesized using the HWE methodology (Scheme 13):

38.125 mmol of (E)-2-benzylidene-4-(tert-butoxy)-4-oxobutanoic acid, 75 mL of methanol and 38.125 mmol of DCA were successively introduced into a Schlenck tube under Ar. The solution was degassed using three argon/vacuum cycles, and subsequently transferred into the reaction vessel under inert atmosphere. To this degassed solution was added, under argon flow, 0.121 mmol of the RuCl₂-[(S)-BINAP] catalyst. The reaction vessel was then transferred into a Parr autoclave, under Ar flow. The Parr vessel was purged 3 times with H₂ with a pressure up to 20 sbars; the pressure was then adjusted to 10 bars. The Parr autoclave was put into an oil bath at 55° C. The reaction mixture was stirred at this temperature for 3 days. The reaction mixture was allowed to cool to RT and the hydrogen pressure was released carefully and the Parr vessel opened. The crude reaction mixture was concentrated to dryness using rotary evaporator to afford 16.74 g of a colored solid. An aliquot of the solid was diluted with water and acidified with HCl 6N to pH 1; then, the solution was extracted with EtOAc. The organic layer was dried over magnesium sulfate, concentrated using rotary evaporator to yield the desired intermediate (ee=82.6%, determined by chiral HPLC). Solid (16.74 g) was recrystallized from an ACN/water mixture. Recrystallized product was diluted with water and acidified with 6N HCl to pH 1, the solution was extracted with EtOAc. The organic layer was dried over magnesium sulfate, concentrated at rotavap to yield the desired intermediate 1b (ee=96.6%, determined by chiral HPLC).

### Example 217

compound n^{o}217: (R)-3-benzyl-4-(methyl(4-(2-(1-methyl-1H-pyrrolo[2,3-b]pyridin-5-yl)phenyl)thiazol-2-yl)amino)-4-oxobutanoic acid was synthesized from intermediates 1b and 2f3 (N-methyl-4-(2-(1-methyl-1H-pyrrolo[2,3-b]pyridin-5-yl)phenyl)thiazol-2-amine) using general method E. Intermediate 2f3 was synthesized from 5-bromo-1-methyl-1-H-pyrrolo[2,3-b]pyridine (which was obtained by treatment of 5-bromo-1H-pyrrolo[2,3-b]pyridine with NaH in DMF and MeI) and 2l using the methodology described in Scheme 17.

### Example 218

compound n^{o}218: (R)-3-(cyclopentylmethyl)-4-(cyclopropyl(4-(2-(6-(dimethylamino)pyridin-3-yl)phenyl)thiazol-2-yl)amino)-4-oxobutanoic acid was synthesized from intermediates 1f1 and 2g3 (N-cyclopropyl-4-(2-(6-(dimethylamino)pyridin-3-yl)phenyl)thiazol-2-amine) using general method E and preparative HPLC purification. 2g3 was synthesized from 4-(2-bromophenyl)-N-cyclopropylthiazol-2-amine and 6-(dimethylamino)pyridin-3-ylboronic acid by Suzuki coupling with the conditions described in Scheme 15. 4-(2-bromophenyl)-N-cyclopropylthiazol-2-amine was synthesized using general method C.

### Example 219

compound n^{o}219: (R)-4-((4-(2-(5-chloro-6-methoxypyridin-3-yl)phenyl)thiazol-2-yl)(cyclopropyl)amino)-3-(cyclopentylmethyl)-4-oxobutanoic acid was synthesized from intermediates 1f1 and 2h3 (4-(2-(5-chloro-6-methoxypyridin-3-yl)phenyl)-N-cyclopropylthiazol-2-amine) using general method E. 2g3 was synthesized from 5-bromo-3-chloro-2-methoxypyridine and 4-(2-bromophenyl)-N-cyclopropylthiazol-2-amine using the methodology described in Scheme 17. 4-(2-bromophenyl)-N-cyclopropylthiazol-2-amine was synthesized using general method C.

### Example 220

compound n^{o}220: (R)-3-(cyclopentylmethyl)-4-(cyclopropyl(4-(2-(5-fluoro-6-methoxypyridin-3-yl)phenyl)thiazol-2-yl)amino)-4-oxobutanoic acid was synthesized from intermediates 1f1 and 2i3 (N-cyclopropyl-4-(2-(5-fluoro-6-methoxypyridin-3-yl)phenyl)thiazol-2-amine) using general method E. 2i3 was synthesized from 5-bromo-3-fluoro-2-methoxypyridine and 4-(2-bromophenyl)-N-cyclopropylthiazol-2-amine using the methodology described in Scheme 17. 4-(2-bromophenyl)-N-cyclopropylthiazol-2-amine was synthesized using general method C.

### Example 221

compound n^{o}221: (R)-3-benzyl-4-((4-(2-chloro-5-(difluoromethoxy)phenyl)thiazol-2-yl)(methyl)amino)-4-oxobutanoic acid was synthesized from intermediates 1b and 2j3 using general method E.

### Example 222

compound n^{o}222: (R)-3-benzyl-4-((4-(5-chloro-2-(5-chloro-6-methoxypyridin-3-yl)phenyl)thiazol-2-yl)(methyl)amino)-4-oxobutanoic acid was synthesized from intermediates 1b and 2k3 (4-(5-chloro-2-(5-chloro-6-methoxypyridin-3-yl)phenyl)-N-methylthiazol-2-amine) using general method E. 2k3 was synthesized from 5-bromo-3-chloro-2-methoxypyridine and 4-(2-bromo-5-chlorophenyl)-N-methylthiazol-2-amine using the methodology described in Scheme 17. 4-(2-bromo-5-chlorophenyl)-N-methylthiazol-2-amine was synthesized using general method C.

### Example 223

compound n^{o}223: (R)-4-((4-(5-chloro-2-(5-chloro-6-methoxypyridin-3-yl)phenyl)thiazol-2-yl)(cyclopropyl)amino)-3-(cyclopentylmethyl)-4-oxobutanoic acid was synthesized from intermediates 1f1 and 2l3 (4-(5-chloro-2-(5-chloro-6-methoxypyridin-3-yl)phenyl)-N-cyclopropylthiazol-2-amine) using general method E. 2l3 was synthesized from 5-bromo-3-chloro-2-methoxypyridine and 4-(2-bromo-5-chlorophenyl)-N-cyclopropylthiazol-2-amine using the methodology described in Scheme 17. 4-(2-bromo-5-chlorophenyl)-N-cyclopropylthiazol-2-amine was synthesized using general method C.

### Example 224

compound n^{o}224: (R)-4-((4-(5-chloro-2-(5-fluoro-6-methoxypyridin-3-yl)phenyl)thiazol-2-yl)(cyclopropyl)amino)-3-(cyclopentylmethyl)-4-oxobutanoic acid was synthesized from intermediates 1f1 and 2m3 (4-(5-chloro-2-(5-fluoro-6-methoxypyridin-3-yl)phenyl)-N-cyclopropylthiazol-2-amine) using general method E. 2m3 was synthesized from 5-bromo-3-fluoro-2-methoxypyridine and 4-(2-bromo-5-chlorophenyl)-N-cyclopropylthiazol-2-amine using the methodology described in Scheme 17. 4-(2-bromo-5-chlorophenyl)-N-cyclopropylthiazol-2-amine was synthesized using general method C.

### Example 225

compound n^{o}225: (S)-3-benzyl-4-((4-(2-chlorophenyl)thiazol-2-yl)amino)-4-oxobutanoic acid was synthesized from intermediates 1p1 ((S)-2-benzyl-4-(tert-butoxy)-4-oxobutanoic acid) and 2a using general method E. 1p1 was synthesized from (S)-3-benzyl-4-methoxy-4-oxobutanoic acid using the chemistry described in steps 5 and 6 of general method B.

### Example 227

compound n^{o}227: (R)-3-benzyl-4-((4-benzylthiazol-2-yl)amino)-4-oxobutanoic acid was synthesized from intermediates 1b and commercially available 4-benzylthiazol-2-amine using general method E.

### Example 229

compound n^{o}229: (R)-3-benzyl-4-oxo-4-((5-phenyl-4H-1,2,4-triazol-3-yl)amino)butanoic acid was synthesized from intermediates 1b and commercially available 5-phenyl-4H-1,2,4-triazol-3-amine using general method E.

### Example 230

compound n^{o}230: 3-([1,1′-biphenyl]-4-ylmethyl)-4-((4-(2-chlorophenyl)thiazol-2-yl)(methyl)amino)-4-oxobutanoic acid was synthesized from intermediates 1q1 (2-([1,1′-biphenyl]-4-ylmethyl)-4-(tert-butoxy)-4-oxobutanoic acid) and 2c using general method E. 1q1 was synthesized from [1,1′-biphenyl]-4-carbaldehyde using the HWE methodology described in Scheme 13.

### Example 231

compound n^{o}231: (R)-3-benzyl-4-((4-(1-methyl-1H-pyrazol-4-yl)thiazol-2-yl)amino)-4-oxobutanoic acid was synthesized from intermediates 1b and commercially available 4-(1-methyl-1H-pyrazol-4-yl)thiazol-2-amine using general method E.

### Example 232

compound n^{o}232: ((R)-3-benzyl-4-((4-(4-methyl-1,2,5-oxadiazol-3-yl)thiazol-2-yl)amino)-4-oxobutanoic acid was synthesized from intermediates 1b and commercially available 4-(4-methyl-1,2,5-oxadiazol-3-yl)thiazol-2-amine using general method E.

### Example 233

compound n^{o}233: (R)-3-benzyl-4-(methyl(4-(2-(1-methyl-1H-pyrazol-4-yl)phenyl)thiazol-2-yl)amino)-4-oxobutanoic acid was synthesized from intermediates 1b and 2n3 (N-methyl-4-(2-(1-methyl-1H-pyrazol-4-yl)phenyl)thiazol-2-amine) using general method E. 2n3 was synthesized from commercially available 1-methyl-4-(4,4,5,5-tetramethyl-1,3,2-dioxaborolan-2-yl)-1H-pyrazole and 2l using the methodology described in Scheme 15.

### Example 234

compound n^{o}234: (3R)-3-benzyl-4-((4-(2-(3,5-dimethylisoxazol-4-yl)phenyl)thiazol-2-yl)(methyl)amino)-4-oxobutanoic acid was synthesized from intermediates 1b and 2o3 (4-(2-(3,5-dimethylisoxazol-4-yl)phenyl)-N-methylthiazol-2-amine) using general method E. 2o3 was synthesized from commercially available 3,5-dimethyl-4-(4,4,5,5-tetramethyl-1,3,2-dioxaborolan-2-yl)isoxazole and 2l using the methodology described in Scheme 15.

### Example 235

compound n^{o}235: (R)-3-benzyl-4-((4-((2-chlorophenyl)carbamoyl)thiazol-2-yl)amino)-4-oxobutanoic acid was synthesized from intermediates 1b and 2p3 using general method E and preparative HPLC purification. 2p3 was synthesized as described in Scheme 21.

### Example 236

compound n^{o}236: (R)-3-benzyl-4-((6-(2-chlorophenyl)pyridazin-3-yl)amino)-4-oxobutanoic acid was synthesized from intermediates 1b and 2q3 (6-(2-chlorophenyl)pyridazin-3-amine) using general method E. 2q3 was synthesized from 6-bromopyridazin-3-amine and 2-chlorophenylboronic acid by Suzuki coupling with the conditions described in Scheme 8.

### Example 237

compound n^{o}237: (R)-3-benzyl-4-(methyl(4-(2-(2-oxopyrrolidin-1-yl)phenyl)thiazol-2-yl)amino)-4-oxobutanoic acid was synthesized from intermediates 1b and 2r3 (1-(2-(2-(methylamino)thiazol-4-yl)phenyl)pyrrolidin-2-one) using general method E. 2r3 was synthesized as described in Scheme 16.

### Example 238

compound n^{o}238: (S)-2-((1-((4-(2-chlorophenyl)thiazol-2-yl)(methyl)amino)-1-oxo-3-phenylpropan-2-yl)oxy)acetic acid was synthesized as described in Scheme 22.

### Example 239

compound n^{o}239: (R)-3-benzyl-4-((1-methyl-5-phenyl-1H-imidazol-2-yl)amino)-4-oxobutanoic acid was synthesized from intermediates 1b and commercially available 1-methyl-5-phenyl-1H-imidazol-2-amine using general method E.

### Example 240

compound n^{o}240: (R)-3-benzyl-4-((4-(2-(1-(2-methoxyethyl)-6-oxo-1,6-dihydropyridin-3-yl)phenyl)thiazol-2-yl)(methyl)amino)-4-oxobutanoic acid was synthesized from intermediates 1b and 2s3 (1-(2-methoxyethyl)-5-(2-(2-(methylamino)thiazol-4-yl)phenyl)pyridin-2(1H)-one) using general method E. 2s3 was synthesized from 5-bromo-1-(2-methoxyethyl)pyridin-2(1H)-one and 2l using the methodology described in Scheme 17.

### Example 241

compound n^{o}241: (R)-3-benzyl-4-(methyl(4-(2-(1-methyl-6-oxo-1,6-dihydropyridin-3-yl)phenyl)thiazol-2-yl)amino)-4-oxobutanoic acid was synthesized from intermediates 1b and 2t3 (1-methyl-5-(2-(2-(methylamino)thiazol-4-yl)phenyl)pyridin-2(1H)-one) using general method E. 2t3 was synthesized from 5-bromo-1-methylpyridin-2(1H)-one and 2l using the methodology described in Scheme 17.

### Example 242

compound n^{o}242: 4-((4-(2-chlorophenyl)thiazol-2-yl)(methyl)amino)-3-((2,5-dimethyloxazol-4-yl)methyl)-4-oxobutanoic acid was synthesized from intermediates 1r1 (tert-butyl 4-amino-3-((2,5-dimethyloxazol-4-yl)methyl)-4-oxobutanoate) and 2c using general method E. 1r1 was synthesized from 2,5-dimethyloxazole-4-carbaldehyde using the HWE methodology described in Scheme 13.

### Example 243

compound n^{o}243: 4-((4-(2-chlorophenyl)thiazol-2-yl)(methyl)amino)-3-((1-methyl-1H-pyrazol-5-yl)methyl)-4-oxobutanoic acid was synthesized from intermediates 1s1 (4-(tert-butoxy)-2-((1-methyl-1H-pyrazol-5-yl)methyl)-4-oxobutanoic acid) and 2c using general method E. 1s1 was synthesized from 1-methyl-1H-pyrazole-5-carbaldehyde using the HWE methodology described in Scheme 13.

### Example 244

compound n^{o}244: (R)-3-benzyl-4-((4-(2-(6-hydroxypyridin-3-yl)phenyl)thiazol-2-yl)(methyl)amino)-4-oxobutanoic acid was synthesized by debenzylation of compound n^{o}158 with FeCl₃ in DCM and preparative HPLC purification.

### Example 245

compound n^{o}245: (R)-3-benzyl-4-((4-(2-chlorophenyl)thiazol-2-yl)((S)-2-hydroxypropyl)amino)-4-oxobutanoic acid was synthesized from intermediates 1b and 2v3 using general method E and preparative HPLC purification.

### Example 246

compound n^{o}246: (R)-3-benzyl-4-((4-(2-chlorophenyl)thiazol-2-yl)((R)-2-hydroxypropyl)amino)-4-oxobutanoic acid was synthesized from intermediates 1b and 2w3 using general method E and preparative HPLC purification.

### Example 247

compound n^{o}247: (R)-3-(cyclohexylmethyl)-4-(cyclopropyl(4-(2-(6-methoxypyridin-3-yl)phenyl)thiazol-2-yl)amino)-4-oxobutanoic acid was synthesized from intermediates 1w and 2c2 using general method E and preparative HPLC purification.

### Example 248

compound n^{o}248: (R)-3-benzyl-4-((4-(5-fluoro-2-(6-methoxypyridin-3-yl)phenyl)thiazol-2-yl)(methyl)amino)-4-oxobutanoic acid was synthesized from intermediates 1b and 2u3 (4-(5-fluoro-2-(6-methoxypyridin-3-yl)phenyl)-N-methylthiazol-2-amine) using general method E. Intermediate 2u3 was synthesized from (6-methoxypyridin-3-yl)boronic acid and 4-(2-bromo-5-fluorophenyl)-N-methylthiazol-2-amine by Suzuki coupling with the conditions described in Scheme 15. 4-(2-bromo-5-fluorophenyl)-N-methylthiazol-2-amine was synthesized using general method C.

### Example 250

compound n^{o}250: (R)-3-benzyl-4-((4-(4,5-difluoro-2-(6-methoxypyridin-3-yl)phenyl)thiazol-2-yl)(methyl)amino)-4-oxobutanoic acid was synthesized from intermediates 1b and 2x3 (4-(4,5-difluoro-2-(6-methoxypyridin-3-yl)phenyl)-N-methylthiazol-2-amine) using general method E. Intermediate 2x3 was synthesized from (6-methoxypyridin-3-yl)boronic acid and 4-(2-bromo-4,5-difluorophenyl)-N-methylthiazol-2-amine by Suzuki coupling with the conditions described in Scheme 15. 4-(2-bromo-4,5-difluorophenyl)-N-methylthiazol-2-amine was synthesized using general method C.

### Example 251

compound n^{o}251: (R)-4-((4-(2,5-dichlorophenyl)thiazol-2-yl)(methyl)amino)-3-(furan-2-ylmethyl)-4-oxobutanoic acid was synthesized from intermediates 1t1 and 2a1 using general method E.

### Example 252

compound n^{o}252: (R)-4-((4-(2-chloro-5-fluorophenyl)thiazol-2-yl)(methyl)amino)-3-(furan-2-ylmethyl)-4-oxobutanoic acid was synthesized from intermediates 1t1 and 2z1 using general method E.

### Example 253

compound n^{o}253: (R)-3-(furan-2-ylmethyl)-4-((4-(2-(6-methoxypyridin-3-yl)phenyl)thiazol-2-yl)(methyl)amino)-4-oxobutanoic acid was synthesized from intermediates 1t1 and 2m using general method E.

### Example 254

compound n^{o}254: (S)-4-((4-(2-chlorophenyl)thiazol-2-yl)(methyl)amino)-4-oxo-3-(thiophen-2-ylmethyl)butanoic acid was synthesized from intermediates 1u1 and 2c using general method E.

### Example 255

compound n^{o}255: (R)-4-((4-(5-chloro-2-(6-methoxypyridin-3-yl)phenyl)thiazol-2-yl)(cyclopropyl)amino)-3-(cyclopentylmethyl)-4-oxobutanoic acid was synthesized from intermediates 1b and 2y3 (4-(5-chloro-2-(6-methoxypyridin-3-yl)phenyl)-N-cyclopropylthiazol-2-amine) using general method E. Intermediate 2y3 was synthesized from (6-methoxypyridin-3-yl)boronic acid and 4-(2-bromo-5-chlorophenyl)-N-cyclopropylthiazol-2-amine by Suzuki coupling with the conditions described in Scheme 15. 4-(2-bromo-5-chlorophenyl)-N-cyclopropylthiazol-2-amine was synthesized using general method C.

### Example 256

compound n^{o}256: (R)-3-benzyl-4-(cyclopropyl(4-(2-(6-(2-oxopyrrolidin-1-yl)pyridin-3-yl)phenyl)thiazol-2-yl)amino)-4-oxobutanoic acid was synthesized from intermediates 1b and 2k2 using general method E.

### Example 257

compound n^{o}257: (R)-3-benzyl-4-((4-(2,3-dichlorophenyl)thiazol-2-yl)(methyl)amino)-4-oxobutanoic acid was synthesized from intermediates 1b and 2z3 using general method E.

### Example 258

compound n^{o}258: (R)-3-benzyl-4-(methyl(4-(3-(trifluoromethoxy)phenyl)thiazol-2-yl)amino)-4-oxobutanoic acid was synthesized from intermediates 1b and 2a4 using general method E.

### Example 259

compound n^{o}259: (R)-4-(cyclopropyl(4-(3-(difluoromethoxy)phenyl)thiazol-2-yl)amino)-4-oxo-3-((tetrahydro-2H-pyran-4-yl)methyl)butanoic acid was synthesized from intermediates 1g1 and 2n2 using general method E.

### Example 260

compound n^{o}260: (R)-4-((4-(2-chlorophenyl)thiazol-2-yl)(methyl)amino)-3-(furan-2-ylmethyl)-4-oxobutanoic acid was synthesized from intermediates 1t1 and 2c using general method E.

### Example 261

compound n^{o}261: (R)-4-(methyl(4-(3-(trifluoromethoxy)phenyl)thiazol-2-yl)amino)-4-oxo-3-((tetrahydro-2H-pyran-4-yl)methyl)butanoic acid was synthesized from intermediates 1g1 and 2a4 using general method E.

### Example 262

compound n^{o}262: (R)-3-benzyl-4-(cyclopropyl(4-(3-(trifluoromethoxy)phenyl)thiazol-2-yl)amino)-4-oxobutanoic acid was synthesized from intermediates 1b and 2b4 using general method E.

### Example 263

compound n^{o}263: (R)-4-(cyclopropyl(4-(3-(trifluoromethoxy)phenyl)thiazol-2-yl)amino)-4-oxo-3-((tetrahydro-2H-pyran-4-yl)methyl)butanoic acid was synthesized from intermediates 1g1 and 2b4 using general method E.

### BIOLOGY EXAMPLES

### BRIEF DESCRIPTION OF THE DRAWINGS

FIG. 1 represents the effect of compound 9 on glucose-uptake measured in 3T3-L1 adipocyte cells in response to 10 nM of insulin.

FIG. 2 represents the effect of compound 9 on glucose-uptake measured in adipocytes isolated from High-fat diet fed mice

FIG. 3 represents the effect of compound 9 on isoprenaline-induced lipolysis in adipocytes from high-fat diet fed mice.

FIG. 4 represents the inhibition of in-vivo lipolysis following the injection of compound 2 in mice.

FIG. 5 represents the inhibition of in-vivo lipolysis following the injection of compound 9 in mice.

FIG. 6 represents the effect of compound 89 on isoprenaline-induced lipolysis in adipocytes isolated from normal rats.

FIG. 7 represents the effect of compounds 14, 89, 126, 139, 142, 155, 169 and 183 on isoprenaline-induced lipolysis in adipocytes isolated from normal rats.

FIG. 8 represents the inhibition of in-vivo lipolysis following the injection of compound 14, 169 or 183 in mice.

FIG. 9 represents the effect of compound 169 on the GLP-1 release from NCI-H716 cells.

### MEMBRANE BINDING ASSAY: GTPγS BINDING ASSAY

The following assay can be used for determination of GPR43 activation. When a GPCR is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyses the GTP to GDP, at which point the receptor normally is deactivated. Activated receptors continue to exchange GDP for GTP. The non-hydrolysable GTP analog, [³⁵S]GTPγS, was used to demonstrate enhance binding of [³⁵S]GTPγS to membranes expressing receptors. The assay uses the ability of GPCR to stimulate [³⁵S]GTPγS binding to membranes expressing the relevant receptors. The assay can, therefore, be used in the direct identification method to screen candidate compounds to endogenous or not endogenous GPCR.

### Preparation of Membrane Extracts:

Membrane extracts were prepared from cells expressing the human GPR43 receptor (hGPR43) as follows: the medium was aspirated and the cells were scraped from the plates in Ca⁺⁺ and Mg⁺⁺-free Phosphate-buffered saline (PBS). The cells were then centrifuged for 3 min at 1500 g and the pellets were resuspended in buffer A (15 mM Tris-HCl pH 7.5, 2 mM MgCl₂, 0.3 mM EDTA, 1 mM EGTA) and homogenized in a glass homogenizer. The crude membrane fraction was collected by two consecutive centrifugation steps at 40.000×g for 25 min separated by a washing step in buffer A. The final pellet was resuspended in 500 μl of buffer B (75 mM Tris-HCl pH 7.5, 12.5 mM MgCl₂, 0.3 mM EDTA, 1 mM EGTA, 250 mM sucrose) and flash frozen in liquid nitrogen. Protein content was assayed by the Folin method.

### GTPγS Assay (SPA Method):

The assay was performed in the presence of SCFA, and was used to determine the activity of the compounds of the invention.

The [³⁵S]GTPγS assay was incubated in 20 mM HEPES pH7.4, 100 mM NaCl, 10 μg/ml saponin, 30 mM of MgCl₂, 10 μM of GDP, 5 μg membrane-expressing hGPR43, 250 μg of wheatgerm agglutinin beads (Amersham, ref: RPNQ001), a range concentration of compounds (from 30 μM to 1 nM) in a final volume of 100 μl for 30 min at room temperature. The SCFA propionate was used at 1 mM final concentration as positive control. The plates were then centrifuged for 10 minutes at 2000 rpm, incubated for 2 hours at room temperature and counted for 1 min in a scintillation counter (TopCount, PerkinElmer). The results of the tested compounds are reported as the concentration of the compound required to reach 50% (EC₅₀) of the maximum level of the activation induced by these compounds.

When tested in the assay described above and by way of illustration the compounds in Table 3 activate GPR43 receptor with an EC₅₀ ranging from 13 nM to 2910 nM.

TABLE 3
Compounds EC₅₀ values in GTPγ³⁵S assay.

| Compound n° | EC₅₀ (nM) |
|---|---|
| 1 | 109 |
| 2 | 273 |
| 3 | 653 |
| 4 | 759 |
| 5 | 292 |
| 8 | 563 |
| 9 | 60 |
| 10 | 405 |
| 11 | 680 |
| 12 | 424 |
| 13 | 875 |
| 14 | 109 |
| 15 | 426 |
| 16 | 389 |
| 17 | 643 |
| 18 | 104 |
| 19 | 322 |
| 20 | 96 |
| 21 | 260 |
| 22 | 365 |
| 23 | 327 |
| 83 | 98 |
| 89 | 37 |
| 90 | 88 |
| 91 | 354 |
| 92 | 249 |
| 93 | 493 |
| 60 | 95 |
| 94 | 200 |
| 95 | 628 |
| 96 | 249 |
| 97 | 32 |
| 98 | 113 |
| 99 | 567 |
| 100 | 142 |
| 101 | 67 |
| 102 | 442 |
| 103 | 137 |
| 105 | 76 |
| 106 | 216 |
| 107 | 361 |
| 108 | 305 |
| 109 | 457 |
| 110 | 452 |
| 111 | 393 |
| 112 | 538 |
| 113 | 226 |
| 114 | 539 |
| 115 | 72 |
| 116 | 175 |
| 119 | 421 |
| 121 | 766 |
| 123 | 134 |
| 126 | 183 |
| 129 | 858 |
| 131 | 96 |
| 132 | 276 |
| 133 | 241 |
| 135 | 796 |
| 139 | 38 |
| 141 | 996 |
| 142 | 14 |
| 143 | 77 |
| 144 | 897 |
| 149 | 225 |
| 150 | 353 |
| 154 | 832 |
| 155 | 94 |
| 156 | 867 |
| 158 | 49 |
| 160 | 244 |
| 164 | 161 |
| 165 | 30 |
| 166 | 13 |
| 168 | 43 |
| 169 | 19 |
| 171 | 50 |
| 172 | 355 |
| 175 | 57 |
| 177 | 127 |
| 178 | 30 |
| 182 | 92 |
| 183 | 57 |
| 191 | 37 |
| 194 | 30 |
| 195 | 2169 |
| 196 | 1832 |
| 197 | 1910 |
| 199 | 1383 |
| 201 | 1362 |
| 202 | 1509 |
| 203 | 1783 |
| 206 | 2317 |
| 207 | 2910 |
| 209 | 2466 |
| 210 | 2678 |
| 216 | 341 |
| 217 | 126 |
| 218 | 32 |
| 220 | 27 |
| 222 | 102 |
| 223 | 87 |
| 224 | 27 |
| 246 | 628 |

Cell Based Assay: Calcium Flux. The Aequorin-Based Assay

The following assay can be used for determination of GPR43 activation. The aequorin assay uses the responsiveness of mitochondrial apoaequorin to intracellular calcium release induced by the activation of GPCRs (Stables et al., 1997, Anal. Biochem. 252:115-126; Detheux et al., 2000, J. Exp. Med., 192 1501-1508). Briefly, GPCR-expressing clones are transfected to coexpress mitochondrial apoaequorin and Ga16. Cells expressing GPR43 receptor are incubated with 5 μM Coelenterazine H (Molecular Probes) for 4 hours at room temperature, washed in DMEM-F12 culture medium and resuspended at a concentration of 0.5×10⁶ cells/ml (the amount can be changed for optimization). Cells are then mixed with test compounds and light emission by the aequorin is recorded with a luminometer for 30 sec. Results are expressed as Relative Light Units (RLU). Controls include assays using cells not expressing GPR43 (mock transfected), in order to exclude possible non-specific effects of the candidate compound.

Aequorin activity or intracellular calcium levels are “changed” if light intensity increases or decreases by 10% or more in a sample of cells, expressing a GPR43 and treated with a compound of the invention, relative to a sample of cells expressing the GPR43 but not treated with the compound of the invention or relative to a sample of cells not expressing the GPR43 (mock-transfected cells) but treated with the compound of the invention.

Cell based assay: Intracellular Inositol-Phosphate Accumulation Assay. (Gq-Associated Receptor)

The following assay can be used for determination of GPR43 activation. On day 1, GPR43-expressing cells in mid-log phase are detached with PBS-EDTA, centrifuged at 2000×g for 2 min and resuspended in medium without antibiotics. After counting, cells are resuspended at 4×10⁵ cells/ml (the amount can be changed for optimization) in medium without antibiotics, distributed in a 96 well plate (100 μl/well) and the plate is incubated overnight at 37° C. with 5% CO₂. On day 2, the medium is removed and the compounds of the invention, at increasing concentrations, are added (24 μl/well) and the plate is incubated for 30 min. at 37° C. in a humidified atmosphere of 95% air with 5% CO₂. The IP1 concentrations are then estimated using the IP1-HTRF assay kit (Cisbio international, France) following the manufacturer recommendations.

Cell Based Assay: cAMP Accumulation Assay (G_{i/o}, Associated Receptor)

The following assay can be used for determination of GPR43 activation. Cells expressing GPR43 in mid-log phase and grown in media without antibiotics are detached with PBS-EDTA, centrifuged and resuspended in media without antibiotics. Cells are counted and resuspended in assay buffer at 4.2×10⁵ cells/ml. 96 well plates are filled with 12 μl of cells (5×10³ cells/well), 6 μl of compound of the invention at increasing concentrations and 6 μl of Forskolin (final concentration of 10 μM). The plate is then incubated for 30 min. at room temperature. After addition of the lysis buffer, cAMP concentrations are estimated, according to the manufacturer specification, with the HTRF kit from Cis-Bio International.

In vitro Assays to Assess Compound Activity in 3T3-L1 Cell Line

3T3-L1 adipocytes cell line has been described as cellular model to assess compounds mimicking insulin-mediated effect such as inhibition of lipolysis and activation of glucose uptake.

Lipolysis.

3T3-L1 cells (ATCC) are cultured in Dulbecco's modified eagle's medium (DMEM) containing 10% (v/v) bovine serum (fresh regular medium) in 24 well plate. On day 0 (2 days after 3T3-L1 preadipocytes reached confluence), cells are induced to differentiate by insulin (10 μg/ml), IBMX (0.5 mM) and dexamethasone (1 μM). On day 3 and every other 3^{rd} day thereafter, fresh regular medium is substituted until day 14.

On day 14, the medium is removed and cells are washed twice with 1 ml of a wash buffer (Hank's balanced salt solution). The wash solution is removed and the SCFA or the compounds of the invention, or a combination of both, are added at the desired concentration in Hank's buffer supplemented with 2% BSA-FAF and incubated for 10 minutes a 37° C. Then, isoproterenol (100 nM) is added to induce lipolysis and incubate for 30 minutes at 37° C. The supernatants are collected in a glycerol-free container. 25 μl (the amount can be changed for optimization) of cell-free supernatants are dispensed in 96-well microtiter plate, 25 μl of free glycerol assay reagent (Chemicon, the amount can be changed for optimization) is added in each well and the assay plate is incubated for 15 minutes at room temperature. The absorbance is recorded with a spectrophotometer at 540 or 560 nm. Using the supernatants, the free fatty acids amount can be assessed using the NEFA assay kit (Wako) according the manufacturer's recommendations.

Glucose Uptake.

3T3-L1 cells are differentiated as described previously with or without of 30 μM of compound of the invention (the concentration can be changed for optimization) during the 14 days of differentiation. The day of the experiment, the cells are washed twice with a KREBS-Ringer bicarbonate (pH 7.3) supplemented with 2 mM sodium pyruvate and starved for 30 minutes in the same buffer at 37° C. in an atmosphere containing 5% CO2 and 95% O2. Various amount of SCFA, compounds of the invention or combination of both are then added with or without 10 nM of insulin (the amount can be changed for optimization) for 30 minutes at 37° C. in an atmosphere containing 5% CO2 and 95% O2. Then, D-(³H)-2 deoxyglucose (0.2 μCi/well) and D-2-deoxyglucose (0.1 mM) is added for 30 minutes. To stop the reaction, the cells are immersed in ice-cold saline buffer, washed for 30 min, and then dissolved in NaOH 1M at 55° C. for 60 minutes. NaOH is neutralized with HCl 1M. The 3H labeled radioactivity of an aliquot of the extract is counted in the presence of a scintillation buffer.

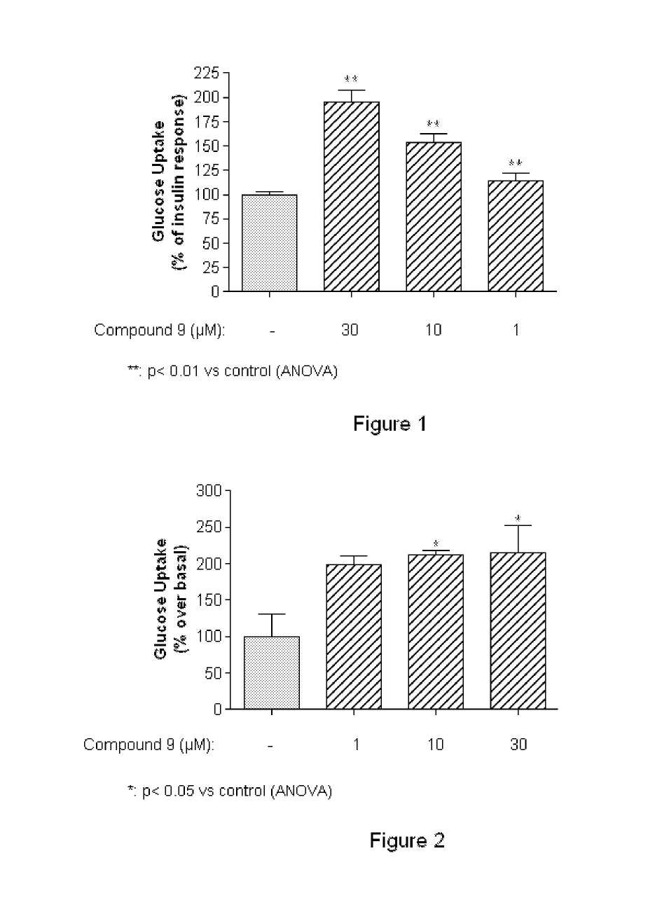

When tested in the glucose-uptake assay described above and by way of illustration the compound n° 9 significantly increases the glucose-uptake in response to 10 nM of insulin (FIG. 1).

It is important to note that in the above-mentioned assay the positive allosteric modulators (PAMs) disclosed in Lee et al., (Mol. Pharmacol. 74(6) pp 1599-1609, 2008) do not increase the glucose uptake. This lack of effect on glucose uptake could be explained by the weak affinity (˜1 μM) of the PAMs disclosed by Lee et al.

In vitro Assays to Assess Compound Activity in NCI-H716 Cell Line

Human intestinal cell line NCI-H716 has been described as cellular model to assess compounds mimicking nutrient-mediated effect such as glucagon-like peptide-1 (GLP-1) secretion.

GLP-1 Release.

NCl-H716 cells (ATCC, Manassas) are cultured in Dulbecco's modified eagle's medium (DMEM) containing 10% (v/v) bovine serum, 2 mM L-glutamine, 100 IU/ml penicillin and 100 μg/ml streptomycin in 75 ml flask. Cell adhesion and endocrine differentiation is initiated by growing cells in 96-well plate coated with matrigel in High Glucose DMEM containing 10% (v/v) bovine serum, 2 mM L-glutamine, 100 IU/ml penicillin and 100 μg/ml streptomycin for 2 days. On day 2, the medium is removed and cells are washed once with a pre-warmed wash buffer (Phosphate Buffered salt solution). The wash solution is removed and the SCFA or the compounds of the invention, or a combination of both, are added at the desired concentration in High Glucose DMEM containing 0.1% (v/v) bovine serum and incubated for 2 hours at 37° C. The supernatants are collected in a container. Using the cell-free supernatants, the GLP-1 amount is assessed using a GLP-1 specific ELISA assay kit according the manufacturer's recommendations (ALPCON).

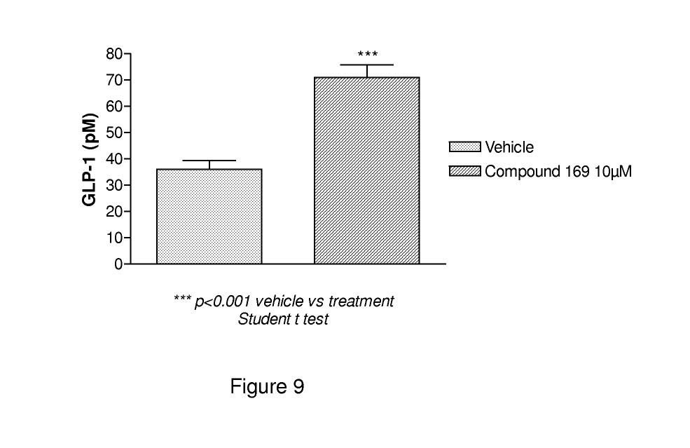

When tested in the GLP-1 release assay described above and by way of illustration the compound n° 169 significantly increases the GLP-1 secretion from NCI-H716 cells (FIG. 9).

Ex vivo Assays to Assess Compound Activity in Adipocytes from Normal and High-Fat Diet Fed Mice

Mice C56Black6 male were housed in Makrolon type IV group housing cages (56×35×20 cm³) throughout the experimental phase. Animals' cages litters were changed once a week. They were housed in groups of 10 animals at 12 light dark (at 8 h30 pm lights off), 22+/−2° C. and 50+/−5% relative humidity. Animals were acclimated one week. During the whole phase, standard diet or diet high in energy from fat (Research Diets, New Brunswick, N.J.) and tap water were provided ad libitum. The animals were 16 weeks old at the time of the study.

For keeping only mice that have responded to the high fat diet, fasted glycemia was measured in these mice just before performing the ex-vivo study.

Glucose Uptake Assay in Isolated Adipocytes.

Animals were killed by cervical dislocation and epididymal fat pads were removed and digested in collagenase buffer at 37° C./120 rpm for approximately 50 minutes. The digest was filtered through gauze to recover the adipocytes, which were washed and resuspended in Krebs-Ringer Hepes (KRH) buffer containing 1% BSA, 200 nM adenosine and 2 mM glucose.

Isolated adipocytes were washed in glucose-free KRH-buffer and resuspended to 30%. Adipocytes were then incubated at 37° C./80 rpm with either compound of the invention (30 μM, 10 μM and 1 μM) in the presence or absence of insulin (10 nM) for 30 min. 2-deoxyglucose and 2-deoxy-D-[1-³H]-glucose (³H-2-DOG) were added and incubation continued for 10 min. The reactions were then stopped by addition of cytochalasin b followed by centrifugation through dinonylphthalate to recover the adipocytes. The uptake of ³H-2-DOG- was measured by scintillation. Each data point was investigated in triplicates in two independent experiments.

When tested in the assay described above and by way of illustration the compound n° 9 significantly increase the glucose uptake in adipocytes isolated from High-fat diet fed mice (FIG. 2).

Lipolysis Assay in Isolated Adipocytes.

Isolated adipocytes were diluted to 5% in KRH-buffer and were pre-treated with compound of the invention (30 μM, 10 μM and 1 μM) for 30 min at 37° C./120 rpm. After the pre-treatment, Isoprenaline (1 μM) was added to the adipocytes followed by 30 min incubation at 37° C./150 rpm. The reactions were put on ice and the buffer was assayed spectrophotometrically for the production of NADH⁺ from glycerol breakdown in reactions catalyzed by glycerol kinase and glycerol-3-phosphate dehydrogenase and/or Non Esterified Fatty Acid (NEFA). Each data point was investigated in triplicates in two independent experiments.

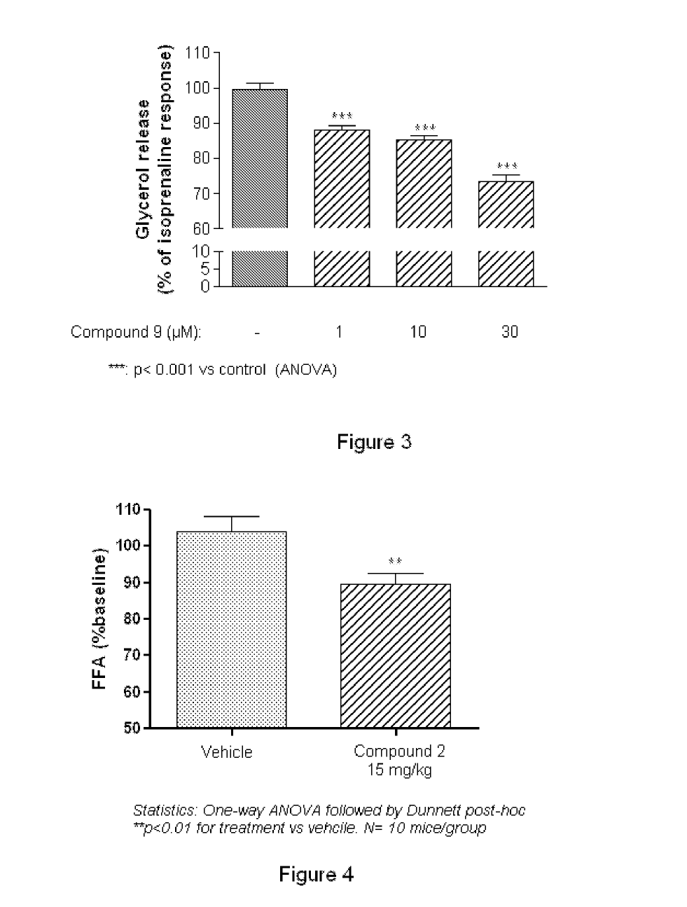

According to the method described above and by way of illustration the compound n° 9 dose-dependently inhibits isoprenaline-induced lipolysis in adipocytes from high-fat diet fed mice (FIG. 3).

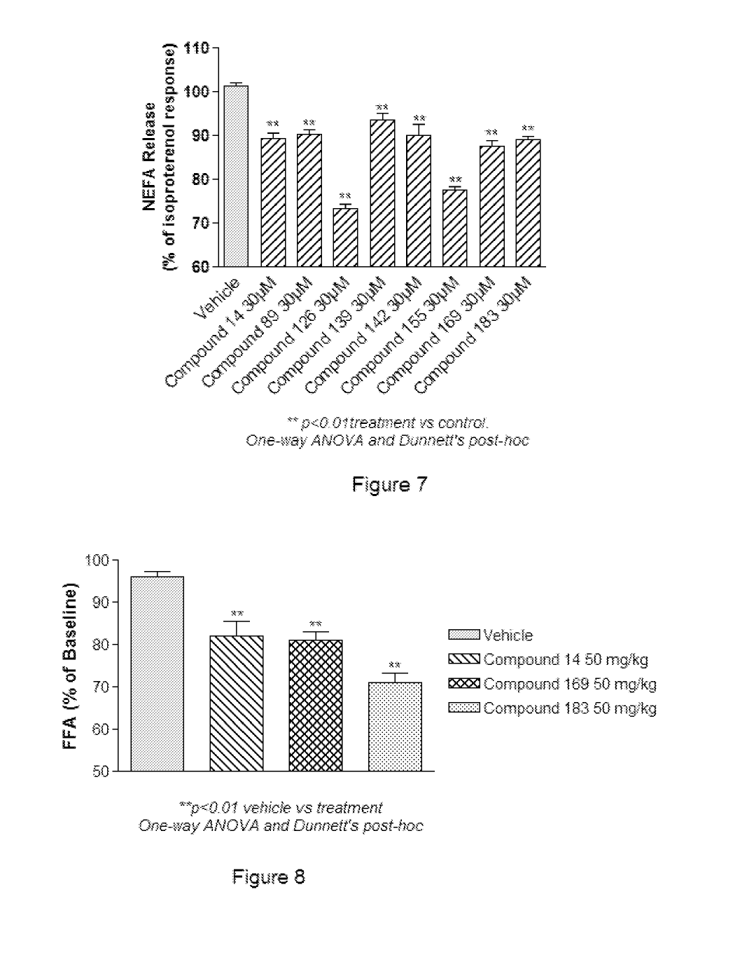

Compounds n° 14, 89, 126, 139, 142, 155, 169 and 183 inhibit isoprenaline-induced lipolysis in adipocytes isolated from normal rats according to the method described above (FIGS. 6 and 7).

It is important to note that in the above-mentioned assay the positive allosteric modulators (PAMs) disclosed in Lee et al., (Mol. Pharmacol. 74(6) pp 1599-1609, 2008) do not display an anti-lipolytic effect on rat adipocytes. This lack of effect could be explained by the weak affinity (˜1 μM) of the PAMs disclosed by Lee et al.

In vivo Assay to Assess Compound Activity in Rodent Diabetes Model

Genetic Rodent Models:

Rodent models of T2D associated with obesity and insulin resistance have been developed. Genetic models such as db/db and ob/ob in mice and fa/fa in Zucker rats have been developed for understanding the pathophysiology of disease and testing candidate therapeutic compounds as compound of the invention. The homozygous animals, C57 Black/6-db/db mice developed by Jackson Laboratory are obese, hyperglycemic, hyperinsulinemic and insulin resistant (J Clin Invest, 1990, 85:962-967), whereas heterozygotes are lean and normoglycemic. In the db/db model, mice progressively develop insulinopenia with age, a feature commonly observed in late stages of human T2D when sugar levels are insufficiently controlled. Since this model resembles that of human T2D, the compounds are tested for activities including, but not limited to, lowering of plasma glucose and triglycerides. Zucker (fa/fa) rats are severely obese, hyperinsulinemic, and insulin resistant, and the fa/fa mutation may be the rat equivalent of the murine db mutation.

Genetically altered obese diabetic mice (db/db) (male, 7-9 weeks old) are housed under standard laboratory conditions at 22° C. and 50% relative humidity, and maintained on a diet of Purina rodent chow and water ad libitum. Prior to treatment, blood is collected from the tail vein of each animal and blood glucose concentrations are determined using one touch basic glucose monitor system (Lifescan). Mice that have plasma glucose levels between 250 to 500 mg/dl are used. Each treatment group consists of several mice that are distributed so that the mean of glucose levels are equivalent in each group at the start of the study. Db/db mice are dosed by micro-osmotic pumps, inserted using isoflurane anesthesia, to provide compounds of the invention, saline, or an irrelevant compound to the mice intravenously (i.v). Blood is sampled from the tail vein at intervals thereafter and analyzed for blood glucose concentrations. Significant differences between groups (comparing compounds of the invention to saline-treated) are evaluated using Student t-test.

The High-Fat Diet Fed Mouse:

This model was originally introduced by Surwit et al. in 1988. The model has shown to be accompanied by insulin resistance, as determined by intravenous glucose tolerance tests, and of insufficient islet compensation to the insulin resistance. The model has, accordingly, been used in studies on pathophysiology of impaired glucose tolerance (IGT) and type 2 diabetes and for development of new treatments.

C57BL/6J mice are maintained in a temperature-controlled room (22° C.) on a 12-h light-dark cycle. One week after arrival, mice are divided into two groups and are fed either a high-fat diet or received continuous feeding of a normal diet for up to 12 months. On caloric basis, the high-fat diet consist of 58% fat from lard, 25.6% carbohydrate, and 16.4% protein (total 23.4 kJ/g), whereas the normal diet contains 11.4% fat, 62.8% carbohydrate, and 25.8% protein (total 12.6 kJ/g). Food intake and body weight are measured once a week, and blood samples are taken at indicated time points from the intraorbital retrobulbar plexus from nonfasted anesthetized mice.

For intravenous glucose tolerance tests (IVGTTs), 4-h fasted mice are anesthetized with 7.2 mg/kg fluanison/fenlanyl and 15.3 mg/kg midazolam.

Thereafter a blood sample is taken from the retrobulbar, intraorbital, capillary plexus, after which D-glucose (1 g/kg) is injected intravenously in a tail vein (volume load 10 l/g). Additional blood samples are taken at 1, 5, 10, 20, 50, and 75 min after injection. Following immediate centrifugation at 4 C, plasma is separated and stored at −20 C until analysis. For oral glucose tolerance tests (OGTTs), 16-h fasted anesthetized mice are given 150 mg glucose by gavage through a gastric tube (outer diameter 1.2 mm), which is inserted in the stomach. Blood samples are taken at 0, 15, 30, 60, 90, and 120 min after glucose administration and handled as above.

Administration of the compounds: Five-week-old mice are fed a high-fat or a normal diet for 8 weeks. After 4 weeks, the mice are additionally given the compound of the invention in their drinking water (0.3 mg/ml, the amount can be changed for optimization. Control groups are given tap water without compound. After another 4 weeks, the mice are subjected to an OGTT as described above.

Insulin and glucose measurements: Insulin is determined enzymatically using an ELISA assay kit (Linco Research, St. Charles, Mo.). Plasma glucose is determined by the glucose oxidase method.

In vivo Assay to Assess Compound Anti-Obesity Activity in Rodent Model

Mouse Acute Food Intake and Weight Change:

Male C57BL/6N wild-type mice are weighed and vehicle or compounds of the invention are administered by oral gavage to male mice approximately 30 min prior to the onset of the dark phase of the light cycle. Mice are fed ad libitum in the dark phase following dosing. A preweighed aliquot of a highly palatable medium high fat diet is provided in the food hopper of the cage 5 min prior to the onset of the dark phase of the light cycle and weighed 2 and 18 h after the onset of the dark phase of the light cycle.

Acute Studies in Diet-Induced Obesity (DIO) Rats:

For acute experiments, male Sprague-Dawley DIO rats from Charles River Laboratories are raised from 4 weeks of age on a diet moderately high fat (32% kcal) and high in sucrose (25% kcal). Animals are used at 12 weeks of age and are maintained on a 12/12 h light dark cycle. The rats are randomized into groups (n=6/group) for compounds of the invention and vehicle dosing. Rats are weighed 17 h after dosing to determine effects on overnight body weight gain. Compounds of the invention are administered orally or s.c. at amount desired 1 h before the start of the dark cycle. Powdered food is provided in food cups which are weighed continuously at 5 min intervals over 18 h and the data are recorded using a computerized system.

Chronic Studies in Diet-Induced Obesity Rats:

For the 14-day chronic experiment, male Sprague-Dawley DIO rats are obtained as described above. Animals are used at 15 weeks of age and are maintained on a 12/12 hour light-dark cycle. Rats are conditioned to dosing for 4 days prior to baseline measurements, using an oral gavage or a s.c. route of vehicle. Thereafter, animals are dosed daily with vehicle or compound by oral gavage or s.c. Compound of the invention or vehicle is administered 1 h before the dark cycle for 14 days. Body composition is measured by dual energy X-ray densitometry (DEXAscan) 5 days prior to the study and at the end of the 14-day study. Daily endpoints included body weight and food intake.

In vivo Assay to Assess Compound Anti-Lipolytic Activity in Rodent Model

Male C57BL/6N wild-type are housed one per cage in a room maintained on a 12 h light/dark cycle under constant temperature (22-25° C.) with ad libitum access to food and water. The anti-lipolytic effects of the compounds of the invention are studied in awake mice. Animals are fasted overnight before experimental use. On the day of the experiment, animals are put in metabolic cages and left undisturbed to acclimate to the environment for 1-2 h. blood samples are taken at indicated time points from the intraorbital retrobulbar plexus. A 1% sodium citrate saline solution is used to flush the lines. A pre-treatment blood sample is obtained from each animal to determine baseline values for free fatty acids (FFA) and triglycerides (TG). Compounds of the invention are given via oral gavage, sc injection, iv injection or ip injection for each different series of experiments. Blood samples are collected into pre-cooled tubes pre-coated with heparin (200 μl blood, Li-heparin, Sarstedt) for determination triglycerides and glycerol and in tri-potassium EDTA added sodium fluoride (200 μl blood, K₃-EDTA, 1.6 mg/mL+1% NaF, Sarstedt) for determination of plasma free fatty acids. The tubes are placed on wet ice pending processing. Blood samples will be centrifuged at 4000×g, at 4° C., 15 min the resulting plasma will be transferred into non-coated tubes and stored at −80° C. until analyses. The plasma is thawed at 4° C. for determinations of FFA and TG using commercial kits (Wako Chemicals).

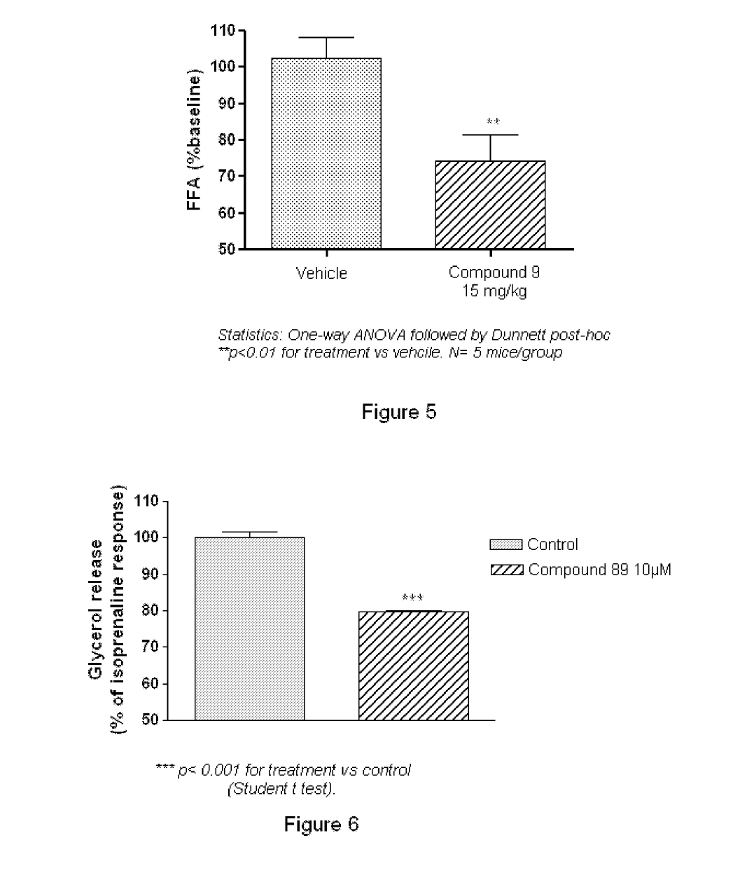

According to the method described above and by way of illustration the compounds n° 2 and 9 administered by ip injection, inhibit, 15 minutes following the injection, in vivo FFA baseline at the concentration of 15 mg/kg from normal diet fed mice in comparison to the vehicle (FIGS. 4 and 5). The compounds n° 14, 169 and 183 orally administered, inhibit, 15 minutes following the dosing, in vivo FFA baseline at the concentration of 50mg/kg from normal diet fed mice in comparison to the vehicle (FIG. 8).

While embodiments of the invention have been illustrated and described, it is not intended that these embodiments illustrate and describe all possible forms of the invention. Rather, the words used in the specification are words of description rather than limitation ant it is understood that various changes may be made without departing from the spirit and scope of the invention.

1-19. (canceled)

20. A compound of formula Ib-4: 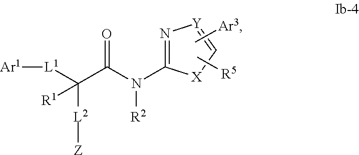
and pharmaceutically acceptable salts, and solvates thereof, wherein
Ar¹ is a 5- to 6-membered aryl or heteroaryl group, 3- to 8-membered cycloalkyl group, a 3- to 8-membered heterocycloalkyl group, or a linear or branched C₃-C₆ alkyl group, each of the aryl, heteroaryl, cycloalkyl, heterocycloalkyl, or alkyl groups being optionally substituted by one or more groups selected from halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, amino, alkoxyalkoxy, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or two substituents form an alkylenedioxy group or a haloalkylenedioxy group, or two substituents form a cycloalkyl or heterocycloalkyl moiety together with the cycloalkyl or heterocycloalkyl group they are attached to, or fused to the aryl, heteroaryl, cycloalkyl or heterocycloalkyl group may be one or more cycloalkyl, aryl, heterocyclyl or heteroaryl moiety, each of said substituents being optionally substituted by one or more further substituents selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, oxo, or sulfonyl;
L¹ is a single bond, C₁-C₂ alkylene, C₁-C₂ alkenylene, each optionally being substituted by one or more substituents selected from halo, C₁-C₂ alkyl, C₁-C₂ haloalkyl; or L¹ is —N(R^{N})—, wherein R^{N} is H or C₁-C₂ alkyl; or L¹ and R¹ together are ═CH—;
R¹ is H, halo, allyl, or a C₁-C₄ alkyl group, which may optionally be substituted by one or more groups selected from halo or C₁-C₄ alkyl;
L² is a C₁-C₃ alkylene, C₂-C₄ alkenylene, C₃-C₆ cylcloalkylene, each of which being optionally substituted by one or more groups selected from halo, alkyl, alkoxy, or haloalkyl;
or L² is —O—CH₂—
R¹ and L² together are ═CH—, under the condition that -L¹-Ar¹ is H; or
R¹ and L² together are a 5- to 6-membered saturated or unsaturated carbocyclic or heterocyclic group, under the condition that -L¹-Ar¹ is H;
Z is selected from the group consisting of —COOR, 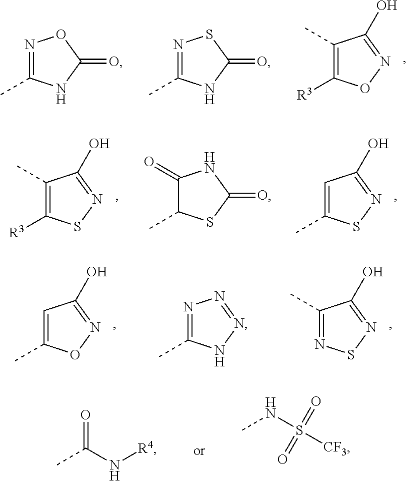
wherein R is H or linear or branched alkyl, aryl, acyloxyalkyl, dioxolene, R³ is H, methyl or ethyl, and R⁴ is hydroxyl —SO₂CH₃, —SO₂cyclopropyl or —SO₂CF₃;
R² is H, linear or branched C₁-C₄ alkyl, C₁-C₄ hydroxyalkyl, C₁-C₄ haloalkyl, C₂-C₄ alkenyl, C₂-C₄ alkynyl, C₃-C₆ cycloalkyl, C₃-C₆ cycloalkylalkyl, aryl, arylalkyl, heteroarylalkyl, alkoxycarbonylalkyl, aminocarbonylalkyl, or aralkyloxyalkyl; each of the alkyl, hydroxyalkyl, alkenyl, alkynyl, cycloalkyl, cycloalkylalkyl, aryl, arylalkyl, heteroarylalkyl, alkoxycarbonylalkyl, aminocarbonylalkyl, and aralkyloxyalkyl groups being optionally substituted by one or more substituents selected from halo, cyano, alkyl, hydroxyalkyl, haloalkyl, alkenyl, alkynyl, heteroalkyl, hydroxyl, alkoxy, haloalkoxy, cycloalkyloxy, amino, alkylamino, aminoalkyl, carboxy, alkoxycarbonyl, alkylcarbonyloxy, alkylcarbonylamino, haloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, alkylsulfonyl, haloalkylsulfonyl, sulfamoyl, alkylsulfamoyl, alkylsulfonylamino, cycloalkylsulfonylamino, haloalkylsulfonylamino, or two substituents form an alkylenedioxy group or a haloalkylenedioxy group,
Ar³ is an aryl or heteroaryl group, each of which being optionally substituted by one or more groups selected from halo, cyano, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, cycloalkylaminocarbamoyl, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or two substituents form an alkylenedioxy group or a haloalkylenedioxy group, or two substituents form a cycloalkyl or heterocycloalkyl moiety together with the cycloalkyl or heterocycloalkyl group they are attached to, or fused to the aryl, heteroaryl, cycloalkyl or heterocycloalkyl group may be one or more cycloalkyl, aryl, heterocyclyl or heteroaryl moiety, each of said substituents being optionally substituted by one or more further substituents selected from halo, alkoxy, alkyl, alkoxyalkyl, alkoxyalkoxy, cycloalkylalkyloxy, amino, alkylamino, alkylaminoalkoxy, cycloalkylamino, aralkylamino, alkylaminoalkyl, alkylaminocarbonyl, alkylcarbonyl, cycloalkylcarbonylamino, alkylheterocyclyl, alkylheteroaryl, alkylsulfonyl, alkylsulfonylamino, aralkyl, aralkyloxy, aryl, arylamino, aryloxy, cyano, haloalkoxy, haloalkyl, heteroaryl, heteroarylalkyl, heteroarylcarbonyl, heterocyclyl, heterocyclyloxy, hydroxyl, oxo, or sulfonyl, or Ar³ form an aryl, or heteroaryl group fused to Ar², wherein each of said aryl or heteroaryl groups fused to Ar² are optionally substituted by one or more halo;
X is S or O;
Y is CH or N;
Ar³ is attached to the heterocyclic group 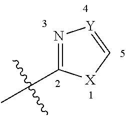
either in position 4 or 5; and
if Y is CH, R⁵ is H, halo, cyano, hydroxyl, linear or branched C₁-C₃ alkyl, C₁-C₃ hydroxyalkyl, C₁-C₃ haloalkyl, and R⁵ is attached to the heterocyclic group either in position 4, if Ar³ is attached in position 5, or in position 5, if Ar³ is attached in position 4;
if Y is N, R⁵ is absent and Ar³ is attached in position 5;
with the following provisos: 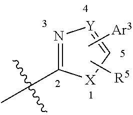
is not 4-(4-butylphenyl)thiazol-2-yl, 4-(4-ethylphenyl)thiazol-2-yl, 4-(para-tolyl)thiazol-2-yl, 4-phenylthiazol-2-yl, 4-(4-propylphenyl)thiazol-2-yl, 4-(4-(sec-butyl)phenyl)thiazol-2-yl, 4-(4-isopropylphenyl)thiazol-2-yl, 4-(4-isobutylphenyl)thiazol-2-yl, 4-(4-(tert-butyl)phenyl)thiazol-2-yl, 4-(4-butylphenyl)-5-methylthiazol-2-yl, 4-(4-ethylphenyl)-5-methylthiazol-2-yl, 5-methyl-4-(para-tolyl)thiazol-2-yl, 5-methyl-4-phenylthiazol-2-yl, 5-methyl-4-(4-propylphenyl)thiazol-2-yl, 4-(4-(sec-butyl)phenyl)-5-methylthiazol-2-yl, 4-(4-isopropylphenyl)-5-methylthiazol-2-yl, 4-(4-isobutylphenyl)-5-methylthiazol-2-yl, 4-(4-(tert-butyl)phenyl)-5-methylthiazol-2-yl, 4-(4-butyl-3-methylphenyl)thiazol-2-yl, 4-(4-ethyl-3-methylphenyl)thiazol-2-yl, 4-(3,4-dimethylphenyl)thiazol-2-yl, 4-(meta-tolyl)thiazol-2-yl, 4-(3-methyl-4-propylphenyl)thiazol-2-yl, 4-(4-(sec-butyl)-3-methylphenyl)thiazol-2-yl, 4-(4-isopropyl-3-methylphenyl)thiazol-2yl, 4-(4-isobutyl-3-methylphenyl)thiazol-2-yl, 4-(4-(tert-butyl)-3-methylphenyl)thiazol-2-yl, 4-(4-butyl-3-methylphenyl)-5-methylthiazol-2-yl, 4-(4-ethyl-3-methylphenyl)-5-methylthiazol-2-yl, 4-(3,4-dimethylphenyl)-5-methylthiazol-2-yl, 5-methyl-4-(meta-tolyl)thiazol-2-yl, 5-methyl-4-(3-methyl-4-propylphenyl)thiazol-2-yl, 4-(4-(sec-butyl)-3-methylphenyl)-5-methylthiazol-2-yl, 4-(4-isopropyl-3-methylphenyl)-5-methylthiazol-2-yl, 4-(4-isobutyl-3-methylphenyl)-5-methylthiazol-2-yl, 4-(4-(tert-butyl)-3-methylphenyl)-5-methylthiazol-2-yl;
Ar³ is not (7H-pyrrolo[2,3-d]pyrimidin)-4yl; 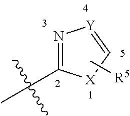
is not 5-cyano-thiazolyl;
the compound of formula I is none of.
2-[[[4-(4-butylphenyl)-5-methyl-2-thiazolyl]amino]carbonyl]-cyclohexane carboxylic acid,
2-[[[4-(4-methoxyphenyl)-5-methyl-2-thiazolyl]amino]carbonyl]-cyclohexanecarboxylic acid,
6-[[[4-(3,4-dimethylphenyl)-5-methyl-2-thiazolyl]amino]carbonyl]-3-cyclohexene-1-carboxylic acid,
6-[[[5-methyl-4-(4-propylphenyl)-2 thiazolyl]amino]carbonyl]-3-cyclohexene-1-carboxylic acid,
2-[[[4-(2,4-dichlorophenyl)-5-methyl-2-thiazolyl]amino]carbonyl]-cyclohexanecarboxylic acid,
2-[[[4-(2,5-dimethylphenyl)-5-methyl-2-thiazolyl]amino]carbonyl]-cyclohexanecarboxylic acid,
6-[[[5-(2-chlorophenyl)-1,3,4-thiadiazol-2-yl]amino]carbonyl]-3-cyclohexene-1-carboxylic acid,
2-[[[5-methyl-4-(4-propylphenyl)-2-thiazolyl]amino]carbonyl]-cyclohexanecarboxybic acid,
6-[[[4[-4-(1,1-dimethylethyl)phenyl]-5-methyl-2-thiazolyl]amino]carbonyl]-3-cyclohexene-1-carboxylic acid,
2-[[(5-methyl-4-phenyl-2-thiazolyl)amino]carbonyl]-cyclohexanecarboxylic acid,
2-[[[5-methyl-4-[4-(2-methylpropyl)phenyl]-2-thiazolyl)amino]carbonyl]-cyclohexanecarboxylic acid,
2-[[[4-(4-chlorophenyl)5-ethyl-2-thiazoly)amino]carbonyl]-cyclohexanecarboxylic acid,
2-[[[4-(3-methoxyphenyl)-5-methyl-2-thiazolyl]amino]carbonyl]-cyclohexanecarboxylic acid,
6-[[[5-methyl-4-(4-methylphenyl)-2-thiazolyl]amino]carbonyl]-3-cyclohexene-1-carboxylic acid,
6-[[[4-(4-chlorophenyl)-5-ethyl-2-thiazolyl]amino]carbonyl]-3-cyclohexene-1-carboxylic acid,
6-[[[4-(2,5-dimethylphenyl)-5-methyl-2-thiazolyl]amino]carbonyl]-3-cyclohexene-1-carboxylic acid,
6-[[(5-phenyl-1,3,4-thiadiazol-2-yl)amino]carbonyl]-3-cyclohexene-1-carboxylic acid,
2-[[[5-(4-methoxyphenyl)-1,3,4-thiadiazol-2yl]amino]carbonyl]-cyclohexanecarboxylic acid,
2-[[(6-carboxy-3-cyclohexen-1-yl)carbonyl]amino]-4-phenyl-5-thiazolecarboxylic acid-5-ethyl ester,
6-[[[5-methyl-4-[4-(2-methylpropyl)phenyl]-2-thiazolyl)amino]carbonyl]-3-cyclohexene-1-carboxylic acid,
6-[[(5-ethyl-4-phenyl-2-thiazolyl)amino]carbonyl]-3-cyclohexene-1-carboxylic acid,
6-[[[4-(2,4-dimethylphenyl)-5-methyl-2-thiazolyl)amino]carbonyl]-3-cyclohexene-1-carboxylic acid,
2-[[[4-(3-chlorophenyl)-5-methyl-2-thiazolyl]amino]carbonyl]-cyclohexanecarboxylic acid,
6-[[[5-(1-ethylphenyl)-1,3,4-thiadiazol-2-yl]amino]carbonyl]-3-cyclohexene-1-carboxylic acid,
2-[[[5-(2-thienyl)-1,3,4-thiadiazol-2-yl]amino]carbonyl]-cyclohexanecarboxylic acid,
2-[[(4,5-diphenyl-2-thiazolyl)amino]carbonyl]-cyclohexanecarboxybic acid,
6-[[[4-(4-ethylphenyl)-5-methyl-2-thiazolyl]amino]carbonyl]-3-cyclohexene-1-carboxylic acid,
2-[[(5-ethyl-4-phenyl-2-thiazolyl)amino]carbonyl]-cyclohexanecarboxylic acid,
2-[[[4-(4-fluorophenyl)-5-methyl-2-thiazolyl]amino]carbonyl]-cyclohexanecarboxylic acid,
2-[[[4-(2,4-dimethylphenyl)-5-methyl-2-thiazolyl]amino]carbonyl]-cyclohexanecarboxylic acid,
6-[[[4-(3-chlorophenyl)-5-methyl-2-thiazolyl]amino]carbonyl]-3-cyclohexene-1-carboxylic acid,
2-[[[5-methyl-4-(4-methylphenyl)-2-thiazolyl]amino]carbonyl]-cyclohexanecarboxylic acid,
6-[[[5-(2-thienyl)-1,3,4-thiadiazol-2-yl]amino]carbonyl]-3-cyclohexene-1-carboxylic acid,
2-[[[4-(4-ethylphenyl)-5-methyl-2-thiazolyl]amino]carbonyl]-cyclohexanecarboxylic acid,
2-[[(2-carboxycyclohexyl)carbonyl]amino]-4-phenyl-5-thiazolecarboxylic acid-5-ethyl ester,
2-[[[5-methyl-4-[4-(1-methylethyl)phenyl]-2-thiazolyl]amino]carbonyl]-cyclohexanecarboxylic acid,
6-[[[4-(2,4-dichlorophenyl)-5-methyl-2-thiazolyl]amino]carbonyl]-3-cyclohexene-1-carboxylic acid,
6-[[[4-(4-chlorophenyl)-5-methyl-2-thiazolyl]amino]carbonyl]-3-cyclohexene-1-carboxylic acid,
2-[[[4-(4-chlorophenyl)-5-methyl-2-thiazolyl]amino]carbonyl-cyclohexanecarboxylic acid,
6-[[[4-(4-fluorophenyl)-5-methyl-2-thiazolyl]amino]carbonyl]-3-cyclohexene-1-carboxylic acid,
2-[[[4[4-(1,1-dimethylethyl)phenyl]-5-methyl-2-thiazolyl]amino]carbonyl-cyclohexanecarboxylic acid,
6-[[(5-methyl-4-phenyl-2-thiazolyl)amino]carbonyl]-3-cyclohexene-1-carboxylic acid,
6-[[(5-(2-thienyl)-1,3,4-thiadiazol-2-yl]amino]carbonyl]-3-cyclohexene-1-carboxylic acid;
and pharmaceutically acceptable salts, and solvates thereof.

21. The compound according to claim 20 having the formula Ib-4a: 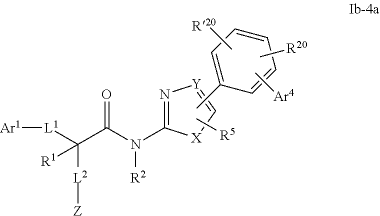
wherein
Ar¹, L¹, L², R¹, R², R⁵, X, Y and Z are as defined in claim 20;
R²° and R′²⁰ are independently selected from halo, cyano, C₁-C₃ alkyl, cyclopropyl, haloalkyl, alkoxy, haloalkoxy, alkoxycarbonylamino, or the two substituents form an alkylenedioxy group or a haloalkylenedioxy group;
Ar⁴ is 5 or 6 membered aryl, 5 or 6 membered heteroaryl, each of said 5 or 6 membered aryl or 5 or 6 membered heteroaryl groups being optionally fused to one or more 5 or 6 membered cycloalkyl, aryl, heterocyclyl or heteroaryl moiety, thus forming a fused ring system, and the latter fused ring system being optionally substituted by one or more further substituents selected from halo, hydroxyl, oxo, alkyl, and/or each of said 5 or 6 membered aryl or 5 or 6 membered heteroaryl groups being optionally substituted by one or more substituents selected from halo, cyano, hydroxyl, alkyl, cycloalkyl, heterocyclyl, aryl, heteroaryl, aralkyl, heteroarylalkyl, haloalkyl, alkoxy, haloalkoxy, alkoxyalkyl, alkoxyalkoxy, alkylaminoalkoxy, cycloalkyloxy, cycloalkylalkyloxy, heterocyclyloxy, aryloxy, aralkyloxy, alkylamino, alkylaminoalkyl, cycloalkylamino, arylamino, aralkylamino, alkylaminocarbonyl, heteroarylcarbonyl, alkylcarbonylamino, cycloalkylcarbonylamino, alkylsulfonyl, haloalkylsulfonyl, alkylsulfonylamino, each of said cycloalkyl, heterocyclyl, aryl, heteroaryl, aralkyl, heteroarylalkyl, cycloalkyloxy, cycloalkylalkyloxy, heterocyclyloxy, aryloxy, aralkyloxy, heteroarylcarbonyl, cycloalkylamino, arylamino, aralkylamino, cycloalkylcarbonylamino being optionally substituted by one or more further substituents selected from halo, oxo or alkyl;
and pharmaceutically acceptable salts, and solvates thereof.

22. The compound according to claim 21 having the formula Ib-4b: 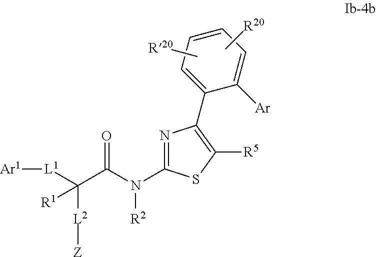
wherein
Ar¹, Ar⁴, L¹, L², R¹, R², R⁵, R²⁰, R′²⁰, and Z are as defined in claim 21;
and pharmaceutically acceptable salts, and solvates thereof.

23. The compound according to claim 22 having formula Ib-4c: 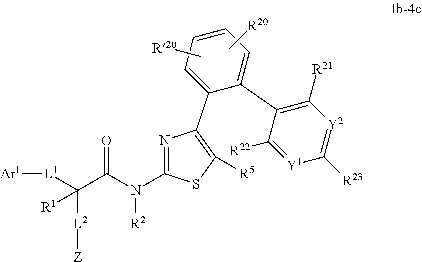
wherein
Ar¹, L¹, L², R¹, R², R⁵, and Z are as defined in claim 20,
R²° and R′²⁰ are independently selected from halo, cyano, C₁-C₃ alkyl, cyclopropyl, haloalkyl, alkoxy, haloalkoxy, alkoxycarbonylamino, or the two substituents form an alkylenedioxy group or a haloalkylenedioxy group;
R²¹ and R²² are independently selected from H, halo, alkoxy;
R²³ is selected from halo, cyano, hydroxyl, alkyl, cycloalkyl, heterocyclyl, aryl, heteroaryl, aralkyl, heteroarylalkyl, haloalkyl, alkoxy, haloalkoxy, alkoxyalkyl, alkoxyalkoxy, alkylaminoalkoxy, cycloalkyloxy, cycloalkylalkyloxy, heterocyclyloxy, aryloxy, aralkyloxy, alkylamino, alkylaminoalkyl, cycloalkylamino, arylamino, aralkylamino, alkylaminocarbonyl, heteroarylcarbonyl, alkylcarbonylamino, cycloalkylcarbonylamino, alkylsulfonyl, each of said cycloalkyl, heterocyclyl, aryl, heteroaryl, aralkyl, heteroarylalkyl, cycloalkyloxy, cycloalkylalkyloxy, heterocyclyloxy, aryloxy, aralkyloxy, heteroarylcarbonyl, cycloalkylamino, arylamino, aralkylamino, cycloalkylcarbonylamino being optionally substituted by one or more further substituents selected from halo, oxo or alkyl;
Y¹ is N or C—R²⁴ where R²⁴ is H, halo, alkoxy, alkyl, heterocyclyl, or
Y¹ is C—R²⁴ and R²⁴ and R²³ together form a 5 or 6 membered cycloalkyl, aryl, heterocyclyl or heteroaryl moiety, thus forming a fused ring system, the latter fused ring system being optionally substituted by one or more group selected from oxo, alkyl or halo; and
Y² is N or C—R²⁵ where R²⁵ is H, halo, alkoxy, alkyl, heterocyclyl, or
Y² is C—R²⁵ and R²⁵ and R²³ together form a 5 or 6 membered cycloalkyl, aryl, heterocyclyl or heteroaryl moiety, thus forming a fused ring system, the latter fused ring system being optionally substituted by one or more group selected from oxo, alkyl or halo, under the condition that R²⁴ and R²³ together do not form a 5 or 6 membered cycloalkyl, aryl, heterocyclyl or heteroaryl moiety;
and pharmaceutically acceptable salts, and solvates thereof.

24. The compound according to claim 23 having formula Ib-4d: 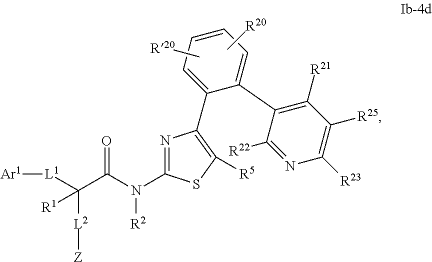
wherein,
Ar¹, L¹, L², R¹, R², R⁵, and Z are as defined in claim 20;
R²⁰ and R′²⁰ are independently selected from halo, cyano, C₁-C₃ alkyl, cyclopropyl, haloalkyl, alkoxy, haloalkoxy, alkoxycarbonylamino, or the two substituents form an alkylenedioxy group or a haloalkylenedioxy group; and
R²¹, R²², R²³, and R²⁵ are as defined above in claim 23;
and pharmaceutically acceptable salts, and solvates thereof.

25. The compound according to claim 24 having formula Ib-4e: 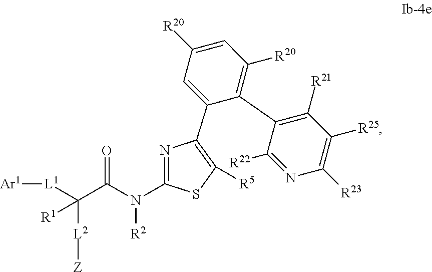
wherein,
Ar¹, L¹, L², R¹, R², R⁵, R²⁰, R′²⁰, R²¹, R²², R²³, R²⁵ and Z are as defined in claim 24;
and pharmaceutically acceptable salts, and solvates thereof.

26. The compound according to claim 20 having formula Ib-4k: 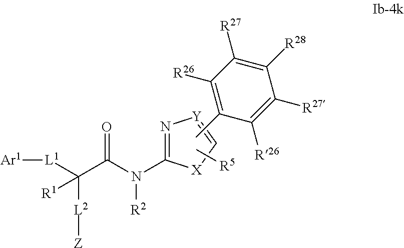
wherein
Ar¹, L¹, L², R¹, R², R⁵, X, Y, and Z are defined as in claim 20;
R²⁶, R′²⁶, R²⁷, R′²⁷, R²⁸ are independently selected from H, halo, cyano, alkyl, haloalkyl, cycloalkyl, cycloalkylalkyl, hydroxyl, alkoxy, haloalkoxy, cycloalkyloxy, alkylamino, carboxy, alkoxycarbonyl, alkylcarbonylamino, haloalkylcarbonylamino, cycloalkylcarbonylamino, acylamino, carbamoyl, alkoxycarbamoyl, cycloalkylcarbamoyl, alkylcarbamoylamino, cycloalkylaminocarbamoyl, alkylsulfonyl, haloalkylsulfonyl, sulfamoyl, alkylsulfamoyl, alkylsulfonylamino, haloalkylsulfonylamino, or two substituents form an alkylenedioxy group or a haloalkylenedioxy group;
and pharmaceutically acceptable salts, and solvates thereof.

27. The compound according to claim 26 having formula Ib-41: 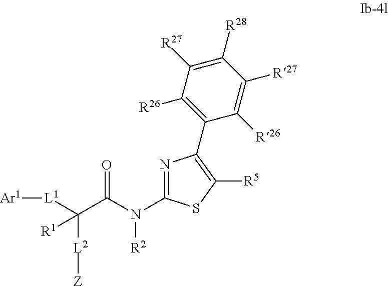
wherein
Ar¹, L², L², R¹, R², R⁵ R²⁶, R′²⁶, R^{27, R′}²⁷, R²⁸ and Z are as defined in claim 26;
and pharmaceutically acceptable salts, and solvates thereof.

28. The compound according to claim 20 having the formula Ib-4m: 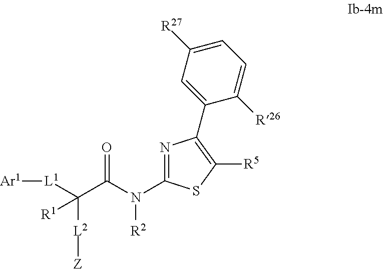
wherein
Ar¹, L¹, L², R¹, R², R⁵, and Z are as defined in respect claim 20; and
R′²⁶ and R²⁷ are independently selected from H, halo, cyano, alkyl, haloalkyl, cycloalkyl, cycloalkylalkyl, hydroxyl, alkoxy, haloalkoxy, cycloalkyloxy, alkylamino, carboxy, alkoxycarbonyl, alkylcarbonylamino, haloalkylcarbonylamino, cycloalkylcarbonylamino, acylamino, carbamoyl, alkoxycarbamoyl, cycloalkylcarbamoyl, alkylcarbamoylamino, cycloalkylaminocarbamoyl, alkylsulfonyl, haloalkylsulfonyl, sulfamoyl, alkylsulfamoyl, alkylsulfonylamino, haloalkylsulfonylamino, or the two substituents form an alkylenedioxy group or a haloalkylenedioxy group; and
pharmaceutically acceptable salts, and solvates thereof.

29. The compound according to claim 20 selected from the group consisting of:
1 6-((4-(2-chlorophenyl)thiazol-2-yl)carbamoyl)cyclohex-3-enecarboxylic acid
2 (R)-3-benzyl-4-((4-(2-chlorophenyl)thiazol-2-yl)amino)-4-oxobutanoic acid
3 (R)-3-benzyl-4-((4-(2,4-dichlorophenyl)thiazol-2-yl)amino)-4-oxobutanoic acid
4 (R)-4-benzyl-4-((4-(2-fluorophenyl)thiazol-2-yl)amino)-4-oxobutanoic acid
5 (R)-3-benzyl-4-((4-(3,4-dichlorophenyl)thiazol-2-yl)amino)-4-oxobutanoic acid
8 (R)-3-benzyl-4-((4-(4-cyanophenyl)thiazol-2-yl)amino)-4-oxobutanoic acid
9 (S)-4-((4-(2-chlorophenyl)thiazol-2-yl)amino)-4-oxo-3-phenylbutanoic acid
10 (Z)-4-((4-(2-chlorophenyl)thiazol-2-yl)amino)-4-oxobut-2-enoic acid
11 (R)-3-benzyl-4-oxo-4-((3-phenyl-1,2,4-thiadiazol-5-yl)amino)butanoic acid
12 (R)-3-benzyl-4-((4-(3-chlorophenyl)thiazol-2-yl)amino)-4-oxobutanoic acid
13 (R)-3-benzyl-4-oxo-4-((4-(3-(trifluoromethyl)phenyl)thiazol-2-yl)amino)butanoic acid
14 (R)-3-benzyl-4-((4-(2-chlorophenyl)thiazol-2-yl)(methyl)amino)-4-oxobutanoic acid
15 (R)-3-benzyl-4-((5-(2-chlorophenyl)pyridin-2-yl)amino)-4-oxobutanoic acid
16 (R)-3-((4-(2-chlorophenyl)thiazol-2-yl)carbamoyl)heptanoic acid
17 (R)-4-((4-(2-chlorophenyl)thiazol-2-yl)amino)-3-(4-fluorobenzyl)-4-oxobutanoic acid
18 (R)-4-((4-(2-chlorophenyl)thiazol-2-yl)amino)-3-(cyclohexylmethyl)-4-oxobutanoic acid
19 (R)-3-((4-(2-chlorophenyl)thiazol-2-yl)carbamoyl)-5-methylhexanoic acid
20 (R)-3-benzyl-4-((4-(2-chlorophenyl)-5-fluorothiazol-2-yl)amino)-4-oxobutanoic acid
21 (R)-3-benzyl-4-((5-chloro-4-(2-chlorophenyl)thiazol-2-yl)(methyl)amino)-4-oxobutanoic acid
22 (R)-4-(allyl(4-(2-chlorophenyl)thiazol-2-yl)amino)-3-benzyl-4-oxobutanoic acid
23 (R)-3-benzyl-4-((4-(2-chlorophenyl)thiazol-2-yl)(2-methoxy-2-oxoethyl)amino)-4-oxobutanoic acid
24 (R)-methyl-3-benzyl-4-((4-(2-chlorophenyl)thiazol-2-yl)amino)-4-oxobutanoate
26 (R)-3-(4-(2-chlorophenyl)thiazol-2-ylcarbamoyl)-5-phenylpentanoic acid
27 (S)-3-(4-(2-chlorophenyl)thiazol-2-ylcarbamoyl)-5-phenylpentanoic acid
28 (R)-4-(4-(2-chlorophenyl)thiazol-2-ylamino)-4-oxo-3-(4-(trifluoromethyl)benzyl)butanoic acid
29 (R)-4-((4-(2-chlorophenyl)thiazol-2-yl)amino)-4-oxo-3-(3-(trifluoromethyl)benzyl)butanoic acid
30 (R)-4-((4-(2-chlorophenyl)thiazol-2-yl)amino)-3-(2-cyanobenzyl)-4-oxobutanoic acid
31 (R)-4-((4-(2-chlorophenyl)thiazol-2-yl)amino)-3-(3-cyanobenzyl)-4-oxobutanoic acid
32 (R)-4-((4-(2-chlorophenyl)thiazol-2-yl)amino)-3-(4-cyanobenzyl)-4-oxobutanoic acid
33 (R)-4-((4-(2-chlorophenyl)thiazol-2-yl)amino)-3-(4-methoxybenzyl)-4-oxobutanoic acid
34 (R)-4-((4-(2-chlorophenyl)thiazol-2-yl)amino)-3-(3-methoxybenzyl)-4-oxobutanoic acid
35 (R)-4-((4-(2-chlorophenyl)thiazol-2-yl)amino)-3-(2-methoxybenzyl)-4-oxobutanoic acid
36 (R)-3-benzyl-4-((4-(2-methoxyphenyl)thiazol-2-yl)amino)-4-oxobutanoic acid
37 (R)-3-benzyl-4-oxo-4-(4-(2,4,6-trichlorophenyl)thiazol-2-ylamino)butanoic acid
38 (R)-4-benzyl-5-((4-(2-chlorophenyl)thiazol-2-yl)(methyl)amino)-5-oxopentanoic acid
39 (S)-4-benzyl-5-((4-(2-chlorophenyl)thiazol-2-yl)(methyl)amino)-5-oxopentanoic acid
40 (R)-methyl 4-benzyl-5-(4-(2-chlorophenyl)thiazol-2-ylamino)-5-oxopentanoate
41 (S)-methyl 4-benzyl-5-(4-(2-chlorophenyl)thiazol-2-ylamino)-5-oxopentanoate
42 (R)-3-benzyl-4-((4-(2-chlorophenyl)thiazol-2-yl)(cyclopropylmethyl)amino)-4-oxobutanoic acid
43 (R)-3-benzyl-4-(benzyl(4-(2-chlorophenyl)thiazol-2-yl)amino)-4-oxobutanoic acid
44 (R)-3-benzyl-4-((4-(2-chlorophenyl)thiazol-2-yl)(2,2,2-trifluoroethyl)amino)-4-oxobutanoic acid
45 (R)-4-((4-(2-chlorophenyl)thiazol-2-yl)(methyl)amino)-3-(4-methoxybenzyl)-4-oxobutanoic acid
46 (R)-4-((4-(2-chlorophenyl)thiazol-2-yl)(methyl)amino)-3-(3-methoxybenzyl)-4-oxobutanoic acid
47 (R)-4-((4-(2-chlorophenyl)thiazol-2-yl)(methyl)amino)-3-(2-methoxybenzyl)-4-oxobutanoic acid
48 (R)-4-((4-(2-chlorophenyl)thiazol-2-yl)(methyl)amino)-3-(4-cyanobenzyl)-4-oxobutanoic acid
49 (R)-4-((4-(2-chlorophenyl)thiazol-2-yl)(methyl)amino)-3-(3-cyanobenzyl)-4-oxobutanoic acid
50 (R)-4-((4-(2-chlorophenyl)thiazol-2-yl)(methyl)amino)-3-(2-cyanobenzyl)-4-oxobutanoic acid
51 (R)-3-(4-chlorobenzyl)-4-((4-(2-chlorophenyl)thiazol-2-yl)(methyl)amino)-4-oxobutanoic acid
52 (R)-3-(3-chlorobenzyl)-4-((4-(2-chlorophenyl)thiazol-2-yl)(methyl)amino)-4-oxobutanoic acid
53 (R)-3-(2-chlorobenzyl)-4-((4-(2-chlorophenyl)thiazol-2-yl)(methyl)amino)-4-oxobutanoic acid
54 (3S)-4-((4-(2-chlorophenyl)thiazol-2-yl)(methyl)amino)-3-(2,3-dihydro-1H-inden-1-yl)-4-oxobutanoic acid
55 (S)-4-((4-(2-chlorophenyl)thiazol-2-yl)(methyl)amino)-3-(2,3-dihydro-1H-inden-2-yl)-4-oxobutanoic acid
56 (R)-4-(benzo[d]thiazol-2-yl(methyl)amino)-3-benzyl-4-oxobutanoic acid
57 (R)-4-(benzo[d]oxazol-2-yl(methyl)amino)-3-benzyl-4-oxobutanoic acid
58 (R)-2-((1 H-tetrazol-5-yl)methyl)-N-(4-(2-chlorophenyl)thiazol-2-yl)-N-methyl-3-phenylpropanamide
59 (R)-2-benzyl-N-(4-(2-chlorophenyl)thiazol-2-yl)-N-methyl-3-(5-oxo-4,5-dihydro-1,2,4-oxadiazol-3-yl)propanamide
60 (R)-3-benzyl-4-((4-(2-chlorophenyl)-5-fluorothiazol-2-yl)(methyl)amino)-4-oxobutanoic acid
61 (S)-4-(4-(2-chlorophenyl)thiazol-2-ylamino)-3-cyclohexyl-4-oxobutanoic acid
62 (S)-4-((4-(2-chlorophenyl)thiazol-2-yl)(methyl)amino)-3-cyclohexyl-4-oxobutanoic acid
63 (S)-4-((4-(2-chlorophenyl)thiazol-2-yl)(methyl)amino)-4-oxo-3-phenylbutanoic acid
64 (3R)-3-(4-(2-chlorophenyl)thiazol-2-ylcarbamoyl)-4-phenylpentanoic acid
65 (R)-2-((1H-tetrazol-5-yl)methyl)-N-(4-(2-chlorophenyl)thiazol-2-yl)-3-phenylpropanamide
66 (R)-2-benzyl-N-(4-(2-chlorophenyl)thiazol-2-yl)-3-(5-oxo-4,5-dihydro-1,2,4-oxadiazol-3-yl)propanamide
68 (3R)-3-benzyl-4-(4-(2-chlorophenyl)thiazol-2-ylamino)-2-methyl-4-oxobutanoic acid
69 (R)-2-benzyl-N-(4-(2-chlorophenyl)thiazol-2-yl)-3-(3-hydroxyisoxazol-5-yl)propanamide
72 (E)-3-(4-(2-chlorophenyl)thiazol-2-ylcarbamoyl)-4-phenylbut-3-enoic acid
74 (Z)-4-((4-(2-chlorophenyl)thiazol-2-yl)(methyl)amino)-4-oxo-3-phenylbut-2-enoic acid
79 (R)-3-benzyl-4-(4-(2-chlorophenyl)thiazol-2-ylamino)-3-fluoro-4-oxobutanoic acid
80 (R)-3-benzyl-3-(4-(2-chlorophenyl)thiazol-2-ylcarbamoyl)hex-5-enoic acid
81 (E)-3-((4-(2-chlorophenyl)thiazol-2-yl)(methyl)carbamoyl)-4-phenylbut-3-enoic acid
82 (35)-3-((4-(2-chlorophenyl)thiazol-2-yl)(methyl)carbamoyl)-4-phenylpentanoic acid
83 (R)-3-benzyl-4-((3-(2-chlorophenyl)-1,2,4-thiadiazol-5-yl)(methyl)amino)-4-oxobutanoic acid
84 (R)-3-benzyl-4-((3-(2-chlorophenyl)-1,2,4-oxadiazol-5-yl)(methyl)amino)-4-oxobutanoic acid
86 (R)-2-benzyl-N-(4-(2-chlorophenyl)thiazol-2-yl)-3-(3-hydroxyisoxazol-5-yl)-N-methylpropanamide
89 (R)-4-((4-(2-chlorophenyl)thiazol-2-yl)(methyl)amino)-3-(cyclohexylmethyl)-4-oxobutanoic acid
90 (R)-3-((4-(2-chlorophenyl)thiazol-2-yl)(methyl)carbamoyl)-5-methylhexanoic acid
91 (R)-3-benzyl-4-((4-(2-cyanophenyl)thiazol-2-yl)(methyl)amino)-4-oxobutanoic acid
92 (R)-4-(4-(2-chlorophenyl)thiazol-2-ylamino)-4-oxo-3-phenylbutanoic acid
93 (R)-4-(4-(2-chlorophenyl)thiazol-2-ylamino)-3-(3-fluorobenzyl)-4-oxobutanoic acid
94 (S)-3-((4-(2-chlorophenyl)thiazol-2-yl)(methyl)carbamoyl)-4-methylpentanoic acid
95 4-((4-(2-chlorophenyl)thiazol-2-yl)(methyl)amino)-4-oxo-3-((tetrahydro-2H-pyran-4-yl)methyl)butanoic acid
96 (R)-3-benzyl-4-((4-(2-chlorophenyl)thiazol-2-yl)(ethyl)amino)-4-oxobutanoic acid
97 (R)-3-benzyl-4-((4-(2-chlorophenyl)thiazol-2-yl)(cyclopropyl)amino)-4-oxobutanoic acid
98 cis-6-(4-(2-chlorophenyl)thiazol-2-ylcarbamoyl)cyclohex-3-enecarboxylic acid
99 4-((4-(2-chlorophenyl)thiazol-2-yl)(methyl)amino)-3-(4-methoxybenzyl)-4-oxobutanoic acid
100 cis-6-((4-(2-chlorophenyl)thiazol-2-yl)(methyl)carbamoyl)cyclohex-3-enecarboxylic acid
101 cis-2-((4-(2-chlorophenyl)thiazol-2-yl)(methyl)carbamoyl)cyclohexanecarboxylic acid
102 (R)-3-benzyl-4-(4-(2,5-dimethylthiophen-3-yl)thiazol-2-ylamino)-4-oxobutanoic acid
103 4-((4-(2-chlorophenyl)thiazol-2-yl)(methyl)amino)-3-(cyclohexylmethyl)-4-oxobutanoic acid
105 4-((4-(2-chlorophenyl)thiazol-2-yl)(methyl)amino)-3-(cyclopentylmethyl)-4-oxobutanoic acid
106 (3S,4R)-3-((4-(2-chlorophenyl)thiazol-2-yl)(methyl)carbamoyl)-4-phenylpentanoic acid
107 (R)-3-benzyl-4-(methyl(4-(2-(thiophen-3-yl)phenyl)thiazol-2-yl)amino)-4-oxobutanoic acid
108 (R)-3-benzyl-4-((4-(2-(6-chloropyridin-3-yl)phenyl)thiazol-2-yl)(methyl)amino)-4-oxobutanoic acid
109 (R)-4-((4-(2-chlorophenyl)thiazol-2-yl)(methyl)amino)-4-oxo-3-(phenylamino)butanoic acid
110 4-((4-(2-chlorophenyl)thiazol-2-yl)(methyl)amino)-3-(4-methylbenzyl)-4-oxobutanoic acid
111 (R)-4-((4-([1,1′-biphenyl]-2-yl)thiazol-2-yl)(methyl)amino)-3-benzyl-4-oxobutanoic acid
112 (R)-3-benzyl-4-(4-(2,5-dichlorothiophen-3-yl)thiazol-2-ylamino)-4-oxobutanoic acid
113 4-((4-(2-chlorophenyl)thiazol-2-yl)(methyl)amino)-3-(cyclopropylmethyl)-4-oxobutanoic acid
114 4-((4-(2-chlorophenyl)thiazol-2-yl)(methyl)amino)-4-oxo-3-(thiazol-4-ylmethyl)butanoic acid
115 (R)-3-benzyl-4-((4-(2-(6-(dimethylamino)pyridin-3-yl)phenyl)thiazol-2-yl)(methyl)amino)-4-oxobutanoic acid
116 (R)-3-benzyl-4-((4-(2-(6-methoxypyridin-3-yl)phenyl)thiazol-2-yl)(methyl)amino)-4-oxobutanoic acid
117 (R)-3-benzyl-4-((4-(2-(2-methoxypyridin-3-yl)phenyl)thiazol-2-yl)(methyl)amino)-4-oxobutanoic acid
118 (R)-3-benzyl-4-((4-(2-((ethoxycarbonyl)amino)phenyl)thiazol-2-yl)(methyl)amino)-4-oxobutanoic acid
119 (R)-3-benzyl-4-((4-(2-(6-fluoropyridin-3-yl)phenyl)thiazol-2-yl)(methyl)amino)-4-oxobutanoic acid
120 (R)-3-benzyl-4-(methyl(4-(2-(6-methylpyridin-3-yl)phenyl)thiazol-2-yl)amino)-4-oxobutanoic acid
121 (R)-4-((2-amino-2-oxoethyl)(4-(2-chlorophenyl)thiazol-2-yl)amino)-3-benzyl-4-oxobutanoic acid
122 (R)-3-benzyl-4-oxo-4-((4-(3-(trifluoromethoxy)phenyl)thiazol-2-yl)amino)butanoic acid
123 (R)-3-benzyl-4-((4-(2,5-dichlorophenyl)thiazol-2-yl)amino)-4-oxobutanoic acid
124 (R)-3-benzyl-4-((4-(3-chloro-4-fluorophenyl)thiazol-2-yl)amino)-4-oxobutanoic acid
125 (R)-3-benzyl-4-((4-(3-chloro-4-methoxyphenyl)thiazol-2-yl)amino)-4-oxobutanoic acid
126 (R)-3-benzyl-4-((4-(2-chlorophenyl)thiazol-2-yl)(3-methoxy-3-oxopropyl)amino)-4-oxobutanoic acid
127 3-(bicyclo[2.2.1]heptan-2-ylmethyl)-4-((4-(2-chlorophenyl)thiazol-2-yl)(methyl)amino)-4-oxobutanoic acid
128 (R)-3-benzyl-4-((4-(2-(6-ethoxypyridin-3-yl)phenyl)thiazol-2-yl)(methyl)amino)-4-oxobutanoic acid
129 (R)-3-benzyl-4-((4-(4′-methoxy-[1,1′-biphenyl]-2-yl)thiazol-2-yl)(methyl)amino)-4-oxobutanoic acid
130 (R)-3-benzyl-4-((4-(2,5-dichlorophenyl)thiazol-2-yl)(methyl)amino)-4-oxobutanoic acid
131 (R)-1-(5-(2-(2-(2-benzyl-3-carboxy-N-methylpropanamido)thiazol-4-yl)phenyl)pyridin-2-yl)pyrrolidin-1-ium 2,2,2-trifluoroacetate
132 (R)-4-(2′-(2-(2-benzyl-3-carboxy-N-methylpropanamido)thiazol-4-yl)-[1,1′-biphenyl]-4-yl)morpholin-4-ium 2,2,2-trifluoroacetate
133 (R)-3-benzyl-4-(methyl(4-(2-(6-morpholinopyridin-3-yl)phenyl)thiazol-2-yl)amino)-4-oxobutanoic acid
134 (R)-3-benzyl-4-((4-(3′-chloro-[-1,1′-biphenyl]-2-yl)thiazol-2-yl)(methyl)amino)-4-oxobutanoic acid
135 (R)-3-benzyl-4-((4-(2-(furan-3-yl)phenyl)thiazol-2-yl)(methyl)amino)-4-oxobutanoic acid
136 (R)-3-benzyl-4-((4-(2-(6-(2-methoxyethoxy)pyridin-3-yl)phenyl)thiazol-2-yl)(methyl)amino)-4-oxobutanoic acid
138 (R)-3-benzyl-4-((4-(4′-isopropyl-[1,1′-biphenyl]-2-yl)thiazol-2-yl)(methyl)amino)-4-oxobutanoic acid
139 (R)-3-(cyclopentylmethyl)-4-((4-(2-(6-methoxypyridin-3-yl)phenyl)thiazol-2-yl)(methyl)amino)-4-oxobutanoic acid
140 (R)-3-benzyl-4-((4-(2-(5-fluoro-6-methoxypyridin-3-yl)phenyl)thiazol-2-yl)(methyl)amino)-4-oxobutanoic acid
141 (R)-3-benzyl-4-(methyl(4-(2-(6-((tetrahydro-2H-pyran-4-yl)oxy)pyridin-3-yl)phenyl)thiazol-2-yl)amino)-4-oxobutanoic acid
142 (R)-3-benzyl-4-(cyclopropyl(4-(2,5-dichlorophenyl)thiazol-2-yl)amino)-4-oxobutanoic acid
143 4-((4-(2-chlorophenyl)thiazol-2-yl)(methyl)amino)-3-(furan-2-ylmethyl)-4-oxobutanoic acid
144 (R)-3-benzyl-4-((4-(2-cyclopropylphenyl)thiazol-2-yl)(methyl)amino)-4-oxobutanoic acid
145 (R)-3-benzyl-4-((4-(4′-(dimethylamino)41,1′-biphenyl]-2-yl)thiazol-2-yl)(methyl)amino)-4-oxobutanoic acid
146 (R)-3-benzyl-4-((4-(3′-fluoro-[1,1′-biphenyl]-2-yl)thiazol-2-yl)(methyl)amino)-4-oxobutanoic acid
147 (R)-3-benzyl-4-((4-(3′,5′-difluoro-[1,1′-biphenyl]-2-yl)thiazol-2-yl)(methyl)amino)-4-oxobutanoic acid
148 (R)-3-benzyl-4-((4-(2-chloro-6-fluorophenyl)thiazol-2-yl)amino)-4-oxobutanoic acid
149 (R)-3-benzyl-4-((4-(4′-chloro-[1,1′-biphenyl]-2-yl)thiazol-2-yl)(methyl)amino)-4-oxobutanoic acid
150 (R)-3-benzyl-4-(methyl(4-(2-(6-(2-oxopyrrolidin-1-yl)pyridin-3-yl)phenyl)thiazol-2-yl)amino)-4-oxobutanoic acid
151 (R)-3-benzyl-4-((4-(4-chloro-2-(6-methoxypyridin-3-yl)phenyl)thiazol-2-yl)(methyl)amino)-4-oxobutanoic acid
152 (R)-3-benzyl-4-((4-(5-chloro-2-(6-methoxypyridin-3-yl)phenyl)thiazol-2-yl)(methyl)amino)-4-oxobutanoic acid
153 (R)-3-benzyl-4-((4-(3-fluoro-2-(6-methoxypyridin-3-yl)phenyl)thiazol-2-yl)(methyl)amino)-4-oxobutanoic acid
154 (3R)-4-((4-(2-chlorophenyl)thiazol-2-yl)(methyl)amino)-4-oxo-3-((tetrahydrofuran-2-yl)methyl)butanoic acid
155 (R)-3-benzyl-4-((4-(2-chlorophenyl)thiazol-2-yl)(2-hydroxyethyl)amino)-4-oxobutanoic acid
156 (R)-3-benzyl-4-((4-(2-chlorophenyl)thiazol-2-yl)(3-hydroxypropyl)amino)-4-oxobutanoic acid
157 (R)-3-benzyl-4-((4-(2-(5-chloro-6-methoxypyridin-3-yl)phenyl)thiazol-2-yl)(methyl)amino)-4-oxobutanoic acid
158 (R)-3-benzyl-4-((4-(2-(6-(benzyloxy)pyridin-3-yl)phenyl)thiazol-2-yl)(methyl)amino)-4-oxobutanoic acid
159 (R)-3-(cyclopentylmethyl)-4-((4-(2,5-dichlorophenyl)thiazol-2-yl)(methyl)amino)-4-oxobutanoic acid
160 (R)-4-((4-(2-(6-methoxypyridin-3-yl)phenyl)thiazol-2-yl)(methyl)amino)-4-oxo-3-((tetrahydro-2H-pyran-4-yl)methyl)butanoic acid
161 (R)-3-benzyl-4-((4-(2-chloro-5-(trifluoromethyl)phenyl)thiazol-2-yl)(methyl)amino)-4-oxobutanoic acid
162 (R)-3-benzyl-4-((4-(2-chloro-5-fluorophenyl)thiazol-2-yl)(methyl)amino)-4-oxobutanoic acid
163 (R)-3-benzyl-4-((4-(3,5-dichlorophenyl)thiazol-2-yl)(methyl)amino)-4-oxobutanoic acid
164 (R)-3-benzyl-4-((4-(3-(difluoromethoxy)phenyl)thiazol-2-yl)(methyl)amino)-4-oxobutanoic acid
165 (R)-4-((4-(2-chlorophenyl)thiazol-2-yl)(methyl)amino)-3-(cyclopentylmethyl)-4-oxobutanoic acid
166 (R)-3-(cyclopentylmethyl)-4-(cyclopropyl(4-(2,5-dichlorophenyl)thiazol-2-amino)-4-oxobutanoic acid
167 (R)-4-(cyclopropyl(4-(2,5-dichlorophenyl)thiazol-2-yl)amino)-4-oxo-3-((tetrahydro-2H-pyran-4-yl)methyl)butanoic acid
168 (R)-4-((4-(2,5-dichlorophenyl)thiazol-2-yl)(methyl)amino)-4-oxo-3-((tetrahydro-2H-pyran-4yl)methyl)butanoic acid
169 (R)-3-(cyclopentylmethyl)-4-(cyclopropyl(4-(2-(6-methoxypyridin-3-yl)phenyl)thiazol-2-yl)amino)-4-oxobutanoic acid
170 (R)-3-benzyl-4-((2-hydroxyethyl)(4-(2-(6-methoxypyridin-3-yl)phenyl)thiazol-2-yl)amino)-4-oxobutanoic acid
171 (R)-3-(cyclopentylmethyl)-4-(methyl(4-(2-(6-morpholinopyridin-3-yl)phenyl)thiazol-2-yl)amino)-4-oxobutanoic acid
172 (R)-3-(cyclopentylmethyl)-4-((4-(2,5-dichlorophenyl)thiazol-2-yl)(2-hydroxyethypamino)-4-oxobutanoic acid
173 (R)-4-((4-(2-chlorophenyl)thiazol-2-yl)(methyl)amino)-4-oxo-3-((tetrahydro-2H-pyran-4-yl)methyl)butanoic acid
174 (R)-3-benzyl-4-((4-(5-chloro-2-(trifluoromethyl)phenyl)thiazol-2-yl)(methyl)amino)-4-oxobutanoic acid
175 (R)-3-benzyl-4-(methyl(4-(2,3,5-trichlorophenyl)thiazol-2-yl)amino)-4-oxobutanoic acid
176 (R)-3-benzyl-4-((4-(4-chloro-[1,1′-biphenyl]-3-yl)thiazol-2-yl)(methyl)amino)-4-oxobutanoic acid
177 (R)-3-benzyl-4-((4-(2-chloro-5-(6-methoxypyridin-3-yl)phenyl)thiazol-2-yl)(methyl)amino)-4-oxobutanoic acid
178 (R)-3-benzyl-4-(cyclopropyl(4-(2-(6-methoxypyridin-3-yl)phenyl)thiazol-2-yl)amino)-4-oxobutanoic acid
179 (R)-4-(cyclopropyl(4-(2-(6-methoxypyridin-3-yl)phenyl)thiazol-2-yl)amino)-4-oxo-3-((tetrahydro-2H-pyran-4-yl)methyl)butanoic acid
180 (R)-3-benzyl-4-(cyclopropyl(4-(2-(6-morpholinopyridin-3-yl)phenyl)thiazol-2-yl)amino)-4-oxobutanoic acid
181 (R)-3-(cyclopentylmethyl)-4-(cyclopropyl(4-(2-(6-morpholinopyridin-3-yl)phenyl)thiazol-2-yl)amino)-4-oxobutanoic acid
182 (R)-3-benzyl-4-(methyl(4-(2-(4-methyl-3,4-dihydro-2H-pyrido[3,2-13][1,4]oxazin-7-yl)phenyl)thiazol-2-yl)amino)-4-oxobutanoic acid
183 (R)-3-(cyclopentylmethyl)-4-(cyclopropyl(4-(2-(6-(2-oxopyrrolidin-1-yl)pyridin-3-yl)phenyl)thiazol-2-yl)amino)-4-oxobutanoic acid
184 (R)-4-(cyclopropyl(4-(2-(6-morpholinopyridin-3-yl)phenyl)thiazol-2-yl)amino)-4-oxo-3-((tetrahydro-2H-pyran-4-yl)methyl)butanoic acid
185 (R)-3-benzyl-4-(methyl(4-(2-(trifluoromethoxy)phenyl)thiazol-2-yl)amino)-4-oxobutanoic acid
186 (R)-4-((4-(2-chloro-5-fluorophenyl)thiazol-2-yl)(cyclopropyl)amino)-3-(cyclopentylmethyl)-4-oxobutanoic acid
187 (R)-3-(cyclopentylmethyl)-4-(methyl(4-(2-(6-(2-oxopyrrolidin-1-yl)pyridin-3-yl)phenyl)thiazol-2-yl)amino)-4-oxobutanoic acid
188 (R)-3-benzyl-4-(cyclopropyl(4-(3-(difluoromethoxy)phenyl)thiazol-2-yl)amino)-4-oxobutanoic acid
189 (R)-3-benzyl-4-((4-(2-chloro-5-fluorophenyl)thiazol-2-yl)(cyclopropyl)amino)-4-oxobutanoic acid
190 (R)-4-((4-(2-chloro-5-fluorophenyl)thiazol-2-yl)(cyclopropyl)amino)-4-oxo-3-((tetrahydro-2H-pyran-4-yl)methyl)butanoic acid
191 (R)-3-benzyl-4-((4-(2-chloro-5-(trifluoromethyl)phenyl)thiazol-2-yl)(cyclopropyl)amino)-4-oxobutanoic acid
192 (R)-3-benzyl-4-((4-(2-(difluoromethoxy)phenyl)thiazol-2-yl)(methyl)amino)-4-oxobutanoic acid
193 (R)-4-((4-(2-chloro-5-(trifluoromethyl)phenyl)thiazol-2-yl)(cyclopropyl)amino)-4-oxo-3-((tetrahydro-2H-pyran-4-yl)methyl)butanoic acid
194 (R)-3-(cyclopentylmethyl)-4-(cyclopropyl(4-(2-(4-methyl-3,4-dihydro-2H-pyrido[3,2-b][1,4]oxazin-7-yl)phenyl)thiazol-2-yl)amino)-4-oxobutanoic acid
195 (3R,45)-3-((4-(2-chlorophenyl)thiazol-2-yl)(methyl)carbamoyl)-4-phenylpentanoic acid
198 4-((4-(2-chlorophenyl)thiazol-2-yl)(methyl)amino)-3-(morpholinomethyl)-4-oxobutanoic acid
199 (R)-3-benzyl-4-((4-(2-chlorophenyl)thiazol-2-yl)(2-methoxyethyl)amino)-4-oxobutanoic acid
200 (R)-4-((4-(2-chlorophenyl)thiazol-2-yl)(methyl)amino)-3-(cyclopentylamino)-4-oxobutanoic acid
201 (R)-3-benzyl-4-((2-(benzyloxy)ethyl)(4-(2-chlorophenyl)thiazol-2-yl)amino)-4-oxobutanoic acid
202 (R)-3-benzyl-4-((4-(5-methylfuran-2-yl)thiazol-2-yl)amino)-4-oxobutanoic acid
204 (R)-3-benzyl-4-((4-(5-chloro-2-methoxyphenyl)thiazol-2-yl)amino)-4-oxobutanoic acid
205 4-((4-(2-chlorophenyl)thiazol-2-yl)(methyl)amino)-3-(4-hydroxybenzyl)-4-oxobutanoic acid
206 (R)-3-benzyl-4-((4-(4′-cyano-[1,1′-biphenyl]-2-yl)thiazol-2-yl)(methyl)amino)-4-oxobutanoic acid
208 (R)-3-benzyl-4-((4-(3′-methoxy-[1,1′-biphenyl]-2-yl)thiazol-2-yl)(methyl)amino)-4-oxobutanoic acid
209 4-((4-(2-chlorophenyl)thiazol-2-yl)(methyl)amino)-3-((2-methylthiazol-4-yl)methyl)-4-oxobutanoic acid
210 4-((4-(2-chlorophenyl)thiazol-2-yl)(methyl)amino)-3-((5-methylisoxazol-3-yl)methyl)-4-oxobutanoic acid
211 (R)-3-benzyl-4-((4-(2′-chloro-[1,1′-biphenyl]-2-yl)thiazol-2-yl)(methyl)amino)-4-oxobutanoic acid
212 (R)-3-benzyl-4-((4-(2-(2-methoxypyrimidin-5-yl)phenyl)thiazol-2-yl)(methyl)amino)-4-oxobutanoic acid
213 (R)-3-benzyl-4-((4-(2,5-difluorophenyl)thiazol-2-yl)amino)-4-oxobutanoic acid
214 4-((4-(2-chlorophenyl)thiazol-2-yl)(methyl)amino)-3-(oxazol-4-ylmethyl)-4-oxobutanoic acid
215 (3R)-4-((4-(2-chlorophenyl)thiazol-2-yl)(methyl)amino)-4-oxo-3-((tetrahydrofuran-3-yl)methyl)butanoic acid
216 (R)-3-benzyl-4-(methyl(4-(2-(8-methyl-7-oxo-5,6,7,8-tetrahydro-1,8-naphthyridin-3-yl)phenyl)thiazol-2-yl)amino)-4-oxobutanoic acid
217 (R)-3-benzyl-4-(methyl(4-(2-(1-methyl-1H-pyrrolo[2,3-b]pyridin-5-yl)phenyl)thiazol-2-yl)amino)-4-oxobutanoic acid
218 (R)-3-(cyclopentylmethyl)-4-(cyclopropyl(4-(2-(6-(dimethylamino)pyridin-3-yl)phenyl)thiazol-2-yl)amino)-4-oxobutanoic acid
219 (R)-4-((4-(2-(5-chloro-6-methoxypyridin-3-yl)phenyl)thiazol-2-yl)(cyclopropyl)amino)-3-(cyclopentylmethyl)-4-oxobutanoic acid
220 (R)-3-(cyclopentylmethyl)-4-(cyclopropyl(4-(2-(5-fluoro-6-methoxypyridin-3-yl)phenyl)thiazol-2-yl)amino)-4-oxobutanoic acid
221 (R)-3-benzyl-4-((4-(2-chloro-5-(difluoromethoxy)phenyl)thiazol-2-yl)(methyl)amino)-4-oxobutanoic acid
222 (R)-3-benzyl-4-((4-(5-chloro-2-(5-chloro-6-methoxypyridin-3-yl)phenyl)thiazol-2-yl)(methyl)amino)-4-oxobutanoic acid
223 (R)-4-((4-(5-chloro-2-(5-chloro-6-methoxypyridin-3-yl)phenyl)thiazol-2-yl)(cyclopropyl)amino)-3-(cyclopentylmethyl)-4-oxobutanoic acid
224 (R)-4-((4-(5-chloro-2-(5-fluoro-6-methoxypyridin-3-yl)phenyl)thiazol-2-yl)(cyclopropyl)amino)-3-(cyclopentylmethyl)-4-oxobutanoic acid
225 (S)-3-benzyl-4-((4-(2-chlorophenyl)thiazol-2-yl)amino)-4-oxobutanoic acid
230 3-([1,1′-biphenyl]-4-ylmethyl)-4-((4-(2-chlorophenyl)thiazol-2-yl)(methyl)amino)-4-oxobutanoic acid
231 (R)-3-benzyl-4-((4-(1-methyl-1H-pyrazol-4-yl)thiazol-2-yl)amino)-4-oxobutanoic acid
232 (R)-3-benzyl-4-((4-(4-methyl-1,2,5-oxadiazol-3-yl)thiazol-2-yl)amino)-4-oxobutanoic acid
233 (R)-3-benzyl-4-(methyl(4-(2-(1-methyl-1H-pyrazol-4-yl)phenyl)thiazol-2-yl)amino)-4-oxobutanoic acid
234 (3R)-3-benzyl-4-((4-(2-(3,5-dimethylisoxazol-4-yl)phenyl)thiazol-2-yl)(methyl)amino)-4-oxobutanoic acid
235 (R)-3-benzyl-4-((4-((2-chlorophenyl)carbamoyl)thiazol-2-yl)amino)-4-oxobutanoic acid
237 (R)-3-benzyl-4-(methyl(4-(2-(2-oxopyrrolidin-1-yl)phenyl)thiazol-2-yl)amino)-4-oxobutanoic acid
238 (S)-2-((1-((4-(2-chlorophenyl)thiazol-2-yl)(methyl)amino)-1-oxo-3-phenylpropan-2-yl)oxy)acetic acid
240 (R)-3-benzyl-4-((4-(2-(1-(2-methoxyethyl)-6-oxo-1,6-dihydropyridin-3-yl)phenyl)thiazol-2-yl)(methyl)amino)-4-oxobutanoic acid
241 (R)-3-benzyl-4-(methyl(4-(2-(1-methyl-6-oxo-1,6-dihydropyridin-3-yl)phenyl)thiazol-2-yl)amino)-4-oxobutanoic acid
242 4-((4-(2-chlorophenyl)thiazol-2-yl)(methyl)amino)-3-((2,5-dimethyloxazol-4-yl)methyl)-4-oxobutanoic acid
243 4-((4-(2-chlorophenyl)thiazol-2-yl)(methyl)amino)-3-((1-methyl-1H-pyrazol-5-yl)methyl)-4-oxobutanoic acid
244 (R)-3-benzyl-4-((4-(2-(6-hydroxypyridin-3-yl)phenyl)thiazol-2-yl)(methyl)amino)-4-oxobutanoic acid
245 (R)-3-benzyl-4-((4-(2-chlorophenyl)thiazol-2-yl)((S)-2-hydroxypropyl)amino)-4-oxobutanoic acid
246 (R)-3-benzyl-4-((4-(2-chlorophenyl)thiazol-2-yl)((R)-2-hydroxypropyl)amino)-4-oxobutanoic acid
247 (R)-3-(cyclohexylmethyl)-4-(cyclopropyl(4-(2-(6-methoxypyridin-3-yl)phenyl)thiazol-2-yl)amino)-4-oxobutanoic acid
248 (R)-3-benzyl-4-((4-(5-fluoro-2-(6-methoxypyridin-3-yl)phenyl)thiazol-2-yl)(methyl)amino)-4-oxobutanoic acid
250 (R)-3-benzyl-4-((4-(4,5-difluoro-2-(6-methoxypyridin-3-yl)phenyl)thiazol-2-yl)(methyl)amino)-4-oxobutanoic acid
251 (R)-4-((4-(2,5-dichlorophenyl)thiazol-2-yl)(methyl)amino)-3-(furan-2-ylmethyl)-4-oxobutanoic acid
252 (R)-4-((4-(2-chloro-5-fluorophenyl)thiazol-2-yl)(methyl)amino)-3-(furan-2-ylmethyl)-4-oxobutanoic acid
253 (R)-3-(furan-2-ylmethyl)-4-((4-(2-(6-methoxypyridin-3-yl)phenyl)thiazol-2-yl)(methyl)amino)-4-oxobutanoic acid
254 (S)-4-((4-(2-chlorophenyl)thiazol-2-yl)(methyl)amino)-4-oxo-3-(thiophen-2-ylmethyl)butanoic acid
255 (R)-4-((4-(5-chloro-2-(6-methoxypyridin-3-yl)phenyl)thiazol-2-yl)(cyclopropyl)amino)-3-(cyclopentylmethyl)-4-oxobutanoic acid
256 (R)-3-benzyl-4-(cyclopropyl(4-(2-(6-(2-oxopyrrolidin-1-yl)pyridin-3-yl)phenyl)thiazol-2-yl)amino)-4-oxobutanoic acid
257 (R)-3-benzyl-4-((4-(2,3-dichlorophenyl)thiazol-2-yl)(methyl)amino)-4-oxobutanoic acid
258 (R)-3-benzyl-4-(methyl(4-(3-(trifluoromethoxy)phenyl)thiazol-2-yl)amino)-4-oxobutanoic acid
259 (R)-4-(cyclopropyl(4-(3-(difluoromethoxy)phenyl)thiazol-2-yl)amino)-4-oxo-3-((tetrahydro-2H-pyran-4-yl)methyl)butanoic acid
260 (R)-4-((4-(2-chlorophenyl)thiazol-2-yl)(methyl)amino)-3-(furan-2-ylmethyl)-4-oxobutanoic acid
261 (R)-4-(methyl(4-(3-(trifluoromethoxy)phenyl)thiazol-2-yl)amino)-4-oxo-3-((tetrahydro-2H-pyran-4-yl)methyl)butanoic acid
262 (R)-3-benzyl-4-(cyclopropyl(4-(3-(trifluoromethoxy)phenyl)thiazol-2-yl)amino)-4-oxobutanoic acid
263 (R)-4-(cyclopropyl(4-(3-(trifluoromethoxy)phenyl)thiazol-2-yl)amino)-4-oxo-3-((tetrahydro-2H-pyran-4-yl)methyl)butanoic acid
264 (R)-3-benzyl-4-((4-(2-(6-isopropoxypyridin-3-yl)phenyl)thiazol-2-yl)(methyl)amino)-4-oxobutanoic acid
265 (R)-3-benzyl-4-((4-(2-(6-(cyclopropylmethoxy)pyridin-3-yl)phenyl)thiazol-2-yl)(methyl)amino)-4-oxobutanoic acid
266 (R)-3-benzyl-4-((4-(2-(6-(methoxymethyl)pyridin-3-yl)phenyl)thiazol-2-yl)(methyl)amino)-4-oxobutanoic acid
267 (R)-3-benzyl-4-((4-(2-(6-((dimethylamino)methyl)pyridin-3-yl)phenyl)thiazol-2-yl)(methyl)amino)-4-oxobutanoic acid
268 (R)-3-benzyl-4-(methyl(4-(2-(6-(N-methylcyclopropanecarboxamido)pyridin-3-yl)phenyl)thiazol-2-yl)amino)-4-oxobutanoic acid
269 (R)-3-benzyl-4-((4-(2-(6-(dimethylcarbamoyl)pyridin-3-yl)phenyl)thiazol-2-yl)(methyl)amino)-4-oxobutanoic acid
270 (R)-4-((4-(2-(6-(4H-1,2,4-triazol-4-yl)pyridin-3-yl)phenyl)thiazol-2-yl)(methyl)amino)-3-benzyl-4-oxobutanoic acid
271 (R)-3-benzyl-4-(methyl(4-(2-(6-(3-methyl-2-oxoimidazolidin-1-yl)pyridin-3-yl)phenyl)thiazol-2-yl)amino)-4-oxobutanoic acid
272 (R)-3-benzyl-4-(methyl(4-(2-(1-methyl-2-oxo-2,3-dihydro-1H-pyrrolo[2,3-b]pyridin-5-yl)phenyl)thiazol-2-yl)amino)-4-oxobutanoic acid
273 (R)-3-benzyl-4-(methyl(4-(2-(3-methyl-3H-imidazo[4,5-b]pyridin-6-yl)phenyl)thiazol-2-yl)amino)-4-oxobutanoic acid
274 (R)-3-benzyl-4-((4-(2-(6-(benzyl(methyl)amino)pyridin-3-yl)phenyl)thiazol-2-yl)(methyl)amino)-4-oxobutanoic acid
275 (R)-3-benzyl-4-((4-(2-(6-(cyclohexyl(methyl)amino)pyridin-3-yl)phenyl)thiazol-2-yl)(methyl)amino)-4-oxobutanoic acid
276 (R)-3-benzyl-4-(methyl(4-(2-(6-(4-methylpiperazin-1-yl)pyridin-3-yl)phenyl)thiazol-2-yl)amino)-4-oxobutanoic acid
277 (R)-4-((4-(5-chloro-2-(6-methoxypyridin-3-yl)phenyl)thiazol-2-yl)(cyclopropyl)amino)-3-(cyclopentylmethyl)-4-oxobutanoic acid
278 (R)-3-(cyclopentylmethyl)-4-(cyclopropyl(4-(3-fluoro-2-(6-methoxypyridin-3-yl)phenyl)thiazol-2-yl)amino)-4-oxobutanoic acid
279 (R)-3-benzyl-4-((4-(2-(5-chloro-6-methoxypyridin-3-yl)-3-fluorophenyl)thiazol-2-yl)(methyl)amino)-4-oxobutanoic acid
280 (R)-3-benzyl-4-((4-(3-fluoro-2-(5-fluoro-6-methoxypyridin-3-yl)phenyl)thiazol-2-yl)(methyl)amino)-4-oxobutanoic acid
281 (R)-3-benzyl-4-((4-(5-chloro-2-(5-fluoro-6-methoxypyridin-3-yl)phenyl)thiazol-2-yl)(methyl)amino)-4-oxobutanoic acid
282 (R)-3-benzyl-4-((4-(3,5-difluoro-2-(6-methoxypyridin-3-yl)phenyl)thiazol-2-yl)(methyl)amino)-4-oxobutanoic acid
283 (R)-4-((4-(2-chlorophenyl)thiazol-2-yl)(methyl)amino)-4-oxo-3-(((S)-tetrahydrofuran-2-yl)methyl)butanoic acid
284 (R)-4-((4-(2-chlorophenyl)thiazol-2-yl)(methyl)amino)-4-oxo-3-(((R)-tetrahydrofuran-2-yl)methyl)butanoic acid
285 (R)-4-((4-(2-chloro-5-(trifluoromethyl)phenyl)thiazol-2-yl)(methyl)amino)-3-(furan-2-ylmethyl)-4-oxobutanoic acid
286 (R)-4-((4-(2-chloro-5-(trifluoromethoxy)phenyl)thiazol-2-yl)(methyl)amino)-3-(furan-2-ylmethyl)-4-oxobutanoic acid
287 (R)-4-((4-(2-chloro-5-(difluoromethoxy)phenyl)thiazol-2-yl)(methyl)amino)-3-(furan-2-ylmethyl)-4-oxobutanoic acid
288 (R)-4-((4-(2-chlorophenyl)thiazol-2-yl)(cyclopropyl)amino)-3-(furan-2-ylmethyl)-4-oxobutanoic acid
289 (R)-4-((4-(5-chloro-2-(6-methoxypyridin-3-yl)phenyl)thiazol-2-yl)(methyl)amino)-3-(furan-2-ylmethyl)-4-oxobutanoic acid
290 (R)-4-((4-(2-chloro-5-(6-methoxypyridin-3-yl)phenyl)thiazol-2-yl)(methyl)amino)-3-(furan-2-ylmethyl)-4-oxobutanoic acid
291 (R)-3-(furan-2-ylmethyl)-4-((4-(2-(6-methoxypyridin-3-yl)-5-(trifluoromethyl)phenyl)thiazol-2-yl)(methyl)amino)-4-oxobutanoic acid
292 (R)-3-(furan-2-ylmethyl)-4-((4-(2-(6-methoxypyridin-3-yl)-5-(trifluoromethoxy)phenyl)thiazol-2-yl)(methyl)amino)-4-oxobutanoic acid
293 (R)-4-((4-(5-(difluoromethoxy)-2-(6-methoxypyridin-3-yl)phenyl)thiazol-2-yl)(methyl)amino)-3-(furan-2-ylmethyl)-4-oxobutanoic acid
294 (R)-4-(cyclopropyl(4-(2,5-dichlorophenyl)thiazol-2-yl)amino)-3-(furan-2-ylmethyl)-4-oxobutanoic acid
295 (R)-4-((4-(2-chloro-5-fluorophenyl)thiazol-2-yl)(cyclopropyl)amino)-3-(furan-2-ylmethyl)-4-oxobutanoic acid
296 (R)-4-((4-(2-chloro-5-(trifluoromethyl)phenyl)thiazol-2-yl)(cyclopropyl)amino)-3-(furan-2-ylmethyl)-4-oxobutanoic acid
297 (R)-4-((4-(2-chloro-5-(trifluoromethoxy)phenyl)thiazol-2-yl)(cyclopropyl)amino)-3-(furan-2-ylmethyl)-4-oxobutanoic acid
298 (R)-4-((4-(2-chloro-5-(difluoromethoxy)phenyl)thiazol-2-yl)(cyclopropyl)amino)-3-(furan-2-ylmethyl)-4-oxobutanoic acid
299 4-((4-(2-chlorophenyl)thiazol-2-yl)(methyl)amino)-3-((5-methylfuran-2-yl)methyl)-4-oxobutanoic acid
300 4-((4-(2-chlorophenyl)thiazol-2-yl)(methyl)amino)-3-((4,5-dimethylfuran-2-yl)methyl)-4-oxobutanoic acid
301 3-(benzofuran-2-ylmethyl)-4-((4-(2-chlorophenyl)thiazol-2-yl)(methyl)amino)-4-oxobutanoic acid
302 (R)-4-((4-(2-chlorophenyl)thiazol-2-yl)(methyl)amino)-4-oxo-3-(pyridin-2-ylmethyl)butanoic acid
303 (R)-4-((4-(2-chlorophenyl)thiazol-2-yl)(methyl)amino)-4-oxo-3-(pyrimidin-2-ylmethyl)butanoic acid
304 (3 R)-4-((4-(2,5-dichlorophenyl)thiazol-2-yl)(methyl)amino)-4-oxo-3-((tetrahydro-2H-pyran-2-yl)methyl)butanoic acid
305 (3R)-4-((4-(2,5-dichlorophenyl)thiazol-2-yl)(methyl)amino)-4-oxo-3-((tetrahydro-2H-pyran-3-yl)methyl)butanoic acid
306 (R)-4-((4-(2,5-dichlorophenyl)thiazol-2-yl)(methyl)amino)-3-(((2R,3R)-2-methyltetrahydro-2H-pyran-3-yl)methyl)-4-oxobutanoic acid
307 (3 R)-4-((4-(2,5-dichlorophenyl)thiazol-2-yl)(methyl)amino)-3-(((2R)-2-methyltetrahydro-2H-pyran-4-yl)methyl)-4-oxobutanoic acid
308 (3 R)-4-((4-(2,5-dichlorophenyl)thiazol-2-yl)(methyl)amino)-3-(((2R,6S)-2,6-dimethyltetrahydro-2H-pyran-4-yl)methyl)-4-oxobutanoic acid
309 (3 R)-4-((4-(2,5-dichlorophenyl)thiazol-2-yl)(methyl)amino)-3-(((3S)-3-methyltetrahydro-2H-pyran-4-yl)methyl)-4-oxobutanoic acid
310 (3 R)-4-((4-(2,5-dichlorophenyl)thiazol-2-yl)(methyl)amino)-3-(((3R,5S)-3,5-dimethyltetrahydro-2H-pyran-4-yl)methyl)-4-oxobutanoic acid
311 (R)-2-benzyl-N-(4-(2-chlorophenyl)thiazol-2-yl)-3-(4-hydroxy-1,2,5-thiadiazol-3-yl)-N-methylpropanamide
312 (R)-2-benzyl-N-(4-(2-chlorophenyl)thiazol-2-yl)-3-(3-hydroxy-5-methylisoxazol-4-yl)-N-methylpropanamide
313 (R)-4-((4-(5-chloro-2-(6-(2-oxopiperidin-1-yl)pyridin-3-yl)phenyl)thiazol-2-yl)(cyclopropyl)amino)-4-oxo-3-((tetrahydro-2H-pyran-4-yl)methyl)butanoic acid
314 (R)-4-((4-(5-chloro-2-(6-(2-oxopiperidin-1-yl)pyridin-3-yl)phenyl)-5-fluorothiazol-2-yl)(cyclopropyl)amino)-4-oxo-3-((tetrahydro-2H-pyran-4-yl)methyl)butanoic acid
315 (R)-4-((4-(5-chloro-2-(6-methoxypyridin-3-yl)phenyl)thiazol-2-yl)(cyclopropyl)amino)-4-oxo-3-((tetrahydro-2H-pyran-4-yl)methyl)butanoic acid
316 (R)-4-((4-(5-chloro-2-(6-methoxypyridin-3-yl)phenyl)-5-fluorothiazol-2-yl)(cyclopropyl)amino)-4-oxo-3-((tetrahydro-2H-pyran-4-yl)methyl)butanoic acid
317 (R)-4-((4-(5-chloro-2-(6-(2-oxopyrrolidin-1-yl)pyridin-3-yl)phenyl)thiazol-2-yl)(cyclopropyl)amino)-4-oxo-3-((tetrahydro-2H-pyran-4-yl)methyl)butanoic acid
318 (R)-4-((4-(5-chloro-2-(6-(2-oxopyrrolidin-1-yl)pyridin-3-yl)phenyl)-5-fluorothiazol-2-yl)(cyclopropyl)amino)-4-oxo-3-((tetrahydro-2H-pyran-4-yl)methyl)butanoic acid
319 (R)-4-(cyclopropyl(4-(5-(difluoromethoxy)-2-(6-(2-oxopiperidin-1-yl)pyridin-3-yl)phenyl)thiazol-2-yl)amino)-4-oxo-3-((tetrahydro-2H-pyran-4-yl)methyl)butanoic acid
320 (R)-4-(cyclopropyl(4-(5-(difluoromethoxy)-2-(6-(2-oxopiperidin-1-yl)pyridin-3-yl)phenyl)-5-fluorothiazol-2-yl)amino)-4-oxo-3-((tetrahydro-2H-pyran-4-yl)methyl)butanoic acid
321 (R)-4-(cyclopropyl(4-(5-(difluoromethoxy)-2-(6-methoxypyridin-3-yl)phenyl)thiazol-2-yl)amino)-4-oxo-3-((tetrahydro-2H-pyran-4-yl)methyl)butanoic acid
322 (R)-4-(cyclopropyl(4-(5-(difluoromethoxy)-2-(6-methoxypyridin-3-yl)phenyl)-5-fluorothiazol-2-yl)amino)-4-oxo-3-((tetrahydro-2H-pyran-4-yl)methyl)butanoic acid
323 (R)-4-(cyclopropyl(4-(5-(difluoromethoxy)-2-(6-(2-oxopyrrolidin-1)pyridin-3₁1)phenyl)thiazol-2-yl)amino)-4-oxo-3-((tetrahydro-2H-pyran-4-yl)methyl)butanoic acid
324 (R)-4-(cyclopropyl(4-(5-(difluoromethoxy)-2-(6-(2-oxopyrrolidin-1-yl)pyridin-3-yl)phenyl)-5-fluorothiazol-2-yl)amino)-4-oxo-3-((tetrahydro-2H-pyran-4-yl)methyl)butanoic acid
325 (R)-4-(cyclopropyl(4-(2-(6-(2-oxopiperidin-1-yl)pyridin-3-yl)phenyl)thiazol-2-yl)amino)-4-oxo-3-((tetrahydro-2H-pyran-4-yl)methyl)butanoic acid
326 (R)-4-(cyclopropyl(5-fluoro-4-(2-(6-(2-oxopiperidin-1-yl)pyridin-3-yl)phenyl)thiazol-2-yl)amino)-4-oxo-3-((tetrahydro-2H-pyran-4-yl)methyl)butanoic acid
327 (R)-4-(cyclopropyl(5-fluoro-4-(2-(6-methoxypyridin-3-yl)phenyl)thiazol-2-yl)amino)-4-oxo-3-((tetrahydro-2H-pyran-4-yl)methyl)butanoic acid
328 (R)-4-(cyclopropyl(4-(2-(6-(2-oxopyrrolidin-1-yl)pyridin-3-yl)phenyl)thiazol-2-yl)amino)-4-oxo-3-((tetrahydro-2H-pyran-4-yl)methyl)butanoic acid
329 (R)-4-(cyclopropyl(5-fluoro-4-(2-(6-(2-oxopyrrolidin-1-yl)pyridin-3-yl)phenyl)thiazol-2-yl)amino)-4-oxo-3-((tetrahydro-2H-pyran-4-yl)methyl)butanoic acid
330 (R)-4-(cyclopropyl(4-(5-fluoro-2-(6-(2-oxopiperidin-1-yl)pyridin-3-yl)phenyl)thiazol-2-yl)amino)-4-oxo-3-((tetrahydro-2H-pyran-4-yl)methyl)butanoic acid
331 (R)-4-(cyclopropyl(5-fluoro-4-(5-fluoro-2-(6-(2-oxopiperidin-1-yl)pyridin-3-yl)phenyl)thiazol-2-yl)amino)-4-oxo-3-((tetrahydro-2H-pyran-4-yl)methyl)butanoic acid
332 (R)-4-(cyclopropyl(4-(5-fluoro-2-(6-methoxypyridin-3-yl)phenyl)thiazol-2-yl)amino)-4-oxo-3-((tetrahydro-2H-pyran-4-yl)methyl)butanoic acid
333 (R)-4-(cyclopropyl(5-fluoro-4-(5-fluoro-2-(6-methoxypyridin-3-yl)phenyl)thiazol-2-yl)amino)-4-oxo-3-((tetrahydro-2H-pyran-4-yl)methyl)butanoic acid
334 (R)-4-(cyclopropyl(4-(5-fluoro-2-(6-(2-oxopyrrolidin-1-yl)pyridin-3-yl)phenyl)thiazol-2-yl)amino)-4-oxo-3-((tetrahydro-2H-pyran-4-yl)methyl)butanoic acid
335 (R)-4-(cyclopropyl(5-fluoro-4-(5-fluoro-2-(6-(2-oxopyrrolidin-1-yl)pyridin-3-yl)phenyl)thiazol-2-yl)amino)-4-oxo-3-((tetrahydro-2H-pyran-4-yl)methyl)butanoic acid
336 (R)-4-((4-(5-chloro-2-(6-(2-oxopiperidin-1-yl)pyridin-3-yl)phenyl)thiazol-2-yl)(cyclopropyl)amino)-3-(cyclopentylmethyl)-4-oxobutanoic acid
337 (R)-4-((4-(5-chloro-2-(6-(2-oxopiperidin-1-yl)pyridin-3-yl)phenyl)-5-fluorothiazol-2-yl)(cyclopropyl)amino)-3-(cyclopentylmethyl)-4-oxobutanoic acid
338 (R)-4-((4-(5-chloro-2-(6-methoxypyridin-3-yl)phenyl)-5-fluorothiazol-2-yl)(cyclopropyl)amino)-3-(cyclopentylmethyl)-4-oxobutanoic acid
339 (R)-4-((4-(5-chloro-2-(6-(2-oxopyrrolidin-1-yl)pyridin-3-yl)phenyl)thiazol-2-yl)(cyclopropyl)amino)-3-(cyclopentylmethyl)-4-oxobutanoic acid
340 (R)-4-((4-(5-chloro-2-(6-(2-oxopyrrolidin-1-yl)pyridin-3-yl)phenyl)-5-fluorothiazol-2-yl)(cyclopropyl)amino)-3-(cyclopentylmethyl)-4-oxobutanoic acid
341 (R)-3-(cyclopentylmethyl)-4-(cyclopropyl(4-(5-(difluoromethoxy)-2-(6-(2-oxopiperidin-1-yl)pyridin-3-yl)phenyl)thiazol-2-yl)amino)-4-oxobutanoic acid
342 (R)-3-(cyclopentylmethyl)-4-(cyclopropyl(4-(5-(difluoromethoxy)-2-(6-(2-oxopiperidin-1-yl)pyridin-3-yl)phenyl)-5-fluorothiazol-2-yl)amino)-4-oxobutanoic acid
343 (R)-3-(cyclopentylmethyl)-4-(cyclopropyl(4-(5-(difluoromethoxy)-2-(6-methoxypyridin-3-yl)phenyl)thiazol-2-yl)amino)-4-oxobutanoic acid
344 (R)-3-(cyclopentylmethyl)-4-(cyclopropyl(4-(5-(difluoromethoxy)-2-(6-methoxypyridin-3-yl)phenyl)-5-fluorothiazol-2-yl)amino)-4-oxobutanoic acid
345 (R)-3-(cyclopentylmethyl)-4-(cyclopropyl(4-(5-(difluoromethoxy)-2-(6-(2-oxopyrrolidin-1-yl)pyridin-3-yl)phenyl)thiazol-2-yl)amino)-4-oxobutanoic acid
346 (R)-3-(cyclopentylmethyl)-4-(cyclopropyl(4-(5-(difluoromethoxy)-2-(6-(2-oxopyrrolidin-1-yl)pyridin-3-yl)phenyl)-5-fluorothiazol-2-yl)amino)-4-oxobutanoic acid
347 (R)-3-(cyclopentylmethyl)-4-(cyclopropyl(4-(2-(6-(2-oxopiperidin-1-yl)pyridin-3₁1)phenyl)thiazol-2-yl)amino)-4-oxobutanoic acid
348 (R)-3-(cyclopentylmethyl)-4-(cyclopropyl(5-fluoro-4-(2-(6-(2-oxopiperidin-1-yl)pyridin-3-yl)phenyl)thiazol-2-yl)amino)-4-oxobutanoic acid
349 (R)-3-(cyclopentylmethyl)-4-(cyclopropyl(5-fluoro-4-(2-(6-methoxypyridin-3-yl)phenyl)thiazol-2-yl)amino)-4-oxobutanoic acid
350 (R)-3-(cyclopentylmethyl)-4-(cyclopropyl(5-fluoro-4-(2-(6-(2-oxopyrrolidin-1-yl)pyridin-3-yl)phenyl)thiazol-2-yl)amino)-4-oxobutanoic acid
351 (R)-3-(cyclopentylmethyl)-4-(cyclopropyl(4-(5-fluoro-2-(6-(2-oxopiperidin-1-yl)pyridin-3-yl)phenyl)thiazol-2-yl)amino)-4-oxobutanoic acid
352 (R)-3-(cyclopentylmethyl)-4-(cyclopropyl(5-fluoro-4-(5-fluoro-2-(6-(2-oxopiperidin-1-yl)pyridin-3-yl)phenyl)thiazol-2-yl)amino)-4-oxobutanoic acid
353 (R)-3-(cyclopentylmethyl)-4-(cyclopropyl(4-(5-fluoro-2-(6-methoxypyridin-3-yl)phenyl)thiazol-2-yl)amino)-4-oxobutanoic acid
354 (R)-3-(cyclopentylmethyl)-4-(cyclopropyl(5-fluoro-4-(5-fluoro-2-(6-methoxypyridin-3-yl)phenyl)thiazol-2-yl)amino)-4-oxobutanoic acid
355 (R)-3-(cyclopentylmethyl)-4-(cyclopropyl(4-(5-fluoro-2-(6-(2-oxopyrrolidin-1yl)pyridin-3-yl)phenyl)thiazol-2-yl)amino)-4-oxobutanoic acid
356 (R)-3-(cyclopentylmethyl)-4-(cyclopropyl(5-fluoro-4-(5-fluoro-2-(6-(2-oxopyrrolidin-1-yl)pyridin-3-yl)phenyl)thiazol-2-yl)amino)-4-oxobutanoic acid
357 (R)-3-benzyl-4-((4-(5-chloro-2-(6-(2-oxopiperidin-1-yl)pyridin-3-yl)phenyl)thiazol-2-yl)(cyclopropyl)amino)-4-oxobutanoic acid
358 (R)-3-benzyl-4-((4-(5-chloro-2-(6-(2-oxopiperidin-1-yl)pyridin-3-yl)phenyl)-5-fluorothiazol-2-yl)(cyclopropyl)amino)-4-oxobutanoic acid
359 (R)-3-benzyl-4-((4-(5-chloro-2-(6-methoxypyridin-3-yl)phenyl)thiazol-2-yl)(cyclopropyl)amino)-4-oxobutanoic acid
360 (R)-3-benzyl-4-((4-(5-chloro-2-(6-methoxypyridin-3-yl)phenyl)-5-fluorothiazol-2-yl)(cyclopropyl)amino)-4-oxobutanoic acid
361 (R)-3-benzyl-4-((4-(5-chloro-2-(6-(2-oxopyrrolidin-1-yl)pyridin-3-yl)phenyl)thiazol-2-yl)(cyclopropyl)amino)-4-oxobutanoic acid
362 (R)-3-benzyl-4-((4-(5-chloro-2-(6-(2-oxopyrrolidin-1-yl)pyridin-3-yl)phenyl)-5-fluorothiazol-2-yl)(cyclopropyl)amino)-4-oxobutanoic acid
363 (R)-3-benzyl-4-(cyclopropyl(4-(5-(difluoromethoxy)-2-(6-(2-oxopiperidin-1-yl)pyridin-3-yl)phenyl)thiazol-2-yl)amino)-4-oxobutanoic acid
364 (R)-3-benzyl-4-(cyclopropyl(4-(5-(difluoromethoxy)-2-(6-(2-oxopiperidin-1-yl)pyridin-3-yl)phenyl)-5-fluorothiazol-2-yl)amino)-4-oxobutanoic acid
365 (R)-3-benzyl-4-(cyclopropyl(4-(5-(difluoromethoxy)-2-(6-methoxypyridin-3-yl)phenyl)thiazol-2-yl)amino)-4-oxobutanoic acid
366 (R)-3-benzyl-4-(cyclopropyl(4-(5-(difluoromethoxy)-2-(6-methoxypyridin-3-yl)phenyl)-5-fluorothiazol-2-yl)amino)-4-oxobutanoic acid
367 (R)-3-benzyl-4-(cyclopropyl(4-(5-(difluoromethoxy)-2-(6-(2-oxopyrrolidin-1-yl)pyridin-3-yl)phenyl)thiazol-2-yl)amino)-4-oxobutanoic acid
368 (R)-3-benzyl-4-(cyclopropyl(4-(5-(difluoromethoxy)-2-(6-(2-oxopyrrolidin-1-yl)pyridin-3-yl)phenyl)-5-fluorothiazol-2-yl)amino)-4-oxobutanoic acid
369 (R)-3-benzyl-4-(cyclopropyl(4-(2-(6-(2-oxopiperidin-1-yl)pyridin-3-yl)phenyl)thiazol-2-yl)amino)-4-oxobutanoic acid
370 (R)-3-benzyl-4-(cyclopropyl(5-fluoro-4-(2-(6-(2-oxopiperidin-1-yl)pyridin-3-yl)phenyl)thiazol-2-yl)amino)-4-oxobutanoic acid
371 (R)-3-benzyl-4-(cyclopropyl(5-fluoro-4-(2-(6-methoxypyridin-3-yl)phenyl)thiazol-2-yl)amino)-4-oxobutanoic acid
372 (R)-3-benzyl-4-(cyclopropyl(5-fluoro-4-(2-(6-(2-oxopyrrolidin-1-yl)pyridin-3-yl)phenyl)thiazol-2-yl)amino)-4-oxobutanoic acid
373 (R)-3-benzyl-4-(cyclopropyl(4-(5-fluoro-2-(6-(2-oxopiperidin-1-yl)pyridin-3-yl)phenyl)thiazol-2-yl)amino)-4-oxobutanoic acid
374 (R)-3-benzyl-4-(cyclopropyl(5-fluoro-4-(5-fluoro-2-(6-(2-oxopiperidin-1-yl)pyridin-3-yl)phenyl)thiazol-2-yl)amino)-4-oxobutanoic acid
375 (R)-3-benzyl-4-(cyclopropyl(4-(5-fluoro-2-(6-methoxypyridin-3-yl)phenyl)thiazol-2-yl)amino)-4-oxobutanoic acid
376 (R)-3-benzyl-4-(cyclopropyl(5-fluoro-4-(5-fluoro-2-(6-methoxypyridin-3-yl)phenyl)thiazol-2-yl)amino)-4-oxobutanoic acid
377 (R)-3-benzyl-4-(cyclopropyl(4-(5-fluoro-2-(6-(2-oxopyrrolidin-1-yl)pyridin-3-yl)phenyl)thiazol-2-yl)amino)-4-oxobutanoic acid
378 (R)-3-benzyl-4-(cyclopropyl(5-fluoro-4-(5-fluoro-2-(6-(2-oxopyrrolidin-1-yl)pyridin-3-yl)phenyl)thiazol-2-yl)amino)-4-oxobutanoic acid
379 (R)-4-((4-(5-chloro-2-(6-(2-oxopiperidin-1-yl)pyridin-3-yl)phenyl)thiazol-2-yl)(methyl)amino)-4-oxo-3-((tetrahydro-2H-pyran-4-yl)methyl)butanoic acid
380 (R)-4-((4-(5-chloro-2-(6-(2-oxopiperidin-1-yl)pyridin-3-yl)phenyl)-5-fluorothiazol-2-yl)(methyl)amino)-4-oxo-3-((tetrahydro-2H-pyran-4-yl)methyl)butanoic acid
381 (R)-4-((4-(5-chloro-2-(6-methoxypyridin-3-yl)phenyl)thiazol-2-yl)(methyl)amino)-4-oxo-3-((tetrahydro-2H-pyran-4-yl)methyl)butanoic acid
382 (R)-4-((4-(5-chloro-2-(6-methoxypyridin-3-yl)phenyl)-5-fluorothiazol-2-yl)(methyl)amino)-4-oxo-3-((tetrahydro-2H-pyran-4-yl)methyl)butanoic acid
383 (R)-4-((4-(5-chloro-2-(6-(2-oxopyrrolidin-1-yl)pyridin-3-yl)phenyl)thiazol-2-yl)(methyl)amino)-4-oxo-3-((tetrahydro-2H-pyran-4-yl)methyl)butanoic acid
384 (R)-4-((4-(5-chloro-2-(6-(2-oxopyrrolidin-1-yl)pyridin-3-yl)phenyl)-5-fluorothiazol-2-yl)(methyl)amino)-4-oxo-3-((tetrahydro-2H-pyran-4-yl)methyl)butanoic acid
385 (R)-4-((4-(5-(difluoromethoxy)-2-(6-(2-oxopiperidin-1-yl)pyridin-3-yl)phenyl)thiazol-2-yl)(methyl)amino)-4-oxo-3-((tetrahydro-2H-pyran-4-yl)methyl)butanoic acid
386 (R)-4-((4-(5-(difluoromethoxy)-2-(6-(2-oxopiperidin-1-yl)pyridin-3-yl)phenyl)-5-fluorothiazol-2-yl)(methyl)amino)-4-oxo-3-((tetrahydro-2H-pyran-4-yl)methyl)butanoic acid
387 (R)-4-((4-(5-(difluoromethoxy)-2-(6-methoxypyridin-3-yl)phenyl)thiazol-2-yl)(methyl)amino)-4-oxo-3-((tetrahydro-2H-pyran-4-yl)methyl)butanoic acid
388 (R)-4-((4-(5-(difluoromethoxy)-2-(6-methoxypyridin-3-yl)phenyl)-5-fluorothiazol-2-yl)(methyl)amino)-4-oxo-3-((tetrahydro-2H-pyran-4-yl)methyl)butanoic acid
389 (R)-4-((4-(5-(difluoromethoxy)-2-(6-(2-oxopyrrolidin-1-yl)pyridin-3-yl)phenyl)thiazol-2-yl)(methyl)amino)-4-oxo-3-((tetrahydro-2H-pyran-4-yl)methyl)butanoic acid
390 (R)-4-((4-(5-(difluoromethoxy)-2-(6-(2-oxopyrrolidin-1-yl)pyridin-3-yl)phenyl)-5-fluorothiazol-2-yl)(methyl)amino)-4-oxo-3-((tetrahydro-2H-pyran-4-yl)methyl)butanoic acid
391 (R)-4-(methyl(4-(2-(6-(2-oxopiperidin-1-yl)pyridin-3-yl)phenyl)thiazol-2-yl)amino)-4-oxo-3-((tetrahydro-2H-pyran-4-yl)methyl)butanoic acid
392 (R)-4-((5-fluoro-4-(2-(6-(2-oxopiperidin-1-yl)pyridin-3-yl)phenyl)thiazol-2-yl)(methyl)amino)-4-oxo-3-((tetrahydro-2H-pyran-4-yl)methyl)butanoic acid
393 (R)-4-((5-fluoro-4-(2-(6-methoxypyridin-3-yl)phenyl)thiazol-2-yl)(methyl)amino)-4-oxo-3-((tetrahydro-2H-pyran-4-yl)methyl)butanoic acid
394 (R)-4-(methyl(4-(2-(6-(2-oxopyrrolidin-1-yl)pyridin-3-yl)phenyl)thiazol-2-yl)amino)-4-oxo-3-((tetrahydro-2H-pyran-4-yl)methyl)butanoic acid
395 (R)-4-((5-fluoro-4-(2-(6-(2-oxopyrrolidin-1-yl)pyridin-3-yl)phenyl)thiazol-2-yl)(methyl)amino)-4-oxo-3-((tetrahydro-2H-pyran-4-yl)methyl)butanoic acid
396 (R)-4-((4-(5-fluoro-2-(6-(2-oxopiperidin-1-yl)pyridin-3-yl)phenyl)thiazol-2-yl)(methyl)amino)-4-oxo-3-((tetrahydro-2H-pyran-4-yl)methyl)butanoic acid
397 (R)-4-((5-fluoro-4-(5-fluoro-2-(6-(2-oxopiperidin-1-yl)pyridin-3-yl)phenyl)thiazol-2-yl)(methyl)amino)-4-oxo-3-((tetrahydro-2H-pyran-4-yl)methyl)butanoic acid
398 (R)-4-((4-(5-fluoro-2-(6-methoxypyridin-3-yl)phenyl)thiazol-2-yl)(methyl)amino)-4-oxo-3-((tetrahydro-2H-pyran-4-yl)methyl)butanoic acid
399 (R)-4-((5-fluoro-4-(5-fluoro-2-(6-methoxypyridin-3-yl)phenyl)thiazol-2-yl)(methyl)amino)-4-oxo-3-((tetrahydro-2H-pyran-4-yl)methyl)butanoic acid
400 (R)-4-((4-(5-fluoro-2-(6-(2-oxopyrrolidin-1-yl)pyridin-3-yl)phenyl)thiazol-2-yl)(methyl)amino)-4-oxo-3-((tetrahydro-2H-pyran-4-yl)methyl)butanoic acid
401 (R)-4-((5-fluoro-4-(5-fluoro-2-(6-(2-oxopyrrolidin-1-yl)pyridin-3-yl)phenyl)thiazol-2-yl)(methyl)amino)-4-oxo-3-((tetrahydro-2H-pyran-4-yl)methyl)butanoic acid
402 (R)-4-((4-(5-chloro-2-(6-(2-oxopiperidin-1-yl)pyridin-3-yl)phenyl)thiazol-2-yl)(methyl)amino)-3-(cyclopentylmethyl)-4-oxobutanoic acid
403 (R)-4-((4-(5-chloro-2-(6-(2-oxopiperidin-1-yl)pyridin-3-yl)phenyl)-5-fluorothiazol-2-yl)(methyl)amino)-3-(cyclopentylmethyl)-4-oxobutanoic acid
404 (R)-4-((4-(5-chloro-2-(6-methoxypyridin-3-yl)phenyl)thiazol-2-yl)(methyl)amino)-3-(cyclopentylmethyl)-4-oxobutanoic acid
405 (R)-4-((4-(5-chloro-2-(6-methoxypyridin-3-yl)phenyl)-5-fluorothiazol-2-yl)(methyl)amino)-3-(cyclopentylmethyl)-4-oxobutanoic acid
406 (R)-4-((4-(5-chloro-2-(6-(2-oxopyrrolidin-1-yl)pyridin-3-yl)phenyl)thiazol-2-yl)(methyl)amino)-3-(cyclopentylmethyl)-4-oxobutanoic acid
407 (R)-4-((4-(5-chloro-2-(6-(2-oxopyrrolidin-1-yl)pyridin-3-yl)phenyl)-5-fluorothiazol-2-yl)(methyl)amino)-3-(cyclopentylmethyl)-4-oxobutanoic acid
408 (R)-3-(cyclopentylmethyl)-4-((4-(5-(difluoromethoxy)-2-(6-(2-oxopiperidin-1-yl)pyridin-3-yl)phenyl)thiazol-2-yl)(methyl)amino)-4-oxobutanoic acid
409 (R)-3-(cyclopentylmethyl)-4-((4-(5-(difluoromethoxy)-2-(6-(2-oxopiperidin-1-yl)pyridin-3-yl)phenyl)-5-fluorothiazol-2-yl)(methyl)amino)-4-oxobutanoic acid
410 (R)-3-(cyclopentylmethyl)-4-((4-(5-(difluoromethoxy)-2-(6-methoxypyridin-3-yl)phenyl)thiazol-2-yl)(methyl)amino)-4-oxobutanoic acid
411 (R)-3-(cyclopentylmethyl)-4-((4-(5-(difluoromethoxy)-2-(6-methoxypyridin-3-yl)phenyl)-5-fluorothiazol-2-yl)(methyl)amino)-4-oxobutanoic acid
412 (R)-3-(cyclopentylmethyl)-4-((4-(5-(difluoromethoxy)-2-(6-(2-oxopyrrolidin-1-yl)pyridin-3-yl)phenyl)thiazol-2-yl)(methyl)amino)-4-oxobutanoic acid
413 (R)-3-(cyclopentylmethyl)-4-((4-(5-(difluoromethoxy)-2-(6-(2-oxopyrrolidin-1-yl)pyridin-3-yl)phenyl)-5-fluorothiazol-2-yl)(methyl)amino)-4-oxobutanoic acid
414 (R)-3-(cyclopentylmethyl)-4-(methyl(4-(2-(6-(2-oxopiperidin-1-yl)pyridin-3-yl)phenyl)thiazol-2-yl)amino)-4-oxobutanoic acid
415 (R)-3-(cyclopentylmethyl)-4-((5-fluoro-4-(2-(6-(2-oxopiperidin-1-yl)pyridin-3-yl)phenyl)thiazol-2-yl)(methyl)amino)-4-oxobutanoic acid
416 (R)-3-(cyclopentylmethyl)-4-((4-(2-(6-methoxypyridin-3-yl)phenyl)thiazol-2-yl)(methyl)amino)-4-oxobutanoic acid
417 (R)-3-(cyclopentylmethyl)-4-((5-fluoro-4-(2-(6-methoxypyridin-3-yl)phenyl)thiazol-2-yl)(methyl)amino)-4-oxobutanoic acid
418 (R)-3-(cyclopentylmethyl)-4-((5-fluoro-4-(2-(6-(2-oxopyrrolidin-1-yl)pyridin-3-yl)phenyl)thiazol-2-yl)(methyl)amino)-4-oxobutanoic acid
419 (R)-3-(cyclopentylmethyl)-4-((4-(5-fluoro-2-(6-(2-oxopiperidin-1-yl)pyridin-3-yl)phenyl)thiazol-2-yl)(methyl)amino)-4-oxobutanoic acid
420 (R)-3-(cyclopentylmethyl)-4-((5-fluoro-4-(5-fluoro-2-(6-(2-oxopiperidin-1-yl)pyridin-3-yl)phenyl)thiazol-2-yl)(methyl)amino)-4-oxobutanoic acid
421 (R)-3-(cyclopentylmethyl)-4-((4-(5-fluoro-2-(6-methoxypyridin-3-yl)phenyl)thiazol-2-yl)(methyl)amino)-4-oxobutanoic acid
422 (R)-3-(cyclopentylmethyl)-4-((5-fluoro-4-(5-fluoro-2-(6-methoxypyridin-3-yl)phenyl)thiazol-2-yl)(methyl)amino)-4-oxobutanoic acid
423 (R)-3-(cyclopentylmethyl)-4-((4-(5-fluoro-2-(6-(2-oxopyrrolidin-1-yl)pyridin-3-yl)phenyl)thiazol-2-yl)(methyl)amino)-4-oxobutanoic acid
424 (R)-3-(cyclopentylmethyl)-4-((5-fluoro-4-(5-fluoro-2-(6-(2-oxopyrrolidin-1-yl)pyridin-3-yl)phenyl)thiazol-2-yl)(methyl)amino)-4-oxobutanoic acid
425 (R)-3-benzyl-4-((4-(5-chloro-2-(6-(2-oxopiperidin-1-yl)pyridin-3-yl)phenyl)thiazol-2-yl)(methyl)amino)-4-oxobutanoic acid
426 (R)-3-benzyl-4-((4-(5-chloro-2-(6-(2-oxopiperidin-1-yl)pyridin-3-yl)phenyl)-5-fluorothiazol-2-yl)(methyl)amino)-4-oxobutanoic acid
427 (R)-3-benzyl-4-((4-(5-chloro-2-(6-methoxypyridin-3-yl)phenyl)-5-fluorothiazol-2-yl)(methyl)amino)-4-oxobutanoic acid
428 (R)-3-benzyl-4-((4-(5-chloro-2-(6-(2-oxopyrrolidin-1-yl)pyridin-3-yl)phenyl)thiazol-2-yl)(methyl)amino)-4-oxobutanoic acid
429 (R)-3-benzyl-4-((4-(5-chloro-2-(6-(2-oxopyrrolidin-1-yl)pyridin-3-yl)phenyl)-5-fluorothiazol-2-yl)(methyl)amino)-4-oxobutanoic acid
430 (R)-3-benzyl-4-((4-(5-(difluoromethoxy)-2-(6-(2-oxopiperidin-1-yl)pyridin-3-yl)phenyl)thiazol-2-yl)(methyl)amino)-4-oxobutanoic acid
431 (R)-3-benzyl-4-((4-(5-(difluoromethoxy)-2-(6-(2-oxopiperidin-1-yl)pyridin-3-yl)phenyl)-5-fluorothiazol-2-yl)(methyl)amino)-4-oxobutanoic acid
432 (R)-3-benzyl-4-((4-(5-(difluoromethoxy)-2-(6-methoxypyridin-3-yl)phenyl)thiazol-2-yl)(methyl)amino)-4-oxobutanoic acid
433 (R)-3-benzyl-4-((4-(5-(difluoromethoxy)-2-(6-methoxypyridin-3-yl)phenyl)-5-fluorothiazol-2-yl)(methyl)amino)-4-oxobutanoic acid
434 (R)-3-benzyl-4-((4-(5-(difluoromethoxy)-2-(6-(2-oxopyrrolidin-1-yl)pyridin-3-yl)phenyl)thiazol-2-yl)(methyl)amino)-4-oxobutanoic acid
435 (R)-3-benzyl-4-((4-(5-(difluoromethoxy)-2-(6-(2-oxopyrrolidin-1-yl)pyridin-3-yl)phenyl)-5-fluorothiazol-2-yl)(methyl)amino)-4-oxobutanoic acid
436 (R)-3-benzyl-4-(methyl(4-(2-(6-(2-oxopiperidin-1-yl)pyridin-3-yl)phenyl)thiazol-2-yl)amino)-4-oxobutanoic acid
437 (R)-3-benzyl-4-((5-fluoro-4-(2-(6-(2-oxopiperidin-1-yl)pyridin-3-yl)phenyl)thiazol-2-yl)(methyl)amino)-4-oxobutanoic acid
438 (R)-3-benzyl-4-((5-fluoro-4-(2-(6-methoxypyridin-3-yl)phenyl)thiazol-2-yl)(methyl)amino)-4-oxobutanoic acid
439 (R)-3-benzyl-4-((5-fluoro-4-(2-(6-(2-oxopyrrolidin-1-yl)pyridin-3-yl)phenyl)thiazol-2-yl)(methyl)amino)-4-oxobutanoic acid
440 (R)-3-benzyl-4-((4-(5-fluoro-2-(6-(2-oxopiperidin-1-yl)pyridin-3-yl)phenyl)thiazol-2-yl)(methyl)amino)-4-oxobutanoic acid
441 (R)-3-benzyl-4-((5-fluoro-4-(5-fluoro-2-(6-(2-oxopiperidin-1-yl)pyridin-3-yl)phenyl)thiazol-2-yl)(methyl)amino)-4-oxobutanoic acid
442 (R)-3-benzyl-4-((5-fluoro-4-(5-fluoro-2-(6-methoxypyridin-3-yl)phenyl)thiazol-2-yl)(methyl)amino)-4-oxobutanoic acid
443 (R)-3-benzyl-4-((4-(5-fluoro-2-(6-(2-oxopyrrolidin-1-yl)pyridin-3-yl)phenyl)thiazol-2-yl)(methyl)amino)-4-oxobutanoic acid
444 (R)-3-benzyl-4-((5-fluoro-4-(5-fluoro-2-(6-(2-oxopyrrolidin-1-yl)pyridin-3-yl)phenyl)thiazol-2-yl)(methyl)amino)-4-oxobutanoic acid
445 (R)-3-(cyclopentylmethyl)-4-((4-(5-fluoro-2-(8-methyl-7-oxo-5,6,7,8-tetrahydro-1,8-naphthyridin-3-yl)phenyl)thiazol-2-yl)(methyl)amino)-4-oxobutanoic acid
446 (R)-3-(cyclopentylmethyl)-4-(cyclopropyl(4-(5-fluoro-2-(8-methyl-7-oxo-5,6,7,8-tetrahydro-1,8-naphthyridin-3-yl)phenyl)thiazol-2-yl)amino)-4-oxobutanoic acid
447 (R)-4-((4-(5-fluoro-2-(8-methyl-7-oxo-5,6,7,8-tetrahydro-1,8-naphthyridin-3-yl)phenyl)thiazol-2-yl)(methyl)amino)-4-oxo-3-((tetrahydro-2H-pyran-4-yl)methyl)butanoic acid
448 (R)-4-(cyclopropyl(4-(5-fluoro-2-(8-methyl-7-oxo-5,6,7,8-tetrahydro-1,8-naphthyridin-3-yl)phenyl)thiazol-2-yl)amino)-4-oxo-3-((tetrahydro-2H-pyran-4-yl)methyl)butanoic acid
449 (R)-3-benzyl-4-((4-(5-fluoro-2-(8-methyl-7-oxo-5,6,7,8-tetrahydro-1,8-naphthyridin-3-yl)phenyl)thiazol-2-yl)(methyl)amino)-4-oxobutanoic acid
450 (R)-3-benzyl-4-(cyclopropyl(4-(5-fluoro-2-(8-methyl-7-oxo-5,6,7,8-tetrahydro-1,8-naphthyridin-3-yl)phenyl)thiazol-2-yl)amino)-4-oxobutanoic acid
451 (R)-3-(cyclopentylmethyl)-4-((4-(5-fluoro-2-(1-methyl-2-oxo-2,3-dihydro-1H-pyrrolo[2,3-b]pyridin-5-yl)phenyl)thiazol-2-yl)(methyl)amino)-4-oxobutanoic acid
452 (R)-3-(cyclopentylmethyl)-4-(cyclopropyl(4-(5-fluoro-2-(1-methyl-2-oxo-2,3-dihydro-1H-pyrrolo[2,3-b]pyridin-5-yl)phenyl)thiazol-2-yl)amino)-4-oxobutanoic acid
453 (R)-4-((4-(5-fluoro-2-(1-methyl-2-oxo-2,3-dihydro-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
454 (R)-4-(cyclopropyl(4-(5-fluoro-2-(1-methyl-2-oxo-2,3-dihydro-1H-pyrrolo[2,3-b]pyridin-5-yl)phenyl)thiazol-2-yl)amino)-4-oxo-3-((tetrahydro-2H-pyran-4-yl)methyl)butanoic acid
455 (R)-3-benzyl-4-((4-(5-fluoro-2-(1-methyl-2-oxo-2,3-dihydro-1H-pyrrolo[2,3-b]pyridin-5-yl)phenyl)thiazol-2-yl)(methyl)amino)-4-oxobutanoic acid
456 (R)-3-benzyl-4-(cyclopropyl(4-(5-fluoro-2-(1-methyl-2-oxo-2,3-dihydro-1H-pyrrolo[2,3-b]pyridin-5-yl)phenyl)thiazol-2-yl)amino)-4-oxobutanoic acid
457 (R)-3-(cyclopentylmethyl)-4-((4-(5-fluoro-2-(4-methyl-3,4-dihydro-2H-pyrido[3,2-b][1,4]oxazin-7-yl)phenyl)thiazol-2-yl)(methyl)amino)-4-oxobutanoic acid
458 (R)-3-(cyclopentylmethyl)-4-(cyclopropyl(4-(5-fluoro-2-(4-methyl-3,4-dihydro-2H-pyrido[3,2-][1,4]oxazin-7-yl)phenyl)thiazol-2-yl)amino)-4-oxobutanoic acid
459 (R)-4-((4-(5-fluoro-2-(4-methyl-3,4-dihydro-2H-pyrido[3,2-b][1,4]oxazin-7-yl)phenyl)thiazol-2-yl)(methyl)amino)-4-oxo-3-((tetrahydro-2H-pyran-4-yl)methyl)butanoic acid
460 (R)-4-(cyclopropyl(4-(5-fluoro-2-(4-methyl-3,4-dihydro-2H-pyrido[3,2-b][1,4]oxazin-7-yl)phenyl)thiazol-2-yl)amino)-4-oxo-3-((tetrahydro-2H-pyran-4-yl)methyl)butanoic acid
461 (R)-3-benzyl-4-((4-(5-fluoro-2-(4-methyl-3,4-dihydro-2H-pyrido[3,2-b][1,4]oxazin-7-yl)phenyl)thiazol-2-yl)(methyl)amino)-4-oxobutanoic acid
462 (R)-3-benzyl-4-(cyclopropyl(4-(5-fluoro-2-(4-methyl-3,4-dihydro-2H-pyrido[3,2-b][1,4]oxazin-7-yl)phenyl)thiazol-2-yl)amino)-4-oxobutanoic acid
463 (R)-3-(cyclopentylmethyl)-4-((4-(5-fluoro-2-(1-methyl-1H-pyrrolo[2,3-b]pyridin-5-yl)phenyl)thiazol-2-yl)(methyl)amino)-4-oxobutanoic acid
464 (R)-3-(cyclopentylmethyl)-4-(cyclopropyl(4-(5-fluoro-2-(1-methyl-1H-pyrrolo[2,3-b]pyridin-5-yl)phenyl)thiazol-2-yl)amino)-4-oxobutanoic acid
465 (R)-4-((4-(5-fluoro-2-(1-methyl-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
466 (R)-4-(cyclopropyl(4-(5-fluoro-2-(1-methyl-1H-pyrrolo[2,3-b]pyridin-5-yl)phenyl)thiazol-2-yl)amino)-4-oxo-3-((tetrahydro-2H-pyran-4-yl)methyl)butanoic acid
467 (R)-3-benzyl-4-((4-(5-fluoro-2-(1-methyl-1H-pyrrolo[2,3-b]pyridin-5-yl)phenyl)thiazol-2-yl)(methyl)amino)-4-oxobutanoic acid
468 (R)-3-benzyl-4-(cyclopropyl(4-(5-fluoro-2-(1-methyl-1H-pyrrolo[2,3-b]pyridin-5-yl)phenyl)thiazol-2-yl)amino)-4-oxobutanoic acid
469 (R)-3-(cyclopentylmethyl)-4-((4-(5-(fluoromethoxy)-2-(8-methyl-7-oxo-5,6,7,8-tetrahydro-1,8-naphthyridin-3-yl)phenyl)thiazol-2-yl)(methyl)amino)-4-oxobutanoic acid
470 (R)-3-(cyclopentylmethyl)-4-(cyclopropyl(4-(5-(fluoromethoxy)-2-(8-methyl-7-oxo-5,6,7,8-tetrahydro-1,8-naphthyridin-3-yl)phenyl)thiazol-2-yl)amino)-4-oxobutanoic acid
471 (R)-4-((4-(5-(fluoromethoxy)-2-(8-methyl-7-oxo-5,6,7,8-tetrahydro-1,8-naphthyridin-3-yl)phenyl)thiazol-2-yl)(methyl)amino)-4-oxo-3-((tetrahydro-2H-pyran-4-yl)methyl)butanoic acid
472 (R)-4-(cyclopropyl(4-(5-(fluoromethoxy)-2-(8-methyl-7-oxo-5,6,7,8-tetrahydro-1,8-naphthyridin-3-yl)phenyl)thiazol-2-yl)amino)-4-oxo-3-((tetrahydro-2H-pyran-4-yl)methyl)butanoic acid
473 (R)-3-benzyl-4-((4-(5-(fluoromethoxy)-2-(8-methyl-7-oxo-5,6,7,8-tetrahydro-1,8-naphthyridin-3-yl)phenyl)thiazol-2-yl)(methyl)amino)-4-oxobutanoic acid
474 (R)-3-benzyl-4-(cyclopropyl(4-(5-(fluoromethoxy)-2-(8-methyl-7-oxo-5,6,7,8-tetrahydro-1,8-naphthyridin-3-yl)phenyl)thiazol-2-yl)amino)-4-oxobutanoic acid
475 (R)-3-(cyclopentylmethyl)-4-((4-(5-(fluoromethoxy)-2-(1-methyl-2-oxo-2,3-dihydro-1H-pyrrolo[2,3-b]pyridin-5-yl)phenyl)thiazol-2-yl)(methyl)amino)-4-oxobutanoic acid
476 (R)-3-(cyclopentylmethyl)-4-(cyclopropyl(4-(5-(fluoromethoxy)-2-(1-methyl-2-oxo-2,3-dihydro-1H-pyrrolo[2,3-b]pyridin-5-yl)phenyl)thiazol-2-yl)amino)-4-oxobutanoic acid
477 (R)-4-((4-(5-(fluoromethoxy)-2-(1-methyl-2-oxo-2,3-dihydro-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
478 (R)-4-(cyclopropyl(4-(5-(fluoromethoxy)-2-(1-methyl-2-oxo-2,3-dihydro-1H-pyrrolo[2,3-b]pyridin-5-yl)phenyl)thiazol-2-yl)amino)-4-oxo-3-((tetrahydro-2H-pyran-4-yl)methyl)butanoic acid
479 (R)-3-benzyl-4-((4-(5-(fluoromethoxy)-2-(1-methyl-2-oxo-2,3-dihydro-1H-pyrrolo[2,3-b]pyridin-5-yl)phenyl)thiazol-2-yl)(methyl)amino)-4-oxobutanoic acid
480 (R)-3-benzyl-4-(cyclopropyl(4-(5-(fluoromethoxy)-2-(1-methyl-2-oxo-2,3-dihydro-1H-pyrrolo[2,3-b]pyridin-5-yl)phenyl)thiazol-2-yl)amino)-4-oxobutanoic acid
481 (R)-3-(cyclopentylmethyl)-4-((4-(5-(fluoromethoxy)-2-(4-methyl-3,4-dihydro-2H-pyrido[3,2-b][1,4]oxazin-7-yl)phenyl)thiazol-2-yl)(methyl)amino)-4-oxobutanoic acid
482 (R)-3-(cyclopentylmethyl)-4-(cyclopropyl(4-(5-(fluoromethoxy)-2-(4-methyl-3,4-dihydro-2H-pyrido[3,2-b][1,4]oxazin-7-yl)phenyl)thiazol-2-yl)amino)-4-oxobutanoic acid
483 (R)-4-((4-(5-(fluoromethoxy)-2-(4-methyl-3,4-dihydro-2H-pyrido[3,2-b][1,4]oxazin-7-yl)phenyl)thiazol-2-yl)(methyl)amino)-4-oxo-3-((tetrahydro-2H-pyran-4-yl)methyl)butanoic acid
484 (R)-4-(cyclopropyl(4-(5-(fluoromethoxy)-2-(4-methyl-3,4-dihydro-2H-pyrido[3,2-b][1,4]oxazin-7-yl)phenyl)thiazol-2-yl)amino)-4-oxo-3-((tetrahydro-2H-pyran-4-yl)methyl)butanoic acid
485 (R)-3-benzyl-4-((4-(5-(fluoromethoxy)-2-(4-methyl-3,4-dihydro-2H-pyrido[3,2-b][1,4]oxazin-7-yl)phenyl)thiazol-2-yl)(methyl)amino)-4-oxobutanoic acid
486 (R)-3-benzyl-4-(cyclopropyl(4-(5-(fluoromethoxy)-2-(4-methyl-3,4-dihydro-2H-pyrido[3,2-b][1,4]oxazin-7-yl)phenyl)thiazol-2-yl)amino)-4-oxobutanoic acid
487 (R)-3-(cyclopentylmethyl)-4-((4-(5-(fluoromethoxy)-2-(1-methyl-1H-pyrrolo[2,3-b]pyridin-5-yl)phenyl)thiazol-2-yl)(methyl)amino)-4-oxobutanoic acid
488 (R)-3-(cyclopentylmethyl)-4-(cyclopropyl(4-(5-(fluoromethoxy)-2-(1-methyl-1H-pyrrolo[2,3-b]pyridin-5-yl)phenyl)thiazol-2-yl)amino)-4-oxobutanoic acid
489 (R)-4-((4-(5-(fluoromethoxy)-2-(1-methyl-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
490 (R)-4-(cyclopropyl(4-(5-(fluoromethoxy)-2-(1-methyl-1H-pyrrolo[2,3-b]pyridin-5-yl)phenyl)thiazol-2-yl)amino)-4-oxo-3-((tetrahydro-2H-pyran-4-yl)methyl)butanoic acid
491 (R)-3-benzyl-4-((4-(5-(fluoromethoxy)-2-(1-methyl-1H-pyrrolo[2,3-b]pyridin-5-yl)phenyl)thiazol-2-yl)(methyl)amino)-4-oxobutanoic acid
492 (R)-3-benzyl-4-(cyclopropyl(4-(5-(fluoromethoxy)-2-(1-methyl-1H-pyrrolo[2,3-b]pyridin-5-yl)phenyl)thiazol-2-yl)amino)-4-oxobutanoic acid
493 (R)-4-((4-(5-chloro-2-(8-methyl-7-oxo-5,6,7,8-tetrahydro-1,8-naphthyridin-3-yl)phenyl)thiazol-2-yl)(methyl)amino)-3-(cyclopentylmethyl)-4-oxobutanoic acid
494 (R)-4-((4-(5-chloro-2-(8-methyl-7-oxo-5,6,7,8-tetrahydro-1,8-naphthyridin-3-yl)phenyl)thiazol-2-yl)(cyclopropyl)amino)-3-(cyclopentylmethyl)-4-oxobutanoic acid
495 (R)-4-((4-(5-chloro-2-(8-methyl-7-oxo-5,6,7,8-tetrahydro-1,8-naphthyridin-3-yl)phenyl)thiazol-2-yl)(methyl)amino)-4-oxo-3-((tetrahydro-2H-pyran-4-yl)methyl)butanoic acid
496 (R)-4-((4-(5-chloro-2-(8-methyl-7-oxo-5,6,7,8-tetrahydro-1,8-naphthyridin-3-yl)phenyl)thiazol-2-yl)(cyclopropyl)amino)-4-oxo-3-((tetrahydro-2H-pyran-4-yl)methyl)butanoic acid
497 (R)-3-benzyl-4-((4-(5-chloro-2-(8-methyl-7-oxo-5,6,7,8-tetrahydro-1,8-naphthyridin-3-yl)phenyl)thiazol-2-yl)(methyl)amino)-4-oxobutanoic acid
498 (R)-3-benzyl-4-((4-(5-chloro-2-(8-methyl-7-oxo-5,6,7,8-tetrahydro-1,8-naphthyridin-3-yl)phenyl)thiazol-2-yl)(cyclopropyl)amino)-4-oxobutanoic acid
499 (R)-4-((4-(5-chloro-2-(1-methyl-2-oxo-2,3-dihydro-1H-pyrrolo[2,3-b]pyridin-5-yl)phenyl)thiazol-2-yl)(methyl)amino)-3-(cyclopentylmethyl)-4-oxobutanoic acid
500 (R)-4-((4-(5-chloro-2-(1-methyl-2-oxo-2,3-dihydro-1H-pyrrolo[2,3-b]pyridin-5-yl)phenyl)thiazol-2-yl)(cyclopropyl)amino)-3-(cyclopentylmethyl)-4-oxobutanoic acid
501 (R)-4-((4-(5-chloro-2-(1-methyl-2-oxo-2,3-dihydro-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
502 (R)-4-((4-(5-chloro-2-(1-methyl-2-oxo-2,3-dihydro-1H-pyrrolo[2,3-b]pyridin-5-yl)phenyl)thiazol-2-yl)(cyclopropyl)amino)-4-oxo-3-((tetrahydro-2H-pyran-4-yl)methyl)butanoic acid
503 (R)-3-benzyl-4-((4-(5-chloro-2-(1-methyl-2-oxo-2,3-dihydro-1H-pyrrolo[2,3-b]pyridin-5-yl)phenyl)thiazol-2-yl)(methyl)amino)-4-oxobutanoic acid
504 (R)-3-benzyl-4-((4-(5-chloro-2-(1-methyl-2-oxo-2,3-dihydro-1H-pyrrolo[2,3-b]pyridin-5-yl)phenyl)thiazol-2-yl)(cyclopropyl)amino)-4-oxobutanoic acid
505 (R)-4-((4-(5-chloro-2-(4-methyl-3,4-dihydro-2H-pyrido[3,2-b][1,4]oxazin-7-yl)phenyl)thiazol-2-yl)(methyl)amino)-3-(cyclopentylmethyl)-4-oxobutanoic acid
506 (R)-4-((4-(5-chloro-2-(4-methyl-3,4-dihydro-2H-pyrido[3,2-b][1,4]oxazin-7-yl)phenyl)thiazol-2-yl)(cyclopropyl)amino)-3-(cyclopentylmethyl)-4-oxobutanoic acid
507 (R)-4-((4-(5-chloro-2-(4-methyl-3,4-dihydro-2H-pyrido[3,2-b][1,4]oxazin-7-yl)phenyl)thiazol-2-yl)(methyl)amino)-4-oxo-3-((tetrahydro-2H-pyran-4-yl)methyl)butanoic acid
508 (R)-4-((4-(5-chloro-2-(4-methyl-3,4-dihydro-2H-pyrido[3,2-b][1,4]oxazin-7-yl)phenyl)thiazol-2-yl)(cyclopropyl)amino)-4-oxo-3-((tetrahydro-2H-pyran-4-yl)methyl)butanoic acid
509 (R)-3-benzyl-4-((4-(5-chloro-2-(4-methyl-3,4-dihydro-2H-pyrido[3,2-b][1,4]oxazin-7-yl)phenyl)thiazol-2-yl)(methyl)amino)-4-oxobutanoic acid
510 (R)-3-benzyl-4-((4-(5-chloro-2-(4-methyl-3,4-dihydro-2H-pyrido[3,2-b][1,4]oxazin-7-yl)phenyl)thiazol-2-yl)(cyclopropyl)amino)-4-oxobutanoic acid
511 (R)-4-((4-(5-chloro-2-(1-methyl-1H-pyrrolo[2,3-b]pyridin-5-yl)phenyl)thiazol-2-yl)(methyl)amino)-3-(cyclopentylmethyl)-4-oxobutanoic acid
512 (R)-4-((4-(5-chloro-2-(1-methyl-1H-pyrrolo[2,3-b]pyridin-5-yl)phenyl)thiazol-2-yl)(cyclopropyl)amino)-3-(cyclopentylmethyl)-4-oxobutanoic acid
513 (R)-4-((4-(5-chloro-2-(1-methyl-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
514 (R)-4-((4-(5-chloro-2-(1-methyl-1H-pyrrolo[2,3-b]pyridin-5-yl)phenyl)thiazol-2-yl)(cyclopropyl)amino)-4-oxo-3-((tetrahydro-2H-pyran-4-yl)methyl)butanoic acid
515 (R)-3-benzyl-4-((4-(5-chloro-2-(1-methyl-1H-pyrrolo[2,3-b]pyridin-5-yl)phenyl)thiazol-2-yl)(methyl)amino)-4-oxobutanoic acid
516 (R)-3-benzyl-4-((4-(5-chloro-2-(1-methyl-1H-pyrrolo[2,3-b]pyridin-5-yl)phenyl)thiazol-2-yl)(cyclopropyl)amino)-4-oxobutanoic acid
517 (R)-3-(cyclopentylmethyl)-4-(methyl(4-(2-(8-methyl-7-oxo-5,6,7,8-tetrahydro-1,8-naphthyridin-3-yl)phenyl)thiazol-2-yl)amino)-4-oxobutanoic acid
518 (R)-3-(cyclopentylmethyl)-4-(cyclopropyl(4-(2-(8-methyl-7-oxo-5,6,7,8-tetrahydro-1,8-naphthyridin-3-yl)phenyl)thiazol-2-yl)amino)-4-oxobutanoic acid
519 (R)-4-(methyl(4-(2-(8-methyl-7-oxo-5,6,7,8-tetrahydro-1,8-naphthyridin-3-yl)phenyl)thiazol-2-yl)amino)-4-oxo-3-((tetrahydro-2H-pyran-4-yl)methyl)butanoic acid
520 (R)-4-(cyclopropyl(4-(2-(8-methyl-7-oxo-5,6,7,8-tetrahydro-1,8-naphthyridin-3-yl)phenyl)thiazol-2-yl)amino)-4-oxo-3-((tetrahydro-2H-pyran-4-yl)methyl)butanoic acid
521 (R)-3-benzyl-4-(cyclopropyl(4-(2-(8-methyl-7-oxo-5,6,7,8-tetrahydro-1,8-naphthyridin-3-yl)phenyl)thiazol-2-yl)amino)-4-oxobutanoic acid
522 (R)-3-(cyclopentylmethyl)-4-(methyl(4-(2-(1-methyl-2-oxo-2,3-dihydro-1H-pyrrolo[2,3-b]pyridin-5-yl)phenyl)thiazol-2-yl)amino)-4-oxobutanoic acid
523 (R)-3-(cyclopentylmethyl)-4-(cyclopropyl(4-(2-(1-methyl-2-oxo-2,3-dihydro-1H-pyrrolo[2,3-b]pyridin-5-yl)phenyl)thiazol-2-yl)amino)-4-oxobutanoic acid
524 (R)-4-(methyl(4-(2-(1-methyl-2-oxo-2,3-dihydro-1H-pyrrolo[2,3-b]pyridin-5-yl)phenyl)thiazol-2-yl)amino)-4-oxo-3-((tetrahydro-2H-pyran-4-yl)methyl)butanoic acid
525 (R)-4-(cyclopropyl(4-(2-(1-methyl-2-oxo-2,3-dihydro-1H-pyrrolo[2,3-b]pyridin-5-yl)phenyl)thiazol-2-yl)amino)-4-oxo-3-((tetrahydro-2H-pyran-4-yl)methyl)butanoic acid
526 (R)-3-benzyl-4-(cyclopropyl(4-(2-(1-methyl-2-oxo-2,3-dihydro-1H-pyrrolo[2,3-b]pyridin-5-yl)phenyl)thiazol-2-yl)amino)-4-oxobutanoic acid
527 (R)-3-(cyclopentylmethyl)-4-(methyl(4-(2-(4-methyl-3,4-dihydro-2H-pyrido[3,2-b][1,4]oxazin-7-yl)phenyl)thiazol-2-yl)amino)-4-oxobutanoic acid
528 (R)-4-(methyl(4-(2-(4-methyl-3,4-dihydro-2H-pyrido[3,2-b][1,4]oxazin-7-yl)phenyl)thiazol-2-yl)amino)-4-oxo-3-((tetrahydro-2H-pyran-4-yl)methyl)butanoic acid
529 (R)-4-(cyclopropyl(4-(2-(4-methyl-3,4-dihydro-2H-pyrido[3,2-b][1,4]oxazin-7-yl)phenyl)thiazol-2-yl)amino)-4-oxo-3-((tetrahydro-2H-pyran-4-yl)methyl)butanoic acid
530 (R)-3-benzyl-4-(cyclopropyl(4-(2-(4-methyl-3,4-dihydro-2H-pyrido[3,2-b][1,4]oxazin-7-yl)phenyl)thiazol-2-yl)amino)-4-oxobutanoic acid
531 (R)-3-(cyclopentylmethyl)-4-(methyl(4-(2-(1-methyl-1H-pyrrolo[2,3-b]pyridin-5-yl)phenyl)thiazol-2-yl)amino)-4-oxobutanoic acid
532 (R)-3-(cyclopentylmethyl)-4-(cyclopropyl(4-(2-(1-methyl-1H-pyrrolo[2,3-b]pyridin-5-yl)phenyl)thiazol-2-yl)amino)-4-oxobutanoic acid
533 (R)-4-(methyl(4-(2-(1-methyl-1H-pyrrolo[2,3-b]pyridin-5-yl)phenyl)thiazol-2-yl)amino)-4-oxo-3-((tetrahydro-2H-pyran-4-yl)methyl)butanoic acid
534 (R)-4-(cyclopropyl(4-(2-(1-methyl-1H-pyrrolo[2,3-b]pyridin-5-yl)phenyl)thiazol-2-yl)amino)-4-oxo-3-((tetrahydro-2H-pyran-4-yl)methyl)butanoic acid
535 (R)-3-benzyl-4-(cyclopropyl(4-(2-(1-methyl-1H-pyrrolo[2,3-b]pyridin-5-yl)phenyl)thiazol-2-yl)amino)-4-oxobutanoic acid
536 (R)-3-(cyclopentylmethyl)-4-((5-fluoro-4-(5-fluoro-2-(8-methyl-7-oxo-5,6,7,8-tetrahydro-1,8-naphthyridin-3-yl)phenyl)thiazol-2-yl)(methyl)amino)-4-oxobutanoic acid
537 (R)-3-(cyclopentylmethyl)-4-(cyclopropyl(5-fluoro-4-(5-fluoro-2-(8-methyl-7-oxo-5,6,7,8-tetrahydro-1,8-naphthyridin-3-yl)phenyl)thiazol-2-yl)amino)-4-oxobutanoic acid
538 (R)-4-((5-fluoro-4-(5-fluoro-2-(8-methyl-7-oxo-5,6,7,8-tetrahydro-1,8-naphthyridin-3-yl)phenyl)thiazol-2-yl)(methyl)amino)-4-oxo-3-((tetrahydro-2H-pyran-4-yl)methyl)butanoic acid
539 (R)-4-(cyclopropyl(5-fluoro-4-(5-fluoro-2-(8-methyl-7-oxo-5,6,7,8-tetrahydro-1,8-naphthyridin-3-yl)phenyl)thiazol-2-yl)amino)-4-oxo-3-((tetrahydro-2H-pyran-4-yl)methyl)butanoic acid
540 (R)-3-benzyl-4-((5-fluoro-4-(5-fluoro-2-(8-methyl-7-oxo-5,6,7,8-tetrahydro-1,8-naphthyridin-3-yl)phenyl)thiazol-2-yl)(methyl)amino)-4-oxobutanoic acid
541 (R)-3-benzyl-4-(cyclopropyl(5-fluoro-4-(5-fluoro-2-(8-methyl-7-oxo-5,6,7,8-tetrahydro-1,8-naphthyridin-3-yl)phenyl)thiazol-2-yl)amino)-4-oxobutanoic acid
542 (R)-3-(cyclopentylmethyl)-4-((5-fluoro-4-(5-fluoro-2-(1-methyl-2-oxo-2,3-dihydro-1H-pyrrolo[2,3-b]pyridin-5-yl)phenyl)thiazol-2-yl)(methyl)amino)-4-oxobutanoic acid
543 (R)-3-(cyclopentylmethyl)-4-(cyclopropyl(5-fluoro-4-(5-fluoro-2-(1-methyl-2-oxo-2,3-dihydro-1H-pyrrolo[2,3-b]pyridin-5-yl)phenyl)thiazol-2-yl)amino)-4-oxobutanoic acid
544 (R)-4-((5-fluoro-4-(5-fluoro-2-(1-methyl-2-oxo-2,3-dihydro-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
545 (R)-4-(cyclopropyl(5-fluoro-4-(5-fluoro-2-(1-methyl-2-oxo-2,3-dihydro-1H-pyrrolo[2,3-b]pyridin-5-yl)phenyl)thiazol-2-yl)amino)-4-oxo-3-((tetrahydro-2H-pyran-4-yl)methyl)butanoic acid
546 (R)-3-benzyl-4-((5-fluoro-4-(5-fluoro-2-(1-methyl-2-oxo-2,3-dihydro-1H-pyrrolo[2,3-b]pyridin-5-yl)phenyl)thiazol-2-yl)(methyl)amino)-4-oxobutanoic acid
547 (R)-3-benzyl-4-(cyclopropyl(5-fluoro-4-(5-fluoro-2-(1-methyl-2-oxo-2,3-dihydro-1H-pyrrolo[2,3-b]pyridin-5-yl)phenyl)thiazol-2-yl)amino)-4-oxobutanoic acid
548 (R)-3-(cyclopentylmethyl)-4-((5-fluoro-4-(5-fluoro-2-(4-methyl-3,4-dihydro-2H-pyrido[3,2-b][1,4]oxazin-7-yl)phenyl)thiazol-2-yl)(methyl)amino)-4-oxobutanoic acid
549 (R)-3-(cyclopentylmethyl)-4-(cyclopropyl(5-fluoro-4-(5-fluoro-2-(4-methyl-3,4-dihydro-2H-pyrido[3,2-b][1,4]oxazin-7-yl)phenyl)thiazol-2-yl)amino)-4-oxobutanoic acid
550 (R)-4-((5-fluoro-4-(5-fluoro-2-(4-methyl-3,4-dihydro-2H-pyrido[3,2-b][1,4]oxazin-7-yl)phenyl)thiazol-2-yl)(methyl)amino)-4-oxo-3-((tetrahydro-2H-pyran-4-yl)methyl)butanoic acid
551 (R)-4-(cyclopropyl(5-fluoro-4-(5-fluoro-2-(4-methyl-3,4-dihydro-2H-pyrido[3,2-b][1,4]oxazin-7-yl)phenyl)thiazol-2-yl)amino)-4-oxo-3-((tetrahydro-2H-pyran-4-yl)methyl)butanoic acid
552 (R)-3-benzyl-4-((5-fluoro-4-(5-fluoro-2-(4-methyl-3,4-dihydro-2H-pyrido[3,2-b][1,4]oxazin-7-yl)phenyl)thiazol-2-yl)(methyl)amino)-4-oxobutanoic acid
553 (R)-3-benzyl-4-(cyclopropyl(5-fluoro-4-(5-fluoro-2-(4-methyl-3,4-dihydro-2H-pyrido[3,2-b][1,4]oxazin-7-yl)phenyl)thiazol-2-yl)amino)-4-oxobutanoic acid
554 (R)-3-(cyclopentylmethyl)-4-((5-fluoro-4-(5-fluoro-2-(1-methyl-1H-pyrrolo[2,3-b]pyridin-5-yl)phenyl)thiazol-2-yl)(methyl)amino)-4-oxobutanoic acid
555 (R)-3-(cyclopentylmethyl)-4-(cyclopropyl(5-fluoro-4-(5-fluoro-2-(1-methyl-1H-pyrrolo[2,3-b]pyridin-5-yl)phenyl)thiazol-2-yl)amino)-4-oxobutanoic acid
556 (R)-4-((5-fluoro-4-(5-fluoro-2-(1-methyl-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
557 (R)-4-(cyclopropyl(5-fluoro-4-(5-fluoro-2-(1-methyl-1H- pyrrolo[2,3-b]pyridin-5-yl)phenyl)thiazol-2-yl)amino)-4-oxo-3-((tetrahydro-2H-pyran-4-yl)methyl)butanoic acid
558 (R)-3-benzyl-4-((5-fluoro-4-(5-fluoro-2-(1-methyl-1H-pyrrolo[2,3-b]pyridin-5-yl)phenyl)thiazol-2-yl)(methyl)amino)-4-oxobutanoic acid
559 (R)-3-benzyl-4-(cyclopropyl(5-fluoro-4-(5-fluoro-2-(1-methyl-1H-pyrrolo[2,3-b]pyridin-5-yl)phenyl)thiazol-2-yl)amino)-4-oxobutanoic acid
560 (R)-3-(cyclopentylmethyl)-4-((5-fluoro-4-(5-(fluoromethoxy)-2-(8-methyl-7-oxo-5,6,7,8-tetrahydro-1,8-naphthyridin-3-yl)phenyl)thiazol-2-yl)(methyl)amino)-4-oxobutanoic acid
561 (R)-3-(cyclopentylmethyl)-4-(cyclopropyl(5-fluoro-4-(5-(fluoromethoxy)-2-(8-methyl-7-oxo-5,6,7,8-tetrahydro-1,8-naphthyridin-3-yl)phenyl)thiazol-2-yl)amino)-4-oxobutanoic acid
562 (R)-4-((5-fluoro-4-(5-(fluoromethoxy)-2-(8-methyl-7-oxo-5,6,7,8-tetrahydro-1,8-naphthyridin-3-yl)phenyl)thiazol-2-yl)(methyl)amino)-4-oxo-3-((tetrahydro-2H-pyran-4-yl)methyl)butanoic acid
563 (R)-4-(cyclopropyl(5-fluoro-4-(5-(fluoromethoxy)-2-(8-methyl-7-oxo-5,6,7,8-tetrahydro-1,8-naphthyridin-3-yl)phenyl)thiazol-2-yl)amino)-4-oxo-3-((tetrahydro-2H-pyran-4-yl)methyl)butanoic acid
564 (R)-3-benzyl-4-((5-fluoro-4-(5-(fluoromethoxy)-2-(8-methyl-7-oxo-5,6,7,8-tetrahydro-1,8-naphthyridin-3-yl)phenyl)thiazol-2-yl)(methyl)amino)-4-oxobutanoic acid
565 (R)-3-benzyl-4-(cyclopropyl(5-fluoro-4-(5-(fluoromethoxy)-2-(8-methyl-7-oxo-5,6,7,8-tetrahydro-1,8-naphthyridin-3-yl)phenyl)thiazol-2-yl)amino)-4-oxobutanoic acid
566 (R)-3-(cyclopentylmethyl)-4-((5-fluoro-4-(5-(fluoromethoxy)-2-(1-methyl-2-oxo-2,3-dihydro-1H-pyrrolo[2,3-b]pyridin-5-yl)phenyl)thiazol-2-yl)(methyl)amino)-4-oxobutanoic acid
567 (R)-3-(cyclopentylmethyl)-4-(cyclopropyl(5-fluoro-4-(5-(fluoromethoxy)-2-(1-methyl-2-oxo-2,3-dihydro-1H-pyrrolo[2,3-b]pyridin-5-yl)phenyl)thiazol-2-yl)amino)-4-oxobutanoic acid
568 (R)-4-((5-fluoro-4-(5-(fluoromethoxy)-2-(1-methyl-2-oxo-2,3-dihydro-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
569 (R)-4-(cyclopropyl(5-fluoro-4-(5-(fluoromethoxy)-2-(1-methyl-2-oxo-2,3-dihydro-1H-pyrrolo[2,3-b]pyridin-5-yl)phenyl)thiazol-2-yl)amino)-4-oxo-3-((tetrahydro-2H-pyran-4-yl)methyl)butanoic acid
570 (R)-3-benzyl-4-((5-fluoro-4-(5-(fluoromethoxy)-2-(1-methyl-2-oxo-2,3-dihydro-1H-pyrrolo[2,3-b]pyridin-5-yl)phenyl)thiazol-2-yl)(methyl)amino)-4-oxobutanoic acid
571 (R)-3-benzyl-4-(cyclopropyl(5-fluoro-4-(5-(fluoromethoxy)-2-(1-methyl-2-oxo-2,3-dihydro-1H-pyrrolo[2,3-b]pyridin-5-yl)phenyl)thiazol-2-yl)amino)-4-oxobutanoic acid
572 (R)-3-(cyclopentylmethyl)-4-((5-fluoro-4-(5-(fluoromethoxy)-2-(4-methyl-3,4-dihydro-2H-pyrido[3,2-b][1,4]oxazin-7-yl)phenyl)thiazol-2-yl)(methyl)amino)-4-oxobutanoic acid
573 (R)-3-(cyclopentylmethyl)-4-(cyclopropyl(5-fluoro-4-(5-(fluoromethoxy)-2-(4-methyl-3,4-dihydro-2H-pyrido[3,2-b][1,4]oxazin-7-yl)phenyl)thiazol-2-yl)amino)-4-oxobutanoic acid
574 (R)-4-((5-fluoro-4-(5-(fluoromethoxy)-2-(4-methyl-3,4-dihydro-2H-pyrido[3,2-b][1,4]oxazin-7-yl)phenyl)thiazol-2-yl)(methyl)amino)-4-oxo-3-((tetrahydro-2H-pyran-4-yl)methyl)butanoic acid
575 (R)-4-(cyclopropyl(5-fluoro-4-(5-(fluoromethoxy)-2-(4-methyl-3,4-dihydro-2H-pyrido[3,2-b][1,4]oxazin-7-yl)phenyl)thiazol-2-yl)amino)-4-oxo-3-((tetrahydro-2H-pyran-4-yl)methyl)butanoic acid
576 (R)-3-benzyl-4-((5-fluoro-4-(5-(fluoromethoxy)-2-(4-methyl-3,4-dihydro-2H-pyrido[3,2-b][1,4]oxazin-7-yl)phenyl)thiazol-2-yl)(methyl)amino)-4-oxobutanoic acid
577 (R)-3-benzyl-4-(cyclopropyl(5-fluoro-4-(5-(fluoromethoxy)-2-(4-methyl-3,4-dihydro-2H-pyrido[3,2-b][1,4]oxazin-7-yl)phenyl)thiazol-2-yl)amino)-4-oxobutanoic acid
578 (R)-3-(cyclopentylmethyl)-4-((5-fluoro-4-(5-(fluoromethoxy)-2-(1-methyl-1H-pyrrolo[2,3-b]pyridin-5-yl)phenyl)thiazol-2-yl)(methyl)amino)-4-oxobutanoic acid
579 (R)-3-(cyclopentylmethyl)-4-(cyclopropyl(5-fluoro-4-(5-(fluoromethoxy)-2-(1-methyl-1H-pyrrolo[2,3-b]pyridin-5-yl)phenyl)thiazol-2-yl)amino)-4-oxobutanoic acid
580 (R)-4-((5-fluoro-4-(5-(fluoromethoxy)-2-(1-methyl-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
581 (R)-4-(cyclopropyl(5-fluoro-4-(5-(fluoromethoxy)-2-(1-methyl-1H-pyrrolo[2,3-b]pyridin-5-yl)phenyl)thiazol-2-yl)amino)-4-oxo-3-((tetrahydro-2H-pyran-4-yl)methyl)butanoic acid
582 (R)-3-benzyl-4-((5-fluoro-4-(5-(fluoromethoxy)-2-(1-methyl-1H-pyrrolo[2,3-b]pyridin-5-yl)phenyl)thiazol-2-yl)(methyl)amino)-4-oxobutanoic acid
583 (R)-3-benzyl-4-(cyclopropyl(5-fluoro-4-(5-(fluoromethoxy)-2-(1-methyl-1H-pyrrolo[2,3-b]pyridin-5-yl)phenyl)thiazol-2-yl)amino)-4-oxobutanoic acid
584 (R)-4-((4-(5-chloro-2-(8-methyl-7-oxo-5,6,7,8-tetrahydro-1,8-naphthyridin-3-yl)phenyl)-5-fluorothiazol-2-yl)(methyl)amino)-3-(cyclopentylmethyl)-4-oxobutanoic acid
585 (R)-4-((4-(5-chloro-2-(8-methyl-7-oxo-5,6,7,8-tetrahydro-1,8-naphthyridin-3-yl)phenyl)-5-fluorothiazol-2-yl)(cyclopropyl)amino)-3-(cyclopentylmethyl)-4-oxobutanoic acid
586 (R)-4-((4-(5-chloro-2-(8-methyl-7-oxo-5,6,7,8-tetrahydro-1,8-naphthyridin-3-yl)phenyl)-5-fluorothiazol-2-yl)(methyl)amino)-4-oxo-3-((tetrahydro-2H-pyran-4-yl)methyl)butanoic acid
587 (R)-4-((4-(5-chloro-2-(8-methyl-7-oxo-5,6,7,8-tetrahydro-1,8-naphthyridin-3-yl)phenyl)-5-fluorothiazol-2-yl)(cyclopropyl)amino)-4-oxo-3-((tetrahydro-2H-pyran-4-yl)methyl)butanoic acid
588 (R)-3-benzyl-4-((4-(5-chloro-2-(8-methyl-7-oxo-5,6,7,8-tetrahydro-1,8-naphthyridin-3-yl)phenyl)-5-fluorothiazol-2-yl)(methyl)amino)-4-oxobutanoic acid
589 (R)-3-benzyl-4-((4-(5-chloro-2-(8-methyl-7-oxo-5,6,7,8-tetrahydro-1,8-naphthyridin-3-yl)phenyl)-5-fluorothiazol-2-yl)(cyclopropyl)amino)-4-oxobutanoic acid
590 (R)-4-((4-(5-chloro-2-(1-methyl-2-oxo-2,3-dihydro-1H-pyrrolo[2,3-b]pyridin-5-yl)phenyl)-5-fluorothiazol-2-yl)(methyl)amino)-3-(cyclopentylmethyl)-4-oxobutanoic acid
591 (R)-4-((4-(5-chloro-2-(1-methyl-2-oxo-2,3-dihydro-1H-pyrrolo[2,3-b]pyridin-5-yl)phenyl)-5-fluorothiazol-2-yl)(cyclopropyl)amino)-3-(cyclopentylmethyl)-4-oxobutanoic acid
592 (R)-4-((4-(5-chloro-2-(1-methyl-2-oxo-2,3-dihydro-1H-pyrrolo[2,3-b]pyridin-5-yl)phenyl)-5-fluorothiazol-2-yl)(methyl)amino)-4-oxo-3-((tetrahydro-2H-pyran-4-yl)methyl)butanoic acid
593 (R)-4-((4-(5-chloro-2-(1-methyl-2-oxo-2,3-dihydro-1H-pyrrolo[2,3-b]pyridin-5-yl)phenyl)-5-fluorothiazol-2-yl)(cyclopropyl)amino)-4-oxo-3-((tetrahydro-2H-pyran-4-yl)methyl)butanoic acid
594 (R)-3-benzyl-4-((4-(5-chloro-2-(1-methyl-2-oxo-2,3-dihydro-1H-pyrrolo[2,3-b]pyridin-5-yl)phenyl)-5-fluorothiazol-2-yl)(methyl)amino)-4-oxobutanoic acid
595 (R)-3-benzyl-4-((4-(5-chloro-2-(1-methyl-2-oxo-2,3-dihydro-1H-pyrrolo[2,3-b]pyridin-5-yl)phenyl)-5-fluorothiazol-2-yl)(cyclopropyl)amino)-4-oxobutanoic acid
596 (R)-4-((4-(5-chloro-2-(4-methyl-3,4-dihydro-2H-pyrido[3,2-b][1,4]oxazin-7-yl)phenyl)-5-fluorothiazol-2-yl)(methyl)amino)-3-(cyclopentylmethyl)-4-oxobutanoic acid
597 (R)-4-((4-(5-chloro-2-(4-methyl-3,4-dihydro-2H-pyrido[3,2-b][1,4]oxazin-7-yl)phenyl)-5-fluorothiazol-2-yl)(cyclopropyl)amino)-3-(cyclopentylmethyl)-4-oxobutanoic acid
598 (R)-4-((4-(5-chloro-2-(4-methyl-3,4-dihydro-2H-pyrido[3,2-b][1,4]oxazin-7-yl)phenyl)-5-fluorothiazol-2-yl)(methyl)amino)-4-oxo-3-((tetrahydro-2H-pyran-4-yl)methyl)butanoic acid
599 (R)-4-((4-(5-chloro-2-(4-methyl-3,4-dihydro-2H-pyrido[3,2-b][1,4]oxazin-7-yl)phenyl)-5-fluorothiazol-2-yl)(cyclopropyl)amino)-4-oxo-3-((tetrahydro-2H-pyran-4-yl)methyl)butanoic acid
600 (R)-3-benzyl-4-((4-(5-chloro-2-(4-methyl-3,4-dihydro-2H-pyrido[3,2-b][1,4]oxazin-7-yl)phenyl)-5-fluorothiazol-2-yl)(methyl)amino)-4-oxobutanoic acid
601 (R)-3-benzyl-4-((4-(5-chloro-2-(4-methyl-3,4-dihydro-2H-pyrido[3,2-b][1,4]oxazin-7-yl)phenyl)-5-fluorothiazol-2-yl)(cyclopropyl)amino)-4-oxobutanoic acid
602 (R)-4-((4-(5-chloro-2-(1-methyl-1H-pyrrolo[2,3-b]pyridin-5-yl)phenyl)-5-fluorothiazol-2-yl)(methyl)amino)-3-(cyclopentylmethyl)-4-oxobutanoic acid
603 (R)-4-((4-(5-chloro-2-(1-methyl-1H-pyrrolo[2,3-b]pyridin-5-yl)phenyl)-5-fluorothiazol-2-yl)(cyclopropyl)amino)-3-(cyclopentylmethyl)-4-oxobutanoic acid
604 (R)-4-((4-(5-chloro-2-(1-methyl-1H-pyrrolo[2,3-b]pyridin-5-yl)phenyl)-5-fluorothiazol-2-yl)(methyl)amino)-4-oxo-3-((tetrahydro-2H-pyran-4-yl)methyl)butanoic acid
605 (R)-4-((4-(5-chloro-2-(1-methyl-1H-pyrrolo[2,3-b]pyridin-5-yl)phenyl)-5-fluorothiazol-2-yl)(cyclopropyl)amino)-4-oxo-3-((tetrahydro-2H-pyran-4-yl)methyl)butanoic acid
606 (R)-3-benzyl-4-((4-(5-chloro-2-(1-methyl-1H-pyrrolo[2,3-b]pyridin-5-yl)phenyl)-5-fluorothiazol-2-yl)(methyl)amino)-4-oxobutanoic acid
607 (R)-3-benzyl-4-((4-(5-chloro-2-(1-methyl-1H-pyrrolo[2,3-b]pyridin-5-yl)phenyl)-5-fluorothiazol-2-yl)(cyclopropyl)amino)-4-oxobutanoic acid
608 (R)-3-(cyclopentylmethyl)-4-((5-fluoro-4-(2-(8-methyl-7-oxo-5,6,7,8-tetrahydro-1,8-naphthyridin-3-yl)phenyl)thiazol-2-yl)(methyl)amino)-4-oxobutanoic acid
609 (R)-3-(cyclopentylmethyl)-4-(cyclopropyl(5-fluoro-4-(2-(8-methyl-7-oxo-5,6,7,8-tetrahydro-1,8-naphthyridin-3-yl)phenyl)thiazol-2-yl)amino)-4-oxobutanoic acid
610 (R)-4-((5-fluoro-4-(2-(8-methyl-7-oxo-5,6,7,8-tetrahydro-1,8-naphthyridin-3-yl)phenyl)thiazol-2-yl)(methyl)amino)-4-oxo-3-((tetrahydro-2H-pyran-4-yl)methyl)butanoic acid
611 (R)-4-(cyclopropyl(5-fluoro-4-(2-(8-methyl-7-oxo-5,6,7,8-tetrahydro-1,8-naphthyridin-3-yl)phenyl)thiazol-2-yl)amino)-4-oxo-3-((tetrahydro-2H-pyran-4-yl)methyl)butanoic acid
612 (R)-3-benzyl-4-((5-fluoro-4-(2-(8-methyl-7-oxo-5,6,7,8-tetrahydro-1,8-naphthyridin-3-yl)phenyl)thiazol-2-yl)(methyl)amino)-4-oxobutanoic acid
613 (R)-3-benzyl-4-(cyclopropyl(5-fluoro-4-(2-(8-methyl-7-oxo-5,6,7,8-tetrahydro-1,8-naphthyridin-3-yl)phenyl)thiazol-2-yl)amino)-4-oxobutanoic acid
614 (R)-3-(cyclopentylmethyl)-4-((5-fluoro-4-(2-(1-methyl-2-oxo-2,3-dihydro-1H-pyrrolo[2,3-b]pyridin-5-yl)phenyl)thiazol-2-yl)(methyl)amino)-4-oxobutanoic acid
615 (R)-3-(cyclopentylmethyl)-4-(cyclopropyl(5-fluoro-4-(2-(1-methyl-2-oxo-2,3-dihydro-1H-pyrrolo[2,3-b]pyridin-5-yl)phenyl)thiazol-2-yl)amino)-4-oxobutanoic acid
616 (R)-4-((5-fluoro-4-(2-(1-methyl-2-oxo-2,3-dihydro-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
617 (R)-4-(cyclopropyl(5-fluoro-4-(2-(1-methyl-2-oxo-2,3-dihydro-1H-pyrrolo[2,3-b]pyridin-5-yl)phenyl)thiazol-2-yl)amino)-4-oxo-3-((tetrahydro-2H-pyran-4-yl)methyl)butanoic acid
618 (R)-3-benzyl-4-((5-fluoro-4-(2-(1-methyl-2-oxo-2,3-dihydro-1H-pyrrolo[2,3-b]pyridin-5-yl)phenyl)thiazol-2-yl)(methyl)amino)-4-oxobutanoic acid
619 (R)-3-benzyl-4-(cyclopropyl(5-fluoro-4-(2-(1-methyl-2-oxo-2,3-dihydro-1H-pyrrolo[2,3-b]pyridin-5-yl)phenyl)thiazol-2-yl)amino)-4-oxobutanoic acid
620 (R)-3-(cyclopentylmethyl)-4-((5-fluoro-4-(2-(4-methyl-3,4-dihydro-2H-pyrido[3,2-b][1,4]oxazin-7-yl)phenyl)thiazol-2-yl)(methyl)amino)-4-oxobutanoic acid
621 (R)-3-(cyclopentylmethyl)-4-(cyclopropyl(5-fluoro-4-(2-(4-methyl-3,4-dihydro-2H-pyrido[3,2-b][1,4]oxazin-7-yl)phenyl)thiazol-2-yl)amino)-4-oxobutanoic acid
622 (R)-4-((5-fluoro-4-(2-(4-methyl-3,4-dihydro-2H-pyrido[3,2-b][1,4]oxazin-7-yl)phenyl)thiazol-2-yl)(methyl)amino)-4-oxo-3-((tetrahydro-2H-pyran-4-yl)methyl)butanoic acid
623 (R)-4-(cyclopropyl(5-fluoro-4-(2-(4-methyl-3,4-dihydro-2H-pyrido[3,2-b][1,4]oxazin-7-yl)phenyl)thiazol-2-yl)amino)-4-oxo-3-((tetrahydro-2H-pyran-4-yl)methyl)butanoic acid
624 (R)-3-benzyl-4-((5-fluoro-4-(2-(4-methyl-3,4-dihydro-2H-pyrido[3,2-b][1,4]oxazin-7-yl)phenyl)thiazol-2-yl)(methyl)amino)-4-oxobutanoic acid
625 (R)-3-benzyl-4-(cyclopropyl(5-fluoro-4-(2-(4-methyl-3,4-dihydro-2H-pyrido[3,2-b][1,4]oxazin-7-yl)phenyl)thiazol-2-yl)amino)-4-oxobutanoic acid
626 (R)-3-(cyclopentylmethyl)-4-((5-fluoro-4-(2-(1-methyl-1H-pyrrolo[2,3-b]pyridin-5-yl)phenyl)thiazol-2-yl)(methyl)amino)-4-oxobutanoic acid
627 (R)-3-(cyclopentylmethyl)-4-(cyclopropyl(5-fluoro-4-(2-(1-methyl-1H-pyrrolo[2,3-b]pyridin-5-yl)phenyl)thiazol-2-yl)amino)-4-oxobutanoic acid
628 (R)-4-((5-fluoro-4-(2-(1-methyl-1H-pyrrolo[2,3-b]pyridin-5-yl)phenyl)thiazol-2-yl)(methyl)amino)-4-oxo-3-((tetrahydro-2H-pyran-4-yl)methyl)butanoic acid
629 (R)-4-(cyclopropyl(5-fluoro-4-(2-(1-methyl-1H-pyrrolo[2,3-b]pyridin-5-yl)phenyl)thiazol-2-yl)amino)-4-oxo-3-((tetrahydro-2H-pyran-4-yl)methyl)butanoic acid
630 (R)-3-benzyl-4-((5-fluoro-4-(2-(1-methyl-1H-pyrrolo[2,3-b]pyridin-5-yl)phenyl)thiazol-2-yl)(methyl)amino)-4-oxobutanoic acid
631 (R)-3-benzyl-4-(cyclopropyl(5-fluoro-4-(2-(1-methyl-1H-pyrrolo[2,3-b]pyridin-5-yl)phenyl)thiazol-2-yl)amino)-4-oxobutanoic acid
632 (R)-3-(cyclopentylmethyl)-4-(cyclopropyl(4-(2-(1-methyl-1H-pyrrolo[3,2-b]pyridin-6-yl)phenyl)thiazol-2-yl)amino)-4-oxobutanoic acid
633 (R)-3-(cyclopentylmethyl)-4-(cyclopropyl(4-(2-(1-methyl-2-oxo-2,3-dihydro-1H-imidazo[4,5-b]pyridin-6-yl)phenyl)thiazol-2-yl)amino)-4-oxobutanoic acid
634 (R)-3-(cyclopentylmethyl)-4-(cyclopropyl(4-(2-(1-methyl-2-oxo-1,2,3,4-tetrahydropyrido[3,2-d]pyrimidin-7-yl)phenyl)thiazol-2-yl)amino)-4-oxobutanoic acid
635 (R)-3-(cyclopentylmethyl)-4-(cyclopropyl(4-(2-(1-methyl-2-oxo-2,3-dihydro-1H-pyrrolo[3,2-b]pyridin-6-yl)phenyl)thiazol-2-yl)amino)-4-oxobutanoic acid
636 (R)-3-(cyclopentylmethyl)-4-(cyclopropyl(4-(2-(6-methyl-5-oxo-5,6,7,8-tetrahydro-1,6-naphthyridin-3-yl)phenyl)thiazol-2-yl)amino)-4-oxobutanoic acid
637 (R)-3-(cyclopentylmethyl)-4-(cyclopropyl(4-(2-(1,3-dimethyl-2-oxo-1,2,3,4-tetrahydropyrido[3,2-d]pyrimidin-7-yl)phenyl)thiazol-2-yl)amino)-4-oxobutanoic acid
638 (R)-3-(cyclopentylmethyl)-4-(cyclopropyl(4-(2-(7-methyl-8-oxo-5,6,7,8-tetrahydro-1,7-naphthyridin-3-yl)phenyl)thiazol-2-yl)amino)-4-oxobutanoic acid
639 (R)-3-(cyclopentylmethyl)-4-(cyclopropyl(4-(2-(6-methyl-5-oxo-6,7-dihydro-5H-pyrrolo[3,4-b]pyridin-3-yl)phenyl)thiazol-2-yl)amino)-4-oxobutanoic acid
640 (R)-4-((4-(2-(5-chloro-6-(2-oxopyrrolidin-1-yl)pyridin-3-yl)phenyl)thiazol-2-yl)(cyclopropyl)amino)-3-(cyclopentylmethyl)-4-oxobutanoic acid
641 (R)-3-(cyclopentylmethyl)-4-(cyclopropyl(4-(2-(3-methyl-3H-imidazo[4,5-b]pyridin-6-yl)phenyl)thiazol-2-yl)amino)-4-oxobutanoic acid
642 (R)-3-(cyclopentylmethyl)-4-(cyclopropyl(4-(2-(1-methyl-2,3-dihydro-1H-pyrido[2,3-b][1,4]oxazin-7-yl)phenyl)thiazol-2-yl)amino)-4-oxobutanoic acid
643 (R)-3-(cyclopentylmethyl)-4-(cyclopropyl(4-(2-(3-methyl-2-oxo-2,3-dihydro-1H-imidazo[4,5-b]pyridin-6-yl)phenyl)thiazol-2-yl)amino)-4-oxobutanoic acid
644 (R)-3-(cyclopentylmethyl)-4-(cyclopropyl(4-(2-(7-methyl-6-oxo-5,6,7,8-tetrahydro-1,7-naphthyridin-3-yl)phenyl)thiazol-2-yl)amino)-4-oxobutanoic acid
645 (R)-3-(cyclopentylmethyl)-4-(cyclopropyl(4-(2-(6-methyl-7-oxo-6,7-dihydro-5H-pyrrolo[3,4-b]pyridin-3-yl)phenyl)thiazol-2-yl)amino)-4-oxobutanoic acid
646 (R)-3-(cyclopentylmethyl)-4-(cyclopropyl(4-(2-(1,3-dimethyl-2-oxo-2,3-dihydro-1H-imidazo[4,5-b]pyridin-6-yl)phenyl)thiazol-2-yl)amino)-4-oxobutanoic acid
647 (R)-3-(cyclopentylmethyl)-4-(cyclopropyl(4-(2-(1-methyl-1H-imidazo[4,5-b]pyridin-6-yl)phenyl)thiazol-2-yl)amino)-4-oxobutanoic acid
648 (R)-3-(cyclopentylmethyl)-4-(cyclopropyl(4-(2-(5-fluoro-6-(2-oxopyrrolidin-1-yl)pyridin-3-yl)phenyl)thiazol-2-yl)amino)-4-oxobutanoic acid
649 (R)-3-(cyclopentylmethyl)-4-(cyclopropyl(4-(2-(1-methyl-2-oxo-1,2,3,4-tetrahydro-1,5-naphthyridin-7-yl)phenyl)thiazol-2-yl)amino)-4-oxobutanoic acid
650 (R)-3-(cyclopentylmethyl)-4-(cyclopropyl(4-(2-(3-methyl-2-oxo-1,2,3,4-tetrahydropyrido[3,2-d]pyrimidin-7-yl)phenyl)thiazol-2-yl)amino)-4-oxobutanoic acid
651 (R)-4-(cyclopropyl(5-fluoro-4-(5-methyl-2-(6-(2-oxopyrrolidin-1-yl)pyridin-3-yl)furan-3-yl)thiazol-2-yl)amino)-4-oxo-3-((tetrahydro-2H-pyran-4-yl)methyl)butanoic acid
652 (R)-4-(cyclopropyl(4-(5-methyl-2-(6-(2-oxopyrrolidin-1-yl)pyridin-3-yl)furan-3-yl)thiazol-2-yl)amino)-4-oxo-3-((tetrahydro-2H-pyran-4-yl)methyl)butanoic acid
653 (R)-4-((5-fluoro-4-(5-methyl-2-(6-(2-oxopyrrolidin-1-yl)pyridin-3-yl)furan-3-yl)thiazol-2-yl)(methyl)amino)-4-oxo-3-((tetrahydro-2H-pyran-4-yl)methyl)butanoic acid
654 (R)-4-(methyl(4-(5-methyl-2-(6-(2-oxopyrrolidin-1-yl)pyridin-3-yl)furan-3-yl)thiazol-2-yl)amino)-4-oxo-3-((tetrahydro-2H-pyran-4-yl)methyl)butanoic acid
655 (R)-3-(cyclopentylmethyl)-4-(cyclopropyl(5-fluoro-4-(5-methyl-2-(6-(2-oxopyrrolidin-1-yl)pyridin-3-yl)furan-3-yl)thiazol-2-yl)amino)-4-oxobutanoic acid
656 (R)-3-(cyclopentylmethyl)-4-(cyclopropyl(4-(5-methyl-2-(6-(2-oxopyrrolidin-1-yl)pyridin-3-yl)furan-3-yl)thiazol-2-yl)amino)-4-oxobutanoic acid
657 (R)-3-(cyclopentylmethyl)-4-((5-fluoro-4-(5-methyl-2-(6-(2-oxopyrrolidin-1-yl)pyridin-3-yl)furan-3-yl)thiazol-2-yl)(methyl)amino)-4-oxobutanoic acid
658 (R)-3-(cyclopentylmethyl)-4-(methyl(4-(5-methyl-2-(6-(2-oxopyrrolidin-1-yl)pyridin-3-yl)furan-3-yl)thiazol-2-yl)amino)-4-oxobutanoic acid
659 (R)-3-benzyl-4-(cyclopropyl(5-fluoro-4-(5-methyl-2-(6-(2-oxopyrrolidin-1-yl)pyridin-3-yl)furan-3-yl)thiazol-2-yl)amino)-4-oxobutanoic acid
660 (R)-3-benzyl-4-(cyclopropyl(4-(5-methyl-2-(6-(2-oxopyrrolidin-1-yl)pyridin-3-yl)furan-3-yl)thiazol-2-yl)amino)-4-oxobutanoic acid
661 (R)-3-benzyl-4-((5-fluoro-4-(5-methyl-2-(6-(2-oxopyrrolidin-1-yl)pyridin-3-yl)furan-3-yl)thiazol-2-yl)(methyl)amino)-4-oxobutanoic acid
662 (R)-3-benzyl-4-(methyl(4-(5-methyl-2-(6-(2-oxopyrrolidin-1-yl)pyridin-3-yl)furan-3-yl)thiazol-2-yl)amino)-4-oxobutanoic acid
663 (R)-3-benzyl-4-(methyl(3-(2-(6-(2-oxopyrrolidin-1-yl)pyridin-3-yl)phenyl)-1,2,4-thiadiazol-5-yl)amino)-4-oxobutanoic acid
664 (R)-3-benzyl-4-(cyclopropyl(3-(2-(6-(2-oxopyrrolidin-1-yl)pyridin-3-yl)phenyl)-1,2,4-thiadiazol-5-yl)amino)-4-oxobutanoic acid
665 (R)-3-(cyclopentylmethyl)-4-(methyl(3-(2-(6-(2-oxopyrrolidin-1-yl)pyridin-3-yl)phenyl)-1,2,4-thiadiazol-5-yl)amino)-4-oxobutanoic acid
666 (R)-3-(cyclopentylmethyl)-4-(cyclopropyl(3-(2-(6-(2-oxopyrrolidin-1-yl)pyridin-3-yl)phenyl)-1,2,4-thiadiazol-5-yl)amino)-4-oxobutanoic acid
667 (R)-4-(methyl(3-(2-(6-(2-oxopyrrolidin-1-yl)pyridin-3-yl)phenyl)-1,2,4-thiadiazol-5-yl)amino)-4-oxo-3-((tetrahydro-2H-pyran-4-yl)methyl)butanoic acid
668 (R)-4-(cyclopropyl(3-(2-(6-(2-oxopyrrolidin-1-yl)pyridin-3-yl)phenyl)-1,2,4-thiadiazol-5-yl)amino)-4-oxo-3-((tetrahydro-2H-pyran-4-yl)methyl)butanoic acid
669 (3R)-3-(cyclopentylmethyl)-4-(cyclopropyl(4-(2,5-dimethyl-4-(6-(2-oxopyrrolidin-1-yl)pyridin-3-yl)furan-3-yl)-5-fluorothiazol-2-yl)amino)-4-oxobutanoic acid
670 (3R)-3-(cyclopentylmethyl)-4-((4-(2,5-dimethyl-4-(6-(2-oxopyrrolidin-1-yl)pyridin-3-yl)furan-3-yl)-5-fluorothiazol-2-yl)(methyl)amino)-4-oxobutanoic acid
671 (R)-3-(cyclopentylmethyl)-4-(cyclopropyl(4-(2,5-dimethyl-4-(6-(2-oxopyrrolidin-1-yl)pyridin-3-yl)furan-3-yl)thiazol-2-yl)amino)-4-oxobutanoic acid
672 (R)-3-(cyclopentylmethyl)-4-((4-(2,5-dimethyl-4-(6-(2-oxopyrrolidin-1-yl)pyridin-3-yl)furan-3-yl)thiazol-2-yl)(methyl)amino)-4-oxobutanoic acid
673 (3R)-4-(cyclopropyl(4-(2,5-dimethyl-4-(6-(2-oxopyrrolidin-1-yl)pyridin-3-yl)furan-3-yl)-5-fluorothiazol-2-yl)amino)-4-oxo-3-((tetrahydro-2H-pyran-4-yl)methyl)butanoic acid
674 (3R)-4-((4-(2,5-dimethyl-4-(6-(2-oxopyrrolidin-1-yl)pyridin-3-yl)furan-3-yl)-5-fluorothiazol-2-yl)(methyl)amino)-4-oxo-3-((tetrahydro-2H-pyran-4-yl)methyl)butanoic acid
675 (R)-4-(cyclopropyl(4-(2,5-dimethyl-4-(6-(2-oxopyrrolidin-1-yl)pyridin-3-yl)furan-3-yl)thiazol-2-yl)amino)-4-oxo-3-((tetrahydro-2H-pyran-4-yl)methyl)butanoic acid
676 (R)-4-((4-(2,5-dimethyl-4-(6-(2-oxopyrrolidin-1-yl)pyridin-3-yl)furan-3-yl)thiazol-2-yl)(methyl)amino)-4-oxo-3-((tetrahydro-2H-pyran-4-yl)methyl)butanoic acid
677 (3R)-3-benzyl-4-(cyclopropyl(4-(2,5-dimethyl-4-(6-(2-oxopyrrolidin-1-yl)pyridin-3-yl)furan-3-yl)-5-fluorothiazol-2-yl)amino)-4-oxobutanoic acid
678 (3R)-3-benzyl-4-((4-(2,5-dimethyl-4-(6-(2-oxopyrrolidin-1-yl)pyridin-3-yl)furan-3-yl)-5-fluorothiazol-2-yl)(methyl)amino)-4-oxobutanoic acid
679 (R)-3-benzyl-4-(cyclopropyl(4-(2,5-dimethyl-4-(6-(2-oxopyrrolidin-1-yl)pyridin-3-yl)furan-3-yl)thiazol-2-yl)amino)-4-oxobutanoic acid
680 (R)-3-benzyl-4-((4-(2,5-dimethyl-4-(6-(2-oxopyrrolidin-1-yl)pyridin-3-yl)furan-3-yl)thiazol-2-yl)(methyl)amino)-4-oxobutanoic acid
681 (3R)-3-(cyclopentylmethyl)-4-(cyclopropyl(4-(2-(6-(1-methyl-6-oxopiperidin-3-yl)pyridin-3-yl)phenyl)thiazol-2-yl)amino)-4-oxobutanoic acid
682 (3R)-3-(cyclopentylmethyl)-4-(cyclopropyl(4-(2-(6-(1-methyl-2-oxopiperidin-4-yl)pyridin-3-yl)phenyl)thiazol-2-yl)amino)-4-oxobutanoic acid
683 (3R)-3-(cyclopentylmethyl)-4-(cyclopropyl(4-(2-(6-(3-methyl-2-oxoimidazolidin-4-yl)pyridin-3-yl)phenyl)thiazol-2-yl)amino)-4-oxobutanoic acid
684 (R)-3-(cyclopentylmethyl)-4-(cyclopropyl(4-(2-(6-(N-methylacetamido)pyridin-3-yl)phenyl)thiazol-2-yl)amino)-4-oxobutanoic acid
685 (R)-3-(cyclopentylmethyl)-4-(cyclopropyl(4-(2-(6-(1,3-dimethyl-2-oxohexahydropyrimidin-5-yl)pyridin-3-yl)phenyl)thiazol-2-yl)amino)-4-oxobutanoic acid
686 (3R)-3-(cyclopentylmethyl)-4-(cyclopropyl(4-(2-(6-(5-oxopyrrolidin-3-yl)pyridin-3-yl)phenyl)thiazol-2-yl)amino)-4-oxobutanoic acid
687 (3R)-3-(cyclopentylmethyl)-4-(cyclopropyl(4-(2-(6-(1-methyl-2-oxoimidazolidin-4-yl)pyridin-3-yl)phenyl)thiazol-2-yl)amino)-4-oxobutanoic acid
688 (R)-3-(cyclopentylmethyl)-4-(cyclopropyl(4-(2-(6-(pyrrolidin-1-yl)pyridin-3-yl)phenyl)thiazol-2-yl)amino)-4-oxobutanoic acid
689 (3R)-3-(cyclopentylmethyl)-4-(cyclopropyl(4-(2-(6-(2-oxoimidazolidin-4-yl)pyridin-3-yl)phenyl)thiazol-2-yl)amino)-4-oxobutanoic acid
690 (3R)-3-(cyclopentylmethyl)-4-(cyclopropyl(4-(2-(6-(1-methyl-5-oxopyrrolidin-2-yl)pyridin-3-yl)phenyl)thiazol-2-yl)amino)-4-oxobutanoic acid
691 (R)-3-(cyclopentylmethyl)-4-(cyclopropyl(4-(2-(6-(4-methyl-3-oxopiperazin-1-yl)pyridin-3-yl)phenyl)thiazol-2-yl)amino)-4-oxobutanoic acid
692 (R)-3-(cyclopentylmethyl)-4-(cyclopropyl(4-(2-(6-(4-methyl-2,5-dioxopiperazin-1-yl)pyridin-3-yl)phenyl)thiazol-2-yl)amino)-4-oxobutanoic acid
693 (R)-3-(cyclopentylmethyl)-4-(cyclopropyl(4-(2-(6-(dimethylcarbamoyl)pyridin-3-yl)phenyl)thiazol-2-yl)amino)-4-oxobutanoic acid
694 (3R)-3-(cyclopentylmethyl)-4-(cyclopropyl(4-(2-(6-(3-methyl-2-oxohexahydropyrimid-4-yl)pyridin-3-yl)phenyl)thiazol-2-yl)amino)-4-oxobutanoic acid
695 (R)-3-(cyclopentylmethyl)-4-(cyclopropyl(4-(2-(6-isopropoxypyridin-3-yl)phenyl)thiazol-2-yl)amino)-4-oxobutanoic acid
696 (3R)-3-(cyclopentylmethyl)-4-(cyclopropyl(4-(2-(6-(1-methyl-6-oxopiperidin-2-yl)pyridin-3-yl)phenyl)thiazol-2-yl)amino)-4-oxobutanoic acid
697 (3R)-3-(cyclopentylmethyl)-4-(cyclopropyl(4-(2-(6-(1-methyl-5-oxopyrrolidin-3-yl)pyridin-3-yl)phenyl)thiazol-2-yl)amino)-4-oxobutanoic acid
698 (R)-3-(cyclopentylmethyl)-4-(cyclopropyl(4-(2-(6-(3-methyl-2-oxotetrahydropyrimidin-1(2H)-yl)pyridin-3-yl)phenyl)thiazol-2-yl)amino)-4-oxobutanoic acid
699 (R)-3-(cyclopentylmethyl)-4-(cyclopropyl(4-(2-(6-(2-oxotetrahydropyrimidin-1(2H)-yl)pyridin-3-yl)phenyl)thiazol-2-yl)amino)-4-oxobutanoic acid
700 (3R)-3-(cyclopentylmethyl)-4-(cyclopropyl(4-(2-(6-(1,3-dimethyl-2-oxoimidazolidin-4-yl)pyridin-3-yl)phenyl)thiazol-2-yl)amino)-4-oxobutanoic acid
701 (3R)-3-(cyclopentylmethyl)-4-(cyclopropyl(4-(2-(6-(1,3-dimethyl-2-oxohexahydropyrimidin-4-yl)pyridin-3-yl)phenyl)thiazol-2-yl)amino)-4-oxobutanoic acid
702 (3R)-3-(cyclopentylmethyl)-4-(cyclopropyl(4-(2-(6-(1-methyl-2-oxohexahydropyrimid-4-yl)pyridin-3-yl)phenyl)thiazol-2-yl)amino)-4-oxobutanoic acid
703 (R)-3-(cyclopentylmethyl)-4-(cyclopropyl(4-(2-(6-(N-methylcyclopropanecarboxamido)pyridin-3-yl)phenyl)thiazol-2-yl)amino)-4-oxobutanoic acid
704 (3R)-3-(cyclopentylmethyl)-4-(cyclopropyl(4-(2-(6-(1-methyl-2-oxopyrrolidin-3-yl)pyridin-3-yl)phenyl)thiazol-2-yl)amino)-4-oxobutanoic acid
705 (R)-3-(cyclopentylmethyl)-4-(cyclopropyl(4-(2-(6-(cyclopropylmethoxy)pyridin-3-yl)phenyl)thiazol-2-yl)amino)-4-oxobutanoic acid
706 (3R)-3-(cyclopentylmethyl)-4-(cyclopropyl(4-(2-(6-(2-oxopyrrolidin-3-yl)pyridin-3-yl)phenyl)thiazol-2-yl)amino)-4-oxobutanoic acid
707 (3R)-3-(cyclopentylmethyl)-4-(cyclopropyl(4-(2-(6-(1-methyl-2-oxopiperidin-3-yl)pyridin-3-yl)phenyl)thiazol-2-yl)amino)-4-oxobutanoic acid
708 (R)-3-(cyclopentylmethyl)-4-(cyclopropyl(4-(2-(6-(methoxymethyl)pyridin-3-yl)phenyl)thiazol-2-yl)amino)-4-oxobutanoic acid
709 (3R)-3-(cyclopentylmethyl)-4-(cyclopropyl(4-(2-(6-(1-methyl-2-oxohexahydropyrimid-5-yl)pyridin-3-yl)phenyl)thiazol-2-yl)amino)-4-oxobutanoic acid
710 (3R)-3-(cyclopentylmethyl)-4-(cyclopropyl(4-(2-(6-(5-oxopyrrolidin-2-yl)pyridin-3-yl)phenyl)thiazol-2-yl)amino)-4-oxobutanoic acid
711 (R)-3-(cyclopentylmethyl)-4-(cyclopropyl(4-(2-(6-(2-oxopyrrolidin-1-yl)pyridin-3-yl)-5-(trifluoromethyl)phenyl)thiazol-2-yl)amino)-4-oxobutanoic acid
712 (R)-3-(cyclopentylmethyl)-4-(cyclopropyl(4-(5-(fluoromethoxy)-2-(6-(2-oxopyrrolidin-1-yl)pyridin-3-yl)phenyl)thiazol-2-yl)amino)-4-oxobutanoic acid
713 (R)-3-(cyclopentylmethyl)-4-(cyclopropyl(4-(2-(6-(2-oxopyrrolidin-1-yl)pyridin-3-yl)-5-(trifluoromethoxy)phenyl)thiazol-2-yl)amino)-4-oxobutanoic acid
714 (R)-4-(cyclopropyl(4-(2-(6-(2-oxopyrrolidin-1-yl)pyridin-3-yl)phenyl)thiazol-2-yl)amino)-4-oxo-3-(pyridin-2-ylmethyl)butanoic acid
715 (R)-2-(2-(carboxymethyl)-3-(cyclopropyl(4-(2-(6-(2-oxopyrrolidin-1-yl)pyridin-3-yl)phenyl)thiazol-2-yl)amino)-3-oxopropyl)pyridine 1-oxide
716 (R)-4-(cyclopropyl(4-(2-(6-(2-oxopyrrolidin-1-yl)pyridin-3-yl)phenyl)thiazol-2-yl)amino)-4-oxo-3-(((R)-tetrahydro-2H-pyran-2-yl)methyl)butanoic acid
717 (R)-4-(cyclopropyl(4-(2-(6-(2-oxopyrrolidin-1-yl)pyridin-3-yl)phenyl)thiazol-2-yl)amino)-4-oxo-3-(pyrimidin-2-ylmethyl)butanoic acid
718 (S)-4-(cyclopropyl(4-(2-(6-(2-oxopyrrolidin-1-yl)pyridin-3-yl)phenyl)thiazol-2-yl)amino)-4-oxo-3-(thiophen-2-ylmethyl)butanoic acid
719 (R)-4-(cyclopropyl(4-(2-(6-(2-oxopyrrolidin-1-yl)pyridin-3-yl)phenyl)thiazol-2-yl)amino)-4-oxo-3-(((S)-tetrahydrofuran-2-yl)methyl)butanoic acid
720 (R)-4-(cyclopropyl(4-(2-(6-(2-oxopyrrolidin-1-yl)pyridin-3-yl)phenyl)thiazol-2-yl)amino)-4-oxo-3-(((S)-tetrahydro-2H-pyran-3-yl)methyl)butanoic acid
721 (3R)-4-(cyclopropyl(4-(2-(6-(2-oxopyrrolidin-1-yl)pyridin-3-yl)phenyl)thiazol-2-yl)amino)-3-(((25)-2-methyltetrahydro-2H-pyran-4-yl)methyl)-4-oxobutanoic acid
722 (R)-4-(cyclopropyl(4-(2-(6-(2-oxopyrrolidin-1-yl)pyridin-3-yl)phenyl)thiazol-2-yl)amino)-4-oxo-3-(((R)-tetrahydro-2H-pyran-3-yl)methyl)butanoic acid
723 (3R)-4-(cyclopropyl(4-(2-(6-(2-oxopyrrolidin-1-yl)pyridin-3-yl)phenyl)thiazol-2-yl)amino)-3-(((3R,55)-3,5-dimethyltetrahydro-2H-pyran-4-yl)methyl)-4-oxobutanoic acid
724 (R)-4-(cyclopropyl(4-(2-(6-(2-oxopyrrolidin-1-yl)pyridin-3-yl)phenyl)thiazol-2-yl)amino)-3-(((2R,3R)-2-methyltetrahydro-2H-pyran-3-yl)methyl)-4-oxobutanoic acid
725 (3R)-4-(cyclopropyl(4-(2-(6-(2-oxopyrrolidin-1-yl)pyridin-3-yl)phenyl)thiazol-2-yl)amino)-3-(((3S)-3-methyltetrahydro-2H-pyran-4-yl)methyl)-4-oxobutanoic acid
726 (R)-4-(cyclopropyl(4-(2-(6-(2-oxopyrrolidin-1-yl)pyridin-3-yl)phenyl)thiazol-2-yl)amino)-4-oxo-3-(((S)-tetrahydro-2H-pyran-2-yl)methyl)butanoic acid
727 (R)-3-(benzofuran-2-ylmethyl)-4-(cyclopropyl(4-(2-(6-(2-oxopyrrolidin-1-yl)pyridin-3-yl)phenyl)thiazol-2-yl)amino)-4-oxobutanoic acid
728 (R)-4-(cyclopropyl(4-(2-(6-(2-oxopyrrolidin-1-yl)pyridin-3-yl)phenyl)thiazol-2-yl)amino)-4-oxo-3-(((R)-tetrahydrofuran-2-yl)methyl)butanoic acid
729 (R)-4-(cyclopropyl(4-(2-(6-(2-oxopyrrolidin-1-yl)pyridin-3-yl)phenyl)thiazol-2-yl)amino)-3-((5-methylfuran-2-yl)methyl)-4-oxobutanoic acid
730 (3R)-4-(cyclopropyl(4-(2-(6-(2-oxopyrrolidin-1-yl)pyridin-3-yl)phenyl)thiazol-2-yl)amino)-4-oxo-3-((tetrahydro-2H-pyran-3-yl)methyl)butanoic acid
731 (3R)-4-(cyclopropyl(4-(2-(6-(2-oxopyrrolidin-1-yl)pyridin-3-yl)phenyl)thiazol-2-yl)amino)-3-(((2R,65)-2,6-dimethyltetrahydro-2H-pyran-4-yl)methyl)-4-oxobutanoic acid
732 (R)-4-(cyclopropyl(4-(2-(6-(2-oxopyrrolidin-1-yl)pyridin-3-yl)phenyl)thiazol-2-yl)amino)-3-(furan-2-ylmethyl)-4-oxobutanoic acid
733 (3R)-4-(cyclopropyl(4-(2-(6-(2-oxopyrrolidin-1-yl)pyridin-3-yl)phenyl)thiazol-2-yl)amino)-4-oxo-3-((tetrahydro-2H-pyran-2-yl)methyl)butanoic acid
734 (R)-3-(cyclopentylmethyl)-4-(cyclopropyl(4-(2-(6-methoxypyridin-3-yl)phenyl)thiazol-2-yl)amino)-4-oxobutanoic acid
735 (R)-4-((4-(5-chloro-2-(6-methoxypyridin-3-yl)phenyl)thiazol-2-yl)(cyclopropyl)amino)-3-(cyclopentylmethyl)-4-oxobutanoic acid
736 (R)-4-(cyclopropyl(4-(2-(6-(2-oxopyrrolidin-1-yl)pyridin-3-yl)phenyl)thiazol-2-yl)amino)-3-(oxetan-3-ylmethyl)-4-oxobutanoic acid
737 (R)-3-(cyclopentylmethyl)-4-(cyclopropyl(4-(2-(6-(oxetan-3-yl)pyridin-3-yl)phenyl)thiazol-2-yl)amino)-4-oxobutanoic acid
738 (R)-4-(cyclopropyl(4-(2-(6-(2-oxopyrrolidin-1-yl)pyridin-3-yl)phenyl)thiazol-2-yl)amino)-3-(oxetan-3-ylmethyl)-4-oxobutanoic acid
739 (R)-3-(cyclopentylmethyl)-4-(cyclopropyl(4-(2-(6-(3-methyloxetan-3-yl)pyridin-3-yl)phenyl)thiazol-2-yl)amino)-4-oxobutanoic acid
740 (R)-4-(cyclopropyl(4-(2-(6-(2-oxopyrrolidin-1-yl)pyridin-3-yl)phenyl)thiazol-2-yl)amino)-3-(oxetan-3-ylmethyl)-4-oxobutanoic acid
741 (R)-3-(cyclopentylmethyl)-4-(cyclopropyl(4-(2-(6-(3-fluorooxetan-3-yl)pyridin-3-yl)phenyl)thiazol-2-yl)amino)-4-oxobutanoic acid
742 (R)-4-(cyclopropyl(4-(2-(6-(2-oxopyrrolidin-1-yl)pyridin-3-yl)phenyl)thiazol-2-yl)amino)-3-((3-methyloxetan-3-yl)methyl)-4-oxobutanoic acid
743 (R)-3-(cyclopentylmethyl)-4-(cyclopropyl(4-(2-(6-(oxetan-3-yl)pyridin-3-yl)phenyl)thiazol-2-yl)amino)-4-oxobutanoic acid
744 (R)-4-(cyclopropyl(4-(2-(6-(2-oxopyrrolidin-1-yl)pyridin-3-yl)phenyl)thiazol-2-yl)amino)-3-((3-methyloxetan-3-yl)methyl)-4-oxobutanoic acid
745 (R)-3-(cyclopentylmethyl)-4-(cyclopropyl(4-(2-(6-(3-methyloxetan-3-yl)pyridin-3-yl)phenyl)thiazol-2-yl)amino)-4-oxobutanoic acid
746 (R)-4-(cyclopropyl(4-(2-(6-(2-oxopyrrolidin-1-yl)pyridin-3-yl)phenyl)thiazol-2-yl)amino)-3-((3-methyloxetan-3-yl)methyl)-4-oxobutanoic acid
747 (R)-3-(cyclopentylmethyl)-4-(cyclopropyl(4-(2-(6-(3-fluorooxetan-3-yl)pyridin-3-yl)phenyl)thiazol-2-yl)amino)-4-oxobutanoic acid
748 (S)-4-(cyclopropyl(4-(2-(6-(2-oxopyrrolidin-1-yl)pyridin-3-yl)phenyl)thiazol-2-yl)amino)-3-((3-fluorooxetan-3-yl)methyl)-4-oxobutanoic acid
749 (R)-3-(cyclopentylmethyl)-4-(cyclopropyl(4-(2-(6-(oxetan-3-yl)pyridin-3-yl)phenyl)thiazol-2-yl)amino)-4-oxobutanoic acid
750 (S)-4-(cyclopropyl(4-(2-(6-(2-oxopyrrolidin-1-yl)pyridin-3-yl)phenyl)thiazol-2-yl)amino)-3-((3-fluorooxetan-3-yl)methyl)-4-oxobutanoic acid
751 (R)-3-(cyclopentylmethyl)-4-(cyclopropyl(4-(2-(6-(3-methyloxetan-3-yl)pyridin-3-yl)phenyl)thiazol-2-yl)amino)-4-oxobutanoic acid
752 (S)-4-(cyclopropyl(4-(2-(6-(2-oxopyrrolidin-1-yl)pyridin-3-yl)phenyl)thiazol-2-yl)amino)-3-((3-fluorooxetan-3-yl)methyl)-4-oxobutanoic acid
753 (R)-3-(cyclopentylmethyl)-4-(cyclopropyl(4-(2-(6-(3-fluorooxetan-3-yl)pyridin-3-yl)phenyl)thiazol-2-yl)amino)-4-oxobutanoic acid

30. A pharmaceutical composition comprising a compound according to claim 20 or a pharmaceutically acceptable salt or solvate thereof and at least one pharmaceutically acceptable carrier, diluent, excipient and/or adjuvant.

31. A method for the treatment and/or prevention of type II diabetes, obesity, dyslipidemia, hypercholesterolemia, low HDL cholesterol, high LDL cholesterol, hyperlipidemia, hypertriglyceridemia, hypoglycemia, hyperglycemia, glucose intolerance, insulin resistance, hyperinsulinemia hypertension, hyperlipoproteinemia, metabolic syndrome, syndrome X, thrombotic disorders, cardiovascular disease, atherosclerosis and its sequelae including angina, claudication, heart attack, stroke and others, kidney diseases, ketoacidosis, nephropathy, diabetic neuropathy, diabetic retinopathy, nonalcoholic fatty liver diseases, said method comprising administering to a subject in need thereof an effective amount of a compound of claim 20 or a pharmaceutically acceptable salt or solvate thereof.

32. The method of claim 31, wherein said compound of claim 20 or a pharmaceutically acceptable salt or solvate thereof is a modulator of GPR43 receptor activity.

33. The method of claim 32, wherein the compound is an agonist or partial agonist of GPR43 receptor activity.

34. The method of claim 31, wherein dyslipidemia is mixed or diabetic dyslipidemia.

35. The method of claim 31, wherein the nonalcoholic fatty liver diseases are steatosis or nonalcoholic steatohepatitis (NASH).
